(19) United States
(12) Patent Application Publication
(10) Pub. No.: US 2016/0058863 A1
(43) Pub. Date: Mar. 3, 2016

(54) LOW VISCOSITY CONCENTRATED PROTEIN DISPERSIONS

(71) Applicant: Board of Regents, The University of Texas System, Austin, TX (US)

(72) Inventors: Keith P. Johnston, Austin, TX (US); Thomas Truskett, Austin, TX (US); Barton Dear, Austin, TX (US); Aileen Dinin, Austin, TX (US); Ameya Borwankar, Austin, TX (US); Jessica Hung, Austin, TX (US)

(21) Appl. No.: 14/843,897

(22) Filed: Sep. 2, 2015

Related U.S. Application Data
(60) Provisional application No. 62/045,318, filed on Sep. 3, 2014.

(51) Int. Cl. A61K 47/18; A61K 47/22; A61K 47/26; A61K 39/395

(52) U.S. Cl. CPC A61K 47/183; A61K 39/39591; A61K 47/22; A61K 47/26

(57) ABSTRACT

Disclosed herein are, inter alia, low viscosity dispersions comprising proteins and viscosity lowering agents; pharmaceutical compositions comprising low viscosity dispersions; and methods of making and using the pharmaceutical compositions and low viscosity dispersions.

### RELATED APPLICATIONS

This application claims priority to U.S. Application No. 62/045,318 filed Sep. 3, 2014, the disclosure of which is incorporated by reference herein in its entirety.

### STATEMENT AS TO RIGHTS TO INVENTIONS MADE UNDER FEDERALLY SPONSORED RESEARCH OR DEVELOPMENT

This invention was made with government support under grants CBET-1247945 and CBET-1065357 awarded by the National Science Foundation. The Government has certain rights in the invention.

### FIELD OF THE INVENTION

Disclosed herein are, inter alia, low viscosity dispersions comprising proteins and viscosity lowering agents; pharmaceutical compositions comprising low viscosity dispersions; and methods of making and using the pharmaceutical compositions and low viscosity dispersions.

### BACKGROUND OF THE INVENTION

Formulation of monoclonal antibodies (mAbs) for pharmaceutical delivery presents a number of challenges; often high dosages of >200 mg are required (for example, ˜700 mg for Rituximab). Currently, this dose is delivered intravenously, which requires an extended clinic visit. In contrast, it would be desirable to administer therapeutic proteins subcutaneously at home. However, for a subcutaneous injection with a maximum volume of ˜1.5 mL, required concentrations can be well above 200 mg/mL, where proteins are often insoluble and prone to aggregation, gelation, and precipitation due to specific short-ranged attractive forces. In cells, a heterogeneous mixture of proteins experience highly crowded environments, favoring folding and stability. Recently, we have shown experimentally that a single type of protein may be assembled into nanoclusters on the order of 100 nm, whereby concentrations within the nanoclusters reach 700 mg/mL. Moreover, the proteins are stable with respect to folding and aggregation upon dilution in buffer. Protein stability against unfolding at concentrations as high as those realized inside of protein nanoclusters was predicted by simulation with coarse-grain models. The nanoclusters were formed near the isoelectric point to weaken electrostatic repulsion between protein molecules, by adding trehalose as a crowding agent to provide depletion attraction. In some cases, the size of the nanocluster was described with an equilibrium free energy model, whereby the growth stopped with the buildup of charge in the nanocluster, as has also been observed for Au colloids. It was argued that this combination of depletion attraction between protein monomer and weak long-ranged repulsion between protein nanoclusters, characteristic of interactions in other nanocluster forming systems, produced the equilibrium size. The nanoclusters were formed with mAb 1b7, Sheep IgG or bovine serum albumin (BSA) with trehalose or an N-methylpyrrolidone (NMP) and PEG mixtures as crowding agents. The viscosities of the dispersions ranged from 40-100 cP for concentrations on the order of 200 to 300 mg/ml. Furthermore, the nanoclusters dissociated reversibly to folded monomers upon dilution. For 1b7, the protein from nanocluster dispersions retained comparable bioavailability relative to solution, after subcutaneous and intravenous doses in two murine model studies.

The concept of forming concentrated aqueous dispersions of nanoclusters with lower viscosities than protein solutions was introduced recently as an alternative to protein suspensions in organic solvent. The viscosity of a dispersion of protein molecules or colloids, η, relative to that of the solvent η₀ is described by the Krieger-Dougherty equation:

$$\frac{η}{{η}_{0}} = {[ 1 - ( \frac{{φ}_{eff}}{{φ}_{max}} ) ]}^{- [ η ] {φ}_{max}}$$

as a function of the effective volume fraction (Φ_{eff}), the maximum packing fraction (Φₘₐₓ) and the intrinsic viscosity [η]. We chose Φₘₐₓ for maximum random spherical packing (0.64), and Φ_{eff} was defined as Φ/Φᵢₙₜ, where Φ is the bulk packing fraction of protein in solution and Φᵢₙₜ is the estimated fraction of nanocluster volume occupied by proteins. Here we determine the intrinsic viscosity to be an effective intrinsic viscosity, thus different than the classical definition. In some case it may be fit from the equation for a single data point. It may also be fit for a set of data points at various concentrations. Thus, this definition is different than the standard definition of the term that is commonly used in the limit of extrapolation to zero concentration. This model described by the question above may be applied to a dispersion of individual protein molecules with a diameter of ˜10 nm or, in examples described herein, for a dispersion of large protein colloids (nanoclusters) upon determining a size of a nanocluster domain. It may be applied to a protein dispersion after choosing the parameters φ_{eff} and ₘₐₓ. The φ_{eff} is influenced by the morphology of the protein nanoclusters. It depends upon the packing of the protein within the nanocluster domains and the interactions between the domains. Described herein are Monoclonal antibody solutions often having an [η] of 11-20; in contrast with only 7 for previous examples of nanocluster dispersions, when φₘₐₓ is chosen as 0.64. Other choices of parameters may be defined as known by practitioners in the field. Furthermore, other viscosity equations may be utilized in a similar manner including the Mooney equation and the Ross-Minton equation. Intrinsic viscosity described herein is calculated using the Krieger-Dougherty equation but one of skill in art will understand that other equations and methods may be used as well (e.g., Ross-Minton equation). Nanocluster domains are further apart, on average, in a dispersion than protein monomers are in a solution at comparable concentration, when scaled by the size of the entities. Furthermore, intercluster attractions in a dispersion are expected to be weaker than interprotein attraction in a solution, which may lead to a decrease in [η] in the dispersions. The weakening of the interactions may be produced by compressing the proteins together into denser domains. It may also be produced by blocking interactions between proteins upon forming interactions between proteins and small molecules. So far, relatively few studies have been reported that relate the [η] of nanoclusters dispersions to the nanocluster geometry and composition, and how they are influenced by the nature and concentration of the crowding agent. The definition of φ_{eff} is the suspected volume fraction of nanoclusters in the dispersion. It is defined as the concentration of protein, as measured by UV-Vis A280, divided by the density of protein, 1350 mg/ml, divided by the suspected cluster internal volume fraction of protein, 0.6 (60% of each nanocluster is composed of protein, rest is solvent). The φₘₐₓ used is 0.64, which is the maximum packing volume fraction of spheres.

The Ross-Minton equation is another type of equation in addition to the Krieger-Dougherty equation for the prediction of the relationship between viscosity and concentration for colloids, including proteins. The Ross-Minton equation is defined as

$$\frac{η}{{η}_{0}} = exp [ \frac{c [ η ]}{1 - \frac{k}{v} [ η ] c} ]$$

Where η is the viscosity, η₀ is the solvent viscosity, c is the protein concentration, [η] is the intrinsic viscosity, k is the crowding factor, and v is the Simha shape parameter. As shown in the equation, the crowding factor and Simha shape parameter are often combined into a single term, which is sometimes referred to as the effective crowding factor. In this case the intrinsic viscosity is different from that used in the Krieger-Dougherty relationship, as the Ross-Minton intrinsic viscosity has units of inverse concentration and therefore represents the effective volume of solute per unit mass. In contrast, the Krieger-Dougherty intrinsic viscosity is unitless. As both the Ross-Minton and Krieger-Dougherty equation are two parameter exponential functions relating viscosity to concentration, they both may be used to fit plots of viscosity versus concentration for protein solutions and dispersions. The parameters that are fit are related to the molecular properties of the protein molecules, the interactions between the protein molecules, and also interactions with other molecules in the system. Therefore, the data presented throughout this application could also be fit with the Ross-Minton equation. For a single viscosity data point, it is necessary to choose a priori one of the parameters and then to fit the other. In embodiments, the [η] may be specified for a protein based on the literature or extrapolation of viscosity data to low concentrations. For a single viscosity data point, a value of k/v may then be regressed to provide a measure of the interactions. This k/v value is smaller for systems with weaker interactions between proteins and this will result in a smaller value of viscosity, for a given solvent viscosity. This approach may be applied to the data described herein to compare various data points. This approach is analogous to solving for a single parameter in the Krieger-Dougherty equation. For a series of viscosities versus concentration, the Ross-Minton may be used to solve for both parameters, the intrinsic viscosity and k/v. A useful way to compare the behavior for various samples is with the inherent viscosity given by

$${η}_{inh} = \frac{ln ( η / {η}_{0} )}{c}$$

where ηᵢₙₕ is the inherent viscosity. The inherent viscosity is useful for normalizing the effects of solvent viscosity and concentration to then examine the effect of protein interactions.

Amino acids have been used to stabilize protein solutions and would be interesting candidates for crowding agents to form protein nanoclusters. Proline is a very small stabilizer for protein solutions, with a molar mass of only 115 and a high solubility of ˜750 mg/mL; it is the only secondary amine among the 20 natural amino acids. Similar to trehalose, proline is produced as an osmolyte in stressed organisms in dry or high temperature environments. It is a natural cryoprotectant, that has been used as an excipient to enhance stabilization in pharmaceutical formulations. Proline has been found to inhibit aggregation during folding and in aggregation-prone proteins in vivo and in vitro. It appears to destabilize partially-folded states and early aggregates, while solubilizing native-state protein. A mechanism by which it stabilizes proteins is through solvophobic interactions between proline and the protein backbone, causing preferential exclusion of the proline solute from the protein-water interface of partially-folded intermediates. This preferential exclusion increases the free energy of the unfolded state, making it thermodynamically unfavorable. When interacting with BSA, preferential exclusion of proline is actually greater than in the case of trehalose, which also has unfavorable backbone interactions.

Protein nanoclusters may be formed by dilution of lyophilized powders in buffer, and by concentrating protein solutions, for example with centrifugal filtration through a membrane. In some cases, the same size was formed by two pathways suggesting the possibility of a metastable equilibrium state. However, little is known about how the pathway of nanocluster formation influences the viscosity. When forming a high concentration dispersion of nanoclusters by filtration, the system traverses intermediate protein concentrations prior to nanocluster formation (so-called “danger zone”) for very short times, which may minimize the tendency for unfolding and formation of aggregates. Here, a sufficiently concentrated crowder may help to mitigate unfolding via preferential exclusion or other osmotic forces. For proline, its high solubility is beneficial for exploring multiple pathways to form nanoclusters without limitations from crowder dissolution. It is possible that certain pathways will lead to dispersions that are trapped in metastable gel states causing undesirable viscosity increases, while other pathways may potentially lead to low viscosities. Currently, very little is known about the relationship between viscosity and nanocluster formation pathways given the complexities of gel states at high concentrations. Described herein are solutions to these and other needs in the art.

### BRIEF SUMMARY OF THE INVENTION

In an aspect is provided a low viscosity dispersion including a protein and a viscosity lowering agent. The viscosity lowering agent is present at a concentration from about 10 mg/mL to about 300 mg/mL. The protein is present at a concentration of greater than about 200 mg/mL. The protein is present within a plurality of protein nanoclusters. The viscosity lowering agent is proline, histidine, lysine, arginine, glutamic acid, betaine, glutamine, asparagine, or imidazole.

In another aspect is provided a method of making a low viscosity dispersion as described herein, including in an embodiments, example, figure, table, or claim.

### BRIEF DESCRIPTION OF THE DRAWINGS

FIG. 1. Measured zeta potential of 2 mg/mL mAb 1 solutions with and without large excess of 150 mg/mL proline or 200 mg/mL trehalose. Samples are diluted in 150 mM pH 4.7 acetate buffer, 50 mM pH 6.4, 7.2 or 8.2 phosphate buffer, or 50 mM pH 9.3 or 11.0 carbonate buffer. Legend: no excipient (diamonds), proline (circle), trehalose (squares), linear (no excipient) (solid line), linear (proline) (dotted line), linear (trehalose) (dashed line).

FIG. 2. Nanocluster size contours for mAb 1 nanoclusters produced with proline and experimental pathways for nanocluster formation including lyophilization dilution (LD), centrifugation filtration (C), and centrifugation filtration over powder (C-D/P) methods

FIG. 3. Comparison between the calculated depletion potentials for proline and trehalose at 150 mg/mL. (Tuinier, R.; Rieger, J.; de Kruif, C. G. Advances in Colloid and Interface Science 2003, 103, 1.) The proline depletion potential has a higher magnitude than trehalose at small surface-to-surface distances, but decays faster. Legend: trehalose (solid line), proline (dashed line).

FIG. 4. pH dependence of nanocluster size for ˜250 mg/mL mAb 1 dispersions manufactured by centrifugation (C) with 150 mg/mL proline and by centrifugation-dispersion over powder (C-D/P) with 250 mg/ml of proline. A slight increase in size is observed approaching the mAb pI. The six samples shown here correspond to samples C-D/P 251:150P (pH 6.4), C-D/P 258:150P (pH 7.2) and C-D/P 257:150P (pH 8.2), found in Table 5. Entries for histogram bins (left to right): 250:150 C; 250-:250 C-D/P. For each histogram bin, entries are in the order (left to right): 250:150C, 250:250 C-D/P.

FIG. 5. pH dependence of the dispersion viscosity for 250 mg/ml dispersions manufactured by both the centrifugation-dispersion over powder (C-D/P) and centrifugation filtration (C) methods. The pH 9.3 dispersion was formulated using 50 mM carbonate buffer, and all other formulations were made in 50 mM sodium phosphate buffer. Entries for histogram bins (left to right): 250:150 C; 250-:250 C-D/P.

FIG. 6. Beer's Law correlation between the proline concentration and measured HPLC-SEC proline peak area for samples of known composition, for quantification of retained proline in centrifugation (C) and centrifugation-dispersion over powder (C-D/P) samples. The proline peak area was normalized by the measured protein peak area to account for deviations in the mass of injected protein between samples.

FIG. 7. Sample SEC curves of 238:250 mg/mL mAb 1:proline and 236:250 mg/mL mAb 1:proline dispersions formulated by the centrifugation filtration over powder (C-D/P) approach. The main (monomer) peak occurs at 32 minutes, while the smaller aggregate peak occurs at 28 minutes. A very small (unlabeled) peak is observed at 52 minutes, corresponding to the proline in the sample.

FIG. 8. Intrinsic viscosity as a function of pH and histidine (His) concentration for mAb1 nanocluster dispersions at ˜250 mg/ml made with ˜50 mg/ml trehalose, varying levels of histidine, as indicated, and enough HCl to control the pH to either 5.5, 6.0 or 6.5. Average values are used for systems with replicates. Legend: 5 mg/mL His (diamonds); 20 mg/mL His (squares); 40 mg/mL His (triangles); 60 mg/mL His (crosses); 80 mg/mL (crosses with vertical line); 120 mg/mL His (circles).

FIG. 9. depicts a 3-D plot of intrinsic viscosity as a function of pH and histidine concentration for mAb1 nanocluster dispersions made at ˜250 mg/ml made with ˜50 mg/ml trehalose, histidine as indicated and enough HCl to control the pH. Black triangles represent actual data points. Average values are used for systems with replicates.

FIG. 10. Intrinsic viscosity as a function of pH and trehalose (Tre) concentration for mAb1 nanocluster dispersions at ˜250 mg/ml made with varying levels of trehalose, as indicated, ˜40 mg/ml histidine and enough HCl to control the pH to either 5.5, 6.0 or 6.5. Average values are used for systems with replicates. Legend: 50 mg/mL Tre (trehalose) (diamonds); 100 mg/mL Tre (squares); 150 mg/mL Tre (triangles).

FIG. 11. depicts a 3-D plot of intrinsic viscosity as a function of pH and trehalose concentration for mAb1 nanocluster dispersions made at ˜250 mg/ml made with trehalose as indicated, 40 mg/ml histidine and enough HCl to control the pH. Black triangles represent actual data points. Average values are used for systems with replicates.

FIG. 12. Intrinsic viscosity as a function of system for dispersions made with trehalose (Tre), histidine (His) and either hydrochloric acid (HCl), citric acid (CA) or phosphoric acid (PA) as indicated. All systems contain ˜250 mg/ml mAb1 and other excipients as listed (numbers are in mg/ml). Average values are used for systems with replicates. Entries for histogram bins, with conditions indicated along x-axis in FIG. 12, appear in order (left to right); HCl, CA, PA.

FIG. 13. Intrinsic viscosity as a function of system for replicates made with trehalose, histidine and either hydrochloric acid (HCl) or citric acid (CA). All systems contain ˜250 mg/ml mAb1, 50 mg/ml trehalose, 40 mg/ml histidine and enough of the listed acid to control the pH. Replicates are side by side. Entries for histogram bins appear in order (left to right) as in FIG. 12.

FIG. 14. Intrinsic viscosity as a function of system for replicates made with trehalose, imidazole (Imid) and either hydrochloric acid (HCl), citric acid (CA) or phosphoric acid (PA). All systems contain ˜250 mg/ml mAb1, 50 mg/ml trehalose and imidazole content and acid as listed (numbers are in mg/ml). Replicates are side by side. Entries for histogram bins appear in order (left to right) as in FIG. 12.

FIG. 15. Autocorrelation functions of a representative nanocluster sample (250 mg/ml mAb1, 78 mg/ml arginine, 72 mg/ml glutamic acid) and a representative monomer sample (200 mg/ml mAb1 in DI water). The longer relaxation time for the dashed curve indicates a larger entity than a protein monomer, with slower diffusion. The larger entity is termed a nanocluster. Nanocluster diameter was determined to be 36 nm and the monomer was determined to be 11 nm from CONTIN analysis.

FIGS. 16A-16B. Viscosity of mAb1 dispersions as a function of the protein concentration. FIG. 16A: The arg glu samples had 78.6 mg/ml arg and 71.4 mg/ml glu while the histidine buffer samples were in pH 5.5 histidine buffer at 20 mM. The lines are fits with the Ross-Minton equation in terms of both [η] and k/v. FIG. 16B: D/D₀ from DLS. For both FIGS. 16A-16B, the open symbols show the data for samples prepared by dilution of the highest concentration samples. Legend: arg+glu 1 (diamonds); arg+glu2 (squares); his buffer 1 (triangles); his buffer 2 (circles).

FIG. 17. Decrease in inherent viscosity of mAb1 dispersions as the concentration of arg+glu is increased from 75 to 150 mg/ml for the data in Tables 2-4 and 2-5 at pH 5.5. The line is a guide to the eye.

FIG. 18. Viscosity of mAb1 dispersions as a function of pH at a constant arg and glu concentration of 150 mg/ml. The ratio of arg to glu was varied to change the pH. Legend: pH 5 (diamonds); pH 7.1 (triangles); pH8.5 (crosses); pH 11.5 (circles).

FIG. 19. Inherent viscosity (diamonds) and viscosity (circles) for mAb 1 dispersions with 150 mg/ml arg titrated with HCl. Centrifugation speed was 10000 ref. The viscosity goes through a maximum near the isoelectric point, pH 9.

FIG. 20. Effect of shear rate on viscosity of mAb1 dispersions at ˜228 mg/ml with 20 mM histidine and 0.05% Tween 80 (circles) and 269 mg/ml with 78.6 mg/ml arg and 71.4 mg/ml glu (crosses). Legend: arg+glu (crosses); his+Tween (circles); arg+glu syringe (crosses with vertical line); his+tween syringe (circles with diagonal line).

FIGS. 21A-21B. mAb dispersion viscosities during ultrafiltration to 250 mg/ml mAb by TFF plotted on (FIG. 21A) linear and (FIG. 21B) log scale. The numbers in the formulation names represent concentration of each excipient in mg/ml. The solid lines represent two-parameter fits to the data by the Ross-Minton equation. Legend: Freezing buffer, pH 6.3 (circles); DI water pH 6.6 (squares); 40:50:17 Tre:His:CitrA, pH 6.0 (circles with diagonal); 50:50:12 Tre:His:PhosA, pH 6.0 (diamonds); 50:17:7 Tre:Imidazole (Im):HCl, pH 6 (triangles); 50:17:10 Tre:Im:CitrA, pH 7 (circles with vertical line).

FIG. 22. Viscosity-concentration profiles of the mAb dispersion with 40:50:17 mg/mL Tre-His-CitrA (pH 6) as a function of four diafiltration conditions as described herein. The lines are guides to the eye. Legend: Rep 1 (triangles); Rep 2 (closed circles); Rep 3 (grayed circles); Rep 4 (triangles).

FIG. 23. Reversibility of Replicate 4 of the Tre-His-CitrA mAb dispersion viscosity between 150 and 280 mg/ml during concentration by TFF (——) and dilution in buffer (- - -). The lines represent two-parameter fits to the data by the Ross-Minton equation. Legend: Concentration by TFF (diamonds); Dilution from TFF (circles).

FIGS. 24A-24B. TFF membrane (FIG. 24A) flux (in L/m²h) and (FIG. 24B) permeation flux resistance Rₜ (in m⁻¹) as a function of mAb concentration during ultrafiltration to 250 mg/ml mAb for low co-solute (- - -) and high co-solute (— —) dispersions. The flux and resistance curves for the 40:50:17 mg/ml Tre:His:Citr dispersion corresponds to Replicate 1. The lines are a guide to the eye, each corresponding to the data series. Legend: Freezing buffer, pH 6.3 (crosses with vertical line); DI water, pH 6.6 (circles); 40:50:17 Tre:His:Citr, pH 6 (diamonds); 50:50:12 Tre:His:Phos, pH 6 (squares).

FIGS. 25A-25B. TFF membrane (FIG. 25A) flux (in L/m²h) and (FIG. 25B) permeation flux resistance Rₜ (in m⁻¹) as a function of dispersion viscosity during ultrafiltration to 250 mg/ml mAb for low co-solute (- - -) and high co-solute (— —) dispersions. The flux and resistance curves for the 40:50:17 mg/ml Tre:His:Citr dispersion corresponds to Replicate 1. The lines are a guide to the eye, each corresponding to the data series. Legend as in FIGS. 24A-24B.

FIGS. 26A-26B. Time evolution of (FIG. 26A) mAb concentration and (FIG. 26B) pressure drop and wall shear stress during TFF ultrafiltration for low co-solute (- - -) and high co-solute dispersions (——). The concentration and pressure/shear stress curves for the 40:50:17 mg/ml Tre:His:Citr dispersion corresponds to Replicate 1. Legend: Freezing buffer (crosses with vertical line); DI water (circles); 40:50:17 Tre:His:CitrA (diamonds); 50:50:12 Tre:His:PHosA (crosses).

FIG. 27. pH shift of mAb dispersions during ultrafiltration by TFF. The pH of the low co-solute (- - -) and high co-solute (— —) mAb dispersions were measured with a Mettler Toledo InLab Micro pH probe (Mettler Toledo, Columbus, Ohio). The pH shift for the 40:50:17 mg/ml Tre:His:Citr dispersion corresponds to Replicate 4 disclosed herein. Legend: Freezing buffer, pH 6.3 (crosses with vertical line); DI water, pH 6.6 (circles); 40:50:17 Tre:His:Citr, pH 6.0 (diamonds); 50:50:12 Tre:His:PhosA, pH 6.0 (squares).

FIGS. 28A-28D. Storage stability of Replicate 4 of the 40:50:17 mg/ml Tre:His:Citr dispersion as characterized by the (FIG. 28A) inherent viscosity and (FIG. 28B) percent monomer by SEC upon dilution to 2 mg/ml mAb. (FIG. 28C) The Replicate 4 dispersion remained as a clear liquid during prolonged storage at 4° C., whereas (FIG. 28D) a low co-solute dispersion (235 mg/ml mAb in DI water) gelled and phase separated within 20 minutes of 4° C. storage. A 240 mg/ml mAb dispersion in freezing buffer (low co-solute) also resembled the gelled dispersion in (FIG. 28D) within minutes of storage at 4° C.

FIG. 29. Effect of mAb concentration and pH on viscosity of conventionally buffered mAb1 dispersions made by CF. Solvents contain 35 mM histidine and enough HCl to control the pH. Error bars are standard deviations of measurements, some error bars are too small to be seen. Lines are best fits of the Ross-Minton equation (equation 5-3) to the data with parameters given in Table 5-S7. The inset highlights the viscosity predicted by the best fit line at a concentration of 200 mg/ml mAb1.

FIG. 30. Effect of mAb concentration and pH on viscosity of mAb1 dispersions containing elevated concentrations of histidine made by CF. Solvents contain 305 mM histidine and enough HCl to control the pH. Error bars are standard deviations of measurements, some error bars are too small to be seen. Lines are best fits of the Ross-Minton equation (equation 5-3) to the data with parameters given in Table 5-S7. The inset highlights the viscosity predicted by the best fit line at a concentration of 200 mg/ml mAb1.

FIG. 31. Effect of histidine concentration and pH on inherent viscosity of ˜250 mg/ml mAb1 dispersions made by CF and LD. Hollow symbols represent samples made by centrifugation filtration (CF), which contain ˜250 mg/ml mAb1, the listed amount of histidine and enough HCl to control the pH. Filled symbols represent samples made by lyophilization dilution (LD), which contain ˜250 mg/ml mAb1, 130 mM trehalose, the listed amount of histidine and enough HCl to control the pH.

FIG. 32. Effect of histidine concentration on inherent viscosity of ˜250 mg/ml mAb1 dispersions made by LD. Samples made by lyophilization dilution (LD), and contain ˜250 mg/ml mAb1, 130 mM trehalose, the listed amount of histidine and enough HCl to control the pH to ˜5.5. The line is a guide to the eye.

FIG. 33. Effect of histidine concentration and pH on inherent viscosity of ˜250 mg/ml mAb1 dispersions made by LD. Samples made by lyophilization dilution (LD), and contain ˜250 mg/ml mAb1, 130 mM trehalose, the listed amount of histidine and enough HCl to control the pH. Replicate runs were averaged. The heat map is a linear interpolation of the data points, marked by black triangles.

FIG. 34. Effect of mAb concentration and pH on viscosity of mAb1 dispersions containing elevated concentrations of arginine made by CF. Solvents contain 305 mM arginine and enough HCl to control the pH. Error bars are standard deviations of measurements, some error bars are too small to be seen. Lines are best fits of the Ross-Minton equation (equation 5-3) to the data with parameters given in Table 5-S7. The inset highlights the viscosity predicted by the best fit line at a concentration of 200 mg/ml mAb1.

FIG. 35. Effect of mAb concentration and buffer composition on viscosity of mAb1 dispersions of similar ionic strength and pH made by CF. Solvents contain 305 mM total co-solute and enough HCl to control the pH, except for NaCl and NaCSA which contain 35 mM histidine with enough HCl to control the pH and 270 mM NaCl or NaCSA, respectively. Error bars are standard deviations of measurements, some error bars are too small to be seen. Lines are best fits of the Ross-Minton equation (equation 5-3) to the data with parameters given in Table 5-S7. The inset highlights the viscosity predicted by the best fit line at a concentration of 200 mg/ml mAb1.

FIG. 36. Correlation between inherent viscosity at ˜200 mg/ml with normalized turbidity for CF samples made with solvents containing 35 mM co-solute versus those made with 305 mM co-solute. Data for plot can be found in Tables 5-1 and 5-3. The line is just a guide to the eye.

FIG. 37. Effect of shear rate on viscosity of mAb1 dispersion. Sample was concentrated by CF to 215±3 mg/ml mAb1 in a solvent containing 320 mM histidine, 80 mM citric acid and 105 mM trehalose. The final pH was 5.92. The error bars are the standard deviations of the measurements. The line is a guide to the eye.

FIG. 38. Viscosity vial volume by height correlation. Plots measured liquid heights for different known volumes for eight different vials. In the legend, the first number is the difference between volume additions per measurement in microliters, and the second number is the replicate number. Fit is the average quadratic fit of the data. The equation of the fit is V=0.426*h²−1.135, where V is the liquid volume in microliters and h is the meniscus height in mm.

FIG. 39. Viscosity of known standards versus flow rate in needle. Plots the flow rate of viscosity standards S60, N44, N35 and N10, and DI water during a viscosity measurement, by their known viscosities. Data were fit to the Hagen-Poiseuille equation where it was determined that the pressure drop is 7686 Pa. Error bars are standard deviations between measurements, some error bars are too small to be seen.

FIG. 40. Predicted shear rate in syringe viscometer by sample viscosity. Shear rate prediction is calculated assuming Newtonian flow from equation 5-S1 set forth herein. Dashed black lines mark the minimum and maximum viscosities measured by this viscometer in this study. The shear rates corresponding to these minimum and maximum viscosities are 861 and 58 s⁻¹, respectively.

FIG. 41. Comparison of background corrected sample turbidity between CF and LD dispersions 500× dilute, 1 cm path length for measuring concentration. The CF sample was concentrated in a solvent containing 305 mM histidine with HCl to 250 mg/ml mAb1 at a pH of 5.01. The LD sample contains 260 mg/ml mAb1 with 250 mM histidine with HCl at pH 5.25 and 130 mM trehalose. The A350 of the CF sample is near zero, signifying minimal turbidity, while the LD sample has visible turbidity.

FIG. 42. Comparison of sample turbidity between CF and LD dispersions without dilution 0.2 cm path length. The CF sample was concentrated in a solvent containing 305 mM histidine with HCl to 250 mg/ml mAb1 at a pH of 5.98. The LD sample contains 266 mg/ml mAb1 with 250 mM histidine with HCl at pH 5.79 and 130 mM trehalose. The difference in buffer turbidity can be accounted for by using different cuvettes.

FIG. 43. Percent of positively charged co-solute as a function of pH. Numbers were calculated by solving Handerson-Hasselbalch equation simultaneously for all of each co-solutes' known pKₐ values.

FIG. 44. Effect of mAb concentration and solvent composition on viscosity of pH ˜5 mAb1 dispersions made by CF. Solvents contain co-solute as listed with enough HCl to control the pH to ˜5. Error bars are standard deviations of measurements. Lines are best fits of the Ross-Minton equation (equation 3) to the data with parameters given in Table 5-S7. The inset highlights the viscosity predicted by the best fit line at 200 mg/ml mAb1.

FIG. 45. Effect of mAb concentration and solvent composition on viscosity of pH ˜6 mAb1 dispersions made by CF. Solvents contain co-solute as listed with enough HCl to control the pH to ˜6. Error bars are standard deviations of measurements. Lines are best fits of the Ross-Minton equation (equation 3) to the data with parameters given in Table 5-S7. The inset highlights the viscosity predicted by the best fit line at 200 mg/ml mAb1.

FIG. 46. Effect of mAb concentration and solvent composition on viscosity of pH 7 mAb1 dispersions made by CF. Solvents contain co-solute as listed with enough HCl to control the pH to ˜7. Error bars are standard deviations of measurements. Lines are best fits of the Ross-Minton equation (equation 3) to the data with parameters given in Table 5-S7. The inset highlights the viscosity predicted by the best fit line at 200 mg/ml mAb1.

FIGS. 47A-47B. Viscosity reductions for select 200-230 mg/mL dispersions at (FIG. 47A) pH 5 and (FIG. 47B) pH 6 after the addition of high concentrations of proline to the mAb solution in histidine or imidazole buffer. The exact mAb and buffer concentrations are listed in mg/mL and mM, respectively.

FIG. 48. Stability of ˜225 mg/mL mAb dispersions before (left bars) and after (right bars) 4 weeks of accelerated storage at 40° C. with addition of proline and/or histidine. The monomer fraction was measured by HP-SEC after dilution of the storage samples to 2 mg/mL in the SEC mobile phase. The corresponding co-solute compositions (in mM) and pH are listed on the x-axis. For each histogram bin, bars appear in the order (left to right): time 0-weeks, time 4-weeks.

### DETAILED DESCRIPTION OF THE INVENTION

The ability to form monoclonal antibody dispersions at high concentrations, with low viscosities for subcutaneous injection, is of broad interest for simplifying treatment of cancer, autoimmune disorders, and many other diseases. Herein, we create highly concentrated antibody dispersions of nanoclusters of a monoclonal antibody (e.g., up to 260 mg/mL) with an amino acid, proline, to modulate the surface charge and to produce depletion attraction (crowding). The nanocluster diameter of the nanoclusters remains relatively constant at 40-50 nm, for a pH range of 7.2-9.3. The viscosities of the dispersions, as low as 35 cP at 258 mg/mL, were up to an order of magnitude lower than similarly concentrated antibody solutions. When diluted, these antibody dispersions return to monomer. The proline lowers the protein zeta potential, thus reducing electrostatic repulsion between protein monomer to a sufficient degree for nanocluster formation, even 3 pH units away from the antibody pI. In addition, proline acts as a depletant, whereby the osmotic force is sufficient to drive the monomers into nanocluster. The objective is to explore the formation, morphology and viscosity of protein nanocluster dispersions with a new type of crowding agent (depletant), an amino acid proline, for a human mAb IgG₁κ (mAb 1) with a pI of 9.3 in various dispersion formation pathways. We would like to understand, relative to previous crowders including trehalose, and polyethylene glycol, how the much smaller size of proline, its zwitterionic charge state, and the strong preferential exclusion may influence the properties of the nanocluster dispersions. These properties will further be shown to be influenced by a large effect of proline on the pI of the protein. At dilute protein concentrations, proline at high concentration will be shown to lower the charge on mAb 1 to nearly zero (isoelectric point (pI) is at pH 9.3) for pH values between 6 and 9, suggesting significant interactions with the surface charges on the protein. Although previous studies have focused on nanoclusters at the pI, we explore a wider range of pH to further understand the mechanism of nanocluster formation. From a stability and safety point of view for subcutaneous injection, it would be desirable to be able to form nanoclusters at a pH of near 7 well below the pI of 9.3 for mAb 1. In addition, the effects of electrostatic interactions on viscosities of concentrated protein solutions are large, and may thus also be expected to have a significant effect on those of nanocluster dispersions. Interestingly, protein nanocluster size will be found to be relatively independent of proline concentration, in contrast to what has been seen for trehalose as a crowder for another protein, sheep IgG.

Three different pathways are examined for protein nanocluster formation: lyophilization followed by dilution of the lyophilized powder (lyophilization dilution LD), addition of dispersions formed by centrifugation to lyophilized powder (C-D/P) and centrifugation filtration to concentrate solutions into nanoclusters (C). All three pathways will be shown to produce similar size nanoclusters, as seen previously between C and LD samples in our previous work with trehalose as a crowder for sheep IgG. However, it will be seen that the viscosities vary modestly for the various processes, despite similar nanocluster sizes.

### DEFINITIONS

As used herein, the term “nanoparticle” refers to a particle having a diameter of between about 1 and 1000 nanometers. A nanoparticle may have a diameter of between about 10 and 1000 nanometers. A nanoparticle may have a diameter of between about 20 and 1000 nanometers. A nanoparticle may be a nanocluster. In embodiments, a nanoparticle may have a diameter between 10 and 900 nm, 10 and 800 nm, 10 and 700 nm, 10 and 600 nm, 10 and 500 nm, 10 and 400 nm, 10 and 300 nm, 10 and 200 nm, 10 and 100 nm, 20 and 900 nm, 20 and 800 nm, 20 and 700 nm, 20 and 600 nm, 20 and 500 nm, 20 and 400 nm, 20 and 300 nm, 20 and 200 nm, 20 and 100 nm, 50 and 1000 nm, 50 and 900 nm, 50 and 800 nm, 50 and 700 nm, 50 and 600 nm, 50 and 500 nm, 50 and 400 nm, 50 and 300 nm, 50 and 200 nm, 50 and 100 nm, 75 and 1000 nm, 75 and 900 nm, 75 and 800 nm, 75 and 700 nm, 75 and 600 nm, 75 and 500 nm, 75 and 400 nm, 75 and 300 nm, 75 and 200 nm, 75 and 100 nm, 100 and 1000 nm, 100 and 900 nm, 100 and 800 nm, 100 and 700 nm, 100 and 600 nm, 100 and 500 nm, 100 and 400 nm, 100 and 300 nm, 100 and 200 nm, 250 and 1000 nm, 250 and 900 nm, 250 and 800 nm, 250 and 700 nm, 250 and 600 nm, 250 and 500 nm, 250 and 400 nm, 250 and 300 nm, 500 and 1000 nm, 500 and 900 nm, 500 and 800 nm, 500 and 700 nm, 500 and 600 nm, 750 and 1000 nm, 750 and 900 nm, or 750 and 800 nm.

As used herein, the term “nanocluster” refers to an assembly of proteins or peptides that are not irreversibly aggregated, which may optionally be associated physically or through specific intermolecular interactions with additional compounds, components, or compositions. In embodiments, a nanocluster is a nanoparticle. In embodiments, a nanocluster is an assembly of proteins or peptides that scatter light differently than a protein or peptide monomer due to the nanocluster being an assembly instead of a monomer. In embodiments, a nanocluster is a protein assembly that may be observed to be an assembly instead of a monomer by microscopy or other procedures used to characterize colloids. A dispersion of nanoclusters may be polydisperse, whereby the polydispersity may be measured by various techniques know in particle sizing. A nanocluster may be a discrete object separate from other nanoclusters. In embodiments, the nanocluster may interact with (e.g., contact) a protein near the nanocluster. In yet another embodiment, a nanocluster may be a region in a dispersion wherein the proteins are more compactly positioned (e.g., closer to each other) than in a uniform solution of a similar protein concentration, for example, as a result of the addition of one or more excipients or crowders. In some embodiments, compaction of proteins in the nanocluster may be at least partially due to depletion attraction. The proteins in the dispersion may be distributed as a first type of nanocluster, optionally with one or more of protein monomers, dimers, multimers, aggregates, and/or protein assemblies that are optionally different types of nanoclusters or aggregates or assemblies having a different size from the first type of nanocluster. Determination of aggregate, monomer, and/or protein assembly sizes is dependent on the characterization technique used. For example, size determination by dynamic light scattering depends on the shape of the autocorrelation function used and the model used to determine the autocorrelation function. In embodiments, nanocluster diameter refers to the volume average size determined from the Stokes Einstein equation when fitting the autocorrelation function using dynamic light scattering measurements. It may also refer to a component in the intensity distribution. Examples of fitting algorithms include, but are not limited to, CONTIN, NNLS, or similar algorithms available to practitioners in the field of light scattering. In some embodiments, the diameter is a nanocluster diameter determined from dynamic light scattering. In some embodiments, the diameter is a hydrodynamic diameter. In some embodiments, the nanocluster may include subclusters of proteins or peptides that form a larger nanocluster. In some embodiments, the nanocluster may be self-crowding, wherein the crowding is caused by the proteins or peptides. In some embodiments, the nanocluster may form in the presence of an extrinsic crowder. In some embodiments, the nanocluster may be mostly self-crowding. In some embodiments, one or more extrinsic crowder may perform a function in which it breaks down larger protein aggregates into smaller aggregates by breaking intermolecular interactions between two or more protein molecules. These interactions may be specific short ranged interactions, electrostatic interactions, acid-base interactions, hydrophobic interactions, interactions between one or more charges on one protein molecule and dipoles or charge on another protein molecule. In some embodiments, these interactions take place at the same time as any of the other interactions above including the compaction of the protein. In this case the extrinsic crowder is simultaneously driving certain protein domains closer together, while breaking other protein interactions. Upon breaking protein aggregate networks, a change may be observed in the autocorrelation functions. The nanoclusters may be distinct species not touching other nanoclusters, with smaller protein domains between the nanoclusters including protein monomer. Or the intermediate smaller protein domains may interconnect the nanoclusters. In embodiments, the nanocluster does not include protein or peptide crystals. In embodiments, a nanocluster may have a diameter between 1 and 1000 nm, 10 and 1000 nm, 10 and 900 nm, 10 and 800 nm, 10 and 700 nm, 10 and 600 nm, 10 and 500 nm, 10 and 400 nm, 10 and 300 nm, 10 and 200 nm, 10 and 100 nm, 20 and 900 nm, 20 and 800 nm, 20 and 700 nm, 20 and 600 nm, 20 and 500 nm, 20 and 400 nm, 20 and 300 nm, 20 and 200 nm, 20 and 100 nm, 50 and 1000 nm, 50 and 900 nm, 50 and 800 nm, 50 and 700 nm, 50 and 600 nm, 50 and 500 nm, 50 and 400 nm, 50 and 300 nm, 50 and 200 nm, 50 and 100 nm, 75 and 1000 nm, 75 and 900 nm, 75 and 800 nm, 75 and 700 nm, 75 and 600 nm, 75 and 500 nm, 75 and 400 nm, 75 and 300 nm, 75 and 200 nm, 75 and 100 nm, 100 and 1000 nm, 100 and 900 nm, 100 and 800 nm, 100 and 700 nm, 100 and 600 nm, 100 and 500 nm, 100 and 400 nm, 100 and 300 nm, 100 and 200 nm, 250 and 1000 nm, 250 and 900 nm, 250 and 800 nm, 250 and 700 nm, 250 and 600 nm, 250 and 500 nm, 250 and 400 nm, 250 and 300 nm, 500 and 1000 nm, 500 and 900 nm, 500 and 800 nm, 500 and 700 nm, 500 and 600 nm, 750 and 1000 nm, 750 and 900 nm, or 750 and 800 nm.

As used herein, the terms “syringable” and “syringeable” are used interchangeably and refer to a final composition for delivery to a subject that is sufficiently fluid to be flowable through a syringe (e.g. a syringe with a needle that is 21 to 27 gauge). For example, a composition that is “syringable” has a low enough viscosity to load the syringe and inject a subject from the syringe without undue force, wherein undue force is an amount in excess of the force exerted by a skilled practitioner in the medical field (e.g. doctor, nurse) to deliver compositions to a patient (e.g. through iv injection, SQ injection) through a syringe (e.g. a syringe with a needle that is 21 to 27 gauge) without adverse effects to the patient solely due to the force applied in the delivery.

As used herein, the term “non-settling” or “redispersible” refers to a composition that remains in solution phase (i.e., does not sediment) after an extended period of time, e.g., 1 hour, 2 hours, 1 day, 3 days, 5 days, 1 week, 1 month, 3 months, 6 months, 1 year or more). For example, a composition is “re-dispersible” if upon re-dispersion it does not flocculate so quickly as to prevent reproducible dosing of a drug.

As used herein, the term “additive(s)” refers to salts, sugars, organics, buffers, amino acids, polymers and other compositions that include: Disodium edetate, Sodium chloride, Sodium citrate, Sodium succinate, Sodium hydroxide, Sodium glucoheptonate, Sodium acetyltryptophanate, Sodium bicarbonate, Sodium caprylate, Sodium pertechnetate, sodium acetate, sodium dodecyl sulfate, aluminum hydroxide, aluminum phosphate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, proline, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, mannitol, lactose, sucrose, agarose, sorbitol, maltose, trehalose, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, Polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol (antiseptic), glyclyglycine, thimerosal (antiseptic, antifungal), Parabens (preservative), Gelatin, Formaldehyde, Dulbecco's modified eagles medium, Hydrocortisone, Neomycin, Von Willebrand factor, Gluteraldehyde, Benzethonium chloride, White petroleum, p-aminopheyl-p-anisate, monosodium glutamate, beta-propiolactone, Acetate, Citrate, Glutamate, Glycinate, Histidine, imidazole, phosphate, Lactate, Maleate, Phosphate, Succinate, Tartrate, Tris, Carbomer 1342 (copolymer of acrylic acid and a long chain alkyl methacrylate cross-linked with allyl ethers of pentaerythritol), Glucose star polymer, Silicone polymer, Polydimethylsiloxane, Polyethylene glycol, carboxymethylcellulose, Poly(glycolic acid), Poly(lactic-co-glycolic acid), Polylactic acid, Dextran 40, Poloxamers (triblock copolymers of ethylene oxide and propylene oxide).

The terms “a” or “an,” as used in herein means one or more. The terms “s.d.,” “std. dev.” and the like refer, in the usual and customary manner, to the standard deviation of a set of data.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. compounds including biomolecules, proteins, antibodies, or cells) to become sufficiently proximal to react, interact or physically touch. It should be appreciated, however, the resulting reaction product can be produced directly from a reaction between the added reagents or from an intermediate from one or more of the added reagents which can be produced in the reaction mixture.

The term “modulator” refers to a composition that increases or decreases the level of a target molecule or the function of a target molecule.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine. and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions (e.g. proteins, crowders, nanoclusters, dispersions) of the invention. One of skill in the art will recognize that other pharmaceutical excipients are useful in the present invention.

As used herein, the term “pharmaceutically acceptable” is used synonymously with “physiologically acceptable” and “pharmacologically acceptable”. A pharmaceutical composition will generally include agents for buffering and preservation in storage, and can include buffers and carriers for appropriate delivery, depending on the route of administration.

The terms “isolated” “purified” or “biologically pure” refer to material that is substantially or essentially free from components which normally accompany it as found in its native state. Purity and homogeneity of biological molecules (e.g. nucleic acids or proteins) are typically determined using analytical chemistry techniques such as polyacrylamide gel electrophoresis or high performance liquid chromatography. A protein that is the predominant species present in a preparation is substantially purified. The term “purified” may denote that a nucleic acid or protein gives rise to essentially one band in an electrophoretic gel. In some embodiments, the nucleic acid or protein is at least 50% pure, optionally at least 65% pure, optionally at least 75% pure, optionally at least 85% pure, optionally at least 95% pure, and optionally at least 99% pure. As an example, an isolated cell or isolated sample cells are a single cell type that is substantially free of many of the components which normally accompany the cells when they are in their native state or when they are initially removed from their native state. In certain embodiments, an isolated cell sample retains those components from its natural state that are required to maintain the cell in a desired state. In some embodiments, an isolated (e.g. purified, separated) cell or isolated cells, are cells that are substantially the only cell type in a sample. A purified cell sample may contain at least 60%, 70%, 75%, 80%, 85%, 90%, 95%, 96%, 97%, 98%, 99%, or 100% of one type of cell. An isolated cell sample may be obtained through the use of a cell marker or a combination of cell markers, either of which is unique to one cell type in an unpurified cell sample. In some embodiments, the cells are isolated through the use of a cell sorter. In some embodiments, antibodies against cell proteins are used to isolate cells.

The term “hydrodynamic diameter” has its plain ordinary meaning within Chemistry and refers to the apparent diameter of a hypothetical hard sphere that diffuses through a medium at the same speed as the molecule under observation (e.g. as measured by dynamic light scattering). In embodiments, a nanocluster diameter is a hydrodynamic diameter. Dynamic light scattering measures autocorrelation functions (AF) from entities that scatter light in a dispersion. Larger entities tend to scatter light more intensely than smaller entities and tend to dominate scattering intensity. Algorithms are utilized to determine contribution to AF derived from the various scattering entities. In some embodiments particle (e.g., nanoparticle, nanocluster) diameter is determined from the Stokes-Einstein equation according to the fit of the AF. However, these fits do not in some cases have the resolution to determine the fraction of protein in particle assemblies(e.g., nanoparticles, nanoclusters) versus the fraction of protein in the individual molecular state, or in smaller assemblies. The fits also may not have the resolution to determine the degree to which the diffusing species are coupled to other assemblies of proteins, which may have a range of aggregation numbers.

As used herein, the term “transparent” refers to the physical property of allowing light to pass through a material. In some embodiments, transparent refers to the property of allowing a majority of the incident light, at a given wavelength(s), to pass through the material. In some embodiments, transparent refers to the property of allowing greater than about 75% of the incident light at specified wavelengths (e.g. visible light, 600 nm, 400-700 nm) to pass through the material. In some embodiments, transparent refers to the property of allowing greater than about 80% of the incident light at specified wavelengths to pass through the material. In some embodiments, transparent refers to the property of allowing greater than about 85% of the incident light at specified wavelengths to pass through the material. In some embodiments, transparent refers to the property of allowing greater than about 90% of the incident light at specified wavelengths to pass through the material. In some embodiments, transparent refers to the property of allowing greater than about 95% of the incident light at specified wavelengths to pass through the material. In some embodiments, transparent refers to the property of allowing greater than about 96% of the incident light at specified wavelengths to pass through the material. In some embodiments, transparent refers to the property of allowing greater than about 97% of the incident light at specified wavelengths to pass through the material. In some embodiments, transparent refers to the property of allowing greater than about 98% of the incident light at specified wavelengths to pass through the material. In some embodiments, transparent refers to the property of allowing greater than about 99% of the incident light at specified wavelengths to pass through the material. In some embodiments, transparent refers to the property of allowing greater than about 99.5% of the incident light at specified wavelengths to pass through the material. In some embodiments, transparent refers to the property of allowing greater than about 99.6% of the incident light at specified wavelengths to pass through the material. In some embodiments, transparent refers to the property of allowing greater than about 99.7% of the incident light at specified wavelengths to pass through the material. In some embodiments, transparent refers to the property of allowing greater than about 99.8% of the incident light at specified wavelengths to pass through the material. In some embodiments, transparent refers to the property of allowing greater than about 99.9% of the incident light at specified wavelengths to pass through the material In some embodiments, when referring to the transparency of a dispersion of protein nanoclusters, the percentages above (e.g. percentage value of any one of 75, 80, 85, 90, 95, 96, 97, 98, 99, 99.5, 99.6, 99.7, 99.8, 99.9), is in comparison to a control sample lacking the protein, which would be assigned the value of 100% incident light at specified wavelengths passing through the material. In some embodiments, transparency is measured by light extinction, wherein the term “light extinction” as used herein refers to the combined absorption and scattering of incident light at zero degrees from the angle of the incident light. In some embodiments, transparent means having a light extinction of less than about 0.05 cm⁻¹. In some embodiments, transparent means having a light extinction of less than about 0.1 cm⁻¹. In some embodiments, transparent means having a light extinction of less than about 0.25 cm⁻¹. In some embodiments, transparent means having a light extinction of less than about 0.5 cm⁻¹. In some embodiments, transparent means having a light extinction of less than about 1%. In some embodiments, transparent means having a light extinction of less than about 2%. In some embodiments, transparent means having a light extinction of less than about 3%. In some embodiments, transparent means having a light extinction of less than about 4%. In some embodiments, transparent means having a light extinction of less than about 5%. In some embodiments, transparent means having a light extinction of less than about 10%. In some embodiments, transparent means having a light extinction of less than about 15%. In some embodiments, transparent means having a light extinction of less than about 20%. In some embodiments, transparent means having a light extinction of less than about 25%. In some embodiments, when referring to the transparency of a dispersion of protein particles (e.g., nanoparticles, nanoclusters) in terms of light extinction, the percentages above (e.g. percentage value of any one of 1, 2, 3, 4, 5, 10, 15, 20, or 25), is in comparison to a control sample lacking the protein, which would be assigned the value of 0% light extinction. A “light extinction measurement” refers to a light extinction value physically measured by a person of ordinary skill.

The term “viscosity” has its plain ordinary meaning within Chemistry, as applied to liquids and fluids.

As used herein, the term “low viscosity” refers to a viscosity that is less than about 100 centipoise. In some embodiments, “low viscosity” refers to a viscosity of less than about 90 centipoise. In some embodiments, “low viscosity” refers to a viscosity of less than about 80 centipoise. In some embodiments, “low viscosity” refers to a viscosity of less than about 70 centipoise. In some embodiments, “low viscosity” refers to a viscosity of less than about 60 centipoise. In some embodiments, “low viscosity” refers to a viscosity of less than about 50 centipoise. In some embodiments, “low viscosity” refers to a viscosity of less than about 40 centipoise. In some embodiments, “low viscosity” refers to a viscosity of less than about 30 centipoise. In some embodiments, “low viscosity” refers to a viscosity of less than about 20 centipoise. In some embodiments, “low viscosity” refers to a viscosity of less than about 10 centipoise. In some embodiments, a low viscosity is measured with a viscometer, rheometer, or syringe loading method as described herein. In some embodiments, a low viscosity is measured with a shear rate that is about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 150000, or 200000 second⁻¹. In some embodiments, an average shear rate may be determined from the flow rate and geometric properties with a syringe loading method as described herein. In some embodiments, “low viscosity” refers to any one of the combinations of viscosity and shear rate shown in the table/matrix below having number 1 to 280, wherein each cell corresponds to the viscosity for that column and the shear rate for that row:

| shear rate | Viscosity less than about(centipoise) | | | | | | | | | |
|---|---|---|---|---|---|---|---|---|---|---|
| about | 100 | 90 | 80 | 70 | 60 | 50 | 40 | 30 | 20 | 10 |
| (second⁻¹) | cP | cP | cP | cP | cP | cP | cP | cP | cP | cP |
| 100 | 1 | 2 | 3 | 4 | 5 | 6 | 7 | 8 | 9 | 10 |
| 200 | 11 | 12 | 13 | 14 | 15 | 16 | 17 | 18 | 19 | 20 |
| 300 | 21 | 22 | 23 | 24 | 25 | 26 | 27 | 28 | 29 | 30 |
| 400 | 31 | 32 | 33 | 34 | 35 | 36 | 37 | 38 | 39 | 40 |
| 500 | 41 | 42 | 43 | 44 | 45 | 46 | 47 | 48 | 49 | 50 |
| 600 | 51 | 52 | 53 | 54 | 55 | 56 | 57 | 58 | 59 | 60 |
| 700 | 61 | 62 | 63 | 64 | 65 | 66 | 67 | 68 | 69 | 70 |
| 800 | 71 | 72 | 73 | 74 | 75 | 76 | 77 | 78 | 79 | 80 |
| 900 | 81 | 82 | 83 | 84 | 85 | 86 | 87 | 88 | 89 | 90 |
| 1000 | 91 | 92 | 93 | 94 | 95 | 96 | 97 | 98 | 99 | 100 |
| 2000 | 101 | 102 | 103 | 104 | 105 | 106 | 107 | 108 | 109 | 110 |
| 3000 | 111 | 112 | 113 | 114 | 115 | 116 | 117 | 118 | 119 | 120 |
| 4000 | 121 | 122 | 123 | 124 | 125 | 126 | 127 | 128 | 129 | 130 |
| 5000 | 131 | 132 | 133 | 134 | 135 | 136 | 137 | 138 | 139 | 140 |
| 6000 | 141 | 142 | 143 | 144 | 145 | 146 | 147 | 148 | 149 | 150 |
| 7000 | 151 | 152 | 153 | 154 | 155 | 156 | 157 | 158 | 159 | 160 |
| 8000 | 161 | 162 | 163 | 164 | 165 | 166 | 167 | 168 | 169 | 170 |
| 9000 | 171 | 172 | 173 | 174 | 175 | 176 | 177 | 178 | 179 | 180 |
| 10000 | 181 | 182 | 183 | 184 | 185 | 186 | 187 | 188 | 189 | 190 |
| 20000 | 191 | 192 | 193 | 194 | 195 | 196 | 197 | 198 | 199 | 200 |
| 30000 | 201 | 202 | 203 | 204 | 205 | 206 | 207 | 208 | 209 | 210 |
| 40000 | 211 | 212 | 213 | 214 | 215 | 216 | 217 | 218 | 219 | 220 |
| 50000 | 221 | 222 | 223 | 224 | 225 | 226 | 227 | 228 | 229 | 230 |
| 60000 | 231 | 232 | 233 | 234 | 235 | 236 | 237 | 238 | 239 | 240 |
| 70000 | 241 | 242 | 243 | 244 | 245 | 246 | 247 | 248 | 249 | 250 |
| 80000 | 251 | 252 | 253 | 254 | 255 | 256 | 257 | 258 | 259 | 260 |
| 90000 | 261 | 262 | 263 | 264 | 265 | 266 | 267 | 268 | 269 | 270 |
| 100000 | 271 | 272 | 273 | 274 | 275 | 276 | 277 | 278 | 279 | 280 |

In some embodiments, the viscosity is the value in the column heading and the shear rate is the value in the row heading that together correspond to any one of the cells number 1 to 280 in the table immediately above. In some embodiments, the viscosity and shear rate combinations in the table above are measured by a syringe loading method as described herein. In embodiments, viscosity is measured at room temperature. In embodiments, viscosity is measured at ambient temperature. In embodiments, viscosity is measured at physiological temperature. In embodiments, viscosity is measured at about 21, 22, 23, 24, 25, 26, or 27 degrees Celsius. In embodiments, viscosity is measured between 20 and 23.5 degrees Celsius. In embodiments, viscosity is measured with a rheometer. In embodiments, viscosity is measured with a viscometer.

In some embodiments, the low viscosity is less than about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 centipoise; at a temperature that is about 21, 22, 23, 24, 25, 26, 27 degrees Celsius, or ambient temperature, or room temperature; at a shear rate within the range represented by a cell in the table below, wherein the shear rate range is between (inclusive) the shear rate in the column value and row value of the cell. For example the cell number 100 corresponds to a shear rate range of 1000 to 10,000 per second.

| a shear rate about | | | | | | | | | | |
|---|---|---|---|---|---|---|---|---|---|---|
| (second⁻¹) between | | | | | | | | | | |
| (and including) the | | | | | | | | | | |
| column value | | | | | | | | | | |
| below and row | | | | | | | | | | |
| value to the right. | 1000 | 2000 | 3000 | 4000 | 5000 | 6000 | 7000 | 8000 | 9000 | 10000 |
| 100 | 1 | 2 | 3 | 4 | 5 | 6 | 7 | 8 | 9 | 10 |
| 200 | 11 | 12 | 13 | 14 | 15 | 16 | 17 | 18 | 19 | 20 |
| 300 | 21 | 22 | 23 | 24 | 25 | 26 | 27 | 28 | 29 | 30 |
| 400 | 31 | 32 | 33 | 34 | 35 | 36 | 37 | 38 | 39 | 40 |
| 500 | 41 | 42 | 43 | 44 | 45 | 46 | 47 | 48 | 49 | 50 |
| 600 | 51 | 52 | 53 | 54 | 55 | 56 | 57 | 58 | 59 | 60 |
| 700 | 61 | 62 | 63 | 64 | 65 | 66 | 67 | 68 | 69 | 70 |
| 800 | 71 | 72 | 73 | 74 | 75 | 76 | 77 | 78 | 79 | 80 |
| 900 | 81 | 82 | 83 | 84 | 85 | 86 | 87 | 88 | 89 | 90 |
| 1000 | | 92 | 93 | 94 | 95 | 96 | 97 | 98 | 99 | 100 |
| 2000 | | | 103 | 104 | 105 | 106 | 107 | 108 | 109 | 110 |
| 3000 | | | | 114 | 115 | 116 | 117 | 118 | 119 | 120 |
| 4000 | | | | | 125 | 126 | 127 | 128 | 129 | 130 |
| 5000 | | | | | | 136 | 137 | 138 | 139 | 140 |
| 6000 | | | | | | | 147 | 148 | 149 | 150 |
| 7000 | | | | | | | | 158 | 159 | 160 |
| 8000 | | | | | | | | | 169 | 170 |
| 9000 | | | | | | | | | | 180 |

As used herein, the term “high protein concentration” or “high protein” refers to a protein concentration of greater than about 200 mg/mL. In some embodiments, the protein concentration is greater than about 300 mg/mL. In some embodiments, the protein concentration is greater than about 400 mg/mL. In some embodiments, the protein concentration is greater than about 500 mg/mL. In some embodiments, the protein concentration is greater than about 600 mg/mL. In some embodiments, the protein concentration is greater than about 700 mg/mL. In some embodiments, the protein concentration is greater than about 800 mg/mL. In some embodiments, the protein concentration is greater than about 900 mg/mL. In some embodiments, the protein concentration is greater than about 1000 mg/mL. In some embodiments, the protein concentration is the concentration of one protein species (proteins substantially identical). In some embodiments, the protein concentration is the concentration of all proteins in a mixture. In some embodiments, “high protein concentration” or “high protein” refers to a protein concentration that is greater than about 200, 300, 400, 500, 600, 700, 800, 900, or 1000 mg/mL. In some embodiments “high protein concentration” or “high protein” refers to a protein concentration range, wherein the range is entirely greater than about 200, 300, 400, 500, 600, 700, 800, 900, or 1000 mg/mL. In some embodiments, “high protein concentration” or “high protein” refers to a protein concentration that is greater than about 250 mg/mL. In some embodiments, “high protein concentration” or “high protein” refers to a protein concentration that is greater than about 200, 210, 220, 230, 240, 250, 260, 270, 280, 290, or 300 mg/mL.

As used herein, the term “dispersion” has its plain ordinary meaning within the field of Chemistry and refers to a system containing particles dispersed in a continuous phase of a different composition (e.g. particles, nanoparticles or nanoclusters dispersed in a liquid phase). In some embodiments, a dispersion may be a suspension, wherein a suspension has its plain ordinary meaning within Chemistry and refers to a dispersion of solid particles in a continuous liquid phase, wherein the solid particles are large enough for sedimentation. In some embodiments, a dispersion may be a colloid, wherein a colloid has its plain ordinary meaning as used within Chemistry. In some embodiments, a dispersion includes nanoparticles dispersed in a continuous liquid phase. In some embodiments the dispersed particles are protein nanoclusters. In some embodiments, the continuous phase of a different composition includes protein in solution. In some embodiments, the protein in solution is less than about 50%, 40%, 30%, 20%, 10%, 9%, 8%, 7%, 6%, 5%, 4%, 3%, 2%, 1%, 0.5%, 0.4%, 0.3%, 0.2%, 0.1%, of the total protein in the dispersion (i.e. particles and continuous phase combined).

The terms “polypeptide,” “peptide” and “protein” are used interchangeably herein to refer to a polymer of amino acid residues, wherein the polymer may optionally be conjugated to a moiety that does not consist of amino acids (e.g. small molecular weight compounds). The terms apply to amino acid polymers in which one or more amino acid residue is an artificial chemical mimetic of a corresponding naturally occurring amino acid, as well as to naturally occurring amino acid polymers and non-naturally occurring amino acid polymer. In some embodiments, a protein includes a non-protein composition (e.g. low molecular weight compound) conjugated (e.g. bonded) to the polymer of amino acid residues (collectively a “conjugate” or “conjugated protein”). In some embodiments, a protein consists of a polymer of amino acids (a “non-conjugated protein”). In some embodiments, a protein is a polymer of about 10, 20, 30, 40, 50, 60, 70, 80, 90, 100, 200, 300, 400, 500, 600, 700, 800, 900, 1000, 2000, 3000, 4000, or 5000 amino acid residues. In some embodiments, a protein is a polymer of 2, 3, 4, 5, 6, 7, 8, 9, 10, 11, 12, 13, 14, 15, 16, 17, 18, 19, or 20 amino acid residues.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. In embodiments, an amino acid is arginine, histidine, lysine, aspartic acid, glutamic acid, serine, threonine, asparagine, glutamine, cysteine, glycine, proline, alanine, valine, isoleucine, leucine, methionine, phenylalanine, tyrosine, or tryptophan.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “crowder” refers to a compound that, when present in a solvent (e.g. a dispersion liquid) with concentrated proteins, aids formation of a stable colloidal dispersion containing particles (e.g., nanoparticles, nanoclusters) of non-irreversibly aggregated proteins. In some embodiments, a crowder may be the protein itself (e.g. self-crowding protein). In some embodiments, the crowder may be an amino acid (e.g., proline, histidine, lysine, glutamic acid, asparagine, glutamine, betaine, or arginine). In some embodiments, the crowder may be a second protein species (e.g. a dipeptide, tripeptide, oligopeptide, conjugated protein, non-conjugated protein). In some embodiments, the crowder may be a non-protein crowder such as a polysaccharide, polyelectrolyte, polyacid, dextran, polaxamer, surfactant, a glycerol, an erythritol, an arabinose, a xylose, a ribose, an inositol, a fructose, a galactose, a maltose, a glucose, a mannose, a trehalose, a sucrose, a poly(ethylene glycol), a carbomer 1342, a glucose polymers, a silicone polymer, a polydimethylsiloxane, a polyethylene glycol, a carboxy methyl cellulose, a poly(glycolic acid), a poly(lactic-co-glycolic acid), a polylactic acid, a dextran, a poloxamers, imidazole, organic co-solvents selected from ethanol, N-methyl-2-pyrrolidone (NMP), PEG 300, PEG 400, PEG 200, PEG 3350, Propylene Glycol, N,N Dimethylacetamide, dimethyl sulfoxide, solketal, tetahydrofurfuryl alcohol, diglyme, ethyl lactate, a salt, a buffer or a combination thereof. In embodiments, a viscosity lowering agent is a crowder. In some embodiments, the crowder may be citric acid.

The terms “polysaccharide”, “polyelectrolyte”, “polyacid”, “polaxamer”, “surfactant”, “buffer” have their plain ordinary meaning within the field of Chemistry.

As used herein, the term “dextran” refers to a branched polysaccharide including glucose molecules. In some embodiments, the dextran has a molecular weight between about one to 2000 kilodaltons. In some embodiments, the dextran is one kilodalton. In some embodiments, the molecular weight is between about one and 10 kilodaltons. In some embodiments, the molecular weight is between about one and 100 kilodaltons. In some embodiments, the molecular weight is between about one and 1000 kilodaltons. In some embodiments, the molecular weight is between about 10 and 100 kilodaltons. In some embodiments, the molecular weight is between about 10 and 50 kilodaltons. In some embodiments, the molecular weight is between about 10 and 2000 kilodaltons. In some embodiments, the molecular weight is between about 100 and 2000 kilodaltons. In some embodiments, the molecular weight is between about 100 and 500 kilodaltons.

As used herein, the term “about”, when modifying a number (e.g. an amount, measurement, size, viscosity, diameter, concentration), refers to a range of values, including the number, and values greater and/or less than the number, wherein the range is an amount that would not affect the function or use of a composition or method, as described herein (including embodiments thereof) when compared to the function applied with exactly the number. In some embodiments the range would not significantly affect the function. In some embodiments, the range would not substantially affect the function. In some embodiments, the composition is a particle (e.g., nanoparticle, nanocluster) as described herein (including embodiments thereof). In some embodiments, the composition is a dispersion of particles (e.g., nanoparticles, nanoclusters) as described herein (including embodiments thereof). In some embodiments, the composition is a pharmaceutical composition, as described herein (including embodiments thereof). In some embodiments, the composition is a kit as described herein (including embodiments thereof). In some embodiments, the method is a method of making a dispersion as described herein (including embodiments thereof). In some embodiments, the method is a method of treating a disease, as described herein (including embodiments thereof). In some embodiments, the method is a method of modifying a dispersion of nanoclusters, as described herein (including embodiments thereof). In some embodiments, about includes 0.1, 0.2, 0.3, 0.4, 0.5, 0.6, 0.7, 0.8, or 0.9 times an increase, decrease, or both, of the number. In some embodiments, about is exactly the number. In some embodiments, about is the standard deviation of the number when measured by a person of ordinary skill in measuring the number, using a common technique or apparatus for taking such measurement. In some embodiments, about includes 0.1, 1.0, or 10 times the number. In some embodiments, about includes plus and minus 0.1 times the number (e.g. about 200 mg/mL is 180-220 mg/mL).

As used herein, the term “average diameter”, when applied to particles (e.g., nanoparticles, nanoclusters), refers to the average diameter of the particles (e.g., nanoparticles, nanoclusters) in a sample. In some embodiments, the average diameter is an average hydrodynamic diameter. In some embodiments, the average diameter is an average nanocluster diameter. In some embodiments, the average diameter is the average length of the longest axis of the particle (e.g., nanoparticle, nanocluster) or nanoparticles. In some embodiments the average diameter is measured by dynamic light scattering. In some embodiments the average diameter is measured by static light scattering. In some embodiments the average diameter is measured by size exclusion chromatography. In some embodiments the average diameter is measured by microscopy. In some embodiments the average diameter is measured by scanning electron microscopy. In some embodiments the average diameter is measured by cryoelectron microscopy. In some embodiments the average diameter is measured by transmission electron microscopy. In some embodiments the average diameter is measured by x-ray scattering (e.g. small angle x-ray scattering).

As used herein, the term “plurality” refers to more than one.

As used herein, the term “irreversibly aggregated” refers to proteins physically associated together in a mixture, including a liquid medium, wherein upon dilution of the concentration of the protein or concentration of crowder if the mixture contains crowder, the proteins do not dissociate from the aggregates to form functional protein possessing the secondary, tertiary, and quaternary structure appropriate for the medium and concentration of protein if the protein had not previously been aggregated. An irreversibly aggregated protein may also be termed an “unstable” protein. A “stable” protein (e.g. antibody) is a protein that dissociates from a protein aggregate or protein nanocluster upon dilution of either the protein concentration or crowder concentration, if a crowder is present and promoted the formation of the protein aggregate or protein nanocluster, to form functional (e.g. active, enzymatically active) proteins possessing the secondary, tertiary, and quaternary structure appropriate for the medium and concentration of protein if the protein had not previously been aggregated.

As used herein, the term “low molecular weight compound” refers to a composition having a molecular weight less than 1 kilodalton. In some embodiments, the low molecular weight compound may be a diagnostic agent, pharmaceutical agent, contrast agent, fluorophore, paramagnetic agent, peptide, or toxin. In some embodiments, the low molecular weight compound may be conjugated (e.g. bonded) to another composition (e.g. protein, antibody).

As used herein, the term “diagnostic agent” refers to a composition that is useful for detecting the presence of a disease state or a symptom of a disease state. In some embodiments, a diagnostic agent may be a label or detectable moiety.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, Bioconjugate Techniques 1996, Academic Press, Inc., San Diego.

As used herein, a “pharmaceutical” refers to a composition that is useful in the treatment of a disease or a symptom of a disease.

As used herein, a “pharmaceutically active protein” refers to a protein that is useful in the treatment of a disease or a symptom of a disease.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein could successfully treat cancer by decreasing the incidence of cancer and or causing remission of cancer. The term “treating,” and conjugations thereof, include prevention of an injury, pathology, condition, or disease.

“Disease” or “condition” refer to a state of being or health status of a patient or subject capable of being treated with the compositions, dispersions, or methods provided herein.

As used herein, the term “contrast agent” refers to a composition that, when administered to a subject, improves the detection limit or detection capability of a method, technique, or apparatus for medical imaging (e.g. radiographic instrument, X-ray, CT, PET, MRI, ultrasound). A contrast agent may enhance the contrast of signals related to different structures or fluids within a subject.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

As used herein, the term “fluorophore” has its plain ordinary meaning within Chemistry and refers to a chromophore used in fluorescent imaging of spectroscopy. A fluorophore absorbs light within a first range of wavelengths and emits light within a second range of wavelengths.

As used herein, the term “shear rate” has its plain ordinary meaning within Chemistry and fluid mechanics and refers to the rate (e.g. seconds⁻¹) of application of a shear, wherein shear refers to a force or pressure applied to an object (e.g. deformable object, liquid, solid object) perpendicular to a given axis with greater value (i.e. greater force or pressure) on one side of the axis compared to the other. For flow through a cylinder, shear rate at the wall is proportional to the flow rate divided by the cube of the radius.

As used herein, the term “syringe loading method” refers to a method of measuring the viscosity of a liquid (e.g. dispersion, solution, suspension, mixture) by using the same pressure drop in a needle attached to a syringe wherein the piston of the syringe is displaced by a set amount, causing flow through the needle, wherein the needle has a known diameter and length. The unknown viscosity of liquid being measured is compared to a plurality of measurements conducted in exactly the same way as the current measurement, wherein the plurality of measurements is conducted on liquids with known viscosities. In some embodiments, the needle has a gauge between 21 and 27. In some embodiments, the needle is a 25 gauge needle. In some embodiments, the syringe is a 1 mL syringe. In some embodiments, the needle is 1.5 inches long. In some embodiments, the time to draw the liquid (e.g. dispersion) from a height in a conical vial, wherein the distance from the liquid meniscus to the bottom of the cone is at 0.4 inches, to a height, wherein the distance from the liquid meniscus to the bottom of the cone is at 0.1 inches, corresponding to a volume of 48 microliters, is measured.

As used herein, the term “packing fraction” refers to a ratio of the volume occupied by a first object or plurality of first objects to the volume of a defined space containing the first object or plurality of objects and a second object or plurality of objects. In some embodiments, a packing fraction is the ratio of the volume of protein within a particle (e.g., nanoparticle, nanocluster) to the volume of the particle (e.g., nanoparticle, nanocluster). In some embodiments, the packing fraction is the average of a plurality of ratios of the volume of protein within a particle (e.g., nanoparticle, nanocluster) to the volume of the particle (e.g., nanoparticle, nanocluster).

As used herein, the term “isotonic” refers to two liquids having the same osmotic pressure. A liquid is isotonic with another if it has the same effective osmotic pressure as the liquid inside the cell across the membrane of a given type of cell. In some embodiments, a dispersion is isotonic with blood. In some embodiments, a dispersion is isotonic with the site of administration of the dispersion in a patient. In some embodiments, a dispersion is isotonic with a subcutaneous site of administration of the dispersion.

As used herein, the term “antibody” refers to a polypeptide including a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of a protein. Such effector moieties include, but are not limited to, an anti-tumor drug, a toxin, a radioactive agent, a cytokine, a second antibody or an enzyme. In some embodiments, the antibody of the invention is linked to an enzyme that converts a prodrug into a cytotoxic agent.

The immunoconjugate can be used for targeting the effector moiety to a target molecule or target molecule positive cell. Such differences can be readily apparent when viewing the bands of gels with approximately similarly loaded with test and controls samples. Examples of cytotoxic agents (e.g. toxins) include, but are not limited to ricin, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, and glucocorticoid and other chemotherapeutic agents, as well as radioisotopes. Suitable detectable markers include, but are not limited to, a radioisotope, a fluorescent compound, a bioluminescent compound, chemiluminescent compound, a metal chelator or an enzyme.

Techniques for conjugating therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

As used herein, the term “protein-crowder liquid combination” refers to a liquid mixture including a plurality of a protein and a plurality of a crowder. In some embodiments, the protein-crowder liquid combination is a dispersion of protein nanoclusters. In some embodiments, the protein-crowder liquid combination is a dispersion of particles (e.g., nanoparticles, nanoclusters) including a plurality of proteins and a plurality of crowder. In some embodiments, the protein-crowder liquid combination is a suspension of particles (e.g., nanoparticles, nanoclusters) including a plurality of protein. In some embodiments, the protein-crowder liquid combination is a solution including a plurality of protein and a plurality of crowder.

As used herein, the term “protein-crowder mixture” refers to a mixture of protein and crowder, which may optionally include additional components or compounds. In some embodiments, a “protein-crowder mixture” is a “protein-crowder liquid combination”.

As used herein, the term “dispersion liquid” refers to the continuous liquid mixture of a dispersion. In some embodiments, a dispersion liquid is a liquid solution in which protein particles (e.g., nanoparticles, nanoclusters) are dispersed. In some embodiments, a dispersion liquid is a non-aqueous liquid in which protein particles (e.g., nanoparticles, nanoclusters) are dispersed. In some embodiments, a dispersion liquid is an aqueous liquid in which protein particles (e.g., nanoparticles, nanoclusters) are dispersed.

As used herein, the term “cryogenic agent” refers to a composition having a temperature below −150 degrees Celsius. In some embodiments, a cryogenic agent is liquid nitrogen. In some embodiments, a cryogenic agent is liquid helium.

As used herein, the term “centrifugal filtration” refers to the process of filtering or separating components in a mixture by flowing one or more, but not all, components of the mixture through a filter, wherein the components are moved through the filter by centrifugal force. In some embodiments, the mixture and filter are spun in a centrifuge. In some embodiments the filtration is carried out in an Amicon, Microcon, or Centricon device (available from Millipore).

As used herein, the term “tangential flow filtration” refers to a method of filtration wherein the majority of movement of the liquid mixture, prior to passing through the filter, is tangential to the surface of the filter. The term “crossflow filtration” may be used interchangeably with “tangential flow filtration”.

As used herein, the term “protein solution” refers to a mixture of a plurality of protein in a liquid medium (e.g. water, buffer), wherein the protein does not form particles (e.g., nanoparticles, nanoclusters) having an average diameter of 20 to 1000 nm. In some embodiments, a protein solution includes proteins having a quaternary state appropriate for the dissociate constant of the protein and concentration of protein mixed in the liquid, without forming nanoclusters of 10 or more proteins. In some embodiments, a protein dispersion may include a dispersion of protein particles (e.g., nanoparticles, nanoclusters) in a protein solution.

As used herein, the term “thin film freezing” refers to a method including freezing a liquid on a cooled solid surface, wherein the liquid forms a thin film on the surface of thickness less than 500 micrometers and a surface area to volume ratio between 25 and 500 cm⁻¹. In some embodiments, the liquid and surface have temperatures differing by about 30 degrees Celsius or more. The liquid may be delivered to the cooled solid surface as droplets. In some embodiments, the droplets freeze within 50, 75, 100, 125, 150, 175, 200, 250, 500, 1,000, or 2,000 milliseconds of contacting the cooled solid surface. In some embodiments, the droplets may have an average diameter of 0.1 mm to 5 mm at room temperature. In some embodiments, the droplets will have a cooling rate of between 50 and 250 K/second. The cooled solid surface may be the interior surface of a vial, a belt, platen, plate, roller, platter, or conveyor surface.

As used herein, the term “viscosity lowering agent” refers to a composition capable of reducing the viscosity of a second composition (e.g., dispersion) when the viscosity lowering agent is contacts the second composition (e.g., is added to the second composition or is mixed with the second composition). Examples of viscosity lowering agents include histidine, arginine, proline, imidazole, trehalose, citric acid, lysine, glutamic acid, glutamine, asparagine, and sucrose. Other additives and crowders may also be viscosity lowering agents. A viscosity lower agent may be a crowder.

An “effective amount” is an amount sufficient to accomplish a stated purpose (e.g. achieve the effect for which it is administered, treat a disease, reduce enzyme activity, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.”

A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The terms “identical” or percent sequence “identity,” or “shares amino acid sequence identity” in the context of two or more nucleic acids or polypeptide sequences, refer to two or more sequences or subsequences that are the same or have a percentage of amino acid residues or nucleotides that are the same over a specified region, or have a specified percentage of amino acid residues or nucleotides that are the same (i.e., about 60% identity, preferably about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site at ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. Employed algorithms can account for gaps and the like.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively.

Aptamers are nucleic acids that are designed to bind to a wide variety of targets in a non-Watson Crick manner. An aptamer can thus be used to detect or otherwise target nearly any molecule of interest, including an autoimmune, inflammatory autoimmune, cancer, infectious disease, or other disease associated protein. Methods of constructing and determining the binding characteristics of aptamers are well known in the art. For example, such techniques are described in U.S. Pat. Nos. 5,582,981, 5,595,877 and 5,637,459. Aptamers are typically at least 5 nucleotides, 10, 20, 30 or 40 nucleotides in length, and can be composed of modified nucleic acids to improve stability. Flanking sequences can be added for structural stability, e.g., to form 3-dimensional structures in the aptamer.

As used herein, the term “controlled release component” refers to a compound that when combined with a composition as described herein (including embodiments) releases the composition at a controlled rate into a patient. Such compounds include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. In some embodiments, the control release component may be a sustained release, sustained action, extended release, time release, timed release, controlled release, modified release, or continuous release compound. In some embodiments, the compound is degraded by the patient at the site of administration (e.g. subcutaneous, intravenous) or within the digestive tract (e.g. stomach, intestines) if the compound and composition are administered orally. In some embodiments, the controlled release component is a polymer and may be called a “controlled release polymer”.

As used herein, the term “paramagnetic agent” refers to a paramagnetic compound useful in diagnostic imaging methods (e.g. magnetic resonance imaging) as a contrast agent. In some embodiments, the paramagnetic agent includes gadolinium, iron oxide, iron platinum, or manganese.

As used herein, the term “saccharide” is used in accordance with its plain ordinary meaning and is synonymous with carbohydrate. Examples of a saccharide include, but are not limited to, monosaccharides, disaccharides, oligosaccharides, and polysaccharides. Examples of saccharides include, but are not limited to, trehalose, sucrose, sorbitol, glucose, fructose, xylose, galactose, mannitol, and raffinose.

### Low Viscosity Dispersions

In an aspect is provided a low viscosity dispersion including a protein and a viscosity lowering agent. The viscosity lowering agent is present at a concentration from about 10 mg/mL to about 300 mg/mL. The protein is present at a concentration of greater than about 200 mg/mL. The protein is present within a plurality of protein nanoclusters (e.g., nanoparticles). The viscosity lowering agent is proline, histidine, arginine, glutamic acid, betaine, glutamine, lysine, asparagine, or imidazole.

In embodiments, the low viscosity dispersion includes the presence of a conjugate acid or base. In embodiments, the low viscosity dispersion includes the presence of a buffer. In embodiments, the conjugate acid is citric acid. In embodiments, the conjugate acid is HCl.

In embodiments, the viscosity is 25 cP or less. The viscosity may be 15 cP or less. The viscosity may be between about 50 and 2 cP. The viscosity may be between about 45 and 2 cP. The viscosity may be between about 40 and 2 cP. The viscosity may be between about 35 and 2 cP. The viscosity may be between about 30 and 2 cP. The viscosity may be between about 25 and 2 cP. The viscosity may be between about 20 and 2 cP. The viscosity may be between about 15 and 2 cP. The viscosity may be between about 10 and 2 cP. The viscosity may be between about 50 and 5 cP. The viscosity may be between about 25 and 5 cP. The viscosity may be between about 15 and 5 cP. The viscosity may be between about 50 and 10 cP. The viscosity may be between about 25 and 10 cP. The viscosity may be between about 15 and 10 cP. The viscosity may be between about 2 and 100 cP. The viscosity may be between about 2 and 90 cP. The viscosity may be between about 2 and 80 cP. The viscosity may be between about 2 and 70 cP. The viscosity may be between about 2 and 60 cP. The viscosity may be between about 10 and 100 cP. The viscosity may be between about 20 and 100 cP. The viscosity may be between about 30 and 100 cP. The viscosity may be between about 40 and 100 cP. The viscosity may be between about 50 and 100 cP. The viscosity may be between about 75 and 100 cP. In embodiments, the viscosity described herein (e.g., above) is with a shear rate of about 5 sec⁻¹ to about 200,000 sec⁻¹. In embodiments, the viscosity described herein is with a shear rate of about 5 sec⁻¹ to about 150,000 sec⁻¹. In embodiments, the viscosity described herein is with a shear rate of about 100 sec⁻¹ to about 10,000 sec⁻¹. In embodiments, the viscosity described herein (e.g., above) is with a shear rate of about 500 sec⁻¹ to about 5,000 sec⁻¹. In embodiments, the viscosity described herein (e.g., above) is with a shear rate of about 1000 sec⁻¹ to about 5,000 sec⁻¹. In embodiments, the viscosity described herein (e.g., above) is with a shear rate of about 2,000 sec⁻¹ to about 4,000 sec⁻¹.

In embodiments, the dispersion further includes a saccharide (e.g., trehalose, sucrose, sorbitol, glucose, fructose, xylose, galactose, mannitol, or raffinose). In embodiments, the dispersion further includes trehalose or sucrose. The dispersion may further include trehalose. The dispersion may further include sucrose. In embodiments, the saccharide (e.g., trehalose, sucrose, sorbitol, glucose, fructose, xylose, galactose, mannitol, or raffinose) is present at a concentration between about 10 mg/mL and about 200 mg/mL (e.g., between about 25 and 150, 50 and 150, 75 and 150, 100 and 200, 125 and 200, 10 and 150, or 10 and 100 mg/mL). In embodiments, the saccharide (e.g., trehalose, sucrose, sorbitol, glucose, fructose, xylose, galactose, mannitol, or raffinose) is present at a concentration between about 30 mg/mL and about 60 mg/mL. In embodiments, the saccharide (e.g., trehalose, sucrose, sorbitol, glucose, fructose, xylose, galactose, mannitol, or raffinose) is present at a concentration between about 30 mg/mL and about 200 mg/mL (e.g., between about 30 and 175, 30 and 160, 30 and 150, 30 and 125, 30 and 100, 30 and 75, 30 and 60, or 30 and 60 mg/mL). In embodiments, the saccharide (e.g., trehalose, sucrose, sorbitol, glucose, fructose, xylose, galactose, mannitol, or raffinose) is present at a concentration between about 10 mg/mL and about 200 mg/mL (e.g., between about 25 and 150, 50 and 150, 75 and 150, 100 and 200, 125 and 200, 10 and 150, or 10 and 100 mg/mL). In embodiments, the saccharide (e.g., trehalose, sucrose, sorbitol, glucose, fructose, xylose, galactose, mannitol, or raffinose) is present at a concentration between about 30 mg/mL and about 60 mg/mL. In embodiments, the saccharide (e.g., trehalose, sucrose, sorbitol, glucose, fructose, xylose, galactose, mannitol, or raffinose) is present at a concentration between about 30 mg/mL and about 200 mg/mL (e.g., between about 30 and 175, 30 and 160, 30 and 150, 30 and 125, 30 and 100, 30 and 75, 30 and 60, or 30 and 60 mg/mL). In embodiments, the saccharide is trehalose. In embodiments, the saccharide is sucrose. In embodiments, the saccharide is sorbitol. In embodiments, the saccharide is glucose. In embodiments, the saccharide is fructose, xylose. In embodiments, the saccharide is galactose. In embodiments, the saccharide is mannitol. In embodiments, the saccharide is raffinose.

In embodiments, the viscosity lowering agent is histidine. In embodiments, the viscosity lowering agent is imidazole. In embodiments, the viscosity lowering agent is proline. In embodiments, the viscosity lowering agent is arginine. In embodiments, the viscosity lowering agent is glutamic acid. In embodiments, the viscosity lowering agent is betaine. In embodiments, the viscosity lowering agent is glutamine. In embodiments, the viscosity lowering agent is asparagine. In embodiments, the viscosity lowering agent is lysine. In embodiments, the dispersion includes a plurality of different viscosity lowering agents. In embodiments, the dispersion includes two different viscosity lowering agents selected from proline, histidine, arginine, glutamic acid, betaine, glutamine, asparagine, lysine, and imidazole. In embodiments, the dispersion includes arginine and glutamate. In embodiments, the arginine is present at a concentration from about 20 mg/mL to about 150 mg/mL and glutamic acid is present at a concentration from about 20 mg/mL to about 150 mg/mL. In embodiments, the arginine is present at a concentration from about 20 mg/mL to about 125 mg/mL and glutamic acid is present at a concentration from about 20 mg/mL to about 125 mg/mL. In embodiments, the arginine is present at a concentration from about 20 mg/mL to about 100 mg/mL and glutamic acid is present at a concentration from about 20 mg/mL to about 100 mg/mL. In embodiments, the arginine is present at a concentration from about 50 mg/mL to about 150 mg/mL and glutamic acid is present at a concentration from about 50 mg/mL to about 150 mg/mL. In embodiments, the arginine is present at a concentration from about 50 mg/mL to about 125 mg/mL and glutamic acid is present at a concentration from about 50 mg/mL to about 125 mg/mL. In embodiments, the arginine is present at a concentration from about 75 mg/mL to about 125 mg/mL and glutamic acid is present at a concentration from about 75 mg/mL to about 125 mg/mL. In embodiments, the arginine is present at a concentration from about 20 mg/mL to about 150 mg/mL and glutamic acid is present at a concentration from about 20 mg/mL to about 150 mg/mL; and wherein the dispersion further includes a saccharide at a concentration from about 20 mg/mL to about 75 mg/mL. In embodiments, the arginine is present at a concentration from about 20 mg/mL to about 150 mg/mL and glutamic acid is present at a concentration from about 20 mg/mL to about 150 mg/mL; and wherein the dispersion further includes trehalose at a concentration from about 20 mg/mL to about 75 mg/mL. In embodiments, the viscosity lowering agent is present at a concentration of about 10 to 300 mg/mL. In embodiments, the concentration of the viscosity lowering agent is between about 20 and 300, 20 and 250, 20 and 200, 20 and 150, 20 and 100, 20 and 50, 25 and 250, 25 and 200, 25 and 150, 50 and 250, 50 and 200, 50 and 150, 75 and 250, 75 and 200, 75 and 150, 100 and 300, 100 and 250, 100 and 200, 125 and 300, 125 and 250, 125 and 200, 10 and 250, 10 and 200, 10 and 150, or 10 and 100 mg/mL. In embodiments, the proline is present at a concentration between about 10 mg/mL and about 300 mg/mL. In embodiments, the proline is present at a concentration between about 100 mg/mL and about 300 mg/mL. In embodiments, the proline is present at a concentration between about 100 mg/mL and about 200 mg/mL (e.g., between about 110 and 190, 110 and 180, 110 and 170, 120 and 160, 130 and 160, or 130 and 150 mg/mL). In embodiments, the histidine is present at a concentration between about 5 mg/mL and about 200 mg/mL (e.g., between about 10 and 180, 10 and 160, 20 and 160, 20 and 150, 20 and 130, 20 and 110, 20 and 100, 20 and 90, 30 and 90, 30 and 80, 30 and 70, 30 and 60, 20 and 60, 20 and 70, 20 and 80, or 20 and 90 mg/mL).

In embodiments, the viscosity lowering agent is present at about 20 to 150, 20 to 100, 20 to 50, 50 to 150, 50 to 100, 75 to 150 or 75 to 100 mg/mL and the saccharide (e.g., trehalose, sucrose, sorbitol, glucose, fructose, xylose, galactose, mannitol, or raffinose) is present at about 40 to 100, 50 to 100, 60 to 100, 70 to 100, 80 to 100, 90 to 100, 40 to 80, 50 to 80, 60 to 80, 70 to 80, 40 to 60, or 50 to 60 mg/mL. In embodiments, the viscosity lowering agent is present at a concentration from about 20 mg/mL to about 150 mg/mL and the saccharide (e.g., trehalose, sucrose, sorbitol, glucose, fructose, xylose, galactose, mannitol, or raffinose) is present at a concentration from about 40 mg/mL and about 100 mg/mL. In embodiments, the viscosity lowering agent is present at a concentration from about 20 mg/mL to about 150 mg/mL and the saccharide (e.g., trehalose, sucrose, sorbitol, glucose, fructose, xylose, galactose, mannitol, or raffinose) is present at a concentration from about 40 mg/mL to about 60 mg/mL. In embodiments, the viscosity lowering agent is present at a concentration from about 30 mg/mL to about 60 mg/mL and the saccharide (e.g., trehalose, sucrose, sorbitol, glucose, fructose, xylose, galactose, mannitol, or raffinose) is present at a concentration from about 40 mg/mL to about 60 mg/mL. In embodiments, the viscosity lowering agent is present at a concentration from about 30 mg/mL to about 60 mg/mL and the saccharide (e.g., trehalose, sucrose, sorbitol, glucose, fructose, xylose, galactose, mannitol, or raffinose) is present at a concentration from about 40 mg/mL to about 60 mg/mL.

In embodiments, the dispersion includes between about 200 mg/mL and about 400 mg/mL of the protein. In embodiments, the protein concentration is between about 200 and about 350 mg/mL. In embodiments, the protein concentration is between about 200 and about 300 mg/mL. The protein concentration may be between about 200 and about 250 mg/mL. In embodiments, the protein concentration is between about 220 and about 400 mg/mL. The protein concentration may be between about 250 and about 400 mg/mL. In embodiments, the protein concentration is between about 270 and about 400 mg/mL. The protein concentration may be between about 300 and about 400 mg/mL. In embodiments, the protein concentration is between about 220 and about 350 mg/mL. The protein concentration may be between about 250 and about 350 mg/mL. In embodiments, the protein concentration is between about 270 and about 350 mg/mL. The protein concentration may be between about 220 and about 300 mg/mL. In embodiments, the protein concentration is between about 250 and about 300 mg/mL. The protein concentration may be between about 270 and about 300 mg/mL. The protein concentration may be between about 200 and about 1000 mg/mL. The protein concentration may be between about 200 and about 1000 mg/mL, about 250 and about 1000 mg/mL, about 300 and about 1000 mg/mL, about 350 and about 1000 mg/mL, about 400 and about 1000 mg/mL, about 450 and about 1000 mg/mL, about 500 and about 1000 mg/mL, about 550 and about 1000 mg/mL, about 600 and about 1000 mg/mL, about 650 and about 1000 mg/mL, about 700 and about 1000 mg/mL, about 750 and about 1000 mg/mL, about 800 and about 1000 mg/mL, about 850 and about 1000 mg/mL, about 900 and about 1000 mg/mL, about 950 and about 1000 mg/mL, about 200 and about 950 mg/mL, about 200 and about 900 mg/mL, about 200 and about 850 mg/mL, about 200 and about 800 mg/mL, about 200 and about 750 mg/mL, about 200 and about 700 mg/mL, about 200 and about 600 mg/mL, about 200 and about 550 mg/mL, about 200 and about 500 mg/mL, about 200 and about 450 mg/mL, about 200 and about 400 mg/mL, about 200 and about 350 mg/mL, about 200 and about 300 mg/mL, about 200 and about 250 mg/mL, about 250 and about 950 mg/mL, about 300 and about 900 mg/mL, about 350 and about 850 mg/mL, about 400 and about 800 mg/mL, about 450 and about 750 mg/mL, about 500 and about 700 mg/mL, about 550 and about 600 mg/mL, about 250 and about 550 mg/mL, about 250 and about 500 mg/mL, about 250 and about 450 mg/mL, about 250 and about 400 mg/mL, about 250 and about 350 mg/mL, about 250 and about 300 mg/mL, about 300 and about 550 mg/mL, about 300 and about 500 mg/mL, about 300 and about 450 mg/mL, about 300 and about 400 mg/mL, or about 300 and about 350 mg/mL.

The dispersion may be translucent. The dispersion may be transparent.

In embodiments, the nanoclusters are particles. In embodiments, the nanoclusters are nanoclusters. The nanoclusters may have a diameter (e.g., average diameter) between about 20 and 900 nm, 20 and 800 nm, 20 and 700 nm, 20 and 600 nm, 20 and 500 nm, 20 and 400 nm, 20 and 300 nm, 20 and 200 nm, 20 and 100 nm, 50 and 1000 nm, 50 and 900 nm, 50 and 800 nm, 50 and 700 nm, 50 and 600 nm, 50 and 500 nm, 50 and 400 nm, 50 and 300 nm, 50 and 200 nm, 50 and 100 nm, 75 and 1000 nm, 75 and 900 nm, 75 and 800 nm, 75 and 700 nm, 75 and 600 nm, 75 and 500 nm, 75 and 400 nm, 75 and 300 nm, 75 and 200 nm, 75 and 100 nm, 100 and 1000 nm, 100 and 900 nm, 100 and 800 nm, 100 and 700 nm, 100 and 600 nm, 100 and 500 nm, 100 and 400 nm, 100 and 300 nm, 100 and 200 nm, 250 and 1000 nm, 250 and 900 nm, 250 and 800 nm, 250 and 700 nm, 250 and 600 nm, 250 and 500 nm, 250 and 400 nm, 250 and 300 nm, 500 and 1000 nm, 500 and 900 nm, 500 and 800 nm, 500 and 700 nm, 500 and 600 nm, 750 and 1000 nm, 750 and 900 nm, or 750 and 800 nm. The nanoparticles may have a diameter (e.g., average diameter) between about 20 and 900 nm, 20 and 800 nm, 20 and 700 nm, 20 and 600 nm, 20 and 500 nm, 20 and 400 nm, 20 and 300 nm, 20 and 200 nm, 20 and 100 nm, 50 and 1000 nm, 50 and 900 nm, 50 and 800 nm, 50 and 700 nm, 50 and 600 nm, 50 and 500 nm, 50 and 400 nm, 50 and 300 nm, 50 and 200 nm, 50 and 100 nm, 75 and 1000 nm, 75 and 900 nm, 75 and 800 nm, 75 and 700 nm, 75 and 600 nm, 75 and 500 nm, 75 and 400 nm, 75 and 300 nm, 75 and 200 nm, 75 and 100 nm, 100 and 1000 nm, 100 and 900 nm, 100 and 800 nm, 100 and 700 nm, 100 and 600 nm, 100 and 500 nm, 100 and 400 nm, 100 and 300 nm, 100 and 200 nm, 250 and 1000 nm, 250 and 900 nm, 250 and 800 nm, 250 and 700 nm, 250 and 600 nm, 250 and 500 nm, 250 and 400 nm, 250 and 300 nm, 500 and 1000 nm, 500 and 900 nm, 500 and 800 nm, 500 and 700 nm, 500 and 600 nm, 750 and 1000 nm, 750 and 900 nm, or 750 and 800 nm. The nanoclusters may have a diameter (e.g., average diameter) between about 10 and 1000 nm. The nanoclusters may have a diameter (e.g., average diameter) between about 10 and 500 nm. The nanoclusters may have a diameter (e.g., average diameter) between about 10 and 50 nm. The nanoclusters may have a diameter (e.g., average diameter) between about 10 and 30 nm.

In embodiments, the dispersion is approximately isotonic with human blood. The dispersion may be isotonic with human blood. The dispersion may be within 1% of being isotonic with human blood. The dispersion may be within 2% of being isotonic with human blood. The dispersion may be within 3% of being isotonic with human blood. The dispersion may be within 5% of being isotonic with human blood. The dispersion may be within 10% of being isotonic with human blood. The dispersion may be within 20% of being isotonic with human blood. The dispersion may be within 50% of being isotonic with human blood. The dispersion may be within 75% of being isotonic with human blood. The dispersion may be up to 2-fold the tonicity of human blood. The dispersion may be up to 2.5-fold the tonicity of human blood. The dispersion may be up to 3-fold the tonicity of human blood. The dispersion may be up to 3.5-fold the tonicity of human blood. The dispersion may be up to 4-fold the tonicity of human blood. The dispersion may be up to 5-fold the tonicity of human blood. The dispersion may have a tonicity between about isotonic with human blood and about 3 times the tonicity of human blood. The dispersion may have a tonicity between about isotonic with human blood and about 2 times the tonicity of human blood. The dispersion may have a tonicity between about isotonic with human blood and about 1.5 times the tonicity of human blood.

In embodiments, the pH of the dispersion is from about 4.0 to about 10.0. In embodiments, the pH of the dispersion is from about 5.5 to about 10.0. In embodiments, the pH of the dispersion is from about 5.5 to about 6.5. The pH of the dispersion may be between about 4 and 10, 4.5 and 10, 4.5 and 9.5, 4.5 and 9.0, 4.5 and 8.5, 4.5 and 8.0, 4.5 and 7.5, 4.5 and 7.0, 4.5 and 6.5, 4.5 and 6.0, 4.5 and 5.5, 4.5 and 5.0, 5.0 and 10, 5.0 and 9.5, 5.0 and 9.0, 5.0 and 8.5, 5.0 and 8.0, 5.0 and 7.5, 5.0 and 7.0, 5.0 and 6.5, 5.0 and 6.0, 5.0 and 5.5, 5.5 and 10, 5.5 and 9.5, 5.5 and 9.0, 5.5 and 8.5, 5.5 and 8.0, 5.5 and 7.5, 5.5 and 7.0, 5.5 and 6.5, 5.5 and 6.0, 6.0 and 10, 6.0 and 9.5, 6.0 and 9.0, 6.0 and 8.5, 6.0 and 8.0, 6.0 and 7.5, 6.0 and 7.0, 6.0 and 6.5, 6.5 and 10, 6.5 and 9.5, 6.5 and 9.0, 6.5 and 8.5, 6.5 and 8.0, 6.5 and 7.5, 6.5 and 7.0, 7.0 and 10, 7.0 and 9.5, 7.0 and 9.0, 7.0 and 8.5, 7.0 and 8.0, 7.0 and 7.5, 7.5 and 10, 7.5 and 9.5, 7.5 and 9.0, 7.5 and 8.5, 7.5 and 8.0, 8.0 and 10, 8.0 and 9.5, 8.0 and 9.0, 8.0 and 8.5, 8.5 and 10, 8.5 and 9.5, 8.5 and 9.0, 9.0 and 10, 9.0 and 9.5, or 9.5 and 10.

In some embodiments, the particles include a plurality of proteins. The plurality of proteins may share complete amino acid sequence identity. In some embodiments, the plurality of proteins are substantially identical. In some embodiments, the plurality of proteins are about 75% identical. In some embodiments, the plurality of proteins are about 80% identical. In some embodiments, the plurality of proteins are about 85% identical. In some embodiments, the plurality of proteins are about 90% identical. In some embodiments, the plurality of proteins are about 95% identical. In some embodiments, the plurality of proteins are about 96% identical. In some embodiments, the plurality of proteins are about 97% identical. In some embodiments, the plurality of proteins are about 98% identical. In some embodiments, the plurality of proteins are about 99% identical. In some embodiments, the plurality of proteins are about 99.5% identical. In some embodiments, the plurality of proteins are about 99.6% identical. In some embodiments, the plurality of proteins are about 99.7% identical. In some embodiments, the plurality of proteins are about 99.8% identical. In some embodiments, the plurality of proteins are about 99.9% identical. In some embodiments, the plurality of proteins are identical except for drift in the sequence attributable to mistakes in transcription or translation. In some embodiments, the dispersion includes a plurality of particles. In some embodiments, the plurality of particles includes a mixture of proteins with different amino acid sequences. In embodiments, the protein is an antibody. In embodiments, the protein is an antibody fragment. In embodiments, the protein is a therapeutic protein. In embodiments, the protein is a protein drug. In embodiments, the protein is a peptide. In embodiments, the protein is a therapeutic peptide. In embodiments, the protein is a monoclonal antibody. In embodiments, the protein is a chimeric antibody. In embodiments, the protein is a humanized antibody. In embodiments, the protein is an antibody drug conjugate. In embodiments, the protein is a recombinant protein. In embodiments, the protein is a recombinant peptide. In embodiments, the protein includes non-natural amino acids. In embodiments, the protein is an IgG antibody, an IgM antibody, an IgA antibody, an IgD antibody, or an IgE antibody. In embodiments, the protein is an IgG antibody.

In embodiments, the protein is an enzyme, a cytokine, a neurotropic factor, an antibody, a peptide, a hormone, a DNA-binding protein, an aptamer, erythropoietin (EPO), human insulin, interferon-a (Intron A), granulocyte-colony stimulating factor (G-CSF), human growth hormone, interferon-β, recombinant hepatitis B vaccine, glucocerobrosidase, agalsidase, imiglucerase, ogalactosidase A, or a tissue plasminogen activator (tPA).

In embodiments, the antibody is ranibizumab, bevacizumab, cetuximab, infliximab, palivizumab, abciximab, alemtuzumab, altumomab pentetate, atlizumab, basiliximab, daclizumab, eculizumab, muromonab-CD3, natalizumab, ofatumumab, panitumumab, rituximab, tocilizumab, or trastuzumab.

In embodiments, the antibody is Infliximab, Bevacizumab, Ranibizumab, Cetuximab, Ranibizumab, Palivizumab, Abagovomab, Abciximab, Actoxumab, Adalimumab, Afelimomab, Afutuzumab, Alacizumab, Alacizumab pegol, ALD518, Alemtuzumab, Alirocumab, Alemtuzumab, Altumomab, Amatuximab, Anatumomab mafenatox, Anrukinzumab, Apolizumab, Arcitumomab, Aselizumab, Altinumab, Atlizumab, Atorolimiumab, tocilizumab, Bapineuzumab, Basiliximab, Bavituximab, Bectumomab, Belimumab, Benralizumab, Bertilimumab, Besilesomab, Bevacizumab, Bezlotoxumab, Biciromab, Bivatuzumab, Bivatuzumab mertansine, Blinatumomab, Blosozumab, Brentuximab vedotin, Briakinumab, Brodalumab, Canakinumab, Cantuzumab mertansine, Cantuzumab mertansine, Caplacizumab, Capromab pendetide, Carlumab, Catumaxomab, CC49, Cedelizumab, Certolizumab pegol, Cetuximab, Citatuzumab bogatox, Cixutumumab, Clazakizumab, Clenoliximab, Clivatuzumab tetraxetan, Conatumumab, Crenezumab, CR6261, Dacetuzumab, Daclizumab, Dalotuzumab, Daratumumab, Demcizumab, Denosumab, Detumomab, Dorlimomab aritox, Drozitumab, Duligotumab, Dupilumab, Ecromeximab, Eculizumab, Edobacomab, Edrecolomab, Efalizumab, Efungumab, Elotuzumab, Elsilimomab, Enavatuzumab, Enlimomab pegol, Enokizumab, Enokizumab, Enoticumab, Enoticumab, Ensituximab, Epitumomab cituxetan, Epratuzumab, Erlizumab, Ertumaxomab, Etaracizumab, Etrolizumab, Exbivirumab, Exbivirumab, Fanolesomab, Faralimomab, Farletuzumab, Fasinumab, FBTA05, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, GS6624, Ibalizumab, Ibritumomab tiuxetan, lcrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lebrikizumab, Lemalesomab, Lerdelimumab, Lexatumumab, Libivirumab, Ligelizumab, Lintuzumab, Lirilumab, Lorvotuzumab mertansine, Lucatumumab, Lumiliximab, Mapatumumab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pintumomab, Placulumab, Ponezumab, Priliximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Tefibazumab, Telimomab aritox, Tenatumomab, Teneliximab, Teplizumab, Teprotumumab, TGN 1412, tremelimumab, Ticilimumab, Tildrakizumab, Tigatuzumab, TNX-650, Tocilizumab, Toralizumab, Tositumomab, Tralokinumab, Trastuzumab, TRBS07, Tregalizumab, Tremelimumab, Tucotuzumab celmoleukin, Tuvirumab, Ublituximab, Urelumab, Urtoxazumab, Ustekinumab, Vapaliximab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab, or Zolimomab aritox.

In some embodiments of the dispersion, less than 5% of the plurality of proteins in the plurality of particles are irreversibly aggregated. In some embodiments of the dispersion, less than 2% of the plurality of proteins in the plurality of particles are irreversibly aggregated. In some embodiments of the dispersion, less than 1% of the plurality of proteins in the plurality of particles are irreversibly aggregated.

In some embodiments, viscosity (e.g. of a dispersion) is measured by a syringe loading method. In some embodiments, viscosity (e.g. of a dispersion) is measured with a viscometer (e.g. Stormer viscometer, vibrating viscometer, rotating viscometer, Marsh funnel viscometer, U-tube viscometer, falling sphere viscometer, falling piston viscometer, oscillating piston viscometer, Stabinger viscometer, bubble viscometer, or Cannon-Fenske viscometer). In some embodiments, viscosity (e.g. of a dispersion) is measured with a rheometer. In some embodiments, viscosity (e.g. of a dispersion) is measured with a Zahn cup. In some embodiments, viscosity (e.g. of a dispersion) is measured with a Ford viscosity cup. In some embodiments, viscosity (e.g. of a dispersion) is measured with a syringe (e.g. a syringe equipped with a needle having a size between 21 gauge and 27 gauge, or a 25 gauge needle, or a 1.5 inch long needle, or a 25 gauge 1.5 inch long needle). In some embodiments, viscosity (e.g. of a dispersion) is measured with a plastometer.

In some embodiments, the viscosity of the dispersion is less than about 75 centipoise and the shear rate of the dispersion is greater than about 750 second⁻¹. In some embodiments, the viscosity of the dispersion is less than about 75 centipoise and the shear rate of the dispersion is greater than about 1000 second⁻¹. In some embodiments, the viscosity of the dispersion is less than about 75 centipoise and the shear rate of the dispersion is greater than about 2000 second⁻¹. In some embodiments, the viscosity of the dispersion is less than about 75 centipoise and the shear rate of the dispersion is greater than about 3000 second⁻¹. In some embodiments, the viscosity of the dispersion is less than about 75 centipoise and the shear rate of the dispersion is greater than about 4000 second⁻¹. In some embodiments, the viscosity of the dispersion is less than about 75 centipoise and the shear rate of the dispersion is greater than about 5000 second⁻¹. In some embodiments, the viscosity of the dispersion is less than about 75 centipoise and the shear rate of the dispersion is greater than about 7500 second⁻¹. In some embodiments, the viscosity of the dispersion is less than about 75 centipoise and the shear rate of the dispersion is greater than about 10,000 second⁻¹.

In some embodiments, the viscosity of the dispersion is less than about 50 centipoise and the shear rate of the dispersion is greater than about 750 second⁻¹. In some embodiments, the viscosity of the dispersion is less than about 50 centipoise and the shear rate of the dispersion is greater than about 1000 second⁻¹. In some embodiments, the viscosity of the dispersion is less than about 50 centipoise and the shear rate of the dispersion is greater than about 2000 second⁻¹. In some embodiments, the viscosity of the dispersion is less than about 50 centipoise and the shear rate of the dispersion is greater than about 3000 second⁻¹. In some embodiments, the viscosity of the dispersion is less than about 50 centipoise and the shear rate of the dispersion is greater than about 4000 second⁻¹. In some embodiments, the viscosity of the dispersion is less than about 50 centipoise and the shear rate of the dispersion is greater than about 5000 second⁻¹. In some embodiments, the viscosity of the dispersion is less than about 50 centipoise and the shear rate of the dispersion is greater than about 7500 second⁻¹. In some embodiments, the viscosity of the dispersion is less than about 50 centipoise and the shear rate of the dispersion is greater than about 10,000 second⁻¹.

In some embodiments, the viscosity of the dispersion is less than about 50 centipoise and the shear rate of the dispersion is between about 750 second⁻¹ and about 10,000 second⁻¹. In some embodiments, the viscosity of the dispersion is less than about 50 centipoise and the shear rate of the dispersion is between about 1000 second⁻¹ and about 10,000 second⁻¹. In some embodiments, the viscosity of the dispersion is less than about 50 centipoise and the shear rate of the dispersion is between about 2000 second⁻¹ and about 10,000 second⁻¹. In some embodiments, the viscosity of the dispersion is less than about 50 centipoise and the shear rate of the dispersion is between about 3000 second⁻¹ and about 10,000 second⁻¹. In some embodiments, the viscosity of the dispersion is less than about 50 centipoise and the shear rate of the dispersion is between about 4000 second⁻¹ and about 10,000 second⁻¹. In some embodiments, the viscosity of the dispersion is less than about 50 centipoise and the shear rate of the dispersion is between about 5000 second⁻¹ and about 10,000 second⁻¹. In some embodiments, the viscosity of the dispersion is less than about 50 centipoise and the shear rate of the dispersion is between about 7500 second⁻¹ and about 10,000 second⁻¹. In some embodiments, the viscosity of the dispersion is less than about 50 centipoise and the shear rate of the dispersion is between about 1000 second⁻¹ and about 9,000 second⁻¹. In some embodiments, the viscosity of the dispersion is less than about 50 centipoise and the shear rate of the dispersion is between about 1000 second⁻¹ and about 8,000 second⁻¹. In some embodiments, the viscosity of the dispersion is less than about 50 centipoise and the shear rate of the dispersion is between about 1000 second⁻¹ and about 7,000 second⁻¹. In some embodiments, the viscosity of the dispersion is less than about 50 centipoise and the shear rate of the dispersion is between about 1000 second⁻¹ and about 6,000 second⁻¹. In some embodiments, the viscosity of the dispersion is less than about 50 centipoise and the shear rate of the dispersion is between about 1000 second⁻¹ and about 5,000 second⁻¹. In some embodiments, the viscosity of the dispersion is less than about 50 centipoise and the shear rate of the dispersion is between about 1000 second⁻¹ and about 4,000 second⁻¹. In some embodiments, the viscosity of the dispersion is less than about 50 centipoise and the shear rate of the dispersion is between about 1000 second⁻¹ and about 3,000 second⁻¹. In some embodiments, the viscosity of the dispersion is less than about 50 centipoise and the shear rate of the dispersion is between about 1000 second⁻¹ and about 2,000 second⁻¹. In some embodiments, the viscosity of the dispersion is less than about 50 centipoise and the shear rate of the dispersion is between about 2000 second⁻¹ and about 6,000 second⁻¹. In some embodiments, the viscosity of the dispersion is less than about 50 centipoise and the shear rate of the dispersion is between about 2000 second⁻¹ and about 4,000 second⁻¹. In some embodiments, the viscosity of the dispersion is less than about 50 centipoise and the shear rate of the dispersion is between about 500 second⁻¹ and about 3,000 second⁻¹. In some embodiments, the viscosity of the dispersion is less than about 50 centipoise and the shear rate of the dispersion is between about 500 second⁻¹ and about 2000 second⁻¹. In some embodiments, the viscosity of the dispersion is less than about 50 centipoise and the shear rate of the dispersion is between about 750 second⁻¹ and about 2,000 second⁻¹. In some embodiments, the viscosity of the dispersion is less than about 50 centipoise and the shear rate of the dispersion is between about 750 second⁻¹ and about 1,500 second⁻¹.

In some embodiments, the viscosity of the dispersion is about 50 centipoise and the shear rate of the dispersion is about 1000 second⁻¹ (e.g. 50 centipoise and 1000 second⁻¹). In some embodiments, the viscosity of the dispersion is between about 25 centipoise and about 75 centipoise and the shear rate of the dispersion is about 1000 second⁻¹ (e.g. between 25 centipoise and 75 centipoise and 1000 second⁻¹). In some embodiments, the viscosity of the dispersion is between about 10 centipoise and about 90 centipoise and the shear rate of the dispersion is about 1000 second⁻¹ (e.g. between 10 centipoise and 90 centipoise and 1000 second⁻¹). In some embodiments, the viscosity of the dispersion is about 50 centipoise and the shear rate of the dispersion is between about 100 second⁻¹ and about 50000 second⁻¹ (e.g. 50 centipoise and between 100 second⁻¹ and 50000 second⁻¹). In some embodiments, the viscosity of the dispersion is between about 25 centipoise and 75 centipoise and the shear rate of the dispersion is between about 100 second⁻¹ and about 50000 second⁻¹ (e.g. between 25 centipoise and 75 centipoise and between 100 second⁻¹ and 50000 second⁻¹). In some embodiments, the viscosity of the dispersion is between about 25 centipoise and 75 centipoise and the shear rate of the dispersion is between about 500 second⁻¹ and about 10000 second⁻¹ (e.g. between 25 centipoise and 75 centipoise and between 500 second⁻¹ and 10000 second⁻¹). In some embodiments, the dispersion is syringeable and wherein an aqueous solution of the plurality of proteins at an identical concentration is not syringeable. In some embodiments, the dispersion has a viscosity about two fold lower than the viscosity of an aqueous solution of the plurality of proteins at an identical concentration (e.g. 1.6 fold, 1.7 fold, 1.8 fold, 1.9 fold, 2 fold, 2.1 fold, 2.2 fold, 2.3 fold, 2.4 fold). In some embodiments, the dispersion has a viscosity about five fold lower than the viscosity of an aqueous solution of the plurality of proteins at an identical concentration (e.g. 4.6 fold, 4.7 fold, 4.8 fold, 4.9 fold, 5 fold, 5.1 fold, 5.2 fold, 5.3 fold, 5.4 fold). In some embodiments, the dispersion has a viscosity about tenfold lower than the viscosity of an aqueous solution of the plurality of proteins at an identical concentration (e.g. 9.6 fold, 9.7 fold, 9.8 fold, 9.9 fold, 10 fold, 10.1 fold, 10.2 fold, 10.3 fold, 10.4 fold).

In some embodiments, the viscosity of the dispersion is between about the two viscosity values corresponding to any one of the cells in the table/matrix immediately below having number 1 to 240 (i.e. one viscosity for column and one viscosity for row), wherein between includes the two viscosity values):

| Viscosity | viscosity (cP) | | | | | | | | | |
|---|---|---|---|---|---|---|---|---|---|---|
| (cP) | 90 | 80 | 70 | 60 | 50 | 40 | 30 | 20 | 10 | 1 |
| 100 | 1 | 2 | 3 | 4 | 5 | 6 | 7 | 8 | 9 | 10 |
| 95 | 11 | 12 | 13 | 14 | 15 | 16 | 17 | 18 | 19 | 20 |
| 90 | 21 | 22 | 23 | 24 | 25 | 26 | 27 | 28 | 29 | 30 |
| 85 | 31 | 32 | 33 | 34 | 35 | 36 | 37 | 38 | 39 | 40 |
| 80 | 41 | 42 | 43 | 44 | 45 | 46 | 47 | 48 | 49 | 50 |
| 75 | 51 | 52 | 53 | 54 | 55 | 56 | 57 | 58 | 59 | 60 |
| 70 | 61 | 62 | 63 | 64 | 65 | 66 | 67 | 68 | 69 | 70 |
| 65 | 71 | 72 | 73 | 74 | 75 | 76 | 77 | 78 | 79 | 80 |
| 60 | 81 | 82 | 83 | 84 | 85 | 86 | 87 | 88 | 89 | 90 |
| 55 | 91 | 92 | 93 | 94 | 95 | 96 | 97 | 98 | 99 | 100 |
| 50 | 101 | 102 | 103 | 104 | 105 | 106 | 107 | 108 | 109 | 110 |
| 45 | 111 | 112 | 113 | 114 | 115 | 116 | 117 | 118 | 119 | 120 |
| 40 | 121 | 122 | 123 | 124 | 125 | 126 | 127 | 128 | 129 | 130 |
| 35 | 131 | 132 | 133 | 134 | 135 | 136 | 137 | 138 | 139 | 140 |
| 30 | 141 | 142 | 143 | 144 | 145 | 146 | 147 | 148 | 149 | 150 |
| 25 | 151 | 152 | 153 | 154 | 155 | 156 | 157 | 158 | 159 | 160 |
| 20 | 161 | 162 | 163 | 164 | 165 | 166 | 167 | 168 | 169 | 170 |
| 15 | 171 | 172 | 173 | 174 | 175 | 176 | 177 | 178 | 179 | 180 |
| 10 | 181 | 182 | 183 | 184 | 185 | 186 | 187 | 188 | 189 | 190 |
| 9 | 191 | 192 | 193 | 194 | 195 | 196 | 197 | 198 | 199 | 200 |
| 8 | 201 | 202 | 203 | 204 | 205 | 206 | 207 | 208 | 209 | 210 |
| 7 | 211 | 212 | 213 | 214 | 215 | 216 | 217 | 218 | 219 | 220 |
| 6 | 221 | 222 | 223 | 224 | 225 | 226 | 227 | 228 | 229 | 230 |
| 5 | 231 | 232 | 233 | 234 | 235 | 236 | 237 | 238 | 239 | 240 |

In some embodiments, the dispersion includes a light extinction measurement less than about 0.05, about 0.1, about 0.25, or about 0.5 cm⁻¹, wherein the light extinction measurement includes an average light extinction over wavelengths between 400 nm and 700 nm (e.g. less than 0.05, 0.1, 0.25, or 0.5 cm⁻¹). In some embodiments, the dispersion includes a light extinction measurement less than about 0.05, about 0.1, about 0.25, or about 0.5 cm⁻¹, wherein the light extinction measurement is made at a wavelength of 600 nm (e.g. less than 0.05, 0.1, 0.25, or 0.5 cm⁻¹). In some embodiments, the dispersion includes a light extinction measurement less than about 0.05, about 0.1, about 0.25, or about 0.5 cm⁻¹, wherein the light extinction measurement is made at a wavelength of between 400 nm and 700 nm (e.g. less than 0.05, 0.1, 0.25, or 0.5 cm⁻¹, and at a wavelength of 400, 450, 500, 550, 600, 650, or 700 nm or any other intervening wavelength).

In some embodiments of the dispersion, the plurality of particles have an average packing fraction between about 30% and about 80% (e.g. between 30% and 80%). In some embodiments of the dispersion, the plurality of particles have an average packing fraction between about 30% and about 70% (e.g. between 30% and 70%). In some embodiments of the dispersion, the plurality of particles have an average packing fraction between about 30% and about 60% (e.g. between 30% and 60%). In some embodiments of the dispersion, the plurality of particles have an average packing fraction between about 30% and about 50% (e.g. between 30% and 50%). In some embodiments of the dispersion, the plurality of particles have an average packing fraction between about 50% and about 60% (e.g. between 50% and 60%). In some embodiments of the dispersion, the plurality of particles have an average packing fraction between about 60% and about 74% (e.g. between 60% and 74%).

In embodiments, a viscosity lowering agent is a crowder. In some embodiments, the crowder is a monosaccharide. In some embodiments, the crowder is a monosaccharide selected from glucose, mannose, fructose, arabinose, xylose, ribose, and galactose. In some embodiments, the crowder is a disaccharide. In some embodiments, the crowder is a disaccharide selected from trehalose, lactulose, lactose, cellobiose, maltose, or sucrose. In some embodiments, the crowder is a polysaccharide. In some embodiments, the crowder is a polyelectrolyte. In some embodiments, the crowder is a polyacid. In some embodiments, the crowder is a poly(ethylene glycol). In some embodiments, the crowder is a poly(ethylene glycol) with a molecular weight between PEG 200 and PEG 5000. In some embodiments, the crowder is a salt. In some embodiments, the crowder is a dextran. In some embodiments, the crowder is a polaxamer. In some embodiments, the crowder is an alcohol. In some embodiments, the crowder is an amino acid or protein. The crowder may be proline. The crowder may be histidine. The crowder may be imidazole. The crowder may be glutamic acid. The crowder may be betaine. The crowder may be glutamine. The crowder may be asparagine. The crowder may be arginine. In some embodiments, the crowder is a dipeptide, tripeptide, four amino acid peptide, five amino acid peptide, or oligopeptide. In some embodiments, the crowder is a conjugated protein. In some embodiments, the crowder is a non-conjugated protein. In some embodiments, the crowder is a non-protein crowder. In some embodiments, the crowder is a surfactant. In some embodiments, the dispersion includes a crowder selected from the group consisting of a trehalose, a poly(ethylene glycol), ethanol, N-methyl-2-pyrrolidone (NMP), a buffer, or a combination thereof. In some embodiments, the dispersion includes about a 1:1 weight ratio of protein to a crowder (e.g. a 1:1 weight ratio). In some embodiments, the dispersion includes about a 2:1 weight ratio of protein to a crowder (e.g. a 2:1 weight ratio). In some embodiments, the dispersion includes about a 3:1 weight ratio of protein to a crowder (e.g. a 3:1 weight ratio). In some embodiments, the dispersion includes about a 4:1 weight ratio of protein to a crowder (e.g. a 4:1 weight ratio). In some embodiments, the dispersion includes about a 5:1 weight ratio of protein to a crowder (e.g. a 5:1 weight ratio). In some embodiments, the dispersion includes about a 6:1 weight ratio of protein to a crowder (e.g. a 6:1 weight ratio). In some embodiments, the dispersion includes about a 10:1 weight ratio of protein to a crowder (e.g. a 10:1 weight ratio). In some embodiments, the dispersion includes about a 1:2 weight ratio of protein to a crowder (e.g. a 1:2 weight ratio). In some embodiments, the dispersion includes about a 1:3 weight ratio of protein to a crowder (e.g. a 1:3 weight ratio). In some embodiments, the dispersion includes about a 1:4 weight ratio of protein to a crowder (e.g. a 1:4 weight ratio). In some embodiments, the dispersion includes about a 1:5 weight ratio of protein to a crowder (e.g. a 1:5 weight ratio). In some embodiments, the dispersion includes about a 1:10 weight ratio of protein to a crowder (e.g. a 1:10 weight ratio).

In some embodiments of the dispersion, the pH of the dispersion is at about the isoelectric point of the plurality of proteins (e.g. is at the isoelectric point). In some embodiments of the dispersion, the pH of the dispersion is less than about 2.5, 2.0, 1.5, 1.0, 0.8, 0.75, 0.5, 0.3, 0.2, 0.1, or 0.05 pH units different from the isoelectric point of the plurality of proteins (e.g. less than 2.5, 2.4, 2.3, 2.2, 2.1, 2.0, 1.9, 1.8, 1.7, 1.6, 1.5, 1.4, 1.3, 1.2, 1.1, 1.0, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.09, 0.08, 0.07, 0.06, 0.05, 0.04, 0.03, 0.02, or 0.01 pH units). In some embodiments, the pH of the dispersion is about 4, 4.5, 5, 5.5, 6, 6.5, 7, 7.5, 8, 8.5, 9, or 9.5.

In some embodiments, the dispersion is isotonic with human blood. In some embodiments, the dispersion is hypotonic with human blood. In some embodiments, the dispersion is hypertonic with human blood. In some embodiments, the dispersion has an osmolarity of about 300 mOsmo/L (e.g. 300 mOsmo/L). In some embodiments, the dispersion has an osmolarity of between about 250 mOsmo/L and about 350 mOsmol/L (e.g. 250 mOsmo/L and 350 mOsmol/L). In some embodiments, the dispersion has an osmolarity of between about 150 mOsmo/L and about 450 mOsmol/L (e.g. 150 mOsmo/L and 450 mOsmol/L). In some embodiments, the dispersion has an osmolarity of between about 150 mOsmo/L and about 600 mOsmol/L (e.g. between 150 mOsmo/L and 600 mOsmol/L). In some embodiments, the dispersion has an osmolarity of between about 250 mOsmo/L and about 1500 mOsmol/L (e.g. 250 mOsmo/L and 1500 mOsmol/L). In some embodiments, the dispersion has an osmolarity of between about 250 mOsmo/L and about 1200 mOsmol/L (e.g. 250 mOsmo/L and 1200 mOsmol/L). In some embodiments, the dispersion has an osmolarity of between about 250 mOsmo/L and about 900 mOsmol/L (e.g. 250 mOsmo/L and 900 mOsmol/L). In some embodiments, the dispersion has an osmolarity of about 300 mOsmo/kg (e.g. 300 mOsmo/kg). In some embodiments, the dispersion has an osmolarity of between about 250 mOsmo/kg and about 350 mOsmol/kg (e.g. 250 mOsmo/kg and 350 mOsmol/kg). In some embodiments, the dispersion has an osmolarity of between about 150 mOsmo/kg and about 450 mOsmol/kg (e.g. 150 mOsmo/kg and 450 mOsmol/kg). In some embodiments, the dispersion has an osmolarity of between about 150 mOsmo/kg and about 600 mOsmol/kg (e.g. between 150 mOsmo/kg and 600 mOsmol/kg). In some embodiments, the dispersion has an osmolarity of between about 250 mOsmo/kg and about 1500 mOsmol/kg (e.g. 250 mOsmo/kg and 1500 mOsmol/kg). In some embodiments, the dispersion has an osmolarity of between about 250 mOsmo/kg and about 1200 mOsmol/kg (e.g. 250 mOsmo/kg and 1200 mOsmol/kg). In some embodiments, the dispersion has an osmolarity of between about 250 mOsmo/kg and about 900 mOsmol/kg (e.g. 250 mOsmo/kg and 900 mOsmol/kg).

In some embodiments of the dispersion, each of the plurality of proteins is an antibody, an antibody fragment, a pegylated protein, a lipidated protein, a growth factor or growth factor antagonist, a cytokine or cytokine antagonist, a receptor or receptor antagonist, an antigen, a vaccine, or an anti-inflammatory agent. In some embodiments of the dispersion, the plurality of proteins is a plurality of conjugates, wherein each of the conjugates is a protein bonded to low molecular weight compound, wherein the low molecular weight compound is a diagnostic agent, a pharmaceutical agent, a contrast agent, a fluorophore, a radioisotope, a toxin, a paramagnetic agent, or an aptamer. In some embodiments of the dispersion, the plurality of proteins is self-crowding. In some embodiments of the dispersion, the plurality of proteins is not a plurality of conjugates and each of the proteins consists of amino acids (i.e. non-conjugated protein). In some embodiments, the one or more proteins or peptides are selected from an antibody, an antibody fragment (e.g. Fab, Fc, Fv, Fab′), a pegylated protein, a lipidated protein, a growth factor or antagonist, a cytokine or antagonist, a receptor or receptor antagonist, an antigen, a vaccine, an anti-inflammatory agent, a therapeutic polypeptide or peptide, or a combination thereof. In some embodiments, the particle (e.g., nanoparticle, nanocluster) is a reversible particle (e.g., nanoparticle, nanocluster) having primary protein particles that dissociate into stable monomeric proteins upon parenteral administration.

In some embodiments, the plurality of nanoclusters (e.g., nanoparticles) include multiple different protein species. In some embodiments of the dispersion, the plurality of nanoclusters (e.g., nanoparticles) is a first plurality of nanoclusters (e.g., nanoparticles) and the plurality of proteins is a first plurality of proteins, the dispersion further includes a second plurality of nanoclusters (e.g., nanoparticles) wherein each of the second plurality of nanoclusters (e.g., nanoparticles) includes a second plurality of proteins and each of the second plurality of proteins shares amino acid sequence identity, and the second plurality of proteins is different from the first plurality of proteins. In some embodiments of the dispersion, the plurality of nanoclusters (e.g., nanoparticles) further includes a controlled release polymer. In some embodiments of the dispersion, the plurality of nanoclusters (e.g., nanoparticles) further includes a controlled release component. In some embodiments of the dispersion, each of the plurality of nanoclusters (e.g., nanoparticles) further includes a low molecular weight compound and the low molecular weight compound is a diagnostic agent, a pharmaceutical agent, a contrast agent, a fluorophore, a radioisotope, a toxin, a paramagnetic agent, a metal, a metal oxide, or an aptamer.

In some embodiments of the dispersion, the dispersion further includes a second plurality of nanoclusters (e.g., nanoparticles). In some embodiments of the dispersion, the second plurality of nanoclusters (e.g., nanoparticles) include a plurality of a compound selected from Au, a magnetic agent, an optical agent, a diagnostic agent, a pharmaceutical agent, a contrast agent, a fluorophore, a radioisotope, a toxin, a paramagnetic agent, a metal, a metal oxide, or an aptamer.

In some embodiments, the compositions as described herein (including embodiments) (e.g. protein dispersions) may include a pharmaceutical and a diagnostic agent. The term theranostics is commonly used to describe a single composition including both a therapeutic and diagnostic agent. The synergy between treatment and monitoring or diagnostics may be useful for targeting the treatment more effectively and for selecting the proper dosage. In some embodiments, the composition may include a high dosage of a protein therapeutic and a high but non-toxic amount of a diagnostic agent (e.g. imaging agent, contrast agent). The imaging agent may be chemically attached to the protein (e.g. a conjugate) or it may be dispersed with the protein. In some embodiments, the imaging agent may itself be a nanoparticle, for example Au for optical imaging or iron oxide for magnetic imaging. In some embodiments, the nanoparticle may be chemically attached to the protein in the particle, or the particle may include a non-conjugated protein and a diagnostic agent or a particle including the diagnostic agent. In some embodiments of the compositions described herein, a dispersion includes a plurality of protein particles and a plurality of diagnostic agent (e.g. Au, contrast agent, paramagnetic agent, magnetic, optical agents) particles.

With the use of magnetic particles (e.g., nanoparticles), magnetic imaging methods like MRI may be used in conjugation with the therapeutic functionality of the protein. With the use of particles (e.g., nanoparticles) useful in optical techniques, methods such as photoacoustic imaging, fluorescence imaging, or optical coherence tomography, may be used in conjugation with the therapeutic functionality of the protein. In some embodiments of the compositions or methods described herein, multiple functionalities including optical and magnetic imaging functionalities may be combined to create not only a bi-functional but a multi-functional formulation from the protein dispersion.

In some embodiments of the low viscosity, high protein concentration dispersions as described herein (including embodiments), wherein the dispersions have been frozen, stored, and thawed, the average diameter of the plurality of nanoclusters (e.g., nanoparticles) is about the same (e.g. is the same) post-thawing as pre-freezing. In some embodiments of the dispersions, wherein the dispersions have been frozen, stored, and thawed, the post-thawing average diameter of the plurality of nanoclusters (e.g., nanoparticles) is within about 1% (e.g. within 1%) of the pre-freezing average diameter of the plurality of nanoclusters (e.g., nanoparticles). In some embodiments of the dispersions, wherein the dispersions have been frozen, stored, and thawed, the post-thawing average diameter of the plurality of nanoclusters (e.g., nanoparticles) is within about 5% (e.g. within 5%) of the pre-freezing average diameter of the plurality of nanoclusters (e.g., nanoparticles). In some embodiments of the dispersions, wherein the dispersions have been frozen, stored, and thawed, the post-thawing average diameter of the plurality of nanoclusters (e.g., nanoparticles) is within about 10% (e.g. within 10%) of the pre-freezing average diameter of the plurality of nanoclusters (e.g., nanoparticles). In some embodiments of the low viscosity, high protein concentration dispersions as described herein (including embodiments), wherein the dispersions have been frozen and thawed, the average diameter of the plurality of nanoclusters (e.g., nanoparticles) is about the same (e.g. is the same) post-thawing as pre-freezing. In some embodiments of the dispersions, wherein the dispersions have been frozen and thawed, the post-thawing average diameter of the plurality of nanoclusters (e.g., nanoparticles) is within about 1% (e.g. within 1%) of the pre-freezing average diameter of the plurality of nanoclusters (e.g., nanoparticles). In some embodiments of the dispersions, wherein the dispersions have been frozen and thawed, the post-thawing average diameter of the plurality of nanoclusters (e.g., nanoparticles) is within about 5% (e.g. within 5%) of the pre-freezing average diameter of the plurality of nanoclusters (e.g., nanoparticles). In some embodiments of the dispersions, wherein the dispersions have been frozen and thawed, the post-thawing average diameter of the plurality of nanoclusters (e.g., nanoparticles) is within about 10% (e.g. within 10%) of the pre-freezing average diameter of the plurality of nanoclusters (e.g., nanoparticles). As used herein, the term “store” or “storing”, as applied to a frozen dispersion, refers to maintaining the dispersion in a frozen state. In some embodiments, “store” or “storing” refers to maintaining the dispersion at a temperature of about −40 degrees Celsius. In some embodiments, “store” or “storing” refers to maintaining the dispersion at a temperature of less than about −40 degrees Celsius. In some embodiments, “store” or “storing” refers to maintaining the dispersion at a temperature of about −80 degrees Celsius. In some embodiments, “store” or “storing” refers to maintaining the dispersion at a temperature of about −20 degrees Celsius.

In some embodiments of the low viscosity, high protein concentration dispersions as described herein (including embodiments), wherein the dispersions have been frozen, stored, and thawed, the viscosity of the dispersion is about the same (e.g. is the same) post-thawing as pre-freezing. In some embodiments of the dispersions, wherein the dispersions have been frozen, stored, and thawed, the post-thawing viscosity of the dispersion is within about 1% (e.g. within 1%) of the pre-freezing viscosity of the dispersion. In some embodiments of the dispersions, wherein the dispersions have been frozen, stored, and thawed, the post-thawing viscosity of the dispersion is within about 5% (e.g. within 5%) of the pre-freezing viscosity of the dispersion. In some embodiments of the dispersions, wherein the dispersions have been frozen, stored, and thawed, the post-thawing viscosity of the dispersion is within about 10% (e.g. within 10%) of the pre-freezing viscosity of the dispersion. In some embodiments of the low viscosity, high protein concentration dispersions as described herein (including embodiments), wherein the dispersions have been frozen and thawed, the viscosity of the dispersion is about the same (e.g. is the same) post-thawing as pre-freezing. In some embodiments of the dispersions, wherein the dispersions have been frozen and thawed, the post-thawing viscosity of the dispersion is within about 1% (e.g. within 1%) of the pre-freezing viscosity of the dispersion. In some embodiments of the dispersions, wherein the dispersions have been frozen and thawed, the post-thawing viscosity of the dispersion is within about 5% (e.g. within 5%) of the pre-freezing viscosity of the dispersion. In some embodiments of the dispersions, wherein the dispersions have been frozen and thawed, the post-thawing viscosity of the dispersion is within about 10% (e.g. within 10%) of the pre-freezing viscosity of the dispersion.

In some embodiments of the low viscosity, high protein concentration dispersions as described herein (including embodiments), the dispersions are frozen for about one day (e.g. one day) and the average diameter of the plurality of nanoclusters (e.g., nanoparticles) is about the same (e.g. the same) post-thawing as pre-freezing. In some embodiments of the low viscosity, high protein concentration dispersions as described herein (including embodiments), the dispersions are frozen for about three days (e.g. three days) and the average diameter of the plurality of nanoclusters (e.g., nanoparticles) is about the same (e.g. the same) post-thawing as pre-freezing. In some embodiments of the low viscosity, high protein concentration dispersions as described herein (including embodiments), the dispersions are frozen for about one week (e.g. one week) and the average diameter of the plurality of nanoclusters (e.g., nanoparticles) is about the same (e.g. the same) post-thawing as pre-freezing. In some embodiments of the low viscosity, high protein concentration dispersions as described herein (including embodiments), the dispersions are frozen for about one month (e.g. one month) and the average diameter of the plurality of nanoclusters (e.g., nanoparticles) is about the same (e.g. the same) post-thawing as pre-freezing. In some embodiments of the low viscosity, high protein concentration dispersions as described herein (including embodiments), the dispersions are frozen for about one year (e.g. one year) and the average diameter of the plurality of nanoclusters (e.g., nanoparticles) is about the same (e.g. the same) post-thawing as pre-freezing.

In some embodiments of the low viscosity, high protein concentration dispersions as described herein (including embodiments), the dispersions are maintained (e.g. stored) as a frozen solid (e.g. at −40 degrees Celsius) for about one day (e.g. one day) and the average diameter of the plurality of nanoclusters (e.g., nanoparticles) is about the same (e.g. the same) post-thawing as pre-freezing. In some embodiments of the low viscosity, high protein concentration dispersions as described herein (including embodiments), the dispersions are maintained (e.g. stored) as a frozen solid (e.g. at −40 degrees Celsius) for about three days (e.g. three days) and the average diameter of the plurality of nanoclusters (e.g., nanoparticles) is about the same (e.g. the same) post-thawing as pre-freezing. In some embodiments of the low viscosity, high protein concentration dispersions as described herein (including embodiments), the dispersions are maintained (e.g. stored) as a frozen solid (e.g. at −40 degrees Celsius) for about one week (e.g. one week) and the average diameter of the plurality of nanoclusters (e.g., nanoparticles) is about the same (e.g. the same) post-thawing as pre-freezing. In some embodiments of the low viscosity, high protein concentration dispersions as described herein (including embodiments), the dispersions are maintained (e.g. stored) as a frozen solid (e.g. at −40 degrees Celsius) for about one month (e.g. one month) and the average diameter of the plurality of nanoclusters (e.g., nanoparticles) is about the same (e.g. the same) post-thawing as pre-freezing. In some embodiments of the low viscosity, high protein concentration dispersions as described herein (including embodiments), the dispersions are maintained (e.g. stored) as a frozen solid (e.g. at −40 degrees Celsius) for about one year (e.g. one year) and the average diameter of the plurality of nanoclusters (e.g., nanoparticles) is about the same (e.g. the same) post-thawing as pre-freezing.

In some embodiments, the dispersion medium includes a pharmaceutically acceptable solvent including a pharmaceutically acceptable aqueous solvent, a pharmaceutically acceptable non-aqueous solvent, or a combination. In some embodiments, the pharmaceutically acceptable solvents that may be used herein include benzyl benzoate or benzyl benzoate plus one or more oils selected from safflower, sesame, castor, cottonseed, canola, saffron, olive, peanut, sunflower seed, a-tocopherol, Miglyol 812, and ethyl oleate.

Stability of the protein in the composition disclosed hereinabove may be measured by size exclusion chromatography, analytical ultracentrifugation, CD spectroscopy, FTIR spectroscopy, dynamic light scattering, static light scattering, ELISA, native PAGE gel, or biological activity assays. In some embodiments, the composition (e.g. dispersion) exhibits substantially similar pharmacokinetic properties on injection when compared to an injectable solution of the protein or the peptide. In some embodiments of the method, the protein retains native conformation and activity within the dispersion and after dilution as measured by intrinsic tryptophan fluorescence, FTIR (fourier transmission infra-red spectroscopy), SEC, AUC, HPLC, light scattering, mass spectrometry, SEC, DLS, gel electrophoresis, antigen-specific or polyclonal ELISA, and specific in vitro activity assay.

### Pharmaceutical Compositions

In a further aspect a pharmaceutical composition is provided, including any of the dispersions as described herein (including embodiments), wherein the plurality of proteins is a plurality of pharmaceutically active proteins.

In some embodiments, the pharmaceutical composition is within a syringe attached to a 21 to 27 gauge needle. In some embodiments, pharmaceutical composition is within an osmotic pump. In some embodiments, the pharmaceutical composition is within a controlled release component, liposome, or microsphere. In embodiments, the pharmaceutical composition includes a pharmaceutically acceptable excipient. In embodiments, the dispersion, as described herein, is included in an effective amount (e.g., therapeutically effective amount).

### Kits

In a further aspect a kit is provided, wherein the kit includes a dispersion or pharmaceutical composition described herein (including embodiments). In some embodiments, the kit includes instructions for using the included dispersion or pharmaceutical composition. In some embodiments, the kit includes a vessel containing a dispersion or pharmaceutical composition as described herein (including embodiments).

In a further aspect a kit is provided, wherein the kit includes protein in powder form or a protein-crowder mixture in powder form, and a dispersion liquid. In some embodiments, the kit may be used in a method of making a dispersion or pharmaceutical composition described herein (including embodiments). In some embodiments, the kit includes instructions for making a dispersion as described herein (including embodiments). In some embodiments, the kit includes protein in powder form and a dispersion liquid. In some embodiments, the kit includes a protein-crowder mixture in powder form and a dispersion liquid. In some embodiments, the kit includes a syringe and a needle. In some embodiments, the kit includes instructions for mixing the protein in powder form or protein-crowder mixture in powder form with the dispersion liquid. In some embodiments, the kit includes instructions for mixing the protein in powder form or protein-crowder mixture in powder form with the dispersion liquid and self-administering the resulting dispersion.

### Methods of Treatment

In another aspect, a method is provided for treating a disease in a subject in need of such treatment, the method including administering an effective amount of any one of the dispersions described herein (including embodiments) to the subject. In some embodiments of the method of treating a disease, the administered dispersion includes about 0.5, 1, 2, 4, 6, 8, 10 mg of protein for each kg of body weight of the patient (e.g. 0.5, 1, 2, 4, 6, 8, 10 mg of protein for each kg of body weight).

The compositions (e.g. dispersion of protein nanoclusters (e.g., nanoparticles)) of the invention can be administered alone or can be coadministered to the patient. Coadministration is meant to include simultaneous or sequential administration of the compositions individually or in combination (more than one composition). Thus, the preparations can also be combined, when desired, with other active substances (e.g. to reduce metabolic degradation). The compositions described herein can be used in combination with one another, with other active agents known to be useful in treating a disease, or with adjunctive agents that may not be effective alone, but may contribute to the efficacy of the active agent, or with diagnostic agents.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

The compositions (e.g. dispersion of protein nanoclusters (e.g., nanoparticles)) described herein can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. It is also envisioned that multiple routes of administration (e.g., intramuscular, oral, transdermal) can be used to administer the compositions described herein (including embodiments). Accordingly, the present invention also provides pharmaceutical compositions including a pharmaceutically acceptable excipient and one or more compositions of the invention. The compositions disclosed herein can be administered by any means known in the art. For example, compositions may include administration to a subject intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a creme, or in a lipid composition. Administration can be local, e.g., to the site of disease (e.g. tumor in the case of cancer) or systemic.

For preparing pharmaceutical compositions from the compositions as described herein (including embodiments), pharmaceutically acceptable carriers can be either solid or liquid. Solid form preparations include powders, tablets, pills, capsules, cachets, suppositories, and dispersible granules. A solid carrier can be one or more substance, that may also act as diluents, flavoring agents, binders, preservatives, tablet disintegrating agents, or an encapsulating material.

When parenteral application is needed or desired, particularly suitable admixtures for the compositions are injectable, sterile dispersions, optionally oily or aqueous dispersion, as well as suspensions, emulsions, or implants, including suppositories. In particular, carriers for parenteral administration include aqueous solutions of dextrose, saline, pure water, buffers, ethanol, glycerol, propylene glycol, peanut oil, sesame oil, polyoxyethylene-block polymers, and the like. Ampules are convenient unit dosages. The compositions can also be incorporated into liposomes or administered via transdermal pumps or patches. Pharmaceutical admixtures suitable for use are well-known to those of skill in the art and are described, for example, in Pharmaceutical Sciences (17th Ed., Mack Pub. Co., Easton, Pa.) and WO 96/05309, the teachings of both of which are hereby incorporated by reference.

Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity. The aqueous suspension or dispersion can be made in water with a crowder or with a non-aqueous solvent with or without a crowder.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions as described herein (including embodiments) may additionally include components to provide sustained release and/or comfort. Such components include high molecular weight, anionic mucomimetic polymers, gelling polysaccharides and finely-divided drug carrier substrates. These components may serve multiple functions as they may also acts as a crowder to aid particles (e.g., nanoparticles, nanoclusters) formation. These components are discussed in greater detail in U.S. Pat. Nos. 4,911,920; 5,403,841; 5,212,162; and 4,861,760. The entire contents of these patents are incorporated herein by reference in their entirety for all purposes. The dispersions may be loaded into entities known to those in the field of drug delivery to further enable controlled (e.g. sustained) release including liposomes, microspheres, capsules, osmotic pumps, coating of polymer shells, matrices and implantable devices. In another embodiment, the dispersions may be dried and then loaded into these entities.

Pharmaceutical compositions include compositions wherein the active ingredient is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms. Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compositions described herein (including embodiments). Adjustment and manipulation of established dosages (e.g., frequency and duration) are well within the ability of those skilled in the art.

For any composition described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

In another aspect is provided a method of delivering a protein to a subject, the method including administering a dispersion described herein (including embodiments) to the subject. The dispersion includes the protein. In embodiments, the administering is a method of administering as described herein (including embodiments, for example in embodiments of the methods of treatment section herein).

### Methods of Modifying Nanoclusters

In another aspect, a method is provided for modifying the average protein particle (e.g., nanoparticles, nanoclusters) diameter of a low viscosity dispersion of protein nanoclusters (e.g., nanoparticles) including increasing or decreasing the concentration of a viscosity lowering agent or protein in the dispersion. The dispersion includes a plurality of nanoclusters (e.g., nanoparticles) and each of the plurality of nanoclusters (e.g., nanoparticles) includes a plurality of proteins. Each of the plurality of proteins shares amino acid sequence identity.

### Methods of Making Dispersions

In another aspect is provided a method of making a low viscosity dispersion as described herein, including in an embodiments, example, figure, table, or claim.

In embodiments of the method, the low viscosity dispersion includes a protein and a viscosity lowering agent. The viscosity lowering agent may be present at a concentration from about 10 mg/mL to about 300 mg/mL. The protein may be present at a concentration of greater than about 200 mg/mL. The protein may be present within a plurality of protein particles. The viscosity lowering agent may be proline, lysine, betaine, asparagine, glutamine, glutamic acid, histidine, arginine or imidazole.

In embodiments, the method includes the step of concentrating a protein-viscosity lowering agent liquid combination and thereby forming a dispersion described herein. In embodiments, the method includes the step of combining a protein in powder form with a viscosity lowering agent and a dispersion liquid and thereby forming a dispersion described herein. In embodiments, the method includes the step of removing a solvent from a protein mixture thereby forming the protein in powder form. In embodiments, the method includes milling, precipitating, dialyzing, sieving, spray drying, lyophilizing, spray freeze drying, or spray freezing a protein mixture. In embodiments, the method includes applying spiral wound in situ freezing technology (SWIFT) to a protein mixture. In embodiments, the method includes combining a protein in powder form with a dispersion liquid. In embodiments, the method includes filtration. In embodiments, the method includes centrifugal filtration. In embodiments, the method includes positive gas pressure or mechanical pressure. In embodiments, the method includes tangential flow filtration, dialysis, or absorption of buffer. In embodiments, the method includes using a compound capable of absorbing liquid (e.g. a molecular sieve). In embodiments, the method includes lyophilization of a protein followed by dilution of the lyophilized powder. In embodiments, the method includes addition of dispersions formed by centrifugation to lyophilized powder. In embodiments, the method includes centrifugation filtration to concentrate solutions and form nanoparticles (e.g., nanoclusters).

In embodiments, the method is a method of making a low viscosity, high protein dispersion of protein nanoclusters, including concentrating a protein-viscosity lowering agent (e.g., crowder) liquid combination and thereby forming the dispersion. In some embodiments, the method includes, prior to the concentrating, combining a solution of the protein with a viscosity lowering agent (e.g., crowder) in a vessel to form a protein-viscosity lowering agent (e.g., crowder) liquid combination. In some embodiments of the method, the protein-viscosity lowering agent (e.g., crowder) liquid combination includes a dispersion of protein nanoclusters (e.g., nanoparticles) with an average protein nanoclusters (e.g., nanoparticles) diameter different from the average diameter of the plurality of protein nanoclusters (e.g., nanoparticles) formed by the concentrating. In some embodiments of the method of making a low viscosity, high protein dispersion of protein nanoclusters (e.g., nanoparticles), the dispersion is selected from the dispersions described herein (including embodiments).

In embodiments, the method is a method of making a low viscosity, high protein dispersion of protein nanoclusters (e.g., nanoparticles), including the step of combining a protein in powder form with a viscosity lowering agent (e.g., crowder) and a dispersion liquid thereby forming a dispersion including a plurality of nanoclusters (e.g., nanoparticles) including a plurality of the protein. Each of the plurality of proteins shares amino acid sequence identity. In some embodiments, the method includes, prior to the combining, removing a solvent from a protein mixture thereby forming the protein in powder form. In some embodiments of the method, the protein mixture is a protein dispersion or a protein solution. In some embodiments of the method, the removing includes milling, precipitating, dialyzing, sieving, spray drying, lyophilizing, or spray freeze drying, spray freezing the protein mixture; or the removing includes applying spiral wound in situ freezing technology (SWIFT) to the protein mixture. In some embodiments of the method, the removing includes thin film freezing. In some embodiments of the method, the solvent is water. In some embodiments of the method of making a low viscosity, high protein dispersion of protein nanoclusters (e.g., nanoparticles), the dispersion is selected from the dispersions described herein (including embodiments).

In embodiments, the method is a method of making a low viscosity, high protein dispersion of protein nanoclusters (e.g., nanoparticles), including the step of combining a protein in powder form with a dispersion liquid thereby forming a dispersion including a plurality of nanoclusters (e.g., nanoparticles) including a plurality of the protein. Each of the plurality of proteins shares amino acid sequence identity. In some embodiments, the method includes prior to the combining, removing a solvent from a protein-viscosity lowering agent (e.g., crowder) mixture thereby forming the protein in powder form, which may optionally contain a viscosity lowering agent (e.g., crowder). In some embodiments of the method, the protein-viscosity lowering agent (e.g., crowder) mixture is a protein dispersion or a protein solution. In some embodiments of the method, the removing includes milling, precipitating, dialyzing, sieving, spray drying, lyophilizing, or spray freeze drying, spray freezing the protein-viscosity lowering agent (e.g., crowder) mixture; or the removing includes applying spiral wound in situ freezing technology (SWIFT) to the protein-viscosity lowering agent (e.g., crowder) mixture. In some embodiments of the method, the solvent is water. In some embodiments of the method of making a low viscosity, high protein dispersion of protein nanoclusters (e.g., nanoparticles), the dispersion is selected from the dispersions described herein (including embodiments).

In some embodiments of the methods of making a low viscosity, high protein dispersion of protein nanoclusters (e.g., nanoparticles), as described herein (including embodiments), the dispersion liquid is water, an aqueous liquid, or a non-aqueous liquid. In some embodiments of the methods of making a low viscosity, high protein dispersion of protein nanoclusters (e.g., nanoparticles), as described herein (including embodiments), the dispersion liquid is benzyl benzoate or benzyl benzoate plus one or more oils selected from safflower, sesame, castor, cottonseed, canola, saffron, olive, peanut, sunflower seed, a-tocopherol, Miglyol 812, and ethyl oleate.

In some embodiments of the methods of making a low viscosity, high protein dispersion of protein nanoclusters (e.g., nanoparticles), as described herein (including embodiments), the removing includes applying spiral wound in situ freezing technology (SWIFT) to the mixture. In some embodiments of the methods of making a low viscosity, high protein dispersion of protein nanoclusters (e.g., nanoparticles), as described herein (including embodiments), applying SWIFT includes the steps of: (1) rotating a vial, containing the mixture, while contacting the vial with a cryogenic agent; (2) freezing all of the mixture, wherein the freezing results in a thin film of the frozen mixture on the inner side of the vial and one or more subsequent films in a spiral orientation towards the center of the vial; and (3) lyophilizing the frozen mixture. In some embodiments, SWIFT may include contacting the vial with a cold substance (e.g. dry ice) instead of a cryogenic agent.

In some embodiments of the methods of making a low viscosity, high protein dispersion of protein nanoclusters (e.g., nanoparticles), as described herein (including embodiments), the concentrating is performed using filtration. In some embodiments of the methods of making a low viscosity, high protein dispersion of protein nanoclusters (e.g., nanoparticles), as described herein (including embodiments), the concentrating is performed using centrifugal filtration. In some embodiments of the methods, the concentrating is performed using positive gas pressure or mechanical pressure. In some embodiments of the methods, the concentrating is performed using tangential flow filtration, dialysis, or absorption of buffer. In some embodiments of the methods, the concentrating is performed using a compound capable of absorbing liquid (e.g. a molecular sieve). In some embodiments of the methods, the concentrating includes adding a compound (e.g. a molecular sieve) to the protein-viscosity lowering agent (e.g., crowder) mixture, wherein the added compound absorbs liquid. In some embodiments of the methods, the concentrating includes adding a compound (e.g. a molecular sieve) to the protein-viscosity lowering agent (e.g., crowder) mixture, wherein the added compound reduces the water in the protein-viscosity lowering agent (e.g., crowder) mixture by removing it from the bulk solution. In some embodiments of the methods, a viscosity lowering agent (e.g., crowder) or the protein is added to the protein-viscosity lowering agent (e.g., crowder) liquid combination during the concentrating.

In some embodiments, the methods of making a low viscosity, high protein dispersion of protein nanoclusters (e.g., nanoparticles), as described herein (including embodiments), further include sterilizing the dispersion. In some embodiments, the methods of making a low viscosity, high protein dispersion of protein nanoclusters (e.g., nanoparticles), as described herein (including embodiments), further include sterilizing the dispersion by filtration. In some embodiments, the methods of making a low viscosity, high protein dispersion of protein nanoclusters (e.g., nanoparticles), as described herein (including embodiments), further include sterilizing the dispersion by filtration through a filter having pores of about 200 nm diameter (e.g. 200 nm diameter).

In some embodiments, the methods of making a low viscosity, high protein dispersion of protein nanoclusters (e.g., nanoparticles), as described herein (including embodiments), further include freezing, storing, and thawing the dispersion, and the average diameter of the plurality of nanoclusters (e.g., nanoparticles) is about the same (e.g. is the same) post-thawing as pre-freezing. In some embodiments of the methods, further including freezing, storing, and thawing the dispersion, the post-thawing average diameter of the plurality of nanoclusters (e.g., nanoparticles) is within about 1% (e.g. within 1%) of the pre-freezing average diameter of the plurality of nanoclusters (e.g., nanoparticles). In some embodiments of the methods, further including freezing, storing, and thawing the dispersion, the post-thawing average diameter of the plurality of nanoclusters (e.g., nanoparticles) is within about 5% (e.g. within 5%) of the pre-freezing average diameter of the plurality of nanoclusters (e.g., nanoparticles). In some embodiments of the methods, further including freezing, storing, and thawing the dispersion, the post-thawing average diameter of the plurality of nanoclusters (e.g., nanoparticles) is within about 10% (e.g. within 10%) of the pre-freezing average diameter of the plurality of nanoclusters (e.g., nanoparticles). In some embodiments, the methods of making a low viscosity, high protein dispersion of protein nanoclusters (e.g., nanoparticles), as described herein (including embodiments), further include freezing and thawing the dispersion, and the average diameter of the plurality of nanoclusters (e.g., nanoparticles) is about the same (e.g. is the same) post-thawing as pre-freezing. In some embodiments of the methods, further including freezing and thawing the dispersion, the post-thawing average diameter of the plurality of nanoclusters (e.g., nanoparticles) is within about 1% (e.g. within 1%) of the pre-freezing average diameter of the plurality of nanoclusters (e.g., nanoparticles). In some embodiments of the methods, further including freezing and thawing the dispersion, the post-thawing average diameter of the plurality of nanoclusters (e.g., nanoparticles) is within about 5% (e.g. within 5%) of the pre-freezing average diameter of the plurality of nanoclusters (e.g., nanoparticles). In some embodiments of the methods, further including freezing and thawing the dispersion, the post-thawing average diameter of the plurality of nanoclusters (e.g., nanoparticles) is within about 10% (e.g. within 10%) of the pre-freezing average diameter of the plurality of nanoclusters (e.g., nanoparticles).

In some embodiments, the methods of making a low viscosity, high protein dispersion of protein nanoclusters (e.g., nanoparticles), as described herein (including embodiments), further include freezing, storing, and thawing the dispersion, and the viscosity of the dispersion is about the same (e.g. is the same) post-thawing as pre-freezing. In some embodiments of the methods, further including freezing, storing, and thawing the dispersion, the post-thawing viscosity of the dispersion is within about 1% (e.g. within 1%) of the pre-freezing viscosity of the dispersion. In some embodiments of the methods, further including freezing, storing, and thawing the dispersion, the post-thawing viscosity of the dispersion is within about 5% (e.g. within 5%) of the pre-freezing viscosity of the dispersion. In some embodiments of the methods, further including freezing, storing, and thawing the dispersion, the post-thawing viscosity of the dispersion is within about 10% (e.g. within 10%) of the pre-freezing viscosity of the dispersion. In some embodiments, the methods of making a low viscosity, high protein dispersion of protein nanoclusters (e.g., nanoparticles), as described herein (including embodiments), further include freezing and thawing the dispersion, and the viscosity of the dispersion is about the same (e.g. is the same) post-thawing as pre-freezing. In some embodiments of the methods, further including freezing and thawing the dispersion, the post-thawing viscosity of the dispersion is within about 1% (e.g. within 1%) of the pre-freezing viscosity of the dispersion. In some embodiments of the methods, further including freezing and thawing the dispersion, the post-thawing viscosity of the dispersion is within about 5% (e.g. within 5%) of the pre-freezing viscosity of the dispersion. In some embodiments of the methods, further including freezing and thawing the dispersion, the post-thawing viscosity of the dispersion is within about 10% (e.g. within 10%) of the pre-freezing viscosity of the dispersion.

In some embodiments, the methods of making a low viscosity, high protein dispersion of protein nanoclusters (e.g., nanoparticles), as described herein (including embodiments), further includes freezing the dispersion for about one day (e.g. one day) and thawing the dispersion, wherein the average diameter of the plurality of nanoclusters (e.g., nanoparticles) is about the same (e.g. the same) post-thawing as pre-freezing. In some embodiments, the methods of making a low viscosity, high protein dispersion of protein nanoclusters (e.g., nanoparticles), as described herein (including embodiments), further includes freezing the dispersion for about three days (e.g. three days) and thawing the dispersion, wherein the average diameter of the plurality of nanoclusters (e.g., nanoparticles) is about the same (e.g. the same) post-thawing as pre-freezing. In some embodiments, the methods of making a low viscosity, high protein dispersion of protein nanoclusters (e.g., nanoparticles), as described herein (including embodiments), further includes freezing the dispersion for about one week (e.g. one week) and thawing the dispersion, wherein the average diameter of the plurality of nanoclusters (e.g., nanoparticles) is about the same (e.g. the same) post-thawing as pre-freezing. In some embodiments, the methods of making a low viscosity, high protein dispersion of protein nanoclusters (e.g., nanoparticles), as described herein (including embodiments), further include freezing the dispersion for about one month (e.g. one month) and thawing the dispersion, wherein the average diameter of the plurality of nanoclusters (e.g., nanoparticles) is about the same (e.g. the same) post-thawing as pre-freezing. In some embodiments, the methods of making a low viscosity, high protein dispersion of protein nanoclusters (e.g., nanoparticles), as described herein (including embodiments), further includes freezing the dispersion for about one year (e.g. one year) and thawing the dispersion, wherein the average diameter of the plurality of nanoclusters (e.g., nanoparticles) is about the same (e.g. the same) post-thawing as pre-freezing.

In some embodiments, the methods of making a low viscosity, high protein dispersion of protein nanoclusters (e.g., nanoparticles), as described herein (including embodiments), further includes maintaining (e.g. storing) the dispersion as a frozen solid (e.g. at −40 degrees Celsius) for about one day (e.g. one day) and then thawing the dispersion, wherein the average diameter of the plurality of nanoclusters (e.g., nanoparticles) is about the same (e.g. the same) post-thawing as pre-freezing. In some embodiments, the methods of making a low viscosity, high protein dispersion of protein nanoclusters (e.g., nanoparticles), as described herein (including embodiments), further includes maintaining (e.g. storing) the dispersion as a frozen solid (e.g. at −40 degrees Celsius) for about three days (e.g. three days) and then thawing the dispersion, wherein the average diameter of the plurality of nanoclusters (e.g., nanoparticles) is about the same (e.g. the same) post-thawing as pre-freezing. In some embodiments, the methods of making a low viscosity, high protein dispersion of protein nanoclusters (e.g., nanoparticles), as described herein (including embodiments), further includes maintaining (e.g. storing) the dispersion as a frozen solid (e.g. at −40 degrees Celsius) for about one week (e.g. one week) and then thawing the dispersion, wherein the average diameter of the plurality of nanoclusters (e.g., nanoparticles) is about the same (e.g. the same) post-thawing as pre-freezing. In some embodiments, the methods of making a low viscosity, high protein dispersion of protein nanoclusters (e.g., nanoparticles), as described herein (including embodiments), further include maintaining (e.g. storing) the dispersion as a frozen solid (e.g. at −40 degrees Celsius) for about one month (e.g. one month) and then thawing the dispersion, wherein the average diameter of the plurality of nanoclusters (e.g., nanoparticles) is about the same (e.g. the same) post-thawing as pre-freezing. In some embodiments, the methods of making a low viscosity, high protein dispersion of protein nanoclusters (e.g., nanoparticles), as described herein (including embodiments), further includes maintaining (e.g. storing) the dispersion as a frozen solid (e.g. at −40 degrees Celsius) for about one year (e.g. one year) and then thawing the dispersion, wherein the average diameter of the plurality of nanoclusters (e.g., nanoparticles) is about the same (e.g. the same) post-thawing as pre-freezing.

In some embodiments of the method, the proteins or peptides revert into a monomeric form upon dilution from the dispersion medium. In some embodiments of the method, the proteins or peptides retain at least 95%, 96%, 97%, 98%, 99%, and 100% activity upon dilution from the dispersion medium. In some embodiments of the method, the composition exhibits substantially similar pharmacokinetic properties on injection when compared to an injectable solution of the protein or the peptide.

It is contemplated that any embodiment discussed in this specification can be implemented with respect to any method, kit, reagent or composition of the invention, and vice versa. Furthermore, compositions of the invention can be used to achieve methods of the invention.

### Examples

The following examples are for purposes of illustration only and are not intended to limit the scope of the claims or the spirit or scope of the disclosure.

### Example 1

### Characterization of the Protein Nanocluster Dispersion

Buffer Exchange.

After thawing and dilution, the ˜4 mg/mL solution of mAb 1 was buffer exchanged into 50 mM phosphate buffer with the desired amount of dissolved proline (Alfa Aesar), typically 150 mg/mL or none. The buffer exchange was carried out using centrifugal filter tubes (Millipore, Amicon Ultracel 30K centrifugal filters) with a molecular weight cutoff of 30 kDa and a capacity of 12 mL. A desired amount of the protein solution (typically 6-10 mL) was added to the filter tube, and the desired buffer for the dispersion was added to increase the solution volume to 12 mL. The buffer was forced through the membrane by centrifugal filtration at 4500 radial centrifugal force (rcf) for 12 minutes concentrating the protein solution in the retentate until the solution volume dropped to about 2 mL. The retentate protein solution was then diluted to 12 mL in the same buffer as before and concentrated down to 2 mL again. The dilution and centrifugation process was repeated 4 or more times until the permeate volume was 4-5 times the original solution volume, typically 40 mL. After buffer exchange, the solution was further concentrated to 50-80 mg/ml so that the final solution volume was about 0.5 mL.

Centrifugal Filtration of Protein Solution to Form a Dispersion of Nanoclusters Upon Concentration.

Tare weights were taken of a centrifugal filter assembly (Millipore Microcon, Ultracel YM-50 membrane, 50 kDa nominal molecular weight limit, diameter of filter, 0.25″). The desired volume (˜0.5 mL) of protein solution, after buffer exchange and concentration, was pipetted into the retentate chamber. The filter assembly was then centrifuged (Eppendorf Centrifuge 5415D) at 10,000 rcf—typically in 20-40 minute increments until the retentate reached the calculated volume needed to achieve the desired final protein concentration. The volume measurements were done using image analysis (ImageJ software) to determine the height of the liquid column in the retentate chamber. Additionally, the protein concentration in the retentate dispersion was determined by measuring out 2 μL (±0.08 μL) of dispersion using an Eppendorf Research adjustable volume 0.5-10 μL pipette and diluting it into a receiving vessel containing 998 μL of 50 mM pH 6.4 phosphate buffer. For mixing, the solution was cycled 5 times into and back out of the pipette tip followed by light agitation with the pipette tip. The absorbance of the resulting solution at 280 nm was measured using a Cary 3E UV-visible spectrophotometer in a cuvette (Hellma cells) with a path length of 1 cm, and converted to concentration assuming an extinction coefficient of 1.42 mL mg⁻¹ cm⁻¹.

Once the desired concentration had been reached, the dispersion of protein nanoclusters in the retentate chamber was recovered either by inverting the filter assembly into a retentate recovery tube, and centrifuging it for 3-4 minutes at 1,000 RCF (“spin off”) or by inverting it into the retentate recovery tube and allowing the dispersion to flow into the tube under gravity (“pour off”). The resulting dispersion was transferred to a 0.1 mL conical vial (V-Vial, Wheaton), and the concentration was confirmed using 2 μL of the dispersion as described above.

Lyophilization Dilution.

Prior to lyophilization, the mAb 1 solution described above was buffer exchanged into DI water and concentrated to ˜60-80 mg/mL using a 50,000 molecular weight cutoff (MWCO) Centricon filter as described above in the buffer exchange step. Solid proline was then added to the mAb 1 solution at a 1:1 protein: proline weight ratio as a cryoprotectant. The solution was filter-sterilized (0.22 μm), diluted to ˜50 mg/mL protein with DI water, and transferred to a glass vial. It was frozen over 6 hours at −40° C. and then lyophilized (VirTis Advantage Plus Benchtop Freeze Dryer) at 150 mTorr with 24 hours of primary drying at −40° C. followed by a 6 hour ramp to 25° C. and an additional 6 hours of secondary drying at 25° C. To create a dispersion, typically 40 mg of lyophilized protein was compacted into a tared 0.1 mL conical vial (V-Vial, Wheaton). After addition of the desired buffer and/or additional proline, the resulting dispersion was stirred gently with the tip of a 25 gauge needle. The total volume and volume fractions of the components were calculated assuming ideal mixing based on known masses, and hypothetical pure liquid protein (1.35 g/cm³) and proline (1.40 g/cm³) densities, from their partial molar volumes at infinite dilution and a known buffer volume. The final protein concentration was verified using light absorbance at 280 nm as described above.

Centrifugation Dispersion over Powder.

For C-D/P, lyophilized powder was manufactured as described above. In addition, a dispersion with the desired pre-powder addition mAb 1 and proline concentration was manufactured via buffer exchange and centrifugation concentration as previously described. The required amount of lyophilized powder necessary to bring the dispersion to the desired final concentration, calculated assuming ideal mixing based on known masses, and hypothetical pure liquid protein (1.35 g/cm³) and proline (1.40 g/cm³) densities, was weighed into a tared 0.1 mL conical vial. The required volume of dispersion was then pipetted atop the powder, and the resulting mixture was stirred gently with the tip of a 25 gauge needle until the liquid dispersion was optically transparent.

Zeta Potential.

Zeta potential analysis was conducted using a Brookhaven ZetaPlus zeta potential analyzer. Samples were formulated at 1 mg/mL mAb 1 concentration with the desired concentration of excipient in the specified buffer, and the averages for 30 single-cycle measurements are reported.

Hydrodynamic Diameters (e.g., Nanocluster Diameters).

The short-time mutual diffusion coefficient Dₛ(q) of protein nanoclusters was extracted from intensity correlation functions measured using dynamic light scattering. Measurements were taken at an angle of 150° with a 632.8 nm laser (q=0.01918 nm⁻¹) and an avalanche photodiode at ˜23° C. using a custom apparatus (Brookhaven BI-9000AT and 60 μl Beckman Coulter sample cell) and analyzed with the CONTIN algorithm (volume distribution). Hydrodynamic nanocluster diameters D_{c} were estimated from the Dₛ(q) using Beenakker-Mazur theory for Dₛ(q)/D₀, where D₀=kT/3πηD_{c} and η is the shear viscosity of the buffer solvent with added excipients. This approach assumes that the protein nanoclusters act like suspended hard spheres occupying an effective packing fraction φ_{c}^{eff}=φ/φ_{c}^{int}, where φ_{c}^{int} is the protein packing fraction within a nanocluster. In this work, we assumed φ_{c}^{int}=0.60, which is consistent with light scattering data on protein nanoclusters reported previously. We also verified that an alternative approximation, φ_{c}^{int}=(D_{c}/2R)^{δ}^{_{f}}⁻³ (where δ_{f} is the fractal dimension, taken as 2.6), resulted in similar nanocluster size estimates. The measured intensity correlation functions decayed on time scales between ˜10 to 50 μs, consistent with short-time diffusion for nanoclusters with the diameters and mobilities reported here.

Size Exclusion Chromatography.

For analysis of non-covalent aggregates, the sample was diluted to ˜1 mg/mL in 50 mM pH 6.4 sodium phosphate buffer. 20 μg of diluted dispersion was analyzed with a Waters Breeze HPLC, using TOSOH Biosciences TSKgel3000SW_{XL} and TSKgel2000SW columns in series and a mobile phase of 100 mM sodium phosphate, 300 mM sodium chloride, pH 7, with eluate monitored by absorbance at 214 nm. For analysis of proline concentration in retentate samples from ultrafiltration, samples were prepared identically to the non-covalent aggregate analysis, with the exception that final protein concentration was measured in order to determine dilution factor. The area under the curve for the proline peak was measured and compared to a standard curve that had been determined a priori (Proline Beer's Law plot) in order to determine proline concentration.

Viscosity.

The viscosities of the nanocluster dispersions were measured in triplicate using a 25 gauge (ID=0.1 mm) 1.5″ long needle (Becton Dickinson & Co. Precision Glide Needle) attached to a 1 mL syringe (Becton Dickinson & Co. 1 mL syringe with Luer-Lok™ tip), according to the Hagen-Poiseuille equation. The flow rate of the dispersion through the needle was determined by correlating volume to the height of the liquid in the conical vial (using ImageJ software) and measuring the time taken for the dispersion column height to move between two points. The flow rate was correlated to viscosity from a calibration curve derived from a set of standards of known viscosities.

pH Measurement.

The pH of the buffer solutions prepared with and without dissolved proline (for making dispersions by centrifugation and dispersion over powder, respectively) was measured using a pH meter (Mettler-Toledo) with an accuracy of ±0.01 pH units. Due to the small sample size (100 μL) of the final dispersion, the dispersion pH was measured using pH indicator strips (EMD Millipore) with an accuracy of ±0.5 pH units. pH measurements of the same sample by the two methods agreed within 0.5 pH units, which is at the maximum level of sensitivity of the pH indicator strips.

Protein Surface Charge and Free Energy Model for Nanocluster Size.

The human monoclonal antibody used for this study is mAb 1. It was stored frozen in 10 mL aliquots at ˜100 mg/mL and −40° C., and thawed at 2-8° C. Each aliquot had been filtered through a 0.22 μm filter (Pall) prior to freezing. The protein concentration was verified via A280 prior to use. For extended refrigerated storage, mAb 1 was diluted to ˜4 mg/mL in pH 6.4 50 mM phosphate buffer and stored at 2-8° C.

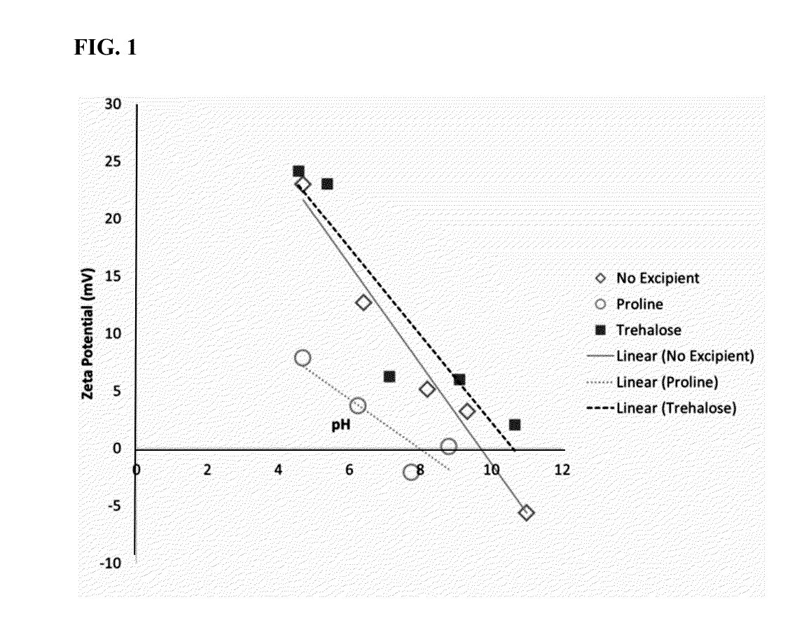

To better understand how interactions of proline with mAb1 moderate the charge, the zeta potential of a 2 mg/mL mAb 1 solution with 150 mg/mL of proline at different pH levels (controlled by buffer solutions) was measured, as shown in FIG. 1. Similar experiments were also performed with trehalose at 200 mg/mL instead of proline. Without added proline, the pI of mAb 1 was reported as 9.3 by the supplier. This was verified within the precision of the instrument, as seen in FIG. 1, whereby the zeta potential of mAb 1 was very close to zero from pH 8 to 10. The addition of proline to the mAb 1 solution reduced the zeta potential on mAb 1 to undetectable levels between pH 7 and 9. In contrast the addition of trehalose did not reduce the zeta potential significantly in the range studied. The reduction of the zeta potential by proline suggests that proline interacts with and screens the positive surface charges on the protein at the protonated sites. It is likely that the zeta potential would be reduced by a smaller extent in concentrated protein solutions, since the ratio of proline to protein would be much smaller. The large amounts of protein required for such experiments in a zeta potential cell volume of 1.5 ml was prohibitive for the current study.

To describe qualitative effects on how the nanoclusters form, it is insightful to examine the roles of the various colloidal forces with a semi-quantitative free energy model previously developed for protein nanoclusters, based on an earlier model for nanoclusters in organic solvents. When the charge on the protein is small (either near the pI in the previously-published trehalose system, or within the minimal charge range of pH 6-10 with proline systems) the electrostatic repulsion between protein monomers is relatively weak. As a consequence of this weak repulsive force, attractive forces between the monomers favor assembly of nanoscale clusters. In order to drive nanocluster formation, an extrinsic crowding agent (depletant) has been added to produce excluded volume (i.e. osmotic depletion) attraction between the protein molecules. The approximate equilibrium free energy and its derivation were described in detail previously, but specifically, the model suggests that nanocluster diameters obey the following proportionality:

$${D}_{c} ∝ {[ \frac{ɛ / kT}{( λ / R ) {q}^{2}} ]}^{\frac{1}{2 {δ}_{f} - 3 }} \qquad ( 2 )$$

where ε is the magnitude of the effective attraction between two adjacent protein molecules in the nanocluster, k is the Boltzmann constant, T is temperature, λ is the Bjerrum length, R is the protein radius, q is the charge per protein molecule, and δ_{f} is the fractal dimension of the nanocluster. As the concentration of depletant is increased, ε increases and the nanoclusters formed by trehalose increased in size as predicted by Eq. 2.

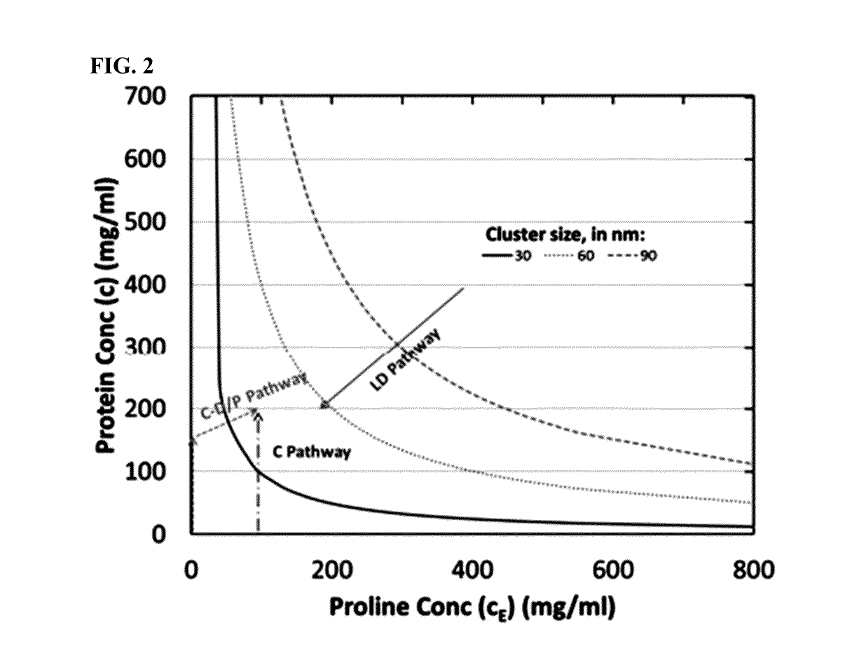

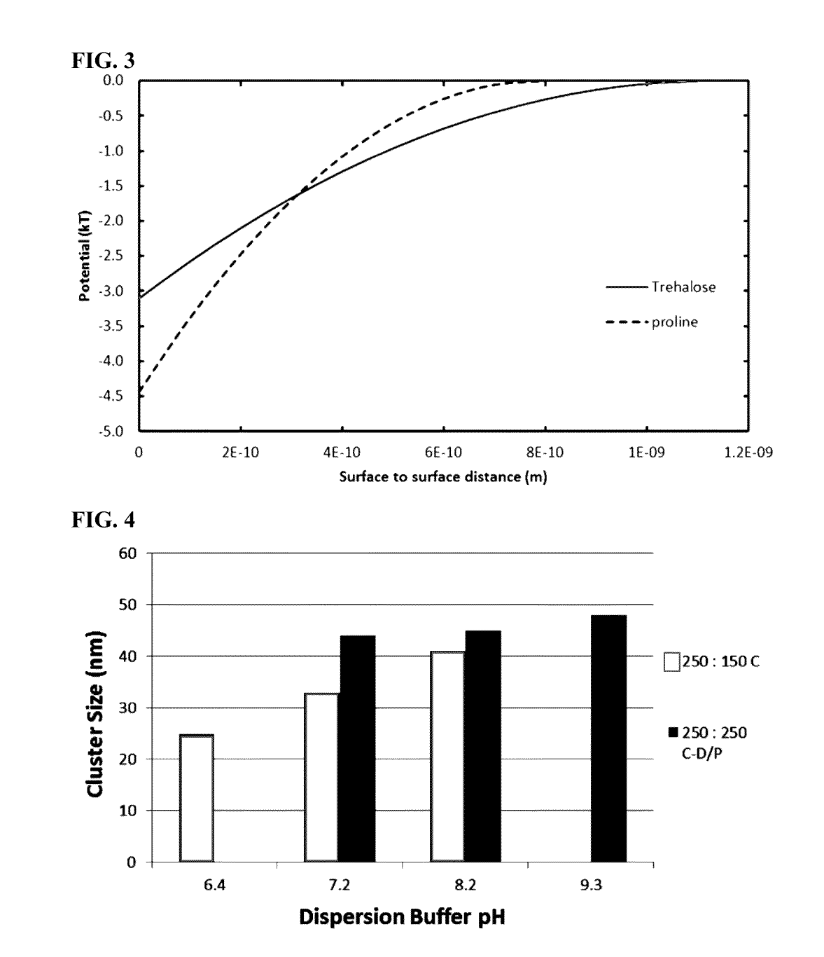

The free energy model was used to estimate nanocluster diameter contours (FIG. 2) for mAb 1 with proline for parameters listed in Table 1. Trehalose has a hydrodynamic radius of 0.5 nm relative to only 0.268 nm for proline. Therefore, in the case of proline which is a smaller molecule than trehalose, the depletion attraction will have a higher strength at contact, but will have a shorter range, as seen in FIG. 3. The model uses the contact value of the depletion attraction. Additionally proline binding lowers the protein charge (q²<<1), producing weaker electrostatic repulsion and larger self-assembled nanoclusters. Therefore, the dual roles of proline as a depletant and a protein surface charge modifier both favor nanocluster formation at a pH of ˜6-7. Qualitative nanocluster size contours are graphed in FIG. 2, along with the differing pathways that were used to form nanoclusters. While this model does not take into account the electrostatic screening by proline due to specific interactions with the protein, nor the preferential hydration of the protein that may be induced by proline, nor effects of pH on specific interactions, it still provides a qualitative explanation for how concentrations of protein and depletant might affect nanocluster size for an assumed charge. Below, we examine our hypothesis that proline's role in reduction of protein surface charge away from the pI may be beneficial for forming nanoclusters, to complement our previous study of protein nanoclusters very near the pI of the protein. The implication of this charge screening effect is that the charge on a mAb with a high isoelectric point may be reduced even 3 pH units to a more favorable pH for injection.

Dilution of Lyophilized Powder in Buffer with Two Processes (LD).

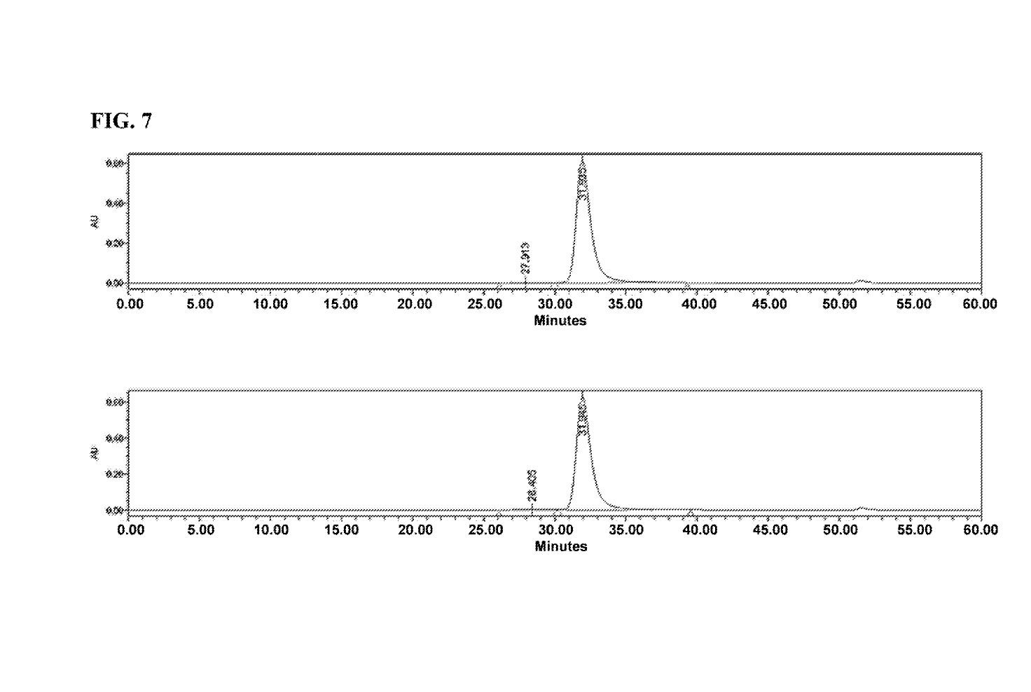

Earlier reported protein nanoclusters were formed by dilution (mixing) of lyophilized powder containing protein and crowder (LD) in buffer, as shown for sheep IgG, mAb 1B7 and BSA with trehalose. In the current study, mAb 1 and proline as a cryoprotectant at a 1:1 mass ratio were lyophilized. As can be seen in Table 2, proline provided sufficient cryoprotection to mAb 1, with aggregation less than 1% as observed by SEC. (A representative SEC curve can be found in FIG. 7).

The stable lyophilized powder was utilized by two different techniques to produce dispersions. In the first approach, powder was packed into a conical vial and diluted by adding buffer was added as done previously. In this study, a second approach was designed in which a dispersion formed by this first approach was then added on top of additional lyophilized powder to achieve the same final protein concentration. In this case approximately ˜50 percent of the protein was introduced by the second increment of powder. The term LD is used to refer to both of these processes, with the additional suffix of ‘-D/P’ to indicate the addition of the dispersion over the powder in the second increment.

The pathway of nanocluster formation (the LD pathway) is shown in FIG. 2. As the lyophilized powder contacted the buffer solution upon gentle stirring, an optically clear dispersion of protein was produced after bubbles formed during mixing dissipated. As can be seen in Table 3, a large variety of nanoclusters were formed with protein concentrations from 235 to 250 mg/mL with proline concentrations from 100 to 400 mg/mL. Despite the large increase in depletion attraction with proline concentration, the nanocluster size did not vary significantly. Additional proline beyond that which was included in the initial lyophilized powder was added to raise the concentration up to 400 mg/mL (sample LD-D/P 234:400P), and the dispersion viscosity increased from 159 to 214 cP. The self-clustering of proline above ˜200 mg/mL presents a complication that may influence the depletion potential at the highest concentrations as well as the viscosity. A weakening of depletion with a loss in number of proline entities upon formation of proline aggregates may contribute to the lack of an increase in nanocluster size with proline concentration. In contrast, for trehalose as a crowder, as the concentration was increased from 100 to 270 mg/mL, the nanocluster size increased from 51 to 95 nm for a protein concentration of ˜250 mg/mL for polyclonal sheep IgG.

The autocorrelation function (ACF) is the true output data of dynamic light scattering (DLS). DLS measures the intensity of scattered light as a function of time and the ACF is a measure of the correlation between the scattered intensity of light at a time of t₀ and a time of t₀+τ, with a value of 1 being perfectly correlated and a value of 0 being no correlation. The ACF of N different species can be expressed by the following equation

$$g ( τ ) = {[ {\sum }_{i = 1}^{N} {A}_{i} exp [ - \frac{τ}{{τ}_{i}} ] ]}^{2}$$

Where g(τ) is the ACF, Aᵢ is the amplitude of scattered light from species i, τ is the delay time after t₀, and τᵢ is the characteristic delay time of species i. For a monodisperse system (all particles are the same size) the autocorrelation is

$$g ( τ ) = {[ {A}_{i} exp [ - \frac{τ}{{τ}_{i}} ] ]}^{2}$$

Where A₁ is the maximum amplitude of the ACF (the value of g(τ=0)) and τ₁ is the characteristic delay time which represents the value oft at which the ACF equals A₁ times the exponential of −2, ˜0.14*A₁. Therefore the further out the curve ACF shifts out on the curve the larger the characteristic delay time. The diameter of the particle, D₁, can be related to the characteristic delay time by the following equation

$${D}_{1} = \frac{{k}_{B} {Tq}^{2}}{6 {πη}_{0}} {τ}_{1}$$

Where k_{B} is the Boltzmann constant, T is the temperature, q is the scattering vector (determined by the laser properties, instrument geometry and solvent) and η₀ is the solvent viscosity. Therefore, if other properties are being held equal an increase in characteristic delay time implies and increase in particle diameter. Therefore the representative cluster sample has larger particles than the representative monomer sample.

Protein nanocluster dispersions are concentrated and may have low or significant polydispersity. There are multiple possible ACF analysis algorithms that can be used to determine particle size distributions; the one chosen for the data in this patent is called CONTIN. The CONTIN algorithm first approximates the ACF by replacing the summation of decays with an integral to form the following equation

$$g ( τ ) = {[ {\int }_{0}^{∞} A ( {τ}_{c} ) exp [ - \frac{τ}{{τ}_{c}} ] d {τ}_{c} ]}^{2}$$

CONTIN then solves this equation for A(τ_{c}) by Laplace transform inversion. (Note, A(τ_{c}) represents the intensity of scattered light for a particle with characteristic delay time, τ_{c}, for all possible values of τ_{c}.) However, since this is an ill-conditioned Laplace transform inversion with further complications from measurement noise there is not one unique solution. Therefore CONTIN has a unique algorithm for selecting what it deems to be the best possible solution to this equation. Each value of τ can then be converted to a diameter, D, by the above equation, yielding A(D), which is the distribution of the intensity of scattered light as a function of particle diameter. Assuming that the particles are spherical and using a Mie theory parameter calculated from the laser wavelength, instrument geometry, and refractive index of solvent and particles, the intensity distribution, A(D), can then be converted into a volume based distribution.

The values of nanocluster diameter described herein are the mean diameters of these volume based distributions as determined from the CONTIN algorithm. It is important to note that CONTIN has limitations in distinguishing between particles of similar size (if the particle size differs by a factor of less than 2 or 3). Therefore CONTIN may have difficulties in separating monomers in solution from clusters.

Utilizing the LD pathway, dispersions formulated at ˜150 mg/mL mAb 1 exhibited low viscosities of ˜10 cP (Samples LD 158:158P and LD 146:146P, Table 3), compared with a value of 28 cP for the lowest viscosity solution control (Table 4). As the protein concentration increased to ˜250 mg/mL the dispersion viscosities were mostly >100 cP. Several different pH buffers (pH 5.5 50 mM succinate, pH 8.2 50 mM phosphate, pH 7.2 50 mM phosphate, and pH 9.3 50 mM carbonate) were used to try to reduce dispersion viscosity. In many cases the [η] was 11-12 for concentrations of ˜250 mg/mL, but only 8 or 9 for the lower protein concentrations. The change in [η], which reflects stronger interactions, may indicate onset of gelation. A particularly remarkable result was obtained for 288 mg/mL mAb 1, where the viscosity was only 86 cP with a low [η] of only 8, despite the extremely high concentration. To further lower the viscosity and [η], other pathways were explored to attempt to attempt to minimize passing into metastable gel states.

Addition of a Dispersion Formed by Centrifugal Filtration Concentration Over Lyophilized Powder Composed of mAb 1 and Proline (C-D/P).

In the second pathway, a stock solution formed by concentrating mAb 1 to 150 mg/mL in DI water via centrifugation filtration, was added to lyophilized powder (1:1 mAb 1:proline) and concentrated inorganic buffer to form a dispersion (C-D/P, Table 5). The aggregate concentration was below 0.5% in the initial stock solution as determined by SEC (Table 2). In this pathway labeled as C-D/P in FIG. 2, the mAb 1 concentration increases without proline during the centrifugation step, and then both mAb 1 and proline concentration increase with the addition of lyophilized powder.

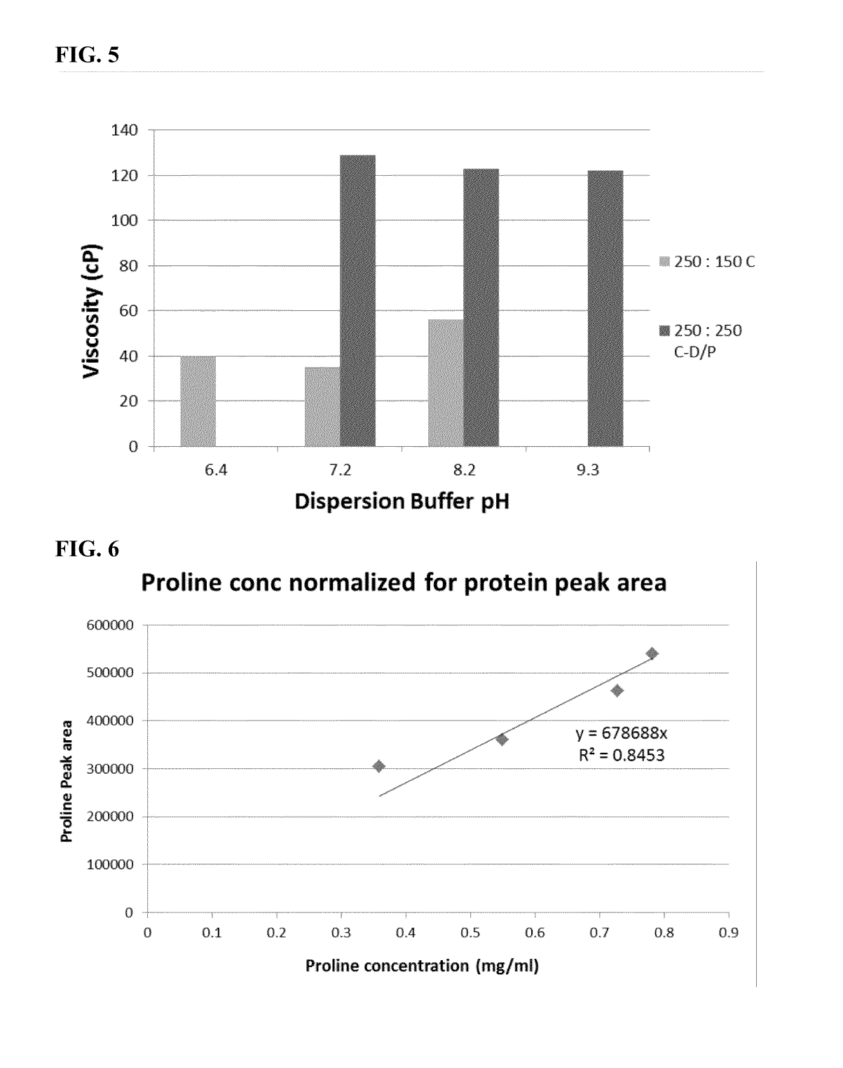

For dilute protein below 200 mg/mL, nanoclusters were formed with diameters below 40 nm. For protein concentrations from ˜230 to 250 mg/mL, the nanocluster diameters for similar conditions were again about 45 nm as seen in Table 3 and in FIG. 4 for various dispersion buffer pH values. With added protein, the pH shifted less than 0.5 units relative to the buffer. The viscosities of formulations produced by centrifugation filtration without added crowder are shown in Table 4. At pH values from 8.2 to 10, the viscosities were the highest when normalized for protein concentration, which may be expected near the isoelectric point where electrostatic repulsion is weak. At either lower or higher pH values as the protein became more charged, the viscosity (normalized by protein concentration) decreased. The dispersion viscosity was lower than that of comparable solutions at similar pH and protein concentration (as seen in Tables 3 and 4). Furthermore, as shown in FIG. 5, the dispersion viscosity was not sensitive to pH from pH 7.2 to 9.3 for the C-D/P pathway. Similarly to dispersions produced by the LD pathway, the stock dispersion over powder method produced very low viscosities (<15 cP) at ˜150 mg/mL mAb 1 concentration, but significantly higher viscosities at the higher 250 mg/mL mAb 1 concentration. However, the [η] of ˜10 was similar at all concentrations.

Centrifugation Filtration.

The third pathway tested was concentration of mAb 1 via centrifugal filtration (C), as shown via the C pathway in FIG. 2, from a solution with the desired final crowder concentration. Ideally, the crowder would permeate the filter non-selectively with the buffer, such that the crowder concentration would decrease about 15-20% from the increase in protein concentration and consequent increase in the protein volume fraction in the system. An SEC study of proline content in dispersions formed by centrifugation was undertaken to determine if proline was retained or excluded at a greater rate than mAb 1 (Table 7 and FIG. 6). The proline content of dispersions at pH 8 and 7 were seen to be on average 12% lower than the feed values, in line with the expected 15-20% decrease in crowder concentration due to the increase in protein volume fraction with increasing protein concentration. (In all other places herein, we report the feed proline value rather than the corrected value.) The SEC measurements suggest relatively non-selective permeation of proline across the membrane without significant complications of specific interactions with the protein, Donnan equilibrium across the membrane, and other effects.

For this method, the retentate was recovered under gravity (the “pour off” method), or by low-speed (1000 RCF) centrifugation (“spin off”). Our hypothesis was that the pour off method may leave behind protein that gelled and stuck to the filter, to enable recovery of a less viscous fraction that flowed out of the vessel. However, no consistent differences were seen in the viscosities between the two methods, despite lower yields in the pour off method (Table 6).

For a protein concentration of ˜250 mg/mL, the nanocluster size increased as the pH increased from 6.4 to 8.2 approaching the pI, as expected from the free energy model, due to lower charge repulsion between nanoclusters and monomer (Table 6, FIG. 4). Even more remarkably, this centrifugation filtration process and depletant selection also produce these low viscosity dispersions at pH's of 6.4, 7.2 and 8.2. The charge suppressing effects of the proline that contribute to clustering also seem to lead to weak interactions and low viscosities. While the size of the nanoclusters decreased with a decrease in dispersion pH (as seen in FIG. 4), the nanoclusters were still formed a full 3 pH units away from the mAb 1 isoelectric point suggesting that the proline may decrease the electrostatic repulsion of the protein monomers, or that nanoclusters may still be formed at higher charges with sufficient depletion attraction. Nanocluster formation this far from the isoelectric point was not investigated in previous studies.

The centrifugation filtration method produced moderately lower viscosities and intrinsic viscosities than the other two methods at a given composition. Unlike the lyophilization dilution and centrifugation dispersion over powder methods, the centrifugal filtration method produces repeatable dispersion viscosities in the 50 cP range with mAb 1 concentrations of ˜250 mg/mL. Despite these differences in viscosities, the nanocluster diameters were fairly similar for the various processes for similar compositions, suggesting equilibrium aspects are present in nanocluster formation. Although the mechanism for the lower viscosities for this method is not well understood, a few factors may be considered. In the centrifugal filtration method, the large number of air bubbles formed in the other two processes is not present. Proteins may adsorb at air/solution interfaces and aggregate and nucleate gels of interacting nanoclusters. Another possibility is that solid entities in the lyophilized powders do not fully dissociate into non-interacting nanoclusters upon stirring but form gel networks of interacting nanoclusters. Despite these complexities in the rheological behavior, it is interesting that the nanocluster hydrodyanamic diameters were similar indicating a driving force toward aspects of equilibrium behavior.

Complications with the centrifugation technique include specific interactions of proline with the charged protein surface, Donnan equilibrium, exclusion, and preferential hydration effects, all of which may cause unequal partitioning of crowder molecules across the filtration membranes. However, it has been demonstrated that with histidine, if diafiltration is done at the amino acid pI, unequal partitioning did not occur. In addition, various previous studies on this topic utilized tangential-flow filtration (TFF) where the feed and permeate were in contact, whereby equilibrium processes may influence permeation. In centrifugal filtration the contact between the retentate and permeate was minimized, given the gap between the filter and the permeate at the bottom of the tube, and thus Donnan equilibrium may play a less significant role.

Low viscosity dispersions of nanoclusters of concentrated protein were formed utilizing proline as a depletant (crowder), with viscosities an order of magnitude lower than those of solutions at comparable concentrations and pH. Despite the much smaller size of proline relative to previously used crowding agents, trehalose and polyethylene glycol, depletion attraction was still sufficient to form nanoclusters. The high preferential exclusion of proline (more significant than trehalose) leads to increased solvophobic effects with any exposed protein backbone, increasing the tendency of proteins to stay folded in this environment. The relatively constant nanocluster size over a proline concentration range from 100 to 300 mg/mL may be contrasted with a significant increase in size for the sheep IgG as the crowder (trehalose) concentration was increased. In addition, above 200 mg/mL, proline self-assembles, which may impede depletion at high concentrations and thus growth in nanocluster size. Furthermore, proline produces additional effects beyond depletion attraction, particularly the reduction of apparent surface charge for dilute mAb 1. If this electrostatic screening is present for concentrated mAb1, it may explain in part why nanoclusters could still be assembled 2-3 pH units away from the mAb pI, at a desirable pH for pharmaceutical use. The formation of these nanoclusters appeared to involve aspects of equilibrium behavior as similar hydrodynamic diameters (e.g., nanocluster diameters) were produced via three different routes: centrifugation filtration, lyophilization dilution, and centrifugation filtration dispersion over lyophilized powder. Moreover, all routes produced similarly sized small nanoclusters over a proline concentration range from 100 to 300 mg/mL. Upon dilution the nanoclusters dissociated back to monomer with less than 1% aggregates as shown by SEC. Dispersions produced via centrifugal filtration were modestly less viscous than those produced by the powder based techniques at similar compositions. Thus, the interactions between the nanoclusters depended upon the formation pathway. For the other cases, formation of air bubbles during mixing may have produced reversible protein adsorption and aggregation that raised the viscosity. In addition, proline was found to serve as a cryoprotectant for lyophilization even at levels as low as 1:0.1 mass of protein to proline, retaining monomeric protein post-lyophilization according to SEC.

Low Viscosity Protein Nanocluster Dispersions of the mAb1 Protein.

Low viscosity protein nanocluster dispersions were made with the mAb1 protein achieving viscosities as low as 35 cP at 250 mg/ml protein. Dispersion formulations included trehalose, which was used as the main system depletant to drive nanocluster formation, either histidine or imidazole to act as a system buffer and supply some specific interactions with the protein to lower viscosities, and either hydrochloric, citric or phosphoric acid to act as the counter-ion to the histidine/imidazole and control the formulation pH. Trehalose/histidine systems consistently formed nanoclusters of ˜30 nm that achieved viscosities less than 50 cP at 250 mg/ml protein at trehalose concentrations of 50 mg/ml (130 mM) and histidine concentrations at or above 40 mg/ml (250 mM) and enough acid to maintain pH 5.5 or 6. Equivalently, similarly low viscosities were seen for ˜30 nm nanocluster dispersions containing 50 mg/ml trehalose, 17 mg/ml (250 mM) imidazole at a pH ranging from 6 to 7. The ability to achieve low viscosities at pH 7 is an important addition to the field. The osmolalities calculated for these dispersions assuming ideal behavior were as low as 640 mOsm/kg for dispersions using HCl and 550 mOsm/kg for those using citric or phosphoric acid. Actual measured osmolalities are often lower than predicted by ideal behavior. We report approximately 60 dispersions formed by this approach. The ability to do a large number of conditions rapidly was useful for guiding the longer runs by tangential flow filtration. Furthermore, comparison of the two techniques provided insight into additional effects based on interactions of protein and excipients with the membrane.

Experiments disclosed herein may be made by a process known as lyophilization dilution (LD). In the LD process, a known mass of lyophilized protein powder of a known composition was diluted into a known volume of buffer of known composition and mixed together until all of the powder was completely dissolved forming either a protein solution or, in the case of these experiments, a protein nanocluster dispersion. The lyophilized powder was made by mixing a fixed level of excipient, usually trehalose, into the 100 mg/ml DI water stock. The mass, volume and concentration of the DI water stock were fully quantified; therefore, the amount of protein in the stock was also known, allowing for the excipient to be added at a known mass or molar ratio to the protein. In the case of all of the experiments described herein, a 0.15:1 or 0.2:1 mass ratio of trehalose to mAb1 protein was used to make the lyophilized powder, except for the few trehalose free experiments, which were made at a 0.2:0.09:1 mass ratio of histidine to citric acid to protein. Any additional excipient, either more mass of the same excipient or other excipients, were added to the dispersion through the buffer, as they were pre-dissolved into buffer solutions then mixed with the lyophilized powder. Size exclusion chromatography (SEC) stability data of the different lyophilized powders used are included herein.

Formulations with Trehalose, Histidine and Hydrochloric Acid.

Table 8 contains the data for all formulations including trehalose, histidine and hydrochloric acid (HCl) with trehalose concentrations ranging from 50 to 150 mg/ml, histidine concentrations ranging from 5 to 120 mg/ml and pH values ranging from 5.5 to 7. The data show that for formulations containing 50 mg/ml trehalose, 40 mg/ml or greater histidine and enough HCl to have a pH of 5.5 or 6 the viscosities are 50 cP or less at ˜250 mg/ml mAb1 protein with osmolalities as low as 640 mOsm/kg. Using the intrinsic viscosity and solvent viscosity, one can back calculate what the predicted protein concentration of the system would have to be to achieve a viscosity of 20 cP and for these formulations this concentration exceeds 200 mg/ml.

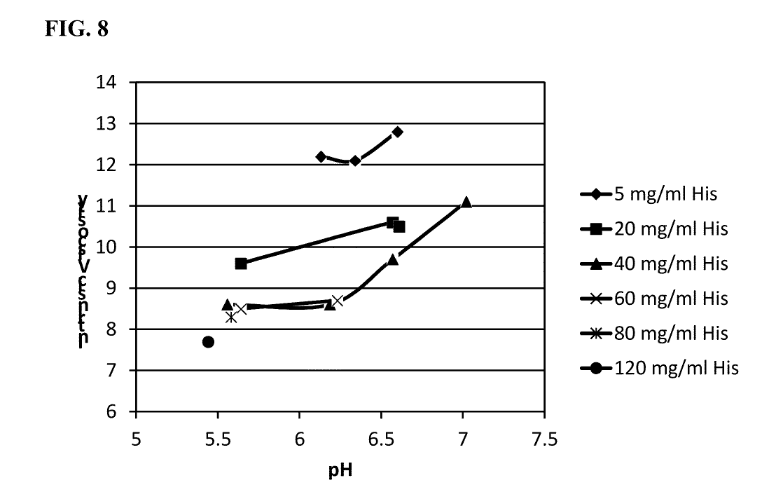

Intrinsic viscosities of formulations containing 50 mg/ml trehalose decrease with increasing histidine content; however, this reduction tapers off after about 40 mg/ml of histidine with only a slight reduction in intrinsic viscosity from 40 to 80 or even 120 mg/ml histidine. The data also shows that the intrinsic viscosity remains approximately constant between pH 5.5 and 6, but increases when raising the pH to 6.5 and further increases upon raising the pH to 7. These trends are displayed in FIG. 8 and FIG. 9.

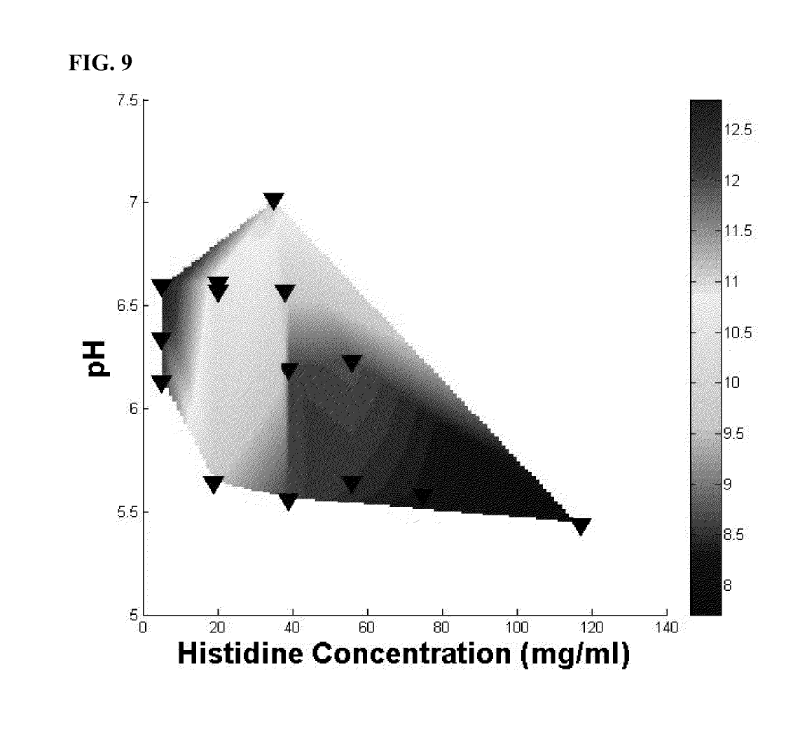

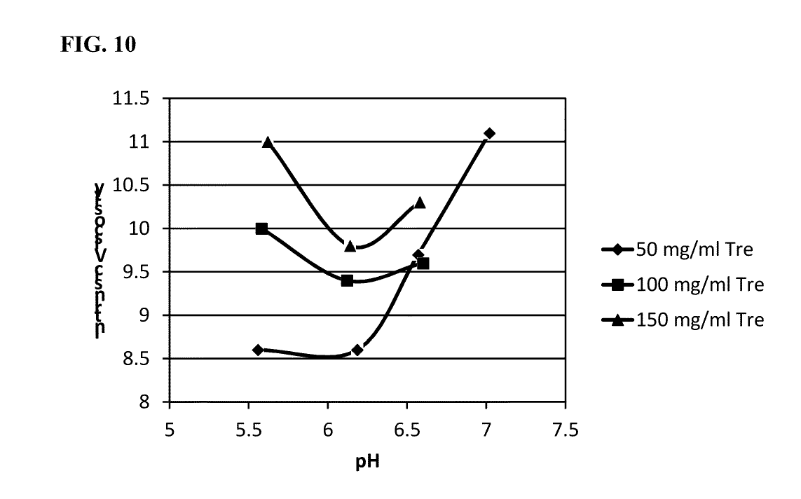

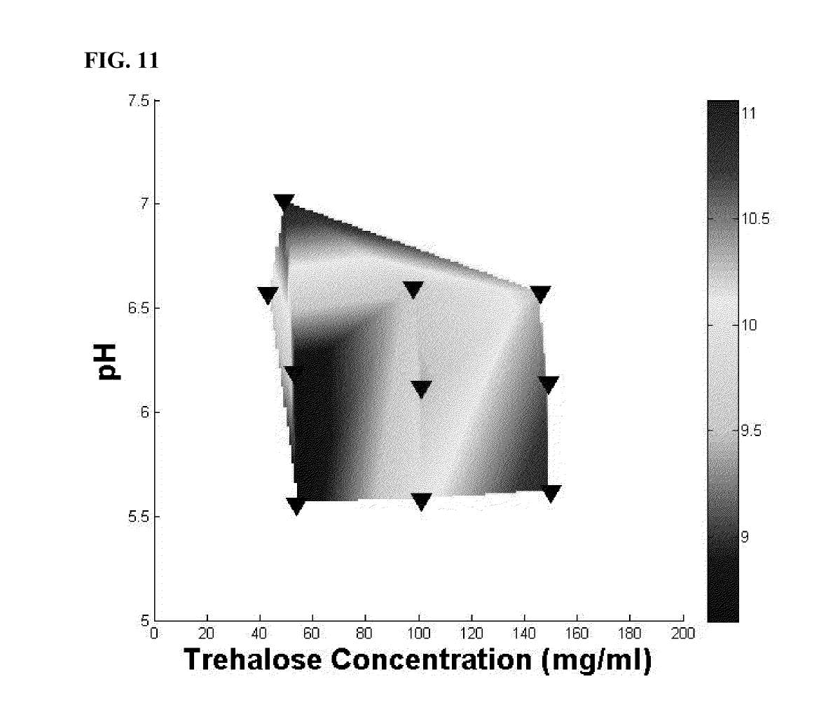

FIG. 10 and FIG. 11, show that for formulations with 40 mg/ml histidine, intrinsic viscosities tend to increase with increasing trehalose concentration from 50 to 100 mg/ml and increasing the trehalose to 150 mg/ml further increases the intrinsic viscosity. The data show that for a 50 mg/ml trehalose level, the intrinsic viscosity remains approximately constant between pH 5.5 and 6, but increases with further increases in pH to 6.5 and 7. However, for the 100 and 150 mg/ml level the intrinsic viscosity has a distinct minimum at pH 6, as it is higher at both pH 5.5 and 6.5.

Formulations with Trehalose, Histidine and Either Citric Acid or Phosphoric Acid.

Table 9 shows data for formulations that contain trehalose, histidine and citric acid; just histidine and citric acid; or trehalose, histidine and phosphoric acid. Results show that all formulations tried with protein concentrations ˜250 mg/ml with 40+mg/ml histidine, 0 to 100 mg/ml trehalose and enough citric acid or phosphoric acid to maintain a pH around 5.5 or 6 yields viscosities of 50 cP or less. One clear advantage of citric or phosphoric acid formulations over HCl systems is that for equivalent histidine levels and final pH values, the osmolalities of these systems are much lower than equivalent HCl systems. This can be seen as the osmolalities of one of these low viscosity systems (50 mg/ml trehalose, 40 mg/ml histidine, pH 6) is 550 mOsm/kg for a citric acid or phosphoric acid system, but are 640 mOsm/kg for an HCl system. This is possible because of the tri-protic nature of citric and phosphoric acid allowing for more H+ions per osmole of acid.

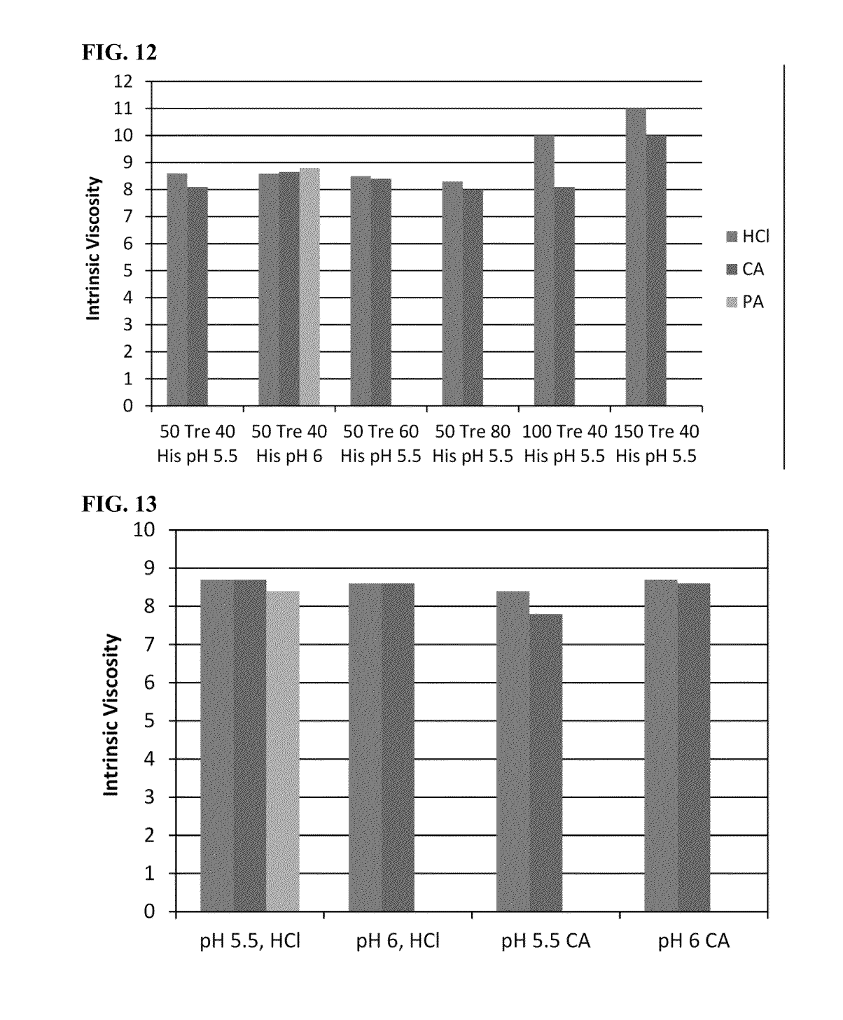

For otherwise identical conditions (same trehalose concentration, histidine concentration and pH) changing the acid from HCl to citric or phosphoric acid seems to have a mostly negligible effect on the intrinsic viscosity of the system for formulations with 50 mg/ml trehalose. However, at higher trehalose concentrations of 100 or 150 mg/ml citric acid yields lower intrinsic viscosities than HCl. This is displayed in FIG. 12.

Formulations with trehalose, imidazole and either HCl, citric acid or phosphoric acid. Table 10 shows the results of mAb1 protein dispersions made with trehalose, imidazole and either HCl, citric acid or phosphoric acid, with the trehalose concentration held constant at 50 mg/ml, imidazole concentrations ranging from 17 to 33 mg/ml and acid concentrations to reach pH values ranging from 6 to 7. All formulations tested with either HCl or citric acid had viscosities below 50 cP at 250 mg/ml protein with the pH 7 citric acid systems reaching the lowest viscosities which were as low as 30 cP at 250 mg/ml protein. The formulations with phosphoric acid were slightly higher viscosity ˜60 cP at 250 mg/ml protein.

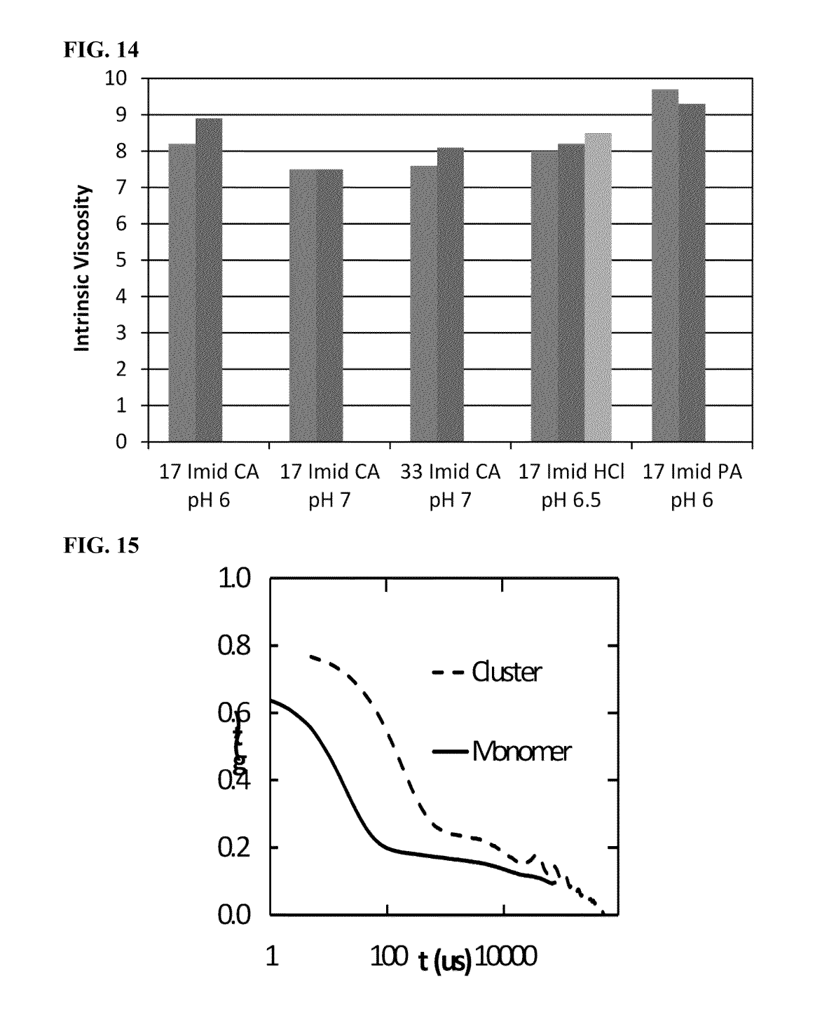

FIG. 13 and FIG. 14, show the intrinsic viscosities of different replicates of identical excipient formulations. FIG. 13 shows the reproducibility of trehalose/histidine formulations and FIG. 14 shows trehalose/imidazole formulations. Both figures show that the intrinsic viscosity is very reproducible within half a unit for a given formulation.

Formulations with Trehalose, Histidine, HCl and Additional Salts.

Nanocluster dispersions were made with varying levels of different salts (NaCl, MgSO₄, NaSCN and NH₄SCN) with 50 mg/ml trehalose either 20 or 40 mg/ml histidine and enough HCl to reach pH 6. In most cases with 40 mg/ml histidine the additional salt significantly increased the intrinsic viscosity as compared to the base case (same formulation except no additional salt), except for the cases of 25 mM MgSO₄ (additional ionic strength of 100 mM), 125 mM MgSO₄ (additional ionic strength of 500 mM) or 100 mM NaCl which had a minor increase in the intrinsic viscosity. At the 20 mg/ml histidine level, 125 mM MgSO₄ lowered the intrinsic viscosity more than any other salt tested, but still not to the levels of 40 mg/ml histidine formulations.

Formulations with Arginine and Glutamic Acid.

The effect of the total concentration of arginine and glutamic acid on the viscosity was also studied by fixing the pH of the sample at 7 by fixing the ratio of arginine to glutamic acid at 1.1 and targeting a mAb concentration of 250 mg/ml. The arginine and glutamic concentration was increased from 75 mg/ml total to 150 mg/ml total as shown in Table 11. The viscosity is seen to decrease with the increased concentration of the excipient from 67 cP at 246 mg/ml with the lowest excipient concentration to 41 cP at 263 mg/ml with the highest excipient concentration. The trend in viscosity is further mirrored by the decreased intrinsic viscosity.

In order to further highlight the lowered viscosities of samples with arginine and glutamic acid, samples of mAb without arginine and glutamic acid were prepared as shown in Table 12. The mAb was formulated in the absence of any excipient in various buffers at a pH of around 6. The measured viscosities were much higher compared to the samples from Table 11 with arginine and glutamic acid. The lowest viscosity obtained in these samples in the absence of arginine and glutamic acid was 80 cP at 200 mg/ml although most of the samples have viscosities >100 cP. The diameter for all of these samples is seen to be within error of 11 nm which would be the expected hydrodynamic diameter (e.g., nanocluster diameter) of an IgG monomer hinting that there are very few larger species present. These samples seemed to permeate through faster that the nanocluster samples initially, but then seemed to slow down significantly once the concentration approached 200 mg/ml at which point the permeation was almost zero probably as a consequence of the elevated viscosity.

Table 13 shows that the phenomenon of the lowering of viscosity by arginine and glutamic acid to polyclonal antibodies. Addition of arginine and glutamic acid lowered the viscosity with a polyclonal sheep IgG with 150 mg/ml of arginine and glutamic acid with an extremely low viscosity of 27 cP at 252 mg/ml. The nanocluster diameter for the sheep IgG sample was seen to be 50 nm which is quite large compared to that seen for the monomer. Increasing the concentration of arginine and glutamic acid to 200 mg/ml did not seem to lower the viscosity for sheep IgG further with the viscosity staying at 30 cP at 258 mg/ml. The diameter for this sample was also seen to be large at 60 nm. The spin time for the higher arginine concentration sample is higher because of the higher solvent viscosity.

Trehalose is often included in mAb formulations to lend additional stability to the mAb molecules, as known to practitioners in the field. Table 14 shows the effect of adding trehalose in a constant ratio to arginine and glutamic acid containing formulations. The viscosity of the formulations decreases from 86 cP to 51 cP at ˜240 mg/ml as the concentration of the excipients is increased from 75 mg/ml to 200 mg/ml. The addition of trehalose increases the viscosity slightly over the formulations only containing arginine and glutamic acid at similar concentrations as can be seen by comparing row 2 of Table 14 with row 1 of Table 11. At similar mAb concentrations of ˜250 mg/ml the viscosity for the trehalose containing samples is slightly higher probably as a result of the elevated solvent viscosity.

Table 15 shows the viscosity and intrinsic viscosity for mAb samples containing arginine titrated with HCl instead of glutamic acid in order to vary the pH. The viscosity with HCl instead of glutamic acid is higher at similar pH and excipient concentrations. Therefore the glutamic acid may have some interactions with the mAb which lowers the viscosity. Additionally the samples with glutamic acid have the benefit of being lower tonicity.

The dissociation of the nanoclusters upon dilution is evidenced by size exclusion chromatography of the mAb at 1 mg/ml in Table 16. The SEC trace of the sample pre-processing shows >99% monomer. The trace for the 250 mg/ml nanocluster sample from row 4 of Table 13 is almost identical to that of the protein monomer pre-processing thus corroborating the evidence from DLS that monomeric mAb is obtained upon dilution. A preliminary study of the stability of the mAb dispersion was also conducted by string the sample of mAb at −40° C., 4° C. and room temperature for 8 weeks as shown in Table 21. The samples stored under all three conditions are seen to be stable with no significant decrease in the % monomer over the course of 8 weeks. Additional examples are given in Table 18 to 22 for dispersions formed by the lyophilization dispersion technique.

### References (Example 1)

Laue T 2012. Journal of Molecular Recognition 25(3):165-173. Sinko P J, Martin A. 2006. Martin's Physical Pharmacy and Pharmaceutical Sciences. 5 ed., Philadelphia: Lippincott Williams & Wilkins Zhou H X, et al. 2008. Annual Review of Biophysics 37(1):375-397. Rosenberg E, et al. 2009. Journal of Membrane Science 342(1-2):50-59. Young™, Roberts C J 2009. The Journal of Chemical Physics 131(12):125104. Fields G B, et al. 1992. J Phys Chem 96(10):3974-3981. Shire S J, et al. 2004. J Pharm Sci 93(6):1390-1402. Rosenbaum D F, et al. 1996. Phys Rev Let 76(1):150-153. ten Wolde P R, Frenkel D 1997. Science 277:1975-1978. Zaccarelli E 2007. J Phys: Condens Matter 19:323101. Hartl F U, Hayer-Hartl M 2002. Science 295(5561):1852-1858. Johnston K P, et al. 2012. Acs Nano. Shen V K, et al. 2006. Biophysical journal 90:1949-1960. Cheung J K, Truskett T M 2005. Biophysical journal 89(4):2372-2384. Murthy A K, et al. 2013. Acs Nano 7(1):239-251. Groenewold J, Kegel W K 2004. J Phys-Condes Matter 16(42):54877-54886. Groenewold J, Kegel W K 2001. J Phys Chem B 105(47):11702-11709. Miller M A, et al. 2012. Journal of pharmaceutical sciences 101(10):3763-3778. Borwankar A U, et al. 2013. Soft Matter 9(6):1766-1771. Srinivasan C, et al. 2013. Pharm Res. Xia Y S, et al. 2011. Nat Nanotechnol 6(9):580-587. Zhang C, et al. 2012. Soft Matter 8(1):86-93. Miller Mass., et al. 2010. Langmuir: the ACS journal of surfaces and colloids 26(2):1067-1074. Hiemenz P C, Rajagopalan R. 1997. Principles of Colloid and Surface Chemistry. 3rd ed., New York: Marcel Dekker, Inc. p 650. Torquato S, et al. 2000. Phys Rev Lett 84(10):2064-2067. Yadav S, et al. 2010. Journal of pharmaceutical sciences 99(3):1152-1168. Civera M, et al. 2005. Chem Phys Lett 415(4-6):274-278. Campbell M K, Farrell S O. 2003. Biochemistry. 4th ed., United States: Thomson-Brooks/Cole. Ignatova Z, Gierasch L M 2006. Proc Natl Acad Sci USA 103(36):13357-13361. Troitzsch R Z, et al. 2008. J Phys Chem B 112(14):4290-4297. Shukla D, et al. 2011. Adv Drug Deliv Rev 63(13):1074-1085. Bolli R, et al. 2010. Biologicals 38(1):150-157. Samuel D, et al. 2000. Protein Sci 9(2):344-352. Auton M, Bolen D W 2005. Proc Natl Acad Sci USA 102(42):15065-15068. Courtenay E S, et al. 2000. Biochemistry 39(15):4455-4471. Xie G, Timasheff S N 1997. Biophysical Chemistry 64:25-43. Cheung Miss., et al. 2005. Proc Natl Acad Sci USA 102(13):4753-4758. Yadav S, et al. 2011. Pharm Res 28(8):1973-1983. Pilz I, et al. 1970 Biochemistry 9(2):211-219. Miller D P, dePablo J J, Corti H 1997. Pharm Res 14(5):578-590. Beenakker C W J, Mazur P 1984. Physica A 126(3):349-370. Lekkerkerker H N W, Tuinier R. 2011. Colloids and the Depletion Interaction. 2011 ed.: Springer. p 268. Xin Y. 2007. Electrokinetic Modeling of Free Solution Electrophoresis. Department of Chemistry, ed.: Georgia State University. Kar K, Kishore N 2007. Biopolymers 87(5-6):339-351. Teeters M, et al. 2011. Biotechnol Bioeng 108(6):1338-1346. Miao F, et al. 2009. Biotechnol Prog 25(4):964-972. Stoner M R, et al. 2004. Journal of pharmaceutical sciences 93(9):2332-2342.

### Tables (Example 1)

TABLE 1
Parameters used in the free energy model (FIG. 2) for predicting
nanocluster size at a given mAb 1 and proline concentration.

| Model parameter | Value |
|---|---|
| Fractal Dimension (δ_{f}) | 2.5 |
| Dielectric constant (∈ᵣ) | 15 |
| No. of dissociable sites per unit area of | 0.2 |
| colloid surface (σₛ, nm⁻²) | |
| Distance between opposite charges in an | 0.22 |
| ionic bond (b, nm) | |
| Radius of the protein monomer (R, nm) | 5.5 |
| Radius of extrinsic crowder (depletant, R_{E}, nm) | 0.268 |

TABLE 2
SEC stability of control (diluted mAb stock as received),
diluted 1:1 mAb 1:proline by mass lyophilized powder, and
dispersions made by addition of a 150 mg/mL mAb dispersion
manufactured by centrifugation concentration to lyophilized
powder (C-D/P) and centrifugation (C). All dispersions were
diluted to 1 mg/mL in pH 6.4 50 mM sodium phosphate.

| Sample ID | % Monomer |
|---|---|
| Control | 99.69 |
| 150 mg/mL stock solution | 99.61 |
| Lyophilized Powder | 99.24 |
| C-D/P 236:250P (Table 5) | 99.42 |
| C-D/P 238:250P (Table 5) | 99.19 |
| C-D/P 227:150P (Table 5) | 99.84 |
| C 262:150P (Table 6) | 99.35 |

TABLE 3
Nanocluster dispersions formed from lyophilized powder composed of equal masses
of mAb 1 and proline. In the LD process, lyophilized powders were dispersed
in buffer. In the LD-D/P process, an intermediate (~150 mg/ml mAb 1) dispersion
(D) made by the LD process was added to additional lyophilized powder.

| | | | | | | Nano- | |
|---|---|---|---|---|---|---|---|
| Protein | Proline | | | | | cluster | |
| Conc. | Conc. | Dispersion | Buffer & | Viscosity | Intrinsic | diameter | |
| (mg/mL) | (mg/mL) | Method | pH | (cP) ± s.d. | Viscosity | (nm) | Sample ID |
| 158 | 158 | Packed vial | 50 mM | 12 (n = 1) | 9 | — | LD |
| | | | pH 7.2 | | | | 158:158P |
| | | | phosphate | | | | |
| 252 | 252 | Dispersion | 50 mM | 159 ± 13 | 11 | — | LD-D/P |
| | | over powder | pH 7.2 | | | | 252:252P |
| | | | phosphate | | | | |
| 146 | 146 | Packed Vial | 50 mM | 8 (n = 1) | 8 | — | LD |
| | | | pH 9.3 | | | | 146:146P |
| | | | carbonate | | | | |
| 258 | 258 | Dispersion | 50 mM | 114 ± 23 | 9 | — | LD-D/P |
| | | over powder | pH 9.3 | | | | 258:258P |
| | | | carbonate | | | | |
| 245 | 100 | Packed Vial + | 50 mM | 135 ± 45 | 11 | 42 | LD-D/P |
| | | incremental | pH 7.2 | | | | 245:100P |
| | | addition* | phosphate | | | | |
| 248 | 100 | Packed Vial + | 50 mM | 167 ± 17 | 12 | 45 | LD-D/P |
| | | incremental | pH 7.2 | | | | 248:100P |
| | | addition* | phosphate | | | | |
| 248 | 150 | Packed Vial + | 50 mM | 163 ± 23 | 11 | 49 | LD-D/P |
| | | incremental | pH 7.2 | | | | 248:150P |
| | | addition* | phosphate | | | | |
| 253 | 150 | Packed Vial + | 50 mM | 172 ± 24 | 11 | 47 | LD-D/P |
| | | incremental | pH 7.2 | | | | 253:150P |
| | | addition* | phosphate | | | | |
| 234 | 400 | Dispersion | 50 mM | 214 ± 37 | 12 | 51 | LD-D/P |
| | | over powder | pH 7.2 | | | | 234:400P |
| | | | phosphate | | | | |
| 244 | 237 | Packed | 50 mM | 128 ± 14 | 11 | 35 | LD |
| | | Vial* | pH 8.2 | | | | 244:237P |
| | | | phosphate | | | | |
| | | | w/200 mg/mL | | | | |
| | | | Proline | | | | |
| 229 | 229 | Packed Vial | 50 mM | 112 ± 11 | 11 | 46 | LD |
| | | | pH 8.2 | | | | 229:229P |
| | | | phospate | | | | |
| 288 | 288 | Packed Vial | 50 mM | 86 ± 13 | 8 | 41 | LD |
| | | | pH 5.5 | | | | 288:288P |
| | | | succinate | | | | |

TABLE 4
Protein solutions formed by centrifugal filtration as in Table 6
except with dilute trehalose as a stabilizer, without any proline.
The trehalose concentration was 30 mg/mL after buffer exchange (with
the exception of the DI water sample, which had no excipient). The
mAb solution formed in DI water equilibrated at pH 5.5.

| Protein | | Viscosity | | |
|---|---|---|---|---|
| Conc. | | (cP) ± | Intrinsic | Diameter |
| (mg/mL) | Buffer Solution | s.d. | Viscosity | (nm) |
| 146 | DI Water | 26 ± 1 | 27 | 13 |
| 248 | 50 mM acetate, pH 4.7 | 100 ± 13 | 21 | — |
| 244 | 50 mM acetate, pH 5.5 | 243 ± 31 | 26 | — |
| 216 | 50 mM sodium | 230 ± 48 | 29 | — |
| | phosphate, pH 7.2 | | | |
| 190 | 50 mM sodium | 247 ± 44 | 35 | — |
| | phosphate, pH 8.2 | | | |
| 173 | 50 mM sodium | Could not be | — | — |
| | carbonate, pH 10 | syringed | | |
| 224 | 50 mM sodium | 113 ± 14 | 25 | — |
| | carbonate, pH 11.5 | | | |

TABLE 5
Nanocluster dispersions formed by adding mAb solution in DI manufactured by centrifugation
filtration at 150 mg/mL to 500 mM phosphate buffer, solid proline powder, and
1:1 mass ratio mAb 1:proline lyophilized powder (with the exception of row 1,
where no powder was added) to further concentrate the protein.

| | | | | Nano- | | | |
|---|---|---|---|---|---|---|---|
| Protein | Proline | % protein | | cluster | | | |
| Conc. | Conc | from | Buffer & | Diameter | Viscosity | Intrinsic | |
| (mg/mL) | (mg/mL) | solution | pH | (nm) | (cP) ± s.d. | Viscosity | Sample ID |
| 133 | 130 | 100 | DI Water | 18 | 10 ± 1 | 10 | C-D/P |
| | | | | | | | 133:130P |
| 156 | 150 | 76 | 50 mM | 32 | 13 ± 1 | 10 | C-D/P |
| | | | pH 7.2 | | | | 156:150P |
| | | | phosphate | | | | |
| 227 | 150 | 53 | 50 mM | — | 47 ± 2 | 10 | C-D/P |
| | | | pH 7.2 | | | | 227:150P |
| | | | phosphate | | | | |
| 252 | 250 | 39 | 50 mM | 44 | 129 ± 17 | 10 | C-D/P |
| | | | pH 7.2 | | | | 252:250P |
| | | | phosphate | | | | |
| 236 | 250 | 39 | 50 mM | 45 | 123 ± 10 | 11 | C-D/P |
| | | | pH 8.2 | | | | 236:250P |
| | | | phosphate | | | | |
| 238 | 250 | 39 | 50 mM | 48 | 122 ± 10 | 10 | C-D/P |
| | | | pH 9.3 | | | | 238:250P |
| | | | carbonate | | | | |

TABLE 6
Nanocluster dispersions formed by centrifugation filtration to concentrate
the protein after buffer exchange at a level of 4 mg/mL mAb 1.

| | Assumed | | | Nano- | | | | |
|---|---|---|---|---|---|---|---|---|
| Protein | Proline | | | cluster | | | | |
| Conc. | Conc. | Buffer & | Recovery | Diameter | Viscosity | Intrinsic | % | |
| (mg/mL) | (mg/mL) | pH | Method | (nm) | (cP) ± s.d. | Viscosity | Yield | Sample ID |
| 199 | 150 | 50 mM | Spin Off | 22 | 25 ± 2 | 9.0 | 59 | C-S |
| | | pH 6.4 | | | | | | 199:150P |
| | | phosphate | | | | | | |
| 211 | 150 | 50 mM | Spin off | 18 | 20 ± 2 | 7.7 | 69 | C-S |
| | | pH 6.4 | | | | | | 211:150P |
| | | phosphate | | | | | | |
| 251 | 150 | 50 mM | Spin Off | 25 | 42 ± 2 | 7.9 | 94 | C-S |
| | | pH 6.4 | | | | | | 251:150P |
| | | phosphate | | | | | | |
| 243 | 150 | 50 mM | Spin Off | — | 47 ± 7.4 | 8.2 | 70 | C-S |
| | | pH 7.2 | | | | | | 243:150P |
| | | phosphate | | | | | | |
| 258 | 150 | 50 mM | Pour off | 33 | 35 ± 5 | 7.1 | 56 | C-P |
| | | pH 7.2 | | | | | | 258:150P |
| | | phosphate | | | | | | |
| 266 | 150 | 50 mM | Pour Off | 37 | 55 ± 2 | 8.0 | — | C-P |
| | | pH 7.2 | | | | | | 266:150P |
| | | phosphate | | | | | | |
| 225 | 150 | 50 mM | Spin off | 44 | 25 ± 2 | 7.7 | 91 | C-S |
| | | pH 8.2 | | | | | | 225:150P |
| | | phosphate | | | | | | |
| 228 | 150 | 50 mM | Spin Off | 40 | 40 ± 12 | 8.9 | 95 | C-S |
| | | pH 8.2 | | | | | | 228:150P |
| | | phosphate | | | | | | |
| 257 | 150 | 50 mM | Pour Off | 41 | 56 ± 3 | 8.0 | 46 | C-P |
| | | pH 8.2 | | | | | | 257:150P |
| | | phosphate | | | | | | |
| 262 | 150 | 50 mM | Spin Off | 36 | 80 ± 4 | 9.0 | 98 | C-S |
| | | pH 8.2 | | | | | | 262:150P |
| | | phosphate | | | | | | |

TABLE 7
SEC measured values of proline concentration from SEC peak area,
normalized by the mass of actual injected protein per sample
(see FIG. 6). Samples in rows 1-4 have fully defined compositions
(made by dispersion-over-powder technique). Samples in rows
5-9 were made by centrifugation concentration (C), and the estimated
proline concentrations were verified by SEC. On average, measured
proline concentrations were within 12% of those estimated by
quantification of mass of proline added (LD-D/P and C-D/P) and
the buffer proline concentration pre-buffer exchange (C), even
post-centrifugal filtering.

| | | | Proline | Proline | % Diff. |
|---|---|---|---|---|---|
| | | Protein | Conc | Conc (by | btw |
| | Table | Conc. | (Esti- | SEC) ± | SEC & |
| | Found | (mg/ | mate, | s.d. | Esti- |
| Sample ID | In | mL) | mg/mL) | (mg/mL) | mate |
| LD-D/P 234:400P | 1 | 234 | 400 | 342 ± 7 | 14% |
| C-D/P 236:250P | 2 | 236 | 250 | 214 ± 4 | 14% |
| C-D/P 238:250P | 2 | 238 | 250 | 225 ± 5 | 10% |
| C-D/P 227:150P | 2 | 227 | 150 | 116 ± 1 | 22% |
| C-S 262:150P | 3 | 262 | 150 | 117 | 22% |
| C 213:150 mg/mL | — | 213 | 150 | 160 | 7% |
| mAb:Pro, 60 cP, | | | | | |
| intrinsic 11, nano- | | | | | |
| cluster diameter | | | | | |
| 39 nm, in pH 8.2 | | | | | |
| phosphate buffer, | | | | | |
| dispersion pH 8 | | | | | |
| C 199:200 mg/mL | — | 199 | 200 | 215 | 8% |
| mAb:Pro, 57 cP, | | | | | |
| intrinsic 11, nano- | | | | | |
| cluster diameter | | | | | |
| 33, in pH 7.2 | | | | | |
| phosphate buffer, | | | | | |
| dispersion pH 7 | | | | | |
| C 210:200 mg/mL | — | 210 | 200 | 225 | 13% |
| mAb:Pro, 47 cP, | | | | | |
| intrinsic 10, nano- | | | | | |
| cluster diameter | | | | | |
| 38, in pH 7.2 | | | | | |
| phosphate buffer, | | | | | |
| dispersion pH 7 | | | | | |
| C 227:150 mg/mL | — | 227 | 150 | 151 | 1% |
| mAb:Pro, 60 cP, | | | | | |
| intrinsic 10, nano- | | | | | |
| cluster diameter | | | | | |
| 41, in pH 8.2 | | | | | |
| phosphate buffer, | | | | | |
| dispersion pH 8 | | | | | |

TABLE 8
mAb1 dispersions made with trehalose, histidine and hydrochloric acid (HCl). Replicates are shaded
together in grey. These dispersions were formed with the lyophilization dilution technique.

| | | | | | | | | | | Calc | | | | | | | Calc | |
|---|---|---|---|---|---|---|---|---|---|---|---|---|---|---|---|---|---|---|
| | Conc | | | | | | | | | Ideal | | | | | | | Conc | Nano- |
| Protein | St | | Trehalose | | Histidine | | HCl | | | Osmo- | | | | Visc | Sol- | | at | cluster |
| Conc | Dev | | Conc | | Conc | | Conc | | Exc: | lality | Ionic | | Vis- | St | vent | Intrin- | 20 cP | Diam- |
| mg/ | mg/ | Yield | mg/ | | mg/ | | mg/ | | Protein | mOsm/ | Strength | | cosity | Dev | Visc | sic | mg/ | eter |
| ml | ml | % | ml | mM | ml | mM | ml | mM | M:M | kg | mM | pH | cP | cP | cP | Visc | ml | nm |
| 268 | 2.44 | 101 | 54 | 141 | 31 | 199 | 0 | 0 | 191 | 420 | 5 | 7.31 | 297 | 15.6 | 1.20 | 11.8 | 161 | |
| 262 | 3.83 | 93 | 56 | 147 | 5 | 31 | 1 | 29 | 118 | 251 | 29 | 6.13 | 271 | 45.3 | 1.10 | 12.2 | 161 | 23 |
| 268 | 13.25 | 100 | 54 | 143 | 5 | 32 | 0.6 | 17 | 107 | 233 | 17 | 6.34 | 307 | 40.0 | 1.09 | 12.1 | 162 | 24 |
| 268 | 6.77 | 99 | 54 | 144 | 5 | 31 | 0.3 | 8 | 103 | 223 | 8 | 6.60 | 416 | 21.0 | 1.09 | 12.8 | 155 | 24 |
| 257 | 7.15 | 94 | 55 | 144 | 19 | 126 | 4 | 115 | 225 | 468 | 115 | 5.64 | 78 | 5.90 | 1.16 | 9.6 | 192 | 27 |
| 258 | 0.02 | 96 | 54 | 143 | 20 | 126 | 2 | 66 | 195 | 408 | 66 | 6.57 | 121 | 15.7 | 1.16 | 10.6 | 178 | 29 |
| 254 | 7.74 | 94 | 54 | 143 | 20 | 126 | 1.2 | 33 | 178 | 365 | 33 | 6.61 | 107 | 10.9 | 1.16 | 10.5 | 179 | 27 |
| 255 | 7.94 | 92 | 55 | 146 | 39 | 250 | 8 | 229 | 367 | 768 | 229 | 5.47 | 53 | 1.88 | 1.23 | 8.7 | 204 | 30 |
| 262 | 2.53 | 95 | 53 | 139 | 39 | 249 | 8 | 214 | 344 | 743 | 214 | 5.61 | 62 | 1.22 | 1.22 | 8.7 | 205 | |
| 262 | 11.33 | 95 | 55 | 146 | 39 | 250 | 8 | 215 | 350 | 755 | 215 | 5.59 | 55 | 2.4 | 1.23 | 8.4 | 210 | |
| 249 | 2.44 | 91 | 53 | 139 | 39 | 252 | 5 | 132 | 315 | 638 | 132 | 6.15 | 45 | 3.27 | 1.19 | 8.6 | 206 | 31 |
| 262 | 2.52 | 95 | 52 | 137 | 39 | 251 | 5 | 131 | 297 | 641 | 131 | 6.22 | 59 | 1.22 | 1.22 | 8.6 | 207 | |
| 255 | 8.44 | 87 | 43 | 113 | 38 | 243 | 2 | 63 | 247 | 517 | 63 | 6.57 | 80 | 7.65 | 1.19 | 9.7 | 189 | 30 |
| 247 | 4.58 | 92 | 49 | 130 | 35 | 223 | 1 | 17 | 226 | 450 | 17 | 7.02 | 117 | 17.6 | 1.20 | 11.1 | 170 | |
| 225 | 1.60 | 86 | 50 | 132 | 56 | 361 | 14 | 383 | 584 | 1061 | 383 | 5.64 | 28 | 1.94 | 1.27 | 8.5 | 206 | |
| 243 | 2.50 | 91 | 50 | 133 | 56 | 360 | 8 | 232 | 453 | 892 | 232 | 6.23 | 43 | 2.08 | 1.27 | 8.7 | 203 | |
| 253 | 2.70 | 96 | 50 | 132 | 75 | 481 | 15 | 414 | 604 | 1294 | 414 | 5.58 | 47 | 3.60 | 1.33 | 8.3 | 207 | |
| 252 | 4.98 | 95 | 52 | 137 | 117 | 751 | 23 | 644 | 912 | 1995 | 644 | 5.44 | 40 | 2.7 | 1.48 | 7.7 | 213 | |
| 268 | 5.60 | 93 | 101 | 268 | 19 | 124 | 4 | 107 | 277 | 625 | 107 | 5.71 | 145 | 7.24 | 1.27 | 10.2 | 178 | |
| 250 | 7.10 | 89 | 101 | 267 | 19 | 125 | 2 | 65 | 269 | 565 | 65 | 6.17 | 167 | 20.2 | 1.27 | 11.6 | 161 | |
| 264 | 5.00 | 96 | 101 | 266 | 19 | 126 | 1.2 | 32 | 241 | 530 | 32 | 6.52 | 216 | 45.4 | 1.27 | 11.3 | 166 | |
| 231 | 6.45 | 84 | 101 | 266 | 39 | 252 | 7 | 201 | 456 | 865 | 201 | 5.58 | 59 | 8.31 | 1.33 | 10 | 179 | |
| 251 | 6.40 | 91 | 101 | 267 | 39 | 251 | 5 | 131 | 388 | 815 | 131 | 6.12 | 71 | 2.34 | 1.33 | 9.4 | 188 | |
| 246 | 1.17 | 92 | 98 | 260 | 39 | 253 | 3 | 70 | 355 | 728 | 70 | 6.60 | 69 | 4.29 | 1.32 | 9.6 | 185 | |
| 261 | 18.80 | 96 | 149 | 394 | 20 | 126 | 1 | 33 | 325 | 708 | 33 | 6.53 | 196 | 40.6 | 1.36 | 11.1 | 163 | |
| 250 | 0.60 | 90 | 150 | 395 | 39 | 251 | 8 | 215 | 506 | 1110 | 215 | 5.62 | 149 | 17.5 | 1.43 | 11 | 162 | |
| 250 | 6.90 | 92 | 149 | 395 | 39 | 252 | 5 | 132 | 458 | 1004 | 132 | 6.14 | 90 | 11.2 | 1.43 | 9.8 | 178 | |
| 240 | 0.45 | 90 | 146 | 386 | 39 | 253 | 3 | 71 | 444 | 906 | 71 | 6.58 | 87 | 9.7 | 1.42 | 10.3 | 171 | |

TABLE 9
mAb1 dispersions made with trehalose, histidine and either citric acid
(CA) or phosphoric acid (PA). Replicates are shaded together in grey. These
dispersions were formed with the lyophilization dilution technique.

| | Conc | | | | | | | | | |
|---|---|---|---|---|---|---|---|---|---|---|
| Protein | St | | Trehalose | | Histidine | | | Acid | | |
| Conc | Dev | | Conc | | Conc | | | Conc | | Exc: |
| mg/ | mg/ | Yield | mg/ | | mg/ | | | mg/ | | Protein |
| ml | ml | % | ml | mM | ml | mM | Acid | ml | mM | M:M |
| 231 | 14.8 | 96 | 34 | 90 | 53 | 339 | CA | 24 | 115 | 353 |
| 270 | 4.89 | 98 | 40 | 107 | 49 | 317 | CA | 16 | 78 | 278 |
| 231 | 4.06 | 89 | 53 | 139 | 39 | 253 | CA | 18 | 86 | 310 |
| 233 | 4.06 | 90 | 52 | 139 | 39 | 254 | CA | 18 | 86 | 308 |
| 246 | 5.40 | 95 | 52 | 136 | 39 | 254 | CA | 13 | 62 | 275 |
| 261 | 2.52 | 95 | 52 | 137 | 39 | 251 | CA | 13 | 61 | 258 |
| 250 | 5.33 | 93 | 53 | 139 | 54 | 347 | CA | 25 | 118 | 363 |
| 237 | 0.02 | 91 | 53 | 140 | 78 | 504 | CA | 36 | 171 | 515 |
| 257 | 16.9 | 98 | 51 | 136 | 77 | 498 | CA | 25 | 121 | 441 |
| 240 | 2.46 | 93 | 102 | 269 | 39 | 254 | CA | 18 | 86 | 380 |
| 235 | 6.85 | 89 | 150 | 398 | 39 | 254 | CA | 18 | 86 | 470 |
| 230 | 1.77 | 89 | 0 | 0 | 55 | 357 | CA | 28 | 133 | 321 |
| 234 | 1.52 | 91 | 0 | 0 | 81 | 524 | CA | 37 | 178 | 450 |
| 229 | 1.59 | 88 | 0 | 0 | 77 | 493 | CA | 28 | 132 | 410 |
| 268 | 12.8 | 98 | 54 | 142 | 39 | 251 | PA | 5 | 49 | 247 |

| | Calc | | | | | | | | |
|---|---|---|---|---|---|---|---|---|---|
| | Ideal | | | | | | | Calc | Nano- |
| Protein | Osmo- | | | | Visc | Sol- | | Conc | cluster |
| Conc | lality | Ionic | | Vis- | St | vent | Intrin- | at | Diam- |
| mg/ | mOsm/ | Strength | | cosity | Dev | Visc | sic | 20 cP | eter |
| ml | kg | mM | pH | cP | cP | cP | Visc | mg/ml | nm |
| 231 | 662 | 440 | 5.57 | 28 | 0.59 | 1.21 | 8.3 | 213 | 33 |
| 270 | 628 | 294 | 6.02 | 53 | 1.37 | 1.22 | 8.0 | 218 | 32 |
| 231 | 581 | 295 | 5.72 | 30 | 3.77 | 1.28 | 8.4 | 208 | 31 |
| 233 | 583 | 296 | 5.73 | 25 | 1.82 | 1.28 | 7.8 | 220 | |
| 246 | 552 | 235 | 6.01 | 44 | 2.03 | 1.26 | 8.7 | 203 | 32 |
| 261 | 557 | 231 | 6.06 | 60 | 2.75 | 1.26 | 8.6 | 205 | |
| 250 | 755 | 404 | 5.61 | 47 | 3.24 | 1.35 | 8.4 | 205 | |
| 237 | 1032 | 586 | 5.64 | 33 | 2.98 | 1.47 | 8.0 | 207 | 32 |
| 257 | 964 | 458 | 5.98 | 53 | 5.91 | 1.42 | 8.2 | 205 | |
| 240 | 763 | 295 | 5.73 | 35 | 1.84 | 1.39 | 8.1 | 209 | 28 |
| 235 | 947 | 295 | 5.60 | 71 | 6.53 | 1.50 | 10.0 | 172 | |
| 230 | 589 | 443 | 5.64 | 30 | 4.41 | 1.22 | 8.5 | 208 | 28 |
| 234 | 863 | 609 | 5.64 | 39 | 3.82 | 1.34 | 8.7 | 199 | 33 |
| 229 | 761 | 487 | 6.01 | 30 | 1.38 | 1.30 | 8.4 | 207 | 28 |
| 268 | 550 | 76 | 6.36 | 72 | 8.3 | 1.22 | 8.8 | 203 | |

TABLE 10
mAb1 dispersions made with trehalose, imidazole and either hydrochloric acid (HCl),
citric acid (CA) or phosphoric acid (PA). Replicates are shaded together in grey.
These dispersions were formed with the lyophilization dilution technique.

| | Conc | | | | | | | | | |
|---|---|---|---|---|---|---|---|---|---|---|
| Protein | St | | Trehalose | | Imidazole | | | Acid | | |
| Conc | Dev | | Conc | | Conc | | | Conc | | Exc: |
| mg/ | mg/ | Yield | mg/ | | mg/ | | | mg/ | | Protein |
| ml | ml | % | ml | mM | ml | mM | Acid | ml | mM | M:M |
| 247 | 6.8 | 92 | 49 | 130 | 17 | 246 | CA | 19 | 91 | 284 |
| 245 | 0.15 | 90 | 49 | 130 | 17 | 244 | CA | 19 | 90 | 283 |
| 268 | 5.55 | 100 | 54 | 142 | 17 | 244 | CA | 10 | 47 | 242 |
| 288 | 10.41 | 103 | 58 | 152 | 16 | 241 | CA | 10 | 47 | 229 |
| 258 | 2.91 | 98 | 52 | 136 | 33 | 492 | CA | 20 | 95 | 421 |
| 257 | 4.38 | 96 | 51 | 136 | 33 | 489 | CA | 20 | 94 | 419 |
| 299 | 2.79 | 93 | 60 | 158 | 17 | 243 | CA | 10 | 47 | 225 |
| 268 | 11.98 | 98 | 54 | 142 | 17 | 244 | CA | 8 | 39 | 238 |
| 255 | 14.24 | 97 | 52 | 137 | 17 | 247 | HCl | 6 | 178 | 331 |
| 260 | 10.81 | 97 | 54 | 142 | 17 | 244 | HCl | 6 | 176 | 325 |
| 230 | 3.21 | 85 | 53 | 139 | 17 | 244 | HCl | 6 | 176 | 365 |
| 249 | 4.78 | 96 | 52 | 137 | 17 | 247 | HCl | 5 | 126 | 307 |
| 239 | 2.12 | 90 | 48 | 126 | 17 | 246 | PA | 16 | 162 | 335 |
| 239 | 3.61 | 90 | 48 | 126 | 17 | 246 | PA | 16 | 161 | 335 |
| 244 | 16.88 | 92 | 49 | 129 | 17 | 246 | PA | 14 | 144 | 319 |

| | Calc | | | | | | | Calc | |
|---|---|---|---|---|---|---|---|---|---|
| | Ideal | | | | | | | Conc | Nano- |
| Protein | Osmo- | | | | Visc | Sol- | | at | cluster |
| Conc | lality | Ionic | | Vis- | St | vent | Intrin- | 20 cP | Diam- |
| mg/ | mOsm/ | Strength | | cosity | Dev | Visc | sic | mg/ | eter |
| ml | kg | mM | pH | cP | cP | cP | Visc | ml | nm |
| 247 | 564 | 349 | 5.88 | 35 | 0.3 | 1.18 | 8.2 | 216 | |
| 245 | 559 | 344 | 5.86 | 46 | 2.81 | 1.18 | 8.9 | 203 | 31.3 |
| 268 | 531 | 249 | 7.01 | 38 | 2.71 | 1.16 | 7.5 | 232 | |
| 288 | 549 | 248 | 6.94 | 58 | 2.97 | 1.17 | 7.5 | 231 | 33.2 |
| 258 | 891 | 503 | 6.98 | 34 | 3.38 | 1.22 | 7.6 | 227 | 31.2 |
| 257 | 886 | 498 | 7.03 | 42 | 1.44 | 1.22 | 8.1 | 216 | |
| 299 | 564 | 250 | 6.92 | 167 | 1.21 | 1.18 | 9 | 201 | |
| 268 | 521 | 216 | 7.09 | 58 | 7.01 | 1.16 | 8.4 | 213 | |
| 255 | 679 | 178 | 6.51 | 36 | 5.39 | 1.12 | 8 | 223 | |
| 260 | 682 | 176 | 6.55 | 43 | 2.54 | 1.13 | 8.2 | 219 | 35 |
| 230 | 663 | 176 | 6.53 | 27 | 1.58 | 1.12 | 8.5 | 213 | 33 |
| 249 | 613 | 126 | 6.97 | 42 | 2.76 | 1.12 | 8.7 | 210 | |
| 239 | 639 | 208 | 6.14 | 51 | 23.4 | 1.11 | 9.7 | 193 | |
| 239 | 638 | 208 | 6.09 | 44 | 3.52 | 1.11 | 9.3 | 200 | 33.3 |
| 244 | 622 | 198 | 6.24 | 59 | 3.96 | 1.11 | 9.7 | 193 | |

TABLE 11
Viscosity and nanocluster size of mAb1 protein. The intrinsic viscosity decreases significantly
with concentration of the excipients. Nanocluster size remains essentially constant. Without
being limited by theory, more interacting crowders may block more sites on the proteins thus
lowering the viscosity. The dispersions were made by centrifugal filtration.

| | | | | | | | | | Nano- | | | |
|---|---|---|---|---|---|---|---|---|---|---|---|---|
| Protein | std | | Exc 1 | | Exc 2 | | | | cluster | | Cfg | Cfg |
| conc | dev | | Conc | | Conc | | Viscosity | Intrinsic | Diameter | % | time | speed |
| (mg/ml) | (mg/ml) | Exc 1 | (mg/ml) | Exc 2 | (mg/ml) | pH | (cP) | Viscosity | (nm) | yield | (min) | (rcf) |
| 246 | 17.7 | arg | 39.3 | glu | 35.7 | 7 | 67 ± 1.9 | 9.7 | 39 ± 7.7 | 87 | 48 | 5000 |
| 245 | 24.4 | arg | 52.4 | glu | 47.6 | 7 | 49 ± 1.8 | 8.8 | 23 ± 3.7 | 81 | 45 | 5000 |
| 241 | 0.2 | arg | 65.5 | glu | 59.5 | 7 | 42 ± 5.2 | 8.4 | 29 ± 4.7 | 89 | 50 | 5000 |
| 263 | 13.4 | arg | 78.6 | glu | 71.4 | 7 | 41 ± 1.7 | 7.2 | 43 ± 4.2 | 92 | 63 | 5000 |

TABLE 12
Viscosity for mAb1 monomer solutions is higher than dispersions for a wide range of pH values. Typical
viscosities for dispersions (Table 3) were at least 4-5 times lower for comparable concentrations
for these solutions. (15 cP vs 100 cP). The dispersions were made by centrifugal filtration.

| | | | | | | | Nano- | | | |
|---|---|---|---|---|---|---|---|---|---|---|
| Protein | std | | Buffer | | | | cluster | | Cfg | Cfg |
| conc | dev | | Conc | | Viscosity | Intrinsic | Diameter | % | time | speed |
| (mg/mL) | (mg/ml) | Buffer | (mM) | pH | (cP) | viscosity | (nm) | yield | (min) | (rcf) |
| 197 | 15.2 | none | — | 5.5 | 80 ± 3.5 | 26.6 | 11.5 ± 3 | 95.5 | 50 | 5000 |
| 192 | 7.2 | His/His. HCl | 20 | 5.5 | 148 ± 8 | 31.2 | 7 ± 3.5 | 63 | 20 | 10000 |
| 191 | 1.0 | phosphate | 50 | 6.5 | 84 ± 5.9 | 27.8 | 14.2 ± 3.9 | 85.5 | 45 | 5000 |

TABLE 13
Low viscosities obtained for sheep IgG. Dispersions for sheep IgG with similar compositions of interacting
crowders show low intrinsic viscosities between 6-7. The dispersions were made by centrifugal filtration.

| | | | | | | | | | Nano- | | | |
|---|---|---|---|---|---|---|---|---|---|---|---|---|
| | Protein | | Exc 1 | | Exc 2 | | | | cluster | | Cfg | Cfg |
| | conc | | Conc | | Conc | | Viscosity | Intrinsic | Diameter | % | time | speed |
| Protein | (mg/ml) | Exc 1 | (mg/ml) | Exc 2 | (mg/ml) | pH | (cP) | Viscosity | (nm) | yield | (min) | (rcf) |
| IgG | 252 | arg | 75 | glu | 75 | 5.5 | 27 | 6.9 | 50.2 ± 13.0 | 100 | 45 | 10000 |
| IgG | 258 | arg | 108.5 | glu | 91.5 | 6.6 | 30 ± 0.3 | 6.6 | 62.6 ± 5.3 | 94 | 55 | 10000 |

TABLE 14
Viscosity and nanocluster size of mAb1 protein decreases with increasing concentration of arg/glu/trehalose. Initially
the intrinsic viscosity decreases with increase in concentration of the excipients. Without being limited by theory,
more interacting crowders may be expected to block more of the interacting sites on the proteins. A similar trend
as seen in earlier table. However, in this case trehalose was added to the dispersions. nanocluster size also decreases
with increased concentration of the crowders. The dispersions were made by centrifugal filtration.

| Protein | | Exc 1 | | Exc 2 | | Exc 3 | | | | Nanocluster | | Cfg | Cfg |
|---|---|---|---|---|---|---|---|---|---|---|---|---|---|
| conc | | Conc | | Conc | | Conc | | Viscosity | Intrinsic | Diameter | Yield | time | speed |
| (mg/ml) | Exc 1 | (mg/ml) | Exc 2 | (mg/ml) | Exc 3 | (mg/ml) | pH | (cP) | Viscosity | (nm) | (%) | (min) | (rcf) |
| 241 | arg | 27.5 | glu | 22.5 | tre | 25 | 7.9 | 86 ± 20 | 11.4 | 48 ± 3.6 | 71 | 95 | 5000 |
| 252 | arg | 36.7 | glu | 30 | tre | 33.3 | 7.9 | 80 ± 16.5 | 9.7 | 41 ± 2.3 | 61 | 75 | 5000 |
| 238 | Arg | 73.4 | glu | 60 | tre | 66.7 | 7.9 | 51 ± 2.0 | 8.5 | 32 ± 2.6 | 80 | 65 | 5000 |

TABLE 15
Viscosity vs pH for mAb1 formulations with 150 mg/ml arginine. Low viscosities
were observed at pH 11.1 and 6.4, but at 10.1, close to the isoelectric point,
the viscosity was higher. The dispersions were made by centrifugal filtration.

| Protein | | Exc 1 | | Exc 2 | | | | | Cfg | Cfg |
|---|---|---|---|---|---|---|---|---|---|---|
| conc | | Conc | | Conc | | Viscosity | Intrinsic | Yield | time | speed |
| (mg/ml) | Exc 1 | (mg/ml) | Exc 2 | (mg/ml) | pH | (cP) | Viscosity | (%) | (min) | (rcf) |
| 234 | arg | 150 | — | | 11.1 | 34 ± 0.5 | 8.3 | 73 | 25 | 10000 |
| 229 | arg | 150 | HCl | 30.1 | 6.4 | 47 ± 10.7 | 9.2 | 67 | 30 | 10000 |

TABLE 16
SEC for mAb1 protein does not show increased aggregation for diluted
mAb dispersion samples compared to the monomer control.

| A. Sample | % monomer in the sample |
|---|---|
| Monomer | 99.89 |
| Cluster | 99.86 |

TABLE 17
SEC for mAb1 protein does not show increased aggregation
for samples stored for up to 8 weeks. The dispersion that
was ~250 mg/ml with 81.4 mg/ml arg and 68.6 mg/ml glu
and was 99.89% monomer pre-storage. All the samples run
on the SEC column were diluted to ~1 mg/ml.

| | % monomer for | % monomer for | % monomer for |
|---|---|---|---|
| | sample stored | sample stored | sample stored |
| | frozen | refrigerated | at room |
| Week | at −40° C. | at 4° C. | temperature |
| 1 | 99.86 | 99.87 | 99.86 |
| 1.5 | 99.87 | 99.90 | 99.84 |
| 2 | 99.85 | 99.91 | 99.83 |
| 4 | 99.77 | 99.86 | n/a |
| 8 | 99.90 | 99.88 | 99.75 |

TABLE 18
mAb1 dispersions made with trehalose, histidine and hydrochloric acid (HCl). Replicates are shaded
together in grey. These dispersions were formed with the lyophilization dilution technique.

| Pro- | Conc | | | | | | | | | Calc | | | | | | | |
|---|---|---|---|---|---|---|---|---|---|---|---|---|---|---|---|---|---|
| tein | St | | Trehalose | | Histidine | | HCl | | Exc: | Ideal | | | | Visc | | | Nano- |
| Conc | Dev | | Conc | | Conc | | Conc | | Pro- | Osmo- | Ionic | | Vis- | St | Solvent | Intrin- | cluster |
| mg/ | mg/ | Yield | mg/ | | mg/ | | mg/ | | tein | lality | Strength | | cosity | Dev | Visc | sic Vis- | Diameter |
| ml | ml | % | ml | mM | ml | mM | ml | mM | M:M | mOsm/kg | mM | pH | cP | cP | cP | cosity | nm |
| 233 | | 93 | 51 | 135 | 0 | 0 | 0 | 0 | 97 | 137 | 0 | 6.54 | 646 | 217 | 1.05 | 16.8 | |
| 267 | 6.7 | 102 | 52 | 138 | 0 | 0 | 0 | 0 | 78 | 168 | 0 | 6.41 | 329 | 2.9 | 1.06 | 12.4 | |
| 270 | 5.9 | 104 | 56 | 147 | 5 | 30 | 1 | 30 | 115 | 243 | 25 | 5.79 | 130 | 5.6 | 1.08 | 10.2 | |
| 257 | 2.2 | 99 | 51 | 134 | 5 | 30 | 1 | 20 | 107 | 222 | 17 | 6.10 | 171 | 4.5 | 1.09 | 11.6 | |
| 252 | 11.2 | 97 | 52 | 137 | 20 | 127 | 3 | 85 | 208 | 423 | 79 | 5.90 | 91 | 10.2 | 1.15 | 10.2 | 44 |
| 258 | 2.6 | 100 | 53 | 139 | 38 | 248 | 9 | 248 | 369 | 781 | 234 | 5.15 | 41 | 1.4 | 1.22 | 8 | 44 |
| 263 | 1.9 | 103 | 54 | 142 | 39 | 249 | 6 | 166 | 318 | 688 | 152 | 5.94 | 46 | 2.6 | 1.22 | 8 | 43 |
| 254 | 10.3 | 99 | 52 | 137 | 38 | 248 | 6 | 166 | 325 | 676 | 153 | 5.92 | 48 | 2.5 | 1.22 | 8.5 | 44 |
| 252 | 2.3 | 97 | 52 | 138 | 40 | 255 | 2 | 54 | 266 | 547 | 52 | 6.66 | 88 | 4.4 | 1.22 | 10 | 47 |
| 248 | 14.8 | 95 | 55 | 145 | 39 | 252 | 1 | 19 | 252 | 508 | 20 | 7.06 | 162 | 13.2 | 1.23 | 11.7 | |

TABLE 19
mAb1 dispersions made with trehalose, and other buffers. These dispersions were formed with the lyophilization dilution technique.

| Pro- | Conc | | | | | | | | | | Calc | | | | | | | Nano- |
|---|---|---|---|---|---|---|---|---|---|---|---|---|---|---|---|---|---|---|
| tein | St | Trehalose | | | Base | | | Acid | | Exc: | Ideal | | | | Visc | Sol- | | cluster |
| Conc | Dev | Conc | | | Conc | | | Conc | | Pro- | Osmo- | Ionic | | Vis- | St | vent | Intrin- | Diam- |
| mg/ | mg/ | mg/ | | | mg/ | | | mg/ | | tein | lality | Strength | | cosity | Dev | Visc | sic Vis- | eter |
| ml | ml | ml | mM | Base | ml | mM | Acid | ml | mM | M:M | mOsm/kg | mM | pH | cP | cP | cP | cosity | nm |
| 247 | 3.7 | 51 | 134 | Imid | 17 | 248 | HCl | 7 | 195 | 350 | 693 | 205 | 6.19 | 36 | 1.9 | 1.12 | 8.4 | 47 |
| 259 | 6.7 | | 140 | Arg | | | HCl | | 250 | | 791 | 249 | 6.36 | 45 | 3.9 | 1.21 | 8.2 | 47 |
| 238 | 5.2 | | 129 | Lys | | | HCl | | 251 | | 762 | 251 | 6.43 | 81 | 4 | 1.2 | 10.7 | 68 |
| 254 | 6.3 | | 138 | NaOH | | 297 | Phos | | 246 | | 827 | 291 | 5.94 | 85 | 3.8 | 1.05 | 10.2 | 74 |

TABLE 20
mAb1 dispersions made with trehalose, histidine, hydrochloric acid (HCl) and additional salts as listed. All systems
contain ~50 mg/ml (130 mM) trehalose. These dispersions were formed with the lyophilization dilution technique.

| Pro- | Conc | | | | | | | | | | Calc | | | | | | Nano- |
|---|---|---|---|---|---|---|---|---|---|---|---|---|---|---|---|---|---|
| tein | St | | Histidine | | HCl | | | Salt | | Exc: | Ideal | | | | Visc | Sol- | cluster |
| Conc | Dev | | Conc | | Conc | | | Conc | | Pro- | Osmo- | Ionic | | Vis- | St | vent | Diam- |
| mg/ | mg/ | Yield | mg/ | | mg/ | | | mg/ | | tein | lality | Strength | | cosity | Dev | Visc | eter |
| ml | ml | % | ml | mM | ml | mM | Salt | ml | mM | M:M | mOsm/kg | mM | pH | cP | cP | cP | nm |
| 242 | 1.5 | 93 | 39 | 254 | 2 | 54 | NaCl | 5 | 90 | 327 | 746 | 143 | 6.62 | 78 | 6.6 | 1.21 | 53 |
| 255 | 8.3 | 97 | 20 | 127 | 3 | 85 | MgSO₄ | 19 | 19 | 212 | 461 | 151 | 5.98 | 54 | 5.1 | 1.14 | |
| 271 | 2.9 | 99 | 20 | 127 | 3 | 85 | NaCl | 4 | 76 | 231 | 606 | 152 | 5.99 | 76 | 7.2 | 1.15 | |
| 247 | 8.7 | 95 | 20 | 128 | 3 | 85 | NH₄SCN | 6 | 74 | 253 | 584 | 150 | 6.01 | 77 | 4.1 | 1.14 | |

TABLE 21
mAb1 dispersions made with sucrose, histidine and hydrochloric acid. These dispersions were formed with the lyophilization dilution technique.

| Protein | Conc | | Sucrose | | Histidine | | HCl | | | | | | Visc | | | Nano- |
|---|---|---|---|---|---|---|---|---|---|---|---|---|---|---|---|---|
| Conc | St Dev | | Conc | | Conc | | Conc | | Exc: | Ionic | | Vis- | St | Solvent | Intrin- | cluster |
| mg/ | mg/ | Yield | mg/ | | mg/ | | mg/ | | Protein | Strength | | cosity | Dev | Visc | sic Vis- | Diameter |
| ml | ml | % | ml | mM | ml | mM | ml | mM | M:M | mM | pH | cP | cP | cP | cosity | nm |
| 250 | 14.5 | 96 | 52 | 152 | 50 | 324 | 9 | 251 | 436 | 251 | 5.74 | 49.5 | 2.87 | 1.22 | 8.8 | 34 |
| 269 | 7.1 | 103 | 52 | 152 | 40 | 255 | 7 | 198 | 337 | 198 | 5.74 | 55.4 | 4.32 | 1.19 | 8.2 | 35 |

TABLE 22
mAb1 dispersions made with trehalose, histidine, citric acid and additional amino acids (AA), Betaine (Bet),
Asparagine (Asn), Glutamine (Gln). These dispersions were formed with the lyophilization dilution technique.

| Protein | Conc | trehalose | | Histidine | | Citric Acid | | | AA | | | | | Visc | | | Nano- |
|---|---|---|---|---|---|---|---|---|---|---|---|---|---|---|---|---|---|
| Conc | St Dev | Conc | | Conc | | Conc | | | Conc | | Ionic | | Vis- | St | Solvent | Intrin- | cluster |
| mg/ | mg/ | mg/ | | mg/ | | mg/ | | | mg/ | | Strength | | cosity | Dev | Visc | sic Vis- | Diameter |
| ml | ml | ml | mM | ml | mM | ml | mM | AA | ml | mM | mM | pH | cP | cP | cP | cosity | nm |
| 232 | | 51 | 135 | 40 | 258 | 18 | 87 | Bet | 40 | 342 | 335 | 5.66 | 48.1 | 8.41 | 1.39 | 9.3 | |
| 242 | 4.0 | 54 | 144 | 39 | 253 | 18 | 86 | Asn | 16 | 118 | 294 | 5.64 | 34.6 | 0.87 | 1.33 | 8.1 | 31 |
| 237 | 8.0 | 53 | 141 | 39 | 255 | 18 | 86 | Gln | 23 | 158 | 295 | 5.59 | 39.7 | 2.24 | 1.35 | 8.6 | 32 |
| 239 | 2.5 | 53 | 139 | 10 | 63 | 5 | 22 | Asn | 16 | 118 | 74 | 5.80 | 48.4 | 4.00 | 1.21 | 9.3 | 31 |
| 250 | 9.4 | 51 | 136 | 10 | 63 | 5 | 21 | Gln | 23 | 156 | 74 | 5.79 | 46.4 | 3.11 | 1.22 | 8.6 | 30 |
| 239 | 9.4 | 51 | 135 | 39 | 254 | 18 | 86 | Asn | 16 | 119 | 295 | 5.71 | 32.2 | 1.11 | 1.32 | 8.1 | 30 |
| 222 | 3.3 | 51 | 134 | 40 | 255 | 18 | 87 | Gln | 23 | 158 | 297 | 5.65 | 26.5 | 2.35 | 1.34 | 8.3 | 30 |

### Example 2

### Reduction of mAb Viscosity at 250 mg/Ml by Addition of High Arg Concentrations to Modify Protein-Protein Interactions

Summary.

Monoclonal antibodies (mAbs) are of great interest in the pharmaceutical field as therapeutics for the treatment of a variety of disorders and diseases. To enable subcutaneous self-injection of mAbs, it is necessary to develop low viscosity dispersions of mAbs at high concentration. The viscosity of mAb1 dispersions at 250 mg/ml was reduced by about 6 times compared to low co-solute protein dispersions in buffer by the addition of high concentrations of co-solutes, namely, arg with either glu or HCl. Arg binds to proteins and reduces both the hydrophobic and localized electrostatic attraction between proteins. Lowered interactions between entities in the dispersion can have the effect of lowering the viscosity. The viscosity was observed to decrease as the total concentration of arg and glu was increased at a fixed pH and fixed ratio of arg to glu as evidenced by the inherent viscosity. The ratio of arg to glu or HCl was also varied to affect the dispersion pH with the viscosity observed to reach a maximum around pH 9 which is the isoelectric point of mAb1. The importance of the binding and interactions of co-solutes with proteins was examined by comparing the protein dispersions with arg to those containing a non-interacting co-solute, namely trehalose which had no impact on the dispersion viscosity. The absence of protein specificity of the effect of co-solutes on the dispersion properties was demonstrated through a viscosity of 17 cP at 258 mg/ml for a polyclonal sheep IgG mixture. For all high co-solute dispersions, the diffusion coefficient as measured by DLS decreased while that for the low co-solute monomer samples remained comparable to that of the monomer in dilute conditions even at high concentrations of protein. The lowered diffusion coefficient was in contrast to the lowering of dispersion viscosity. Upon dilution, the high co-solute dispersions were determined to yield monomeric protein by both DLS and SEC.

Introduction.

Monoclonal antibodies (mAbs) are of great interest for the treatment of many diseases and disorders including various types of cancer, rheumatoid arthritis, Alzheimer's disease, asthma, and Crohn's disease. There are currently more than 40 mAbs that are either currently under review or have already been approved by the drug administrations in either the United States or the European Union. [1] The most common method of administration through an intravenous drip (IV) of a dilute solution is time consuming and requires medical supervision, in contrast with subcutaneous (subQ) self-injection. [2,3] Due to the limited volume of ˜1-2 ml for subQ administration, the recommended dosage may require use of concentrations greater than 150 mg/ml mAb where viscosities can exceed the desired level of 20-50 cP. [2,4] The elevated viscosities often arise as a result of protein-protein interactions between the Fab (antibody binding fragment) regions of mAbs, specifically between CDRs (complementarity determining regions). [4-6] The viscosity at high concentration has been shown to increase markedly for a series of mAbs as the interactions (measured at low concentration by dynamic or static light scattering) change from repulsive to attractive, for a given buffer/co-solute system. [5,7-10]

Often mAbs with isoelectric points between pH 7 and 9 are formulated in a buffer solution at pH 5-6, for example, 20-30 mM his/his.HCl and in relatively few cases with a saccharide. [2,5-9,11,12] The pH is chosen to be a few units away from the isoelectric point such that the net charge provides electrostatic repulsion to favor stability against aggregation. The distribution of charged and hydrophobic patches on proteins in concentrated (150 to 300 mg/ml) dispersions in these buffers may produce highly complex anisotropic interactions, given a small average surface-to-surface separation of ˜1-5 nm. Here, the hydrophobic, electrostatic (between oppositely charged patches) and charge-dipole interactions may produce aggregation, which is of concern with regard to immunogenicity in addition to protein unfolding and gelation. [13-16] These interactions can cause formation of protein oligomers, where dimers have been found in some case to be the most common species. [12,17] These oligomeric structures may produce a large increase in the viscosity given that they occupy a greater volume than monomeric protein as a function of the fractal dimension. [12,17] Here, the viscosity may be elevated at low shear rates and then undergo shear thinning up to shear rates of ˜1000 s⁻¹ encountered in subQ injection, as the network structure of reversible aggregates is disrupted due to the higher shear energy from flow. [6,18] Even irreversible aggregates composed of a few proteins may cause shear thinning behavior as seen for bovine serum albumin (BSA) and for low concentration mAbs. [15,16,19]

A common approach for reducing the viscosity of mAb formulations is to engineer the amino acid sequence to weaken the aforementioned attractive interactions. [7,18] However, this approach is time consuming, highly specific to the particular mAb under consideration and may even influence the mAb therapeutic efficacy. [7,18] Salts may be used to reduce the Debye length and screen electrostatic interactions including charge dipole interactions. [5,12,20-22]. In some cases, strongly chaotropic salts disrupt the water structure at the protein surface and suppress mAb attractive interactions, therefore reducing the viscosity to a much greater degree compared to kosmotropic salts. [6,20] Also, salts containing large hydrophobic ions at concentrations of 0.5 M were shown to lower the viscosity up to 3-fold for BSA and bovine γ-globulin, likely by weakening the hydrophobic interactions, even in cases where hydrophilic salts had little effect. [20] Finally, low viscosities have been observed for micron-sized particulates of mAbs dispersed in organic solvents [3,23] or aqueous buffers containing a high organic solvent fraction [24] which lowers protein solubility so that the suspended particles do not dissolve.

Recently, there has been significant interest in utilizing organic co-solutes to modify protein-protein interactions to attempt to lower the viscosity. [25-28] High concentrations of a neutral co-solute, trehalose, were used to tune the depletion attraction between proteins resulting in a lowered diffusion coefficient as indicated by dynamic light scattering (DLS). [25,26,29] On the basis of a free energy model, it was proposed that self-limited protein nanoclusters may be formed near the isoelectric point for slightly charged proteins, as the short ranged depletion attraction between proteins was balanced by the long ranged repulsion between proteins. [25,26,30,31] Similarly, protein clusters were formed for highly concentrated lysozyme and BSA by light scattering with the measured fraction of clusters ranging from 10⁻⁵ to 0.8. [32-35] Even at low concentrations trehalose is known to be preferentially excluded from the protein surface favoring a compact folded state of the protein sometimes termed as osmotic compression. [36,37] Together, the depletion attraction from the concentrated co-solutes and the high concentration of proteins may be expected to favor folding and thus enhance protein stability. [38,39] After diluting the protein dispersions, aggregates were not observed by SEC and the protein was active in vitro and in vivo upon dilution into buffer. [25,29]

Unlike trehalose, the effect of co-solutes that interact strongly with proteins on protein stability and the viscosity of protein dispersions has received little attention. The addition of arg.HCl at 600 mM to mAb dispersions was hypothesized to reduce hydrophobic interactions in addition to mediating electrostatic interactions via charge screening. [27] The reduction of attractive hydrophobic interactions between mAbs occurs through the binding of arg to hydrophobic patches effectively making them more hydrophilic. [21, 22, 27, 28, 40-45] Similarly, the positively charged arg also binds to charged patches on mAbs through hydrogen bonding which changes the charge distribution on mAbs causing reduced attractive interactions between oppositely charged patches. [8,20-28] Arg.HCl at an even higher concentration of 1 M was seen to lower the viscosity of polyclonal antibody mixtures slightly from 60 cP to ˜45-50 cP through the mediation of attractive interactions in this manner. [28] Alternatively, arg.HCl at 200 mM has been used in mAb formulations to lower interactions and viscosity to some extent at mAb concentrations of 175 mg/ml. [7] It remains to be determined if strongly interacting co-solutes may produce large decreases in viscosity at moderate concentrations.

Herein, we examine how high concentrations of arg and glu or HCl influence the viscosity of protein dispersions from pH 5 to 11 by modifying protein-protein interactions. Specifically, the viscosity of a dispersion of mAb1 is reduced from ˜160 cP at 230 mg/ml in histidine buffer to as low as ˜30 cP at 250 mg/ml in the presence of arg and glu as co-solutes at total concentrations ranging from 75 to 150 mg/ml. The binding and interactions of these co-solutes with proteins may reduce both localized anisotropic electrostatic or hydrophobic protein-protein interactions that otherwise often produce high viscosities. [7, 10, 21, 22, 27, 40, 45, 46] The pH was varied to affect the interactions of the co-solutes with mAbs in order to modify charge distributions of the mAbs. Co-solute concentrations were also varied to modify protein-protein interactions and to add depletion attraction. Trehalose, which does not interact specifically with proteins, was chosen as a control to isolate the effect of depletion attraction alone on viscosity. [25,26] For arg and glu at the high concentrations in this study, depletion attraction may be expected to modify the various protein interactions. The diffusion coefficient was measured by DLS and compared with the calculated value for mAbs in a monomeric state with an effective spherical diameter of 11 nm diffusing through the solvent. The osmotic compression effect arising from co-solutes may also favor folding of proteins to reduce the molecular volume as has been demonstrated before [25,36] which may help reduce aggregation. Finally, we show test the stability of mAb dispersions upon dilution using size exclusion chromatography (SEC) after storage for up to 8 weeks.

### Results and Discussion

Effect of co-solutes on viscosity at ˜150 mg/ml mAb concentration. To provide a basis for understanding the viscosity experiments at ˜250 mg/ml protein we begin with experiments at a lower protein concentration of ˜150 mg/ml. Various co-solutes at ˜150 mg/ml were added to a 150 mg/ml solution of mAb1 in DI water which had been previously prepared by the tangential flow filtration (TFF) to attempt to lower the viscosity (η). To partially remove the effects of protein concentration (c) and solvent viscosity (η₀), the inherent viscosity (ηᵢₙₕ, mg/ml) was determined for all dispersions.

$${η}_{inh} = \frac{ln ( η / {η}_{0} )}{c} \qquad ( 1 )$$

The inherent viscosities at 130 mg/ml mAb1 are shown in Table 2-1 along with η and D/D₀ from DLS (where D₀ is the calculated diffusion coefficient for protein monomer with an effective spherical diameter of 11 nm in the same solvent). mAb1 upon formulation in DI water in the absence of any co-solutes had a η of 19 cP at ˜150 mg/ml and a pH of 5.5 with a D/D₀ of 1.4. The η decreased only a small amount at pH 7 (50 mM phosphate buffer) compared to pH 5.5 where D/D₀ was close to 1. Upon addition of arg titrated with either acetic or hydrochloric acid, the ηᵢₙₕ dropped by 30% and 60% respectively, at a concentration of ˜130 mg/ml. The ηᵢₙₕ was much smaller for arg.HCl than for all of the other systems. In contrast, sodium glutamate did not reduce ηᵢₙₕ compared to the controls. However, for a 1:1 mixture of sodium glutamate and arg.HCl, ηᵢₙₕ and D/D₀ decreased a similar extent as observed for lysine or proline. For all of the high co-solute systems, the D/D₀ was below unity and as low as 0.53 suggesting slower diffusion for the scattering entities. However, these dispersions were not filtered. Also, it was not possible to resolve from DLS what fraction of protein was in the monomer state at these conditions or to determine the polydispersity given how close D/Do was to unity and the complexity of diffusion at high concentrations and viscosities. [48]

TABLE 2-1
Viscosity of mAb1 dispersions at ~130 mg/ml after
addition of 150 mg/ml of amino acids as co-solute.

| Protein | | Co-solute | | | | | |
|---|---|---|---|---|---|---|---|
| conc | | conc | | η₀ | η | ηᵢₙₕ | |
| (mg/ml) | Co-solutes | (mg/ml) | pH | (cP) | (cP) | (ml/mg) | D/D₀ |
| 146 | — | — | 5.5 | 1.0 | 18.5 ± 1.4 | 0.0175 | 1.43 |
| 140 | 50 mM phosphate | — | 7.4 | 1.0 | 15.5 ± 2.0 | 0.0197 | 1.06 |
| | buffer | | | | | | |
| 130 | Arg acetate | 153 | 6 | 1.5 | 7.9 ± 0.3 | 0.0128 | — |
| 132 | Arg•HCl | 144 | 7 | 1.5 | 3.6 ± 0.4 | 0.0067 | 0.62 |
| 129 | Lysine•HCl | 159 | 7 | 1.3 | 5.7 ± 0.5 | 0.0103 | 0.53 |
| 130 | Proline | 130 | 6.5 | 1.25 | 6.0 ± 0.5 | 0.0124 | 0.61 |
| 135 | Sodium glutamate | 144 | 7.5 | 1.5 | 18.4 ± 0.3 | 0.0186 | — |
| 135 | Sodium Glutamate | 72 each | 7 | 1.5 | 5.4 ± 1.2 | 0.0095 | 0.72 |
| | and arg•HCl | | | | | | |
| | (1:1 by wt) | | | | | | |

Additional experiments are presented for systems containing trehalose (tre) in Table 2-2 for a mAb concentration of ˜150 mg/ml. The η and ηᵢₙₕ for the two buffer controls with no co-solutes were relatively high. Tre is well-known to favor folding of proteins by minimizing the surface area because it is preferentially excluded from the protein surface. [11,36,49] However, the addition of tre to the pH 8 solution did not affect η or ηᵢₙₕ significantly while at pH 5.5, it decreased the ηᵢₙₕ˜20%. The mAb concentration was maintained at 150 mg/ml by addition of lyophilized mAb1 powder (with 1:1 mAb1 to tre by weight) to compensate for dilution due to co-solute addition. The addition of arg.HCl along with tre decreased ηᵢₙₕ by almost 30% with η reduced by almost 2 fold. Unlike the case for arg.HCl, the addition of glycine with tre at pH 5.5 did not reduce ηᵢₙₕ significantly more than tre by itself. All the subsequent experiments were conducted with the use of some arg in the dispersion along with an acid such as glutamic acid (glu) or HCl to modulate the sample pH as arg was the most effective co-solute at reducing η.

TABLE 2-2
Viscosity of mAb1 dispersions at ~150 mg/ml containing ~150 mg/ml
mixtures of tre with and without amino acids as co-solutes.

| Protein | | Co-solute | | | | |
|---|---|---|---|---|---|---|
| conc | | conc | | η₀ | η | ηᵢₙₕ |
| (mg/ml) | Co-solutes | (mg/ml) | pH | (cP) | (cP) | (ml/mg) |
| 146 | — | — | 5.5 | 1.0 | 18.5 ± 1.4 | 0.0175 |
| 150 | 50 mM phosphate | — | 8.2 | 1.0 | 12.6 ± 0.2 | 0.0144 |
| | buffer | | | | | |
| 136 | Tre | 137 | 5.5 | 1.3 | 11.8 ± 0.3 | 0.0152 |
| 162 | Tre | 155 | 8 | 1.3 | 14.9 ± 0.5 | 0.0150 |
| 155 | Tre and arg•HCl | 86 tre and | 7.5 | 1.4 | 8.0 ± 1.0 | 0.0111 |
| | | 74 arg•HCl | | | | |
| 135 | Tre and glycine | 72 each | 5.5 | 1.3 | 8.4 ± 0.3 | 0.0138 |
| | (1:1 by weight) | | | | | |

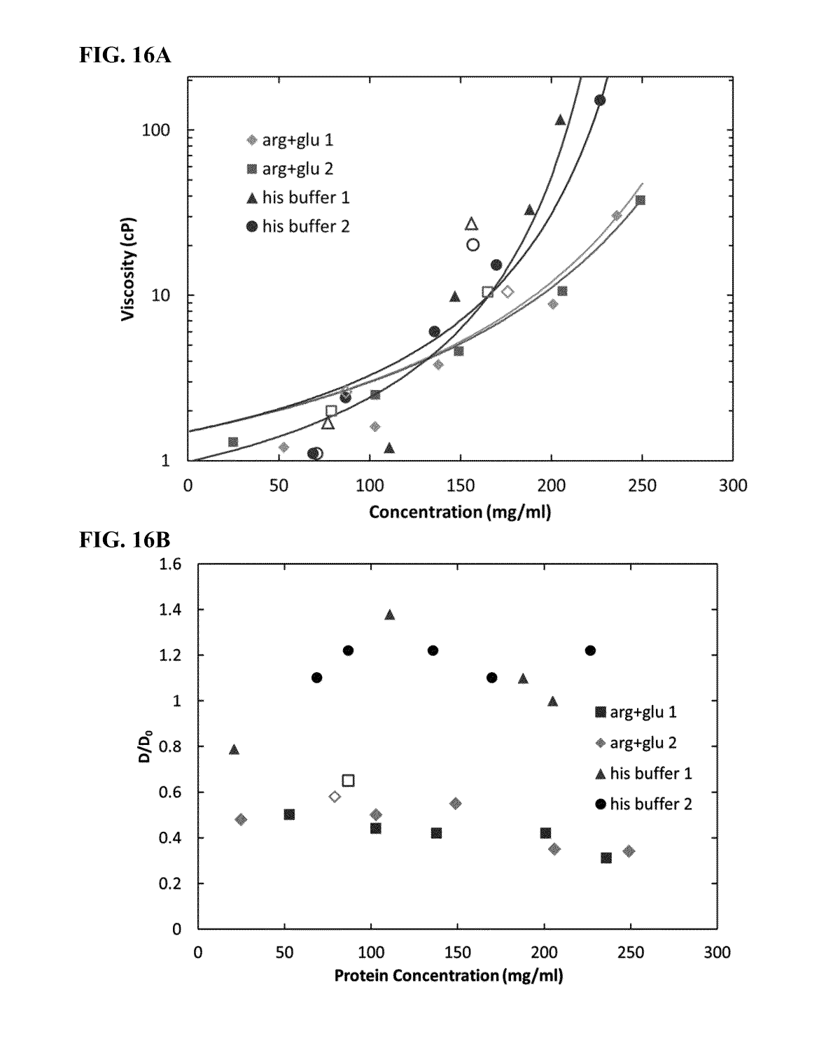

Effect of co-solutes on viscosity at ˜250 mg/ml mAb concentration at pH˜5.5. Experiments were conducted over a wide range of mAb1 concentrations as shown in a semi-log plot in FIG. 16A. for a 20 mM low co-solute his/his.HCl buffer control along with a high co-solute system containing 78.6 mg/ml of arg and 71.4 mg/ml of glu, both at pH 5.5. The detailed data with ηᵢₙₕ are shown in Table 2-S1 and Table 2-S2. The reproducibility for was within ˜10% for the two replicates shown for each sample at higher concentrations. The ηs for both the low co-solute buffer and high co-solute samples were well below 10 cp at low mAb concentrations. The η of high co-solute samples was ˜35 cP at 250 mg/ml and several times lower than 150 cP at ˜230 mg/ml measured for the buffer samples. Two control samples with low histidine at the same pH of 5.5 were prepared to examine the reproducibility as shown in Table 2-3. These samples exhibited trends observed in FIG. 16A with a η>150 cP much larger than for the high co-solute dispersions. Upon adding 150 mM NaCl to screen electrostatic interactions, the ηᵢₙₕ decreased significantly by about 15%, in contrast with a decrease of about 40% for the high co-solute dispersions. The D/D₀ decreased significantly upon the addition of salt probably as a result of lowered overall charge repulsion leading to proteins assembling into larger structures, even though an organic depletant was not added at high concentration. The yield for rows 1 and 3 is lower than row 2 possibly due to more loss to the larger filters used for their preparation compared to the smaller filters used for row 2. The most concentrated samples were diluted to explore the reversibility of the η and are shown by the open symbols. The η of the diluted samples although slightly elevated was within experimental error of 20% for ηᵢₙₕ. For FIG. 16A, curves were fit with the Ross-Minton equation which is a modified version of the Mooney equation. [5,6,50]

$$η = {η}_{0 } exp ( \frac{c [ n ]}{1 - ( k / v ) c [ n ]} ) \qquad ( 2 )$$

where [η] is the intrinsic viscosity in ml/mg, k is the crowding factor and v is the Simha parameter (shape determining factor). The regressed values of the [η] and k/v were 0.0053 ml/mg and 0.46 respectively for the high co-solute samples and 0.0054 ml/mg and 0.61 for the low co-solute samples. The 25% decrease in k/v for the high co-solute samples may suggest a significant reduction in the strength of mAb-mAb interactions based on the results from Kanai et al. [5,6] The [η] is related primarily to the molecular volume and consequently was relatively invariant. [5,6]

TABLE 2-3
Viscosity for mAb1 dispersions in low concentration histidine (his to
his•HCl was 0.18:1 by mass in both cases) buffers at pH 5.5 run at
a centrifugation speed of 5000 rcf. Rows 1 and 3 were run in Centricon
centrifugal concentrator tubes with a centrifugation speed of 4500 rcf.

| Protein | His buffer | | | | | | Cfg |
|---|---|---|---|---|---|---|---|
| conc | conc | η₀ | η | ηᵢₙₕ | | % | time |
| (mg/ml) | (mM) | (cP) | (cP) | (ml/mg) | D/D₀ | yield | (min) |
| 238 ± 7.2 | 30 | 1.0 | 193 ± 8 | 0.0219 | 1.01 | 54 | 54 |
| 228 ± 16.2 | 20 | 1.0 | 152 ± 19.2 | 0.0219 | 0.98 | 78 | 30 |
| 229 ± 2.8 | 30 (±150 | 1.0 | 66.7 ± 3.4 | 0.018 | 0.29 | 51 | 69 |
| | mM NaCl) | | | | | | |

TABLE 2-S1
Viscosity and D/D₀ with increasing protein concentration at 78.6 mg/ml arg
and 71.4 mg/ml glu at pH 5.5. The inherent viscosity remains more or less constant.
D/D₀ seems to decrease with increased protein conc. The data is shown graphically
in FIGS. 16A-16B. The centrifugation speed was 5000 rcf.

| | Protein | std | | Inherent | | | Cfg |
|---|---|---|---|---|---|---|---|
| Replicate and | conc | dev | Viscosity | Viscosity | | % | time |
| pathway | (mg/ml) | (mg/ml) | (cP) | (ml/mg) | D/D₀ | yield | (min) |
| 1 concentration | 53 | 2.0 | 1.8 ± 0.2 | — | 0.50 | N/A | N/A |
| 2 concentration | 25 | 3.0 | 1.3 ± 0.2 | — | 0.48 | N/A | N/A |
| 1 concentration | 103 | 2.0 | 1.6 ± 0.1 | 0.0007 | 0.44 | N/A | N/A |
| 2 concentration | 103 | 0.3 | 2.5 ± 0.2 | 0.0050 | 0.50 | N/A | N/A |
| 1 concentration | 138 | 2.4 | 3.8 ± 0.3 | 0.0067 | 0.42 | 86 | 25 |
| 2 concentration | 149 | 13.6 | 4.6 ± 0.4 | 0.0075 | 0.55 | 99 | 30 |
| 1 concentration | 201 | 8.8 | 8.8 ± 0.4 | 0.0088 | 0.42 | 96 | 35 |
| 2 concentration | 206 | 13.7 | 10.6 ± 0.4 | 0.0095 | 0.35 | 96 | 35 |
| 1 concentration | 236 | 5.7 | 30.4 ± 6.2 | 0.0127 | 0.31 | 90 | 40 |
| 2 concentration | 249 | 11.5 | 37.6 ± 0.7 | 0.0129 | 0.34 | 70 | 40 |
| 1 dilution | 176 | 13.8 | 10.5 ± 0.4 | 0.0111 | — | N/A | N/A |
| 2 dilution | 165 | 4.1 | 9.7 ± 1.5 | 0.0113 | — | N/A | N/A |
| 1 dilution | 87 | 6.0 | 2.6 ± 0.1 | 0.0114 | 0.65 | N/A | N/A |
| 2 dilution | 79 | 3.9 | 2.0 ± 0.2 | 0.0090 | 0.58 | N/A | N/A |

TABLE 2-S2
Viscosity and cluster size with increasing protein concentration in 20 mM pH
5.5 his buffer. The inherent viscosity increases with protein concentration while the
D/D₀ seems to remain constant with increased protein conc. The data is shown
graphically in FIGS. 16A-16B. The centrifugation speed was 5000 rcf.

| | Protein | std | | Inherent | | | Cfg |
|---|---|---|---|---|---|---|---|
| Replicate and | conc | dev | Viscosity | Viscosity | | % | time |
| pathway | (mg/ml) | (mg/ml) | (cP) | (ml/mg) | D/D₀ | yield | (min) |
| 1 concentration | 21 | 6.0 | 0.8 ± 0.1 | — | 0.79 | N/A | N/A |
| 2 concentration | 69 | 0.1 | 1.1 ± 0.1 | 0.0011 | 1.10 | N/A | N/A |
| 1 concentration | 111 | 6.4 | 1.2 ± 0.2 | 0.0018 | 1.38 | N/A | N/A |
| 2 concentration | 87 | 3.5 | 1.5 ± 0.5 | 0.0050 | 1.22 | N/A | N/A |
| 1 concentration | 147 | 2.2 | 9.8 ± 0.6 | 0.0157 | — | 88 | 25 |
| 2 concentration | 136 | 2.2 | 6.0 ± 0.5 | 0.0133 | 1.22 | 67 | 25 |
| 1 concentration | 188 | 2.1 | 33.2 ± 2.2 | 0.0165 | 1.10 | 75 | 30 |
| 2 concentration | 170 | 0.3 | 15.2 ± 0.8 | 0.0137 | 1.10 | 58 | 20 |
| 1 concentration | 205 | 5.5 | 116.4 ± 3.9 | 0.0233 | 1.00 | 76 | 45 |
| 2 concentration | 227 | 4.0 | 151 ± 11.1 | 0.0222 | 1.22 | 64 | 40 |
| 1 dilution | 156 | 9.2 | 27.2 ± 0.1 | 0.0213 | — | N/A | N/A |
| 2 dilution | 157 | 2.0 | 20.1 ± 6.0 | 0.0192 | — | N/A | N/A |
| 1 dilution | 77 | 4.7 | 1.7 ± 0.4 | 0.0072 | — | N/A | N/A |
| 2 dilution | 71 | 0.1 | 1.1 ± 0.3 | 0.0015 | — | N/A | N/A |

The low co-solute samples have D/D₀ values nearly independent of protein concentration (FIG. 16B). Meanwhile, the D/D₀ for high co-solute samples varied from ˜0.5 for the lowest protein concentrations with a gradual decrease to 0.3 as the protein concentration reached 250 mg/ml. It seems counterintuitive that the scattering entities diffused more slowly when the η of the dispersion decreased. It is possible that an unknown fraction of reversible protein aggregates or clusters were present along with protein monomer since these dispersions were not filtered. Upon dilution of the highest concentration samples with high co-solute concentration to 0.5 mg/ml, the D/D₀ reverted to ˜1 as shown in Table 2-4 indicating that any clusters were reversible yielding monomers with additional data shown in Table 2-S3.

TABLE 2-4
D/D₀ for dilution of mAb1 dispersions (0.5 mg/ml) with
78.6 mg/ml arg and 71.4 mg/ml glu from FIGS. 16A-16B.

| Protein | Original | Effective CONTIN Diameter |
|---|---|---|
| conc (mg/ml) | D/D₀ | after dilution (nm) |
| 249 | 0.32 | 13 ± 2.0 |
| 236 | 0.34 | 12 ± 3.0 |

TABLE 2-S3
DLS sizes for dilution of samples
from Table 5 which were at pH 5.5.

| Protein | | | | | Effective CONTIN |
|---|---|---|---|---|---|
| conc | std dev | Arg Conc | Glu Conc | Original | Diameter after |
| (mg/ml) | (mg/ml) | (mg/ml) | (mg/ml) | D/D₀ | dilution (nm) |
| 210 | 21.4 | 39.3 | 35.7 | 0.48 | 7.8 ± 1.7 |
| 233 | 18.7 | 52.4 | 47.6 | 0.42 | 7.5 ± 2.7 |
| 245 | 24.4 | 52.4 | 47.6 | 0.48 | 7.8 ± 1.8 |
| 246 | 7.1 | 65.5 | 59.5 | 0.28 | 14.6 ± 4.2 |
| 250 | 1.7 | 65.5 | 59.5 | 0.34 | 9.0 ± 1.7 |
| 281 | 1.7 | 78.6 | 71.4 | 0.41 | 9.7 ± 2.1 |
| 281 | 1.5 | 78.6 | 71.4 | 0.38 | 8.6 ± 1.7 |

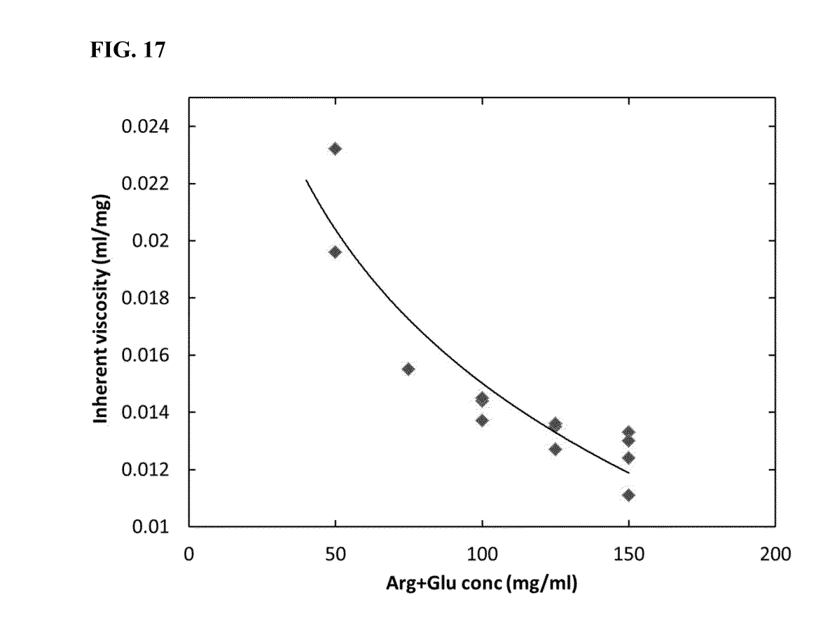

The effect of total concentration of arg and glu (75 to 150 mg/ml) on η was also studied by fixing the pH at 5.5 with a fixed ratio of arg to glu as 1.1:1 with a mAb concentration of ˜250 mg/ml (Table 2-5). The ηᵢₙₕ decreased with increasing total co-solute concentration as shown in FIG. 17 with replicate data shown in Table 2-S4. Additional data in FIG. 17 were taken from Table 2-6. The η was as low as 30 cP at 263 mg/ml for the 150 mg/ml total co-solute. Over this range of co-solute concentration (75 to 150 mg/ml) the overall η decreased by ˜45% despite the ˜25% increase in η₀ suggesting a weakening of protein-protein interactions. The yield for all the samples was >80% which is acceptable considering the potential for protein adsorption to the walls and filter membranes along with the multiple transfer steps that were conducted.

TABLE 2-5
Viscosity of mAb1 dispersions as a function of increasing conc
of arg/glu. The pH of all of the samples was 5.5 and the centrifugation
speed was 5000 rcf. The uncertainty is ±one std. dev. over multiple
measurements in both concentration and viscosity.

| Protein | Arg | Glu | | | | | Cfg |
|---|---|---|---|---|---|---|---|
| conc | conc | conc | η₀ | η | ηᵢₙₕ | % | time |
| (mg/ml) | (mg/ml) | (mg/ml) | (cP) | (cP) | (ml/mg) | yield | (min) |
| 246 ± 17.7 | 39.3 | 35.7 | 1.20 | 54 ± 1.9 | 0.0155 | 87 | 48 |
| 245 ± 24.4 | 52.4 | 47.6 | 1.33 | 38 ± 2.2 | 0.0137 | 81 | 45 |
| 241 ± 0.2 | 65.5 | 59.5 | 1.42 | 39 ± 4.8 | 0.0127 | 89 | 50 |
| 263 ± 13.4 | 78.6 | 71.4 | 1.5 | 30 ± 1.5 | 0.0111 | 92 | 63 |

TABLE 2-S4
Viscosity and cluster size with increasing conc of arg/glu. Replicate
data for Table 2-5 at a spin speed of 5000 rcf and pH 5.5.

| Protein | std | Arg | Glu | | Inherent | | | Cfg |
|---|---|---|---|---|---|---|---|---|
| conc | dev | Conc | Conc | Viscosity | Viscosity | | % | time |
| (mg/ml) | (mg/ml) | (mg/ml) | (mg/ml) | (cP) | (ml/mg) | D/D₀ | yield | (min) |
| 224 | 8.2 | 39.3 | 35.7 | 32 ± 2.0 | 0.0145 | 0.52 | 76 | 40 |
| 210 | 21.4 | 39.3 | 35.7 | 27 ± 2.3 | 0.0146 | 0.48 | 72 | 40 |
| 240 | 2.1 | 52.4 | 47.6 | 43 ± 4.4 | 0.0144 | 0.33 | 88 | 53 |
| 233 | 18.7 | 52.4 | 47.6 | 40 ± 1.7 | 0.0145 | 0.42 | 85 | 45 |
| 246 | 7.1 | 65.5 | 59.5 | 37 ± 2.6 | 0.0135 | 0.28 | 95 | 48 |
| 250 | 1.7 | 65.5 | 59.5 | 42 ± 5.3 | 0.0136 | 0.34 | 98 | 50 |
| 246 | 2.1 | 78.6 | 71.4 | 38 ± 1.6 | 0.0133 | 0.31 | 77 | 45 |
| 281 | 1.7 | 78.6 | 71.4 | 49 ± 5.3 | 0.0124 | 0.41 | 106 | 50 |
| 281 | 1.5 | 78.6 | 71.4 | 58 ± 5.2 | 0.0130 | 0.38 | 103 | 50 |

The experiments were scaled up in Table 2-6 to ˜500 μl samples by using 15 ml filters to enable sterile filtration of the dispersions. Four pairs of rows are shown where the first is before filtration and the second in bold is after sterile filtration. In each case the protein concentration dropped about 10% due to losses from adsorption to the filter while the sterile filtration yield was ˜60% due to losses to filter hold-up. Although this reduction in concentration lowered η, ηᵢₙₕ and D/D₀ did not change significantly. Therefore, η data for all other dispersions in this study was obtained by making ˜120 μl samples, which were not filtered to conserve protein, as they would be expected to follow this behavior. The D/D₀ was larger for the lower co-solute samples (50 mg/ml arg+glu) than for the higher co-solute samples. Additionally the extinction for the dispersions was measured and is reported as the turbidity divided by the mAb concentration at 350 nm with the entire spectrum shown. The turbidities were nearly twice as high for the 50 mg/ml arg+glu samples. It is possible that the higher turbidity was produced by reversible aggregates that also raised the η, but the structures of such aggregates have rarely been measured. [12,17,51]

TABLE 2-6
Viscosity, DLS (D/D₀) and turbidity/concentration at 350 nm of mAb1 dispersions made with the large 15 ml
Centricon centrifugal concentrator tubes with a centrifugation speed of 4500 rcf and a temperature of 20°
C. The second row in each pair shown in bold is the result after sterile filtration of the row above it.

| Protein | Arg | Glu | | | | | | Turbidity/ | | Cfg |
|---|---|---|---|---|---|---|---|---|---|---|
| conc | conc | conc | | η₀ | η | ηᵢₙₕ | | conc (ml | % | time |
| (mg/ml) | (mg/ml) | (mg/ml) | pH | (cP) | (cP) | (ml/mg) | D/D₀ | mg⁻¹ cm⁻¹) | yield | (min) |
| 288 ± 1.5 | 78.6 | 71.4 | 5.49 | 1.5 | 105 ± 29.1 | 0.0148 | 0.33 | 0.0013 | 80 | 105 |
| 261 ± 2.2 | 78.6 | 71.4 | 5.51 | 1.5 | 43 ± 13.8 | 0.0129 | 0.43 | 0.0020 | * | — |
| 288 ± 18.7 | 75 | 75 | 5.01 | 1.5 | 118 ± 22.5 | 0.0152 | 0.33 | 0.0013 | 77 | 105 |
| 269 ± 7.5 | 75 | 75 | 5.02 | 1.5 | 74 ± 4.6 | 0.0145 | 0.35 | 0.0015 | * | — |
| 260 ± 18.2 | 26.2 | 24.8 | 5.5 | 1.2 | 519 ± 51.9 | 0.0232 | 0.58 | 0.0023 | 68 | 90 |
| 241 ± 7.7 | 26.2 | 24.8 | 5.5 | 1.2 | 358 ± 31.6 | 0.0236 | 0.67 | 0.0030 | * | — |
| 283 ±/ 15.4 | 26.2 | 24.8 | 5.49 | 1.2 | 303 ± 29.6 | 0.0196 | 0.72 | 0.0018 | 82 | 90 |
| 239 ± 23.9 | 26.2 | 24.8 | 5.5 | 1.2 | 192 ± 30.8 | 0.0213 | 0.76 | 0.0026 | * | — |

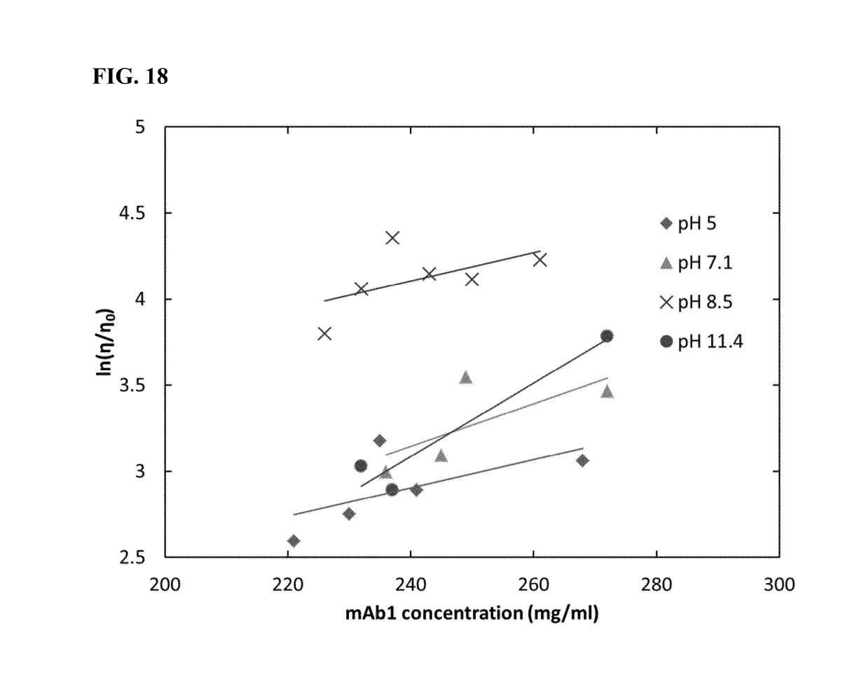

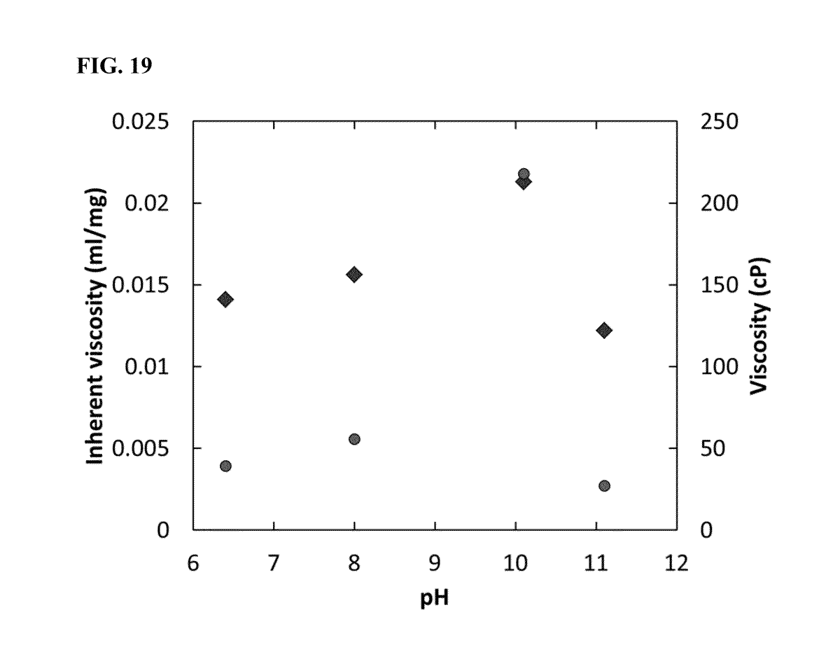

Effect of pH on co-solutes on viscosity at ˜250 mg/ml mAb concentration. The effect of pH was examined for a series of dispersions containing a total concentration of 150 mg/ml arg and glu since arginine properties would be expected to change with pH as its charge varies from +1 from pH 5 to 8, neutral between pH 8-11 and negative above pH 11. The η increases with protein concentration at all pH values as expected (FIG. 18). Apart from pH 8.5 where it was the highest, the η was relatively insensitive to pH and ranged from 30-50 cP at 250 mg/ml in agreement with the samples from FIG. 16A. FIG. 19 shows η and ηᵢₙₕ for mAb1 samples containing arg titrated with HCl instead of glu (additional data in Table 2-S5). The η progressively increases from pH 6.4 to 8 and even more at pH 10 where it peaks and then decreases as the pH is raised to 11, as was also observed for the systems with arg and glu. The lowest η was obtained at pH 11 although this pH is too alkaline for subcutaneous delivery. The ηᵢₙₕ also went through a maximum at pH 10 and was lowest at pH 11. The ηᵢₙₕ for the samples with arg and glu were modestly lower than for those with HCl (highlighted in Table 2-S6). Additionally the samples containing glu have the advantage of a lower tonicity (row 4 in Table 2-5 is 935 mOsm/kg, 283% of isotonic concentration) compared to the samples with HCl (1688 mOsm/kg, 618% of isotonic concentration, for the pH 8 sample with arg.HCl from FIG. 19). [52]

TABLE 2-S5
Visc vs pH for formulations with 150 mg/ml arg.

| Protein | Arg | HCl | | | Inherent | | Cfg | Cfg |
|---|---|---|---|---|---|---|---|---|
| conc | Conc | Conc | | Viscosity | Viscosity | Yield | time | speed |
| (mg/ml) | (mg/ml) | (mg/ml) | pH | (cP) | (ml/mg) | (%) | (min) | (rcf) |
| 238 | 150 | | 11.4 | 19 ± 2.6 | 0.0106 | 74 | 30 | 10000 |
| 241 | 150 | | 11.4 | 27 ± 6.5 | 0.0120 | 71 | 30 | 10000 |
| 272 | 150 | | 11.4 | 66 ± 15.0 | 0.0139 | 81 | 35 | 10000 |
| 280 | 150 | | 11.4 | 73 ± 11.7 | 0.0139 | 79 | 40 | 10000 |
| 208 | 150 | 15.5 | 10.1 | 238 ± 31.8 | 0.0244 | 80 | 60 | 10000 |
| 237 | 150 | 29.4 | 8.0 | 94 ± 8.2 | 0.0174 | 67 | 40 | 10000 |
| 261 | 150 | 29.4 | 8.0 | 216 ± 12.7 | 0.0190 | 84 | 80 | 5000 |
| 242 | 150 | 30.1 | 6.4 | 29 ± 4.1 | 0.0123 | 80 | 30 | 10000 |

TABLE 2-S6
Glu lowers the viscosity compared to HCl. Same amount of interacting
co-solute but glu lowers the osmolality values. The centrifugation
speed for all the samples was 10000 rcf.

| Protein | Arg | | Exc 2 | | | Inherent | | Cfg |
|---|---|---|---|---|---|---|---|---|
| conc | Conc | | Conc | | Viscosity | Viscosity | Yield | time |
| (mg/ml) | (mg/ml) | Exc 2 | (mg/ml) | pH | (cP) | (ml/mg) | (%) | (min) |
| 245 | 81.4 | glu | 68.7 | 7.1 | 29 ± 1.5 | 0.0121 | 89 | 35 |
| 242 | 81.4 | glu | 68.7 | 7.1 | 34 ± 10.2 | 0.0126 | 79 | 50 |
| 249 | 81.4 | glu | 68.7 | 7.1 | 33 ± 5.7 | 0.0124 | 71 | 40 |
| 279 | 81.4 | glu | 68.7 | 7.1 | 46 ± 8.8 | 0.0134 | 92 | 45 |
| 249 | 81.4 | glu | 68.7 | 7.1 | 52 ± 9.1 | 0.0142 | 73 | 35 |
| 232 | 81.4 | glu | 68.7 | 7.1 | 31 ± 2.3 | 0.0130 | 78 | 40 |
| 244 | 81.4 | glu | 68.7 | 7.1 | 42 ± 6.8 | 0.0136 | 89 | 45 |
| 242 | 150 | HCl | 30.1 | 6.4 | 29 ± 4.1 | 0.0123 | 80 | 30 |

The effect of pH on η was also determined for low co-solute samples as shown in Table 2-7. The ηᵢₙₕ decreased to a minimum at pH 6.5 and then increased with pH up to pH 11. At a concentration of 200 mg/ml, η for high co-solute samples was as low as ˜10 cP, which is about 6 fold lower than these low co-solute samples. The D/D₀ was close to 1 at pH 5.5, but then decreased to 0.6-0.73 closer to the isoelectric point where reversible aggregates may have formed. These samples permeated through the filter faster than the high co-solute samples in the lower range of mAb concentrations as expected from the lower η₀. However, the permeation decayed rapidly once the mAb concentration approached 200 mg/ml, down to ˜0.1 μl/min from ˜10 μl/min, as dispersion η increased markedly. As a result, the final concentrations for these samples were only about 200 mg/ml below the intended target concentration of 250 mg/ml.

TABLE 2-7
Viscosity of low co-solute mAb1 dispersions at various
pH values all at a centrifugation speed of 5000 rcf.

| Protein | | Buffer | | | | | | | Cfg |
|---|---|---|---|---|---|---|---|---|---|
| conc | | conc | | η₀ | η | ηᵢₙₕ | | % | time |
| (mg/ml) | Buffer | (mM) | pH | (cP) | (cP) | (ml/mg) | D/D₀ | yield | (min) |
| 197 ± 15.2 | none | — | 5.5 | 1.0 | 64 ± 4.2 | 0.0212 | 0.96 | 95.5 | 50 |
| 191 ± 1.0 | phosphate | 50 | 6.5 | 1.0 | 71 ± 4.5 | 0.0196 | 0.77 | 85.5 | 45 |
| 206 ± 1.0 | phosphate | 50 | 8 | 1.0 | 108 ± 13.9 | 0.0228 | 0.60 | 88.5 | 45 |
| 185 ± 8.2 | carbonate | 50 | 11 | 1.0 | 147.6 ± 9.2 | 0.0271 | 0.73 | 84.7 | 45 |

To quantify the effect of co-solutes on protein-protein interactions, k_{d} measurements were conducted on dilute protein samples (5-20 mg/ml) in the buffers used in the previous measurements in this study with k_{d} values shown in Table 2-8. Protein-protein interactions for mAb1 in 30 mM histidine buffer, were weakly repulsive with a k_{d} of ˜4 ml/mg because proteins would have some net charge being far from its isoelectric point of 9 which would lead to electrostatic repulsion between proteins. The addition of a high concentration of arg and glu decreases k_{d} making the interactions net attractive with a k_{d} of ˜−7 ml/mg.

TABLE 2-8
Diffusion interaction parameter k_{d} values for various dilute protein
solutions as measured by DLS using the CONTIN algorithm.

| Protein formulation | pH | k_{d} (ml/mg) |
|---|---|---|
| 30 mM histidine | 6 | 3.8 |
| 78.6 mg/ml arg 71.4 mg/ml glu | 5.5 | −7.0 |

For polyclonal sheep IgG, the viscosities were well below those for mAb1, down to 17 cp at 258 mg/ml (Table 2-9). Increasing the concentration of arg and glu to 200 mg/ml did not seem to further lower η. The centrifugation time for the higher arg concentration sample is higher probably because of the higher η₀.

TABLE 2-9
Low viscosities for sheep IgG dispersions with high co-solute
concentrations at a centrifugation speed of 10000 rcf.

| Protein | Arg | Glu | | | | | | Cfg |
|---|---|---|---|---|---|---|---|---|
| conc | conc | conc | | η₀ | η | ηᵢₙₕ | % | time |
| (mg/ml) | (mg/ml) | (mg/ml) | pH | (cP) | (cP) | (ml/mg) | yield | (min) |
| 252 | 75 | 75 | 5 | 1.5 | 17 ± 5.6 | 0.0097 | 100 | 45 |
| 258 | 108.5 | 91.5 | 6 | 1.8 | 20 ± 0.3 | 0.0101 | 94 | 55 |

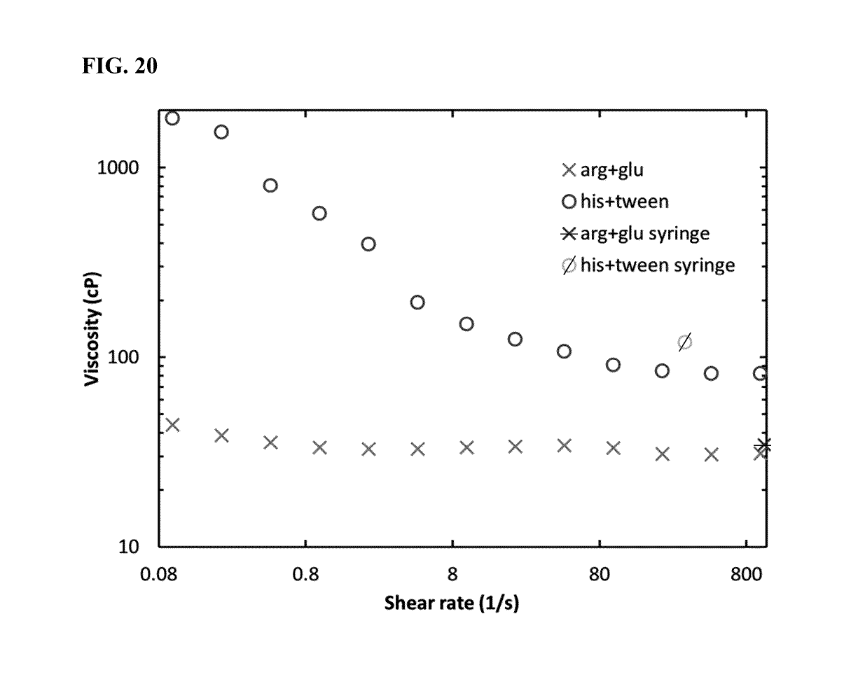

In order to further examine the effect of arg and glu on the interactions of mAb1, the shear rate dependence of η was measured with a cone and plate rheometer. The sample was prepared by combining samples from runs in 4 separate 0.5 ml centrifugal filters together. The mAb1 solution in 20 mM histidine with 0.05% Tween 80 was observed to be shear thinning with η decreasing over 2 orders of magnitude from 0.1 to 1000 s⁻¹. mAb dispersions with low co-solute concentrations are routinely observed to shear thin due to the presence of mAb clusters formed due to associative forces or entanglement of molecules. [6,8,9,18] The Tween-80 was added at a concentration well above its critical micellar concentration (CMC) to saturate the air-water interface and prevent the complication of a viscoelastic protein layer on the interface. [53] There is a possibility that some of the shear thinning observed may be the result of protein adsorption at the air/water interface even with the surfactant present. In contrast, the dispersion containing 78.6 mg/ml arg and 71.4 mg/ml glu was nearly Newtonian and underwent very little shear thinning over the same shear rate range, despite the lack of surfactant. This result suggests weaker interactions between proteins consistent with the lower viscosity. The syringe η data and D/D₀ data for the runs are shown in Table 2-S7 and also plotted in FIG. 20 with the shear rate calculated using the measured flow rate and known needle diameter in the Hagen-Poiseuille equation. The measured η by the syringe viscometer at the same shear rate appears to be ˜20 and 10% higher for the his-tween 80 and arg-glu systems, respectively relative to the cone and plate rheometer.

TABLE 2-S7
Viscosity and D/D₀ for the samples tested for viscosity versus shear rate
in FIG. 20. The sample in row 2 was in 20 mM his buffer at pH 5.48.

| Protein | std | Arg | Glu | | Inherent | | | Cfg |
|---|---|---|---|---|---|---|---|---|
| conc | dev | Conc | Conc | Viscosity | Viscosity | | % | time |
| (mg/ml) | (mg/ml) | (mg/ml) | (mg/ml) | (cP) | (ml/mg) | D/D₀ | yield | (min) |
| 269 | 2.5 | 78.6 | 71.4 | 33 ± 2.6 | 0.0115 | 0.26 | 98 | 30 |
| 228 | 16.2 | 0 | 0 | 152 ± 19.2 | 0.0221 | 0.98 | 78 | 30 |

Effect of a preferentially excluded co-solute trehalose on viscosity. To place the results with arg in perspective, tre was chosen as a non-electrolyte co-solute without acidic or basic sites. In Table 2-10, dispersions contained tre as the only co-solute at pH values ranging from 6.4-8 using a 50 mM phosphate buffer. Tre by itself does not reduce ηᵢₙₕ significantly in contrast with the behavior for arg. The D/D₀ values are very low for the samples with very high tre suggesting that depletion attraction may have produced relatively large reversible aggregates. The lack of a viscosity reduction for mAb1 suggests that tre does not modify the attractive specific protein-protein interaction for mAb1 sufficiently. In contrast with these results low viscosities were observed for a mAb 1B7 and sheep IgG in systems with a high concentration of tre co-solute in previous studies for dispersions buffered with histidine and phosphate together. [25] In that study, depletion attraction was proposed to assemble proteins into nanoclusters based on a free energy model for hard spheres with a uniform charge. This behavior may be part of the reason for the lowered D/D₀. However, the model does not predict the fraction of proteins in the monomeric state surrounding the nanoclusters or provide information about the polydispersity of the nanoclusters. [25,26,30,31]

TABLE 2-10
Viscosity and D/D₀ of mAb1 dispersions with tre as the only
co-solute. All samples were formulated in 50 mM phosphate buffer
to set the pH. The centrifugation speed was 10000 rcf.

| Protein | Tre | | | | | | | Cfg |
|---|---|---|---|---|---|---|---|---|
| conc | conc | | η₀ | η | ηᵢₙₕ | | Yield | time |
| (mg/ml) | (mg/ml) | pH | (cP) | (cP) | (ml/mg) | D/D₀ | (%) | (min) |
| 229 | 70 | 8.2 | 1.2 | 241 ± 41.8 | 0.0230 | 0.25 | 80 | 30 |
| 246 | 200 | 8.2 | 1.4 | 190 ± 8.3 | 0.0198 | 0.13 | 76 | 90 |
| 200 | 200 | 7.2 | 1.4 | 191 ± 15.7 | 0.0244 | 0.18 | 66 | 30 |
| 229 | 200 | 6.4 | 1.4 | 160 ± 77.6 | 0.0206 | — | 82 | 95 |

In order to add charge screening along with depletion attraction, ammonium sulfate was added to mAb1 dispersions containing tre as shown in Table 2-11. The addition of salt did not lower the η or ηᵢₙₕ significantly, in contrast with the behavior in Table 2-3 for NaCl with dilute histidine buffer. Thus, in the former case, the depletion attraction somehow negated the potential lowering of viscosity by electrostatic screening. Given tre or tre with NaCl did not lower the viscosity, we now examine how tre influences protein dispersions containing high concentration of arg and glu. Table 2-12 shows the effect of adding tre to a dispersion with a constant ratio of tre to arg and glu with replicates in Table 2-S8. The η decreases by ˜45% at ˜240 mg/ml mAb1 as the total concentration of co-solutes is increased from 75 mg/ml to 200 mg/ml. The addition of tre increases η slightly compared to the dispersions containing only arg and glu at similar concentrations, as can be seen by comparing row 2 of Table 2-12 with row 1 of Table 2-5 or row 3 of Table 2-12 with row 2 of Table 2-5. The viscosities for these samples containing tre alone at a given concentration are much higher than those for the samples with arg and glu at similar total co-solute concentrations as can be seen by comparing row 3 in Table 2-12 and rows 2-4 in Table 2-10. However, the increase is mainly due to the higher η₀ as ηᵢₙₕ remains essentially constant with added tre. The D/D₀s for the dispersions from Table 2-12 are also much lower than unity. The additional tre affects the osmolality of the sample but to a lesser extent than arg and glu, e.g., for samples with 200 mg/ml co-solute with arg, glu and tre from row 3 in Table 2-12 the osmolality is 1006 mM (311% of isotonic concentration) while that for tre at 200 mg/ml from rows 2-4 in Table 2-10, the osmolality is 529 (119% of isotonic concentration). [52]

TABLE 2-11
Viscosity of mAb1 dispersions with tre as the only co-solute
with ammonium sulfate added to screen electrostatic interactions.
All samples were formulated in 50 mM pH 8.2 phosphate buffer.
The centrifugation speed was 10000 rcf.

| Protein | Tre | (NH₄)₂SO₄ | | | | Cfg |
|---|---|---|---|---|---|---|
| conc | conc | conc | η₀ | η | ηᵢₙₕ | time |
| (mg/ml) | (mg/ml) | (mM) | (cP) | (cP) | (ml/mg) | (min) |
| 228 | 200 | 25 | 1.4 | 130 ± 4.0 | 0.0197 | 20 |
| 243 | 200 | 100 | 1.4 | 195 ± 37.3 | 0.0202 | 40 |

TABLE 2-12
Viscosity of mAb1 dispersions including tre with arg and
glu at pH 7.1. The centrifugation speed was 5000 rcf.

| Protein | Arg | Glu | Tre | | | | | | Cfg |
|---|---|---|---|---|---|---|---|---|---|
| conc | conc | conc | conc | η₀ | η | ηᵢₙₕ | | Yield | time |
| (mg/ml) | (mg/ml) | (mg/ml) | (mg/ml) | (cP) | (cP) | (ml/mg) | D/D₀ | (%) | (min) |
| 241 | 27.5 | 22.5 | 25 | 1.2 | 71 ± 14.0 | 0.0167 | — | 71 | 95 |
| 252 | 36.7 | 30 | 33.3 | 1.4 | 66 ± 16.5 | 0.0154 | 0.27 | 61 | 75 |
| 238 | 73.4 | 60 | 66.7 | 1.7 | 40 ± 2.5 | 0.0137 | 0.34 | 80 | 65 |

TABLE 2-S8
Viscosity and cluster size decrease with increasing conc of arg/glu/tre.
Replicate data for Table 12. Centrifugation speed was 5000 rcf
and pH was 7.1.

| Pro- | | | | | | | | |
|---|---|---|---|---|---|---|---|---|
| tein | Arg | Glu | Tre | | Inherent | | | |
| conc | Conc | Conc | Conc | | Vis- | | | Cfg |
| (mg/ | (mg/ | (mg/ | (mg/ | Viscosity | cosity | | Yield | time |
| ml) | ml) | ml) | ml) | (cP) | (ml/mg) | D/D₀ | (%) | (min) |
| 220 | 27.5 | 22.5 | 25 | 46 ± 11.9 | 0.0166 | 0.21 | 71 | 95 |
| 223 | 36.7 | 30 | 33.3 | 35 ± 1.6 | 0.0145 | 0.24 | 65 | 55 |
| 235 | 73.4 | 60 | 66.7 | 42 ± 1.7 | 0.0138 | 0.32 | 70 | 75 |

Stability of mAb dispersion upon storage and dilution. The dissociation of any reversible aggregates is evidenced by size exclusion chromatography (SEC) of diluted mAb1 dispersions at 1 mg/ml in Table 2-13. The SEC trace of the original material without any processing except for dilution before running SEC indicated >99% monomer. For the 250 mg/ml dispersion from row 4 of Table 2-5, after dilution the % monomer was almost identical to that of the starting material thus corroborating the evidence from DLS that monomeric mAb is obtained upon dilution. A preliminary study of the stability of mAb1 in dispersion was also conducted by storing a high co-solute sample of mAb1 at −40° C., 4° C. and room temperature for 8 weeks as shown in Table 2-14. The samples stored under all three conditions were seen to be stable with no significant decrease in the % monomer over the course of 8 weeks.

TABLE 2-13
SEC for diluted mAb dispersions
compared to the monomer control.

| Sample | % monomer in the sample |
|---|---|
| Monomer control (no processing) | 99.89 |
| Sample with arg:glu (row 4 from Table 4) | 99.86 |

TABLE 2-14
SEC for mAb1 dispersions stored for 8 weeks. The values
in the table are the % monomer in the sample measured
by the area under the curve for the SEC. The ~250
mg/ml dispersion contained 81.4 mg/ml arg and 68.6 mg/ml
glu (pH 7.1) and was 99.89% monomer pre-storage.

| | Frozen storage | Refrigerated storage | Room temperature |
|---|---|---|---|
| Week | at −40 ° C. | at 4° C. | storage |
| 1 | 99.86 | 99.87 | 99.86 |
| 1.5 | 99.87 | 99.90 | 99.84 |
| 2 | 99.85 | 99.91 | 99.83 |
| 4 | 99.77 | 99.86 | — |
| 8 | 99.90 | 99.88 | 99.75 |

mAb dispersions without co-solute. In systems without added co-solutes, η has been studied as a function of the protein sequence, buffer composition or concentration of added salts. [12,17] In particular, Connolly et al. demonstrated that mAb sequence may be modified to decrease the attractive interactions and thus reduce η for the charged mutant compared to the wild-type mAb. [7,8,18] For a series of mAbs in a given buffer, a direct correlation was observed between η at high concentration and the second virial coefficient (B₂) or the diffusion interaction parameter (k_{D}) at low concentration. [7,10] The slightly positive k_{d} for mAb1 (control with no added co-solute) at pH 5.5 in 30 mM histidine buffer (Table 2-8) is in the middle of Connolly's range. Thus the observed relatively high η>150 cp in Table 2-3 is consistent with the measured k_{d}. [7] Since high ηs were seen for mAb1 dispersions with no co-solute over a variety of pH values in Table 2-7, the buffers probably do not modify protein-protein interactions to a sufficient extent for lowering η.

Even when proteins do not aggregate according to small angle neutron scattering (SANS), neutron spin echo (NSE), [54] small angle x-ray scattering (SAXS) or static light scattering (SLS), attraction between proteins may still produce a relatively high η of ˜20 cP at 150-175 mg/ml. The η will likely be even higher when attractive hydrophobic or anisotropic electrostatic and charge-dipole forces form clusters or oligomers of mAbs. [7-9, 12, 17, 18, 27] Since elevated volume fraction of mAb in solution due to the void volume present in oligomers is a major culprit for high η, disrupting the oligomer structure can potentially lower η. [12,32] For a strongly interacting mAb which dimerizes, addition of 200 mM NaCl was shown to lower η five-fold from ˜300 cP to ˜60 cP at 150 mg/ml by breaking up dimers, as shown by NSE measurements of protein diffusion. [12] The oligomers may be disrupted by electrostatic screening of the anisotropic interactions between oppositely charged patches and charge-dipole interactions. [8,9,18,55,56] As shown in Table 2-3 η for mAb1 was seen to decrease by a little more than two-fold upon the addition of 150 mM NaCl to a mAb1 dispersion in 30 mM histidine buffer. However, depending on whether the net or the localized electrostatic interactions are dominant for determining η, addition of salt can lower, [4,6,18,57] not influence [5,12] or in some cases even raise [17] η depending on the particular mAb in question.

Modification of electrostatic interactions between mAbs by arg to lower η. The net charge on mAbs can cause repulsion between mAbs for pH values away from the isoelectric point and is an important factor governing protein-protein interactions. Between pH 5 and 8, the carboxylic acid group is negatively charged while the amino and guanidyl groups are positively charged giving arg a net +1 charge. Screening of the net electrostatic interactions by increased ionic strength can lead to a reduction in repulsive forces between mAbs which would explain the negative k_{d} value for mAb1 upon addition of a high concentration of arg and glu as co-solutes (Table 2-8). At these low concentrations, the anisotropic electrostatic and dipolar interactions will be less important than at the high protein concentrations. Arg and glu raise the ionic strength of mAb1 dispersions and therefore can have a similar η lowering effect as NaCl. However, the carboxylic acid, amino and guanidyl groups of arg can bind to a variety of polar or charged amino acid residues on proteins through hydrogen bonds, electrostatic or charge-dipole interactions. [21,22,40-42,46,58] Therefore between pH 5-8, bound arg can increase the magnitude of positive charge on positively charged sites, lend a positive charge to neutral sites and neutralize the negative charge on negatively charged sites. As a result, a majority of the localized charge patches will be positively charged leading to weak attraction between oppositely charged patches, which may explain the lowered η at pH 5 to 7. Although the overall electrostatic interactions are more attractive as evidenced by the measured k_{d} values via diffusion coefficient (Table 2-8), the localized oppositely charged interactions may be made less attractive by arg as evidenced by the elevated k_{d} measured by self-interaction chromatography [45] and weakened protein binding to ion-exchange columns. [21,22] The specific binding of arg to charged sites on proteins could be partially responsible for the greater decrease in η seen for the dispersions containing arg (Table 2-5 and FIG. 16) compared to those containing NaCl (Table 2-3). At pH values above 8 where arg becomes uncharged, the protein charge modification by arg will be lessened along with the effect of ionic strength. Consequently, an increase in anisotropic electrostatic interactions would be expected to increase η at pH 8.5 as observed in FIGS. 18 and 19. Arg starts becoming negatively charged along with the protein at pH>11 as the guanidyl group becomes deprotonated. Here arg H-bonds to proteins will make the protein surface more negative and increase electrostatic repulsion, which would be expected to lower η as observed in FIGS. 18 and 19.

Modification of hydrophobic interactions between mAbs by salt and arg to lower η. Interactions between hydrophobic patches on proteins are known to produce elevated ηs and ultimately precipitation. [5,20,27] For example, addition of salts with organic ions can reduce η of BSA and γ-globulin solutions by up to 4 times at high protein concentration by binding to the hydrophobic patches. [20] In addition to H-bonding to mAbs, the guanidyl group of arg also interacts with hydrophobic residues allowing arg to interact with hydrophobic patches on proteins. [21,22,27,28,40-44] Guanidium chloride (Gdn.HCl) contains the guanidium cation, a strong base (pkₐ=13.6) that denatures proteins at much higher concentrations (>5M). [59,60] Unlike guanidine, arg binding to hydrophobic sites is much weaker due to the carboxylic acid group, and this weakened binding causes less unfolding. [44,46] Alternatively, simulations of arg.HCl at high concentrations show that three arg ions tend to stack on top of each other with the guanidium groups aligned with each other and the ethylene groups form a hydrophobic patch which binds to hydrophobic patches on proteins. [40,41,44,45] Arg binding to hydrophobic patches on a local scale decreases attraction and can potentially increase repulsion. [27] The reduction in hydrophobic interactions between proteins upon adding arg is evidenced by reduced protein binding on hydrophobic columns [21,22] and an increased k_{d} through addition of arg. [45] Partially because of this blocking of hydrophobic interactions, arg lowers η to a greater extent than screening of electrostatic interactions alone with NaCl, as evidenced by comparing Tables 2-3 and 2-5. Additionally, as the concentration of arg is increased, a greater number of hydrophobic and charged patches can be blocked leading to weaker interactions and therefore a low η explaining the observed trend in FIG. 17 and Table 2-5.

Experimental Section.

Materials.

The monoclonal antibody used in this study (mAb1) was obtained from AbbVie at ˜120 mg/ml in a proprietary buffer composition. Arginine, glutamic acid, lysine, acetic acid, sodium glutamate, arginine hydrochloride, proline, glycine, histidine, sodium monophosphate, sodium biphosphate, sodium bicarbonate and HCl were purchased from Fisher Scientific, Fairlawn, N.J. Trehalose was purchased from Ferro Pfanstiehl Laboratories Inc., Waukegan, Ill. Amicon Ultra-15 Ultracel-30K and Amicon Ultra 0.5 Ultracel-50K centrifugal filters were purchased from Merck Millipore Ltd. Ireland.

Buffer Exchange.

0.4 ml of the 120 mg/ml mAb1 solution was initially diluted to 4 mg/ml in a buffer containing desired concentrations of co-solutes (12 ml total, initial buffer volumetric fraction is 3.33% (0.4 ml out of 12)). The resulting solution was then filtered using a Millipore Centricon centrifugal concentrator tube with a molecular weight cutoff of 30 kDa and a capacity of 12 ml at a spin speed of 4500 rcf for 12 minutes. The protein solution was concentrated till the solution volume dropped to about 5 ml and a protein concentration of ˜10 mg/ml. Then the retained protein solution was again diluted using the desired dispersion buffer to make up the volume to 12 ml (initial buffer volume fraction reduced to 1.4% (3.33% out of 5 ml in 12 ml)) and then centrifuged again. This process was repeated 4 or more times until the volume of flow through was about 40 ml and the volumetric fraction of initial buffer was less than 1% assuming ideal mixing. After this the solution was further concentrated by continuing the centrifugation so that the final volume was about 0.5 ml at about 80 mg/ml. Alternatively, in Tables 2-1 and 2-2, buffer exchange was carried out by Tangential Flow Filtration using a Microkros hollow fiber module manufactured by Spectrum Labs (part no. C02-E050-05-N) with polyethersulfone as the filter material, 50 kD pores and a membrane area of 20 cm². The module was connected to a micokros pump (KROSFLO® Research IIi Tangential Flow Filtration System) using silicone tubing (Masterflex, HV-96410-14) with an ID of 1.6 mm. The buffer exchange was carried out in a continuous manner with continuous addition of DI water until 6 diavolumes of buffer had been permeated through.

Centrifugal Concentration to >200 mg/ml.

Tare weights were taken of the individual components (filter, permeate tube and retentate tube) of the centrifugal filter assembly (Millipore Microcon, Ultracel YM-50 membrane, 50 kD nominal molecular weight limit, 0.5 ml capacity). The desired volume of protein solution in the dispersion buffer post buffer exchange and concentration to 80 mg/ml was pipetted into the retentate chamber. The filter assembly was then centrifuged (Eppendorf Centrifuge 5415D) at either 5000 or 10,000 rcf, typically for about 40 minutes in 5-10 minute increments with volume monitoring at every stop until the calculated final volume for the desired final concentration was reached. The volume measurements were done using image analysis to determine the height of the liquid column in the filter which had been calibrated to correspond to the volume of liquid in the filter using ImageJ software [47] and also by weighing both the retentate and permeate having previously tare weighted filter components.

Once the desired concentration had been reached, the protein dispersion in the retentate was recovered by inverting the filter assembly into a retentate recovery tube, and centrifuging it for 2 minutes at 1,000 rcf. The resulting dispersion was transferred to a 0.1 mL conical vial (V-Vial, Wheaton), and the concentration was verified spectrophotometrically by withdrawing a small sample (2 μl) of the dispersion (described in more detail below). The dispersion composition would not be expected to change significantly during the course of the run for the high co-solute samples. Further evidence supporting this is that there was no change observed in the pH of the high co-solute samples during the concentration step (pH was 5.48 at both the start and end for an example run as measured by a pH meter). In contrast, the pH of an example low co-solute sample drifted by ˜0.2 units from 5.48 at the start to 5.67 at the end of the run.

In cases where a larger sample was needed to examine the effect of sterile filtration on the properties of the dispersions, the entire centrifugal filtration run was conducted in the larger centrifuge tubes. A larger amount of protein (˜170 mg) was initially loaded onto the filter at the start of buffer exchange. Instead of transferring the sample to the smaller 0.5 ml centrifugal filters, it was transferred to another Millipore Centricon centrifugal concentrator tube and then concentrated to the desired final volume. The sample was characterized in the same way as the smaller samples and then sterile filtered using a 1 ml syringe (Becton Dickinson & Co. with LUER-LOK™ tip) and a syringe filter with a 220 nm cutoff. The sample was also weighed before and after filtration to determine the yield for the sterile filtration step. The sample was then characterized after sterile filtration in a similar way to the smaller samples.

Characterization of the Protein Nanocluster Dispersion

Viscosity Measurement.

The viscosities of the nanocluster dispersions were measured in triplicate using a 25 gauge (ID=0.1 mm) 1.5″ long needle (Becton Dickinson & Co. Precision Glide Needle) attached to a 1 ml syringe (Becton Dickinson & Co. 1 mL syringe with LUER-LOK™ tip), on the basis of the Hagen-Pouiselle equation. The flow rate of a dispersion through the needle was determined using the estimated volume which was correlated to the height of the liquid in the conical vial and measuring the time taken for the dispersion meniscus height to move between two points. This flow rate was correlated to viscosity from a calibration curve derived from a set of standards of known viscosities. Cone and plate rheometry experiments were conducted on a standard torsional rheometer (AR2000EX, TA Instruments) with a 40 mm diameter cone with 2° of angle and a truncation gap of 55 μm. The cone-and-plate geometry is selected for the constant shear rate in the tool-plate gap. Sample temperature was controlled by a lower Peltier plate set to 25° C.

DLS and Diffusion Interaction Parameter Measurements.

The effective CONTIN diameters of protein monomers and nanoclusters were measured by dynamic light scattering (DLS) at an angle of 150° with a 632.8 nm laser and an avalanche photodiode at ˜23° C. using the CONTIN algorithm (Brookhaven BI-9000AT) or at an angle of 90° with a Brookhaven ZetaPlus. The samples were pipetted into a 60 μl sample cell for the samples measured at 150° (Beckman Coulter) or a Uvette® (Eppendorf) for the samples measured at 90° which was then mounted on the instrument to conduct three replicate runs of 2 minutes each. All reported D/D₀ values are the average of three runs. Additionally measurements of the diffusion interaction parameter (k_{d}) were conducted on dilute protein samples at 5, 10, 15 and 20 mg/ml. The diffusion coefficient in these dilute protein solutions was measured with the Brookhaven ZetaPlus at a scattering angle of 90° and fit with the equation D=D₀(1+k_{d} c) where D is the measured diffusion coefficient, Do is the diffusion coefficient at infinite dilution and c is the protein concentration.

Size Exclusion Chromatography.

For analysis of non-covalent aggregates, the sample was diluted in mobile phase (100 mM sodium phosphate, 300 mM sodium chloride, pH 7) to 1 mg/ml. A volume of the diluted sample containing 20 μg of mAb1 was analyzed with a Waters Breeze HPLC, using TOSOH Biosciences TSKgel3000SW_{XL} and TSKgel2000SW columns in series, with eluate monitored by absorbance at 214 nm.

Protein Concentration Determination in the Dispersion.

For determining the concentration of the dispersions, 2 μl of dispersion was measured out and diluted into a receiving vessel containing 998 μl of 50 mM pH 6.4 phosphate buffer mixing well with the pipette tip. The diluted samples were prepared in duplicate. The absorbance of the resulting solution was measured using a Cary 3E uv-visible spectrophotometer in a cuvette (Hellma cells) with a path length of 1 cm. Then using Beer's law (A=εb c, where ε=extinction coefficient=1.42 ml mg⁻¹ cm⁻¹, b=path length=1 cm), knowing the absorbance, the concentration of protein in the solution was calculated.

Sample Storage.

Samples were stored in the 0.1 ml conical vials sealed with the lid and parafilm. Samples were stored in a −40° C. freezer, in a refrigerator at 4° C. and on the bench at room temperature. The sample for each time-point was taken from a separate vial which was then discarded.

Conclusion.

This study expands our previous studies of high co-solute protein dispersions [25,26,29] with low viscosities to include interacting co-solutes and for pH values away from the isoelectric point. The viscosity of concentrated ˜250 mg/ml mAb1 dispersions was decreased up to 6 fold by the addition of high concentrations of arg titrated with glu or HCl as co-solutes, compared to a control at the same pH of 5.5 in histidine buffer. At a constant pH of 5.5 (fixed ratio of arg/glu), increasing the total concentration of arg and glu lowered the inherent viscosity and viscosity significantly, reaching 0.0129 ml/mg and 43 cP at 261 mg/ml mAb1 after sterile filtration, which is sufficiently low for injection through a 28G needle. Similar reduction is viscosity for arg/HCl suggesting that arg played a more important role than glu. For a polyclonal sheep IgG mixture at 258 mg/ml, η and ηᵢₙₕ reached 17 cp and 0.0097 ml/mg respectively. The viscosity for mAb1 dispersions with high arg and glu concentrations was seen to actually be the highest near the isoelectric point in this case and low over a wide range of pH values away from the isoelectric point. Specific interactions of arg with proteins reduce the localized electrostatic, hydrogen bonding, charge-dipole and hydrophobic patch interactions between proteins, which lowers the viscosity. [21,22,45] The importance of these specific interactions is evident in the inability of tre, a non-interacting co-solute, to lower the viscosity. The diffusion coefficient ratio D/D₀ by DLS decreased well below 1 with the addition of almost all of the co-solutes, and to a greater extent for tre than arg/glu. Upon dilution in buffer, no significant increase was observed in % aggregates by SEC relative to the initial protein solution, despite exposing the protein to high co-solute and protein concentrations.

### References (Example 2)

[1]. Therapeutic monoclonal antibodies approved or in review in the European Union or United States: see website at antibodysociety.org/news/approved_mabs.php (Sep. 27, 2014); [2]. Shire, S. J.; Shahrokh, Z.; Liu, J. J. Pharm. Sci. 2004, 93, (6), 1390-1402; [3]. Srinivasan, C.; Weight, A. K.; Bussemer, T.; Klibanov, A. M. Pharmaceutical Research 2013, 30, (7), 1749-57; [4]. Saluja, A.; Kalonia, D. S. International Journal of Pharmaceutics 2008, 358, (1-2), 1-15; [5]. Kanai, S.; Liu, J.; Patapoff, T. W.; Shire, S. J. J Pharm Sci 2008, 97, (10), 4219-4227; [6]. Liu, J.; Nguyen, M. D. H.; Andya, J. D.; Shire, S. J. Journal of Pharmaceutical Sciences 2005, 94, (9), 1928-1940; [7]. Connolly, B. D.; Petry, C.; Yadav, S.; Demeule, B.; Ciaccio, N.; Moore, J. M.; Shire, S. J.; Gokarn, Y. R. Biophysical journal 2012, 103, (1), 69-78; [8]. Yadav, S.; Shire, S. J.; Kalonia, D. S. Journal of pharmaceutical sciences 2012, 101, (3), 998-1011; [9]. Yadav, S.; Liu, J.; Shire, S. J.; Kalonia, D. S. J Pharm Sci 2010, 99, (3), 1152-1168; [10]. Saito, S.; Hasegawa, J.; Kobayashi, N.; Kishi, N.; Uchiyama, S.; Fukui, K. Pharmaceutical Research 2012, 29, (2), 397-410; [11]. He, F.; Woods, C. E.; Litowski, J. R.; Roschen, L. A.; Gadgil, H. S.; Razinkov, V. I.; Kerwin, B. A. Pharmaceutical Research 2011, 28, (7), 1552-60; [12]. Yearley, E. J.; Godfrin, P. D.; Perevozchikova, T.; Zhang, H.; Falus, P.; Porcar, L.; Nagao, M.; Curtis, J. E.; Gawande, P.; Taing, R.; Zarraga, I. E.; Wagner, N. J.; Liu, Y. Biophysical journal 2014, 106, (8), 1763-70; [13]. Baglioni, P.; Fratini, E.; Lonetti, B.; Chen, S. H. Journal of Physics: Condensed Matter 2004, 16, (42), S5003-S5022; [14]. Burckbuchler, V.; Mekhloufi, G.; Giteau, A. P.; Grossiord, J. L.; Huille, S.; Agnely, F. European journal of pharmaceutics and biopharmaceutics: official journal of Arbeitsgemeinschaft fur Pharmazeutische Verfahrenstechnik e.V 2010, 76, (3), 351-6; [15]. Castellanos, M. M.; Pathak, J. A.; Colby, R. H. Soft Matter 2014, 10, (1), 122-31; [16]. Castellanos, M. M.; Pathak, J. A.; Leach, W.; Bishop, S. M.; Colby, R. H. Biophysical journal 2014, 107, (2), 469-76; [17]. Lilyestrom, W. G.; Yadav, S.; Shire, S. J.; Scherer, T. M. The journal of physical chemistry. B 2013, 117, (21), 6373-84; [18]. Zarraga, I. E.; Taing, R.; Zarzar, J.; Luoma, J.; Hsiung, J.; Patel, A.; Lim, F. J. Journal of pharmaceutical sciences 2013, 102, (8), 2538-49; [19]. Pathak, J. A.; Sologuren, R. R.; Narwal, R. Biophysical journal 2013, 104, (4), 913-23; [20]. Du, W.; Klibanov, A. M. Biotechnology and bioengineering 2011, 108, (3), 632-6; [21]. Hou, Y.; Cramer, S. M. Journal of chromatography. A 2011, 1218, (43), 7813-20; [22]. Holstein, M. A.; Parimal, S.; McCallum, S. A.; Cramer, S. M. Biotechnology and bioengineering 2012, 109, (1), 176-86; [23]. Miller, M. A.; Engstrom, J. D.; Ludher, B. S.; Johnston, K. P. Langmuir 2010, 26, (2), 1067-1074; [24]. Johnson, H. R.; Lenhoff, A. M. Molecular pharmaceutics 2013, 10, (10), 3582-91; [25]. Johnston, K. P.; Maynard, J. A.; Truskett, T. M.; Borwankar, A.; Miller, M. A.; Wilson, B.; Dinin, A. K.; Khan, T. A.; Kaczorowski, K. J. Acs Nano 2012; [26]. Borwankar, A. U.; Dinin, A. K.; Laber, J. R.; Twu, A.; Wilson, B. K.; Maynard, J. A.; Truskett, T. M.; Johnston, K. P. Soft Matter 2013, 9, (6), 1766-1771; [27]. Scherer, T. M. The journal of physical chemistry. B 2013, 117, (8), 2254-66; [28]. Inoue, N.; Takai, E.; Arakawa, T.; Shiraki, K. Molecular pharmaceutics 2014, 11, (6), 1889-96; [29]. Miller, M. A.; Khan, T. A.; Kaczorowski, K. J.; Wilson, B. K.; Dinin, A. K.; Borwankar, A. U.; Rodrigues, M. A.; Truskett, T. M.; Johnston, K. P.; Maynard, J. A. Journal of Pharmaceutical Sciences 2012, 101, (10), 3763-3778; [30]. Groenewold, J.; Kegel, W. K. Journal of Physical Chemistry B 2001, 105, (47), 11702-11709; [31]. Groenewold, J.; Kegel, W. K. Journal of Physics-Condensed Matter 2004, 16, (42), S4877-S4886; [32]. Godfrin, P. D.; Valadez-Perez, N. E.; Castaneda-Priego, R.; Wagner, N. J.; Liu, Y. Soft Matter 2014, 10, (28), 5061-71; [33]. Li, Y.; Lubchenko, V.; Vekilov, P. G. The Review of scientific instruments 2011, 82, (5), 053106; [34]. Pan, W. C.; Vekilov, P. G.; Lubchenko, V. Journal of Physical Chemistry B 2010, 114, (22), 7620-7630; [35]. Soraruf, D.; Roosen-Runge, F.; Grimaldo, M.; Zanini, F.; Schweins, R.; Seydel, T.; Zhang, F.; Roth, R.; Oettel, M.; Schreiber, F. Soft Matter 2014, 10, (6), 894-902; [36]. Lee, J. C.; Timasheff, S. N. Journal of Biological Chemistry 1981, 256, (14), 7193-7201; [37]. Kreilgaard, L.; Frokjaer, S.; Flink, J. M.; Randolph, T. W.; Carpenter, J. F. Journal of Pharmaceutical Sciences 1999, 88, (3), 281-290; [38]. Shen, V. K.; Cheung, J. K.; Errington, J. R.; Truskett, T. M. Journal of biomechanical engineering 2009, 131, (7), 071002-071002; [39]. Cheung, J. K.; Truskett, T. M. Biophysical Journal 2005, 89, (4), 2372-2384; [40]. Shukla, D.; Trout, B. L. The journal of physical chemistry. B 2011, 115, (5), 1243-53; [41]. Shukla, D.; Schneider, C. P.; Trout, B. L. Advanced drug delivery reviews 2011, 63, (13), 1074-85; [42]. Arakawa, T.; Ejima, D.; Tsumoto, K.; Obeyama, N.; Tanaka, Y.; Kita, Y.; Timasheff, S. N. Biophysical chemistry 2007, 127, (1-2), 1-8; [43]. Schneider, C. P.; Trout, B. L. The journal of physical chemistry. B 2009, 113, (7), 2050-8; [44]. Vagenende, V.; Han, A. X.; Mueller, M.; Trout, B. L. ACS chemical biology 2013, 8, (2), 416-22; [45]. Valente, J. J.; Verma, K. S.; Manning, M. C.; Wilson, W. W.; Henry, C. S. Biophysical Journal 2005, 89, (6), 4211-8; [46]. Shukla, D.; Trout, B. L. The Journal of Physical Chemistry B 2010, 114, (42), 13426-13438; [47]. Schneider, C. A.; Rasband, W. S.; Eliceiri, K. W. Nat Meth 2012, 9, (7), 671-675; [48]. Horn, F. M.; Richtering, W.; Bergenholtz, J.; Willenbacher, N.; Wagner, N. J. Journal of colloid and interface science 2000, 225, (1), 166-178; [49]. Mutch, K. J.; van Duijneveldt, J. S.; Eastoe, J. Soft Matter 2007, 3, (2), 155; [50]. Ross, P. D.; Minton, A. P. Biochemical and biophysical research communications 1977, 76, (4), 971-976; [51]. Rosenberg, E.; Hepbildikler, S.; Kuhne, W.; Winter, G. Journal of Membrane Science 2009, 342, (1-2), 50-59; [52]. Windholz, M.; Editor, The Merck Index: An Encyclopedia of Chemicals and Drugs. 9th Ed. Merck and Co., Publ. Dept.: 1976; p 1937 pp; [53]. Sharma, V.; Jaishankar, A.; Wang, Y.-C.; McKinley, G. H. Soft Matter 2011, 7, (11), 5150; [54]. Grimaldo, M.; Roosen-Runge, F.; Zhang, F. J.; Seydel, T.; Schreiber, F. Journal of Physical Chemistry B 2014, 118, (25), 7203-7209; [55]. Allmendinger, A.; Fischer, S.; Huwyler, J.; Mahler, H. C.; Schwarb, E.; Zarraga, I. E.; Mueller, R. European journal of pharmaceutics and biopharmaceutics: official journal of Arbeitsgemeinschaft fur Pharmazeutische Verfahrenstechnik e.V 2014, 87, (2), 318-28; [56]. Roberts, C. J.; Blanco, M. A. The journal of physical chemistry. B 2014; [57]. Salinas, B. A.; Sathish, H. A.; Bishop, S. M.; Ham, N.; Carpenter, J. F.; Randolph, T. W. Journal of pharmaceutical sciences 2010, 99, (1), 82-93; [58]. Vondrá\check{s}ek, J.; Mason, P. E.; Heyda, J.; Collins, K. D.; Jungwirth, P. The Journal of Physical Chemistry B 2009, 113, (27), 9041-9045; [59]. Chi, E. Y.; Krishnan, S.; Kendrick, B. S.; Chang, B. S.; Carpenter, J. F.; Randolph, T. W. Protein Science 2003, 12, (5), 903-913; [60]. Liu, W.; Cellmer, T.; Keerl, D.; Prausnitz, J. M.; Blanch, H. W. Biotechnology and bioengineering 2005, 90, (4), 482-90.

### Example 3

### Further Studies on Dispersions of Protein Nanoclusters with Proline as a Depletant

Abstract.

The ability to form monoclonal antibody dispersions at high concentrations, with low viscosities for subcutaneous injection, is of broad interest for simplifying treatment of cancer, autoimmune disorders, and many other diseases. Herein, we create highly concentrated antibody dispersions of nanoclusters of a monoclonal antibody (up to 260 mg/mL) with an amino acid, proline, to modulate the surface charge and to produce depletion attraction (crowding). The apparent hydrodynamic diameter of the nanoclusters remains relatively constant at 40-50 nm, for a pH range of 7.2-9.3. The viscosities of the dispersions, as low as 35 cP at 258 mg/mL, were up to an order of magnitude lower than similarly concentrated antibody solutions. When diluted, these antibody dispersions return to monomer. The proline lowers the protein zeta potential and effective protein charge, thereby weakening the attractive anisotropic electrostatic interactions between antibody molecules. In addition, proline acts as a depletant, whereby the osmotic force is sufficient to drive the monomers into nanocluster and reconfigure the antibody into more favorable conformations for protein stability and lower viscosities.

Introduction.

Formulation of monoclonal antibodies (mAbs) for pharmaceutical delivery presents a number of challenges; [1] often high dosages of >200 mg are required (for example, ˜700 mg for Rituximab [2]). Currently, this dose is delivered intravenously, which requires an extended clinic visit. In contrast, it would be desirable to administer therapeutic proteins subcutaneously at home. However, for a subcutaneous injection with a maximum volume of ˜1.5 mL, [3] required concentrations can be well above 200 mg/mL, where proteins are often insoluble and prone to aggregation, [4-7 gelation, and precipitation due to specific short-ranged attractive forces. [8-11] In cells, a heterogeneous mixture of proteins experience highly crowded environments, favoring folding and stability. [4,12] Recently, we have shown experimentally that a single type of protein may be assembled into nanoclusters on the order of 100 nm, whereby concentrations within the clusters reach 700 mg/mL. Moreover, the proteins are stable with respect to folding and aggregation upon dilution in buffer. [13] Protein stability against unfolding at concentrations as high as those realized inside of protein nanoclusters was predicted by simulation with coarse-grain models. [14,15] The clusters were formed near the isoelectric point to weaken electrostatic repulsion between protein molecules, by adding trehalose as a crowding agent (depletant) to provide depletion attraction. In some cases, the size of the cluster was described with an equilibrium free energy model, [13] whereby the growth stopped with the buildup of charge in the cluster, as has also been observed for Au colloids. [16] It was argued [13] that this combination of depletion attraction between protein monomer and weak long-ranged repulsion between protein nanoclusters, characteristic of interactions in other nanocluster forming systems, [22,23] produced the equilibrium size. [17,18] The nanoclusters were formed with mAb 1b7, Sheep IgG or bovine serum albumin (BSA) with trehalose or an N-methylpyrrolidone (NMP) and PEG mixture as depletants. [13,19,20] The viscosities of the dispersions ranged from 40-100 cP for concentrations on the order of 200 to 300 mg/ml. Furthermore, the nanoclusters dissociated reversibly to folded monomers upon dilution. For 1b7, the protein from nanocluster dispersions retained comparable bioavailability relative to solution, after subcutaneous and intravenous doses in two murine model studies. [19,20]

The concept of forming concentrated aqueous dispersions of nanoclusters with lower viscosities than protein solutions was introduced recently [13,20] as an alternative to protein suspensions in organic solvent. [21] The viscosity of a dispersion of protein molecules, η, relative to that of the solvent η₀ is described by Ross-Minton equation [24,25]:

$$η = {η}_{0} exp ( \frac{c [ η ]}{1 - \frac{k}{v} c [ η ]} ) \qquad ( 3 \text{-} 1 )$$

as a function of the protein concentration (c), the crowding factor (k), Simha shape factor (v) and inherent viscosity [η]. [26]. This model may be applied to a dispersion of individual protein molecules with a diameter of ˜10 nm or, in our case, for a dispersion of large protein colloids (nanoclusters). Monoclonal antibody solutions often have an [η] of 11-20; [27] in contrast with only 7 for previous examples of nanocluster dispersions. [13,20] Nanoclusters are further apart, on average, in a dispersion than monomers are in a solution at comparable concentration. Furthermore, intercluster attractions in a dispersion are expected to be weaker than interprotein attraction in a solution, which may lead to a decrease in [η] in the dispersions. So far, relatively few studies have been reported that relate the [η] of nanoclusters dispersions to the nanocluster geometry and composition, and how they are influenced by the nature and concentration of the depletant.

Amino acids have been used to stabilize protein solutions and would be interesting candidates for depletants to form protein nanoclusters. Proline is a very small stabilizer for protein solutions, with a molar mass of only 115 and a high solubility of ˜750 mg/mL; [28] it is also the only secondary amine among the 20 natural amino acids. [29] Similar to trehalose, proline is produced as an osmolyte in stressed organisms in dry or high temperature environments. [30] It is a natural cryoprotectant [31,32] that has been used as an excipient to enhance stabilization in pharmaceutical formulations. [33] Proline has been found to inhibit aggregation during folding and in aggregation-prone proteins in vivo and in vitro. [30,34] It appears to destabilize partially-folded states and early aggregates, while solubilizing native-state protein. [32] A mechanism by which it stabilizes proteins is through solvophobic interactions between proline and the protein backbone, causing preferential exclusion of the proline solute from the protein-water interface of partially-folded intermediates. [35] This preferential exclusion increases the free energy of the unfolded state, making it thermodynamically unfavorable. [35] When interacting with BSA, preferential exclusion of proline is actually greater than in the case of trehalose, [36] which also has unfavorable backbone interactions. [37]

Protein nanoclusters may be formed by dilution of lyophilized powders in buffer, and by concentrating protein solutions, for example with centrifugal filtration through a membrane. [13] In some cases, the same size was formed by two pathways suggesting the possibility of a metastable equilibrium state. [20] However, little is known about how the pathway of nanocluster formation influences the viscosity. When forming a high concentration dispersion of nanoclusters by filtration, the system traverses intermediate protein concentrations prior to nanocluster formation (so-called “danger zone”) for very short times, which may minimize the tendency for unfolding and formation of aggregates. [38] Here, a sufficiently concentrated depletant may help to mitigate unfolding via preferential exclusion or other osmotic forces. [20] For proline, its high solubility is beneficial for exploring multiple pathways to form clusters without limitations from depletant dissolution. It is likely that certain pathways will lead to dispersions that are trapped in metastable gel states [11] causing undesirable viscosity increases, while other pathways may potentially lead to low viscosities. Currently, very little is known about the relationship between viscosity and nanocluster formation pathways given the complexities of gel states at high concentrations.

The objective is to explore the formation, morphology and viscosity of protein nanocluster dispersions with a new type of depletant, an amino acid proline, for a human mAb (mAb 1) with a pI of 9.3 in various dispersion formation pathways. We would like to understand, relative to previous depletants, including trehalose and polyethylene glycol, how the much smaller size of proline, its zwitterionic charge state, and the strong preferential exclusion may influence the properties of the nanocluster dispersions. These properties will further be shown to be influenced by a large effect of proline on the pI of the protein. At dilute protein concentrations, proline at high concentration will be shown to lower the charge on mAb 1 to nearly zero (isoelectric point (pI) is at pH 9.3) for pH values between 6 and 9, suggesting significant interactions with the surface charges on the protein. Although previous studies have focused on nanoclusters at the pI, [13,20] we explore a wider range of pH to further understand the mechanism of nanocluster formation. From a stability and safety point of view for subcutaneous injection, it would be desirable to be able to form nanoclusters near pH 7, well below the pI of 9.3 for mAb 1. In addition, the effects of electrostatic interactions on viscosities of concentrated protein solutions are large, [39] and may thus also be expected to have a significant effect on those of nanocluster dispersions. Interestingly, protein nanocluster size will be found to be relatively independent of proline concentration, in contrast to what has been seen for trehalose as a depletant for another protein, sheep IgG. [13,20]

Three different pathways are examined for protein nanocluster formation: lyophilization followed by dilution of the lyophilized powder (lyophilization dilution, LD), addition of dispersions formed by centrifugation to lyophilized powder (C-D/P) and centrifugation filtration to concentrate solutions into nanoclusters (C). All three pathways will be shown to produce similar size clusters, as seen previously between C and LD samples in our previous work with trehalose as a depletant for sheep IgG. [20] However, it will be seen that the viscosities vary modestly for the various processes, despite similar nanocluster sizes.

### Results and Discussion

Protein Surface Charge and Free Energy Model for Nanocluster Size.

To better understand how interactions of proline with mAb1 moderate the charge, the zeta potential of a 2 mg/mL mAb 1 solution with 150 mg/mL of proline at different pH levels (controlled by buffer solutions) was measured, as shown in FIG. 1. Similar experiments were also performed with trehalose at 200 mg/mL instead of proline. Without added proline, the pI of mAb 1 was reported as 9.3 by the supplier, Abbvie. This was verified within the precision of the instrument, as seen in FIG. 1, whereby the zeta potential of mAb 1 was very close to zero from pH 8 to 10. The addition of proline to the mAb 1 solution reduced the zeta potential on mAb 1 to undetectable levels between pH 7 and 9. In contrast the addition of trehalose did not reduce the zeta potential significantly in the range studied. The reduction of the zeta potential by proline suggests that proline interacts with and screens the positive surface charges on the protein at the protonated sites. It is likely that the zeta potential would be reduced by a smaller extent in concentrated protein solutions, since the ratio of proline to protein would be much smaller. The large amounts of protein required for such experiments in a zeta potential cell volume of 1.5 ml was prohibitive for the current study.

To describe qualitative effects on how the nanoclusters form, it is insightful to examine the roles of the various colloidal forces with a semi-quantitative free energy model previously developed for protein nanoclusters, [20] based on an earlier model for clusters in organic solvents. [18] When the charge on the protein is small (either near the pI in the previously-published trehalose system, [13,20] or within the minimal charge range of pH 6-10 with proline systems) the electrostatic repulsion between protein monomers is relatively weak. As a consequence of this weak repulsive force, attractive forces between the monomers favor assembly of nanoscale clusters. In order to drive nanocluster formation, an extrinsic depletant has been added to create an attractive osmotic depletion force between the protein molecules via excluded volume. [43] The approximate equilibrium free energy and its derivation are described in detail in our previous paper, [20] but specifically, the model suggests that nanocluster diameters obey the following proportionality:

$${D}_{c} ∝ {[ \frac{ɛ / kT}{( λ / R ) {q}^{2}} ]}^{\frac{1}{2 {δ}_{f} - 3}} \qquad ( 3 \text{-} 2 )$$

where ε is the magnitude of the effective attraction between two adjacent protein molecules in the nanocluster, k is the Boltzmann constant, T is temperature, λ is the Bjerrum length, R is the protein radius, q is the charge per protein molecule, and δ_{f} is the fractal dimension of the nanocluster. As the concentration of depletant is increased, & increases and the nanoclusters formed by trehalose increased in size as predicted by Eq. 3-2.

The free energy model was used to estimate nanocluster diameter contours (FIG. 2) for mAb 1 with proline for the parameters listed in Table 3-1. Trehalose has a hydrodynamic radius of 0.5 nm [20] relative to only 0.268 nm [44] for proline. Therefore, in the case of proline which is a smaller molecule than trehalose, the depletion attraction will have a higher strength at contact, but will have a shorter range, as seen in FIG. 3. The model uses the contact value of the depletion attraction. Additionally proline binding lowers the protein charge (q²<<1), producing weaker electrostatic repulsion and larger self-assembled nanoclusters. Therefore, the dual roles of proline as a depletant and a protein surface charge modifier both favor nanocluster formation at a pH of ˜6-7. Qualitative cluster size contours are graphed in FIG. 2, along with the differing pathways that were used to form nanoclusters. While this model does not take into account the electrostatic screening by proline due to specific interactions with the protein, nor the preferential hydration of the protein that may be induced by proline, [45] nor effects of pH on specific interactions, it still provides a qualitative explanation for how concentrations of protein and depletant might affect cluster size for an assumed charge. Below, we examine our hypothesis that proline's role in the reduction of protein surface charge away from the pI may be beneficial for forming nanoclusters, to complement our previous study of protein nanoclusters near the pI of the protein. [13] The implication of this charge screening effect is that the effective pI of a mAb with a high isoelectric point may be reduced even 3 pH units to a more favorable pH for injection.

TABLE 3-1
Parameters used in the free energy model for predicting nanocluster
size at a given mAb 1 and proline concentration.

| Model parameter | Value |
|---|---|
| Fractal Dimension (δ_{f}) | 2.5 |
| Dielectric constant (∈ᵣ) | 15 |
| No. of dissociable sites per unit area of | 0.2 |
| colloid surface (σₛ, nm⁻²) | |
| Distance between opposite charges in an | 0.22 |
| ionic bond (b, nm) | |
| Radius of the protein monomer (R, nm) | 5.5 |
| Radius of extrinsic crowder (depletant, R_{E}, nm) | 0.268 |

Dilution of Lyophilized Powder in Buffer with Two Processes (LD).

The originally reported protein nanoclusters were formed by dilution (mixing) of lyophilized powder containing protein and depletant (LD) in buffer, as shown for sheep IgG, mAb 1B7 and BSA with trehalose. [13,20] In the current study, mAb 1 was lyophilized with proline as a cryoprotectant at a 1:1 mass ratio. As can be seen in Table 3-2, proline provided sufficient cryoprotection to mAb 1, with aggregation less than 1% as observed by SEC. A representative SEC curve of a concentrated mAb 1 dispersion formed from lyophilized powder with proline was constructed.

TABLE 3-2
SEC stability of control (diluted mAb stock as received), diluted
lyophilized powder with 1:1 mAb 1:proline by mass, and dispersions
made by addition of a 150 mg/mL mAb dispersion manufactured
by centrifugation concentration to lyophilized powder (C-D/P)
and by centrifugation (C). All dispersions were diluted to
1 mg/mL in 50 mM pH 6.4 sodium phosphate.

| Sample ID | % Monomer |
|---|---|
| Control | 99.69 |
| 150 mg/mL stock solution | 99.61 |
| Lyophilized Powder | 99.24 |
| C-D/P 236:250P (Table 3-5) | 99.42 |
| C-D/P 238:250P (Table 3-5) | 99.19 |
| C-D/P 227:150P (Table 3-5) | 99.84 |
| C 262:150P (Table 3-6) | 99.35 |

The stable lyophilized powder was utilized in two different techniques to produce dispersions. In the first approach, powder was packed into a conical vial and diluted by adding buffer as done previously. [13] In this study, a second approach was designed in which a dispersion formed by this first approach at an intermediate concentration of 150 mg/ml was then added on top of additional lyophilized powder to achieve the same final protein concentration. In this case approximately ˜50 percent of the protein was introduced by the second increment of powder. The term LD is used to refer to both of these processes, with the additional suffix of ‘−D/P’ to indicate the addition of the dispersion over the powder in the second increment.

The pathway of nanocluster formation (the LD pathway) is shown in FIG. 2. As the lyophilized powder contacted the buffer solution upon gentle stirring, an optically clear dispersion of protein was produced after bubbles formed during mixing dissipated. As can be seen in Table 3-3, a large variety of clusters were formed with protein concentrations from 235 to 250 mg/mL with proline concentrations from 100 to 400 mg/mL. Despite the large increase in depletion attraction with proline concentration, the nanocluster size did not vary significantly. Additional proline beyond that which was included in the initial lyophilized powder was added to raise the concentration up to as high as 400 mg/mL (sample LD-D/P 234:400P), and the dispersion viscosity increased from 159 to 214 cP. The self-clustering of proline above ˜200 mg/mL [34] presents a complication that may influence the depletion potential at the highest concentrations as well as the viscosity. A weakening of depletion with a loss in number of proline entities upon formation of proline aggregates may contribute to the lack of an increase in cluster size with proline concentration. In contrast, for trehalose as a depletant, as the concentration was increased from 100 to 270 mg/mL, the cluster size increased from 51 to 95 nm for a protein concentration of ˜250 mg/mL for polyclonal sheep IgG. [20]

TABLE 3-3
Nanocluster dispersions formed from lyophilized powder
composed of equal masses of mAb 1 and proline. In the LD process,
lyophilized powders were dispersed in buffer. In the LD-D/P process,
an intermediate (~150 mg/ml mAb 1) dispersion (D) made by the LD
process was added to additional lyophilized powder

| mAb | Proline | | | | Inherent | Apparent | |
|---|---|---|---|---|---|---|---|
| Conc. | Conc. | Dispersion | Buffer & | Viscosity | viscosity | hydrodynamic | Sample |
| (mg/mL) | (mg/mL) | Method | pH | (cP) ± s.d. | (ml/g) | diameter (nm) | ID |
| 158 | 158 | Packed vial | 50 mM pH | 12 (n = 1) | 13.6 | — | LD |
| | | | 7.2 | | | | 158:158P |
| | | | phosphate | | | | |
| 252 | 252 | Dispersion | 50 mM pH | 159 ± 13 | 17.8 | — | LD-D/P |
| | | over powder | 7.2 | | | | 252:252P |
| | | | phosphate | | | | |
| 146 | 146 | Packed Vial | 50 mM pH | 8 (n = 1) | 11.9 | — | LD |
| | | | 9.3 | | | | 146:146P |
| | | | carbonate | | | | |
| 258 | 258 | Dispersion | 50 mM pH | 114 ± 23 | 16.1 | — | LD-D/P |
| | | over powder | 9.3 | | | | 258:258P |
| | | | carbonate | | | | |
| 245 | 100 | Packed Vial + | 50 mM pH | 135 ± 45 | 19.1 | 49 | LD-D/P |
| | | incremental | 7.2 | | | | 245:100P |
| | | addition* | phosphate | | | | |
| 248 | 100 | Packed Vial + | 50 mM pH | 167 ± 17 | 19.8 | 47 | LD-D/P |
| | | incremental | 7.2 | | | | 248:100P |
| | | addition* | phosphate | | | | |
| 248 | 150 | Packed Vial + | 50 mM pH | 163 ± 23 | 19.2 | 42 | LD-D/P |
| | | incremental | 7.2 | | | | 248:150P |
| | | addition* | phosphate | | | | |
| 253 | 150 | Packed Vial + | 50 mM pH | 172 ± 24 | 19.0 | 45 | LD-D/P |
| | | incremental | 7.2 | | | | 253:150P |
| | | addition* | phosphate | | | | |
| 234 | 400 | Dispersion | 50 mM pH | 214 ± 37 | 19.4 | 51 | LD-D/P |
| | | over powder | 7.2 | | | | 234:400P |
| | | | phosphate | | | | |
| 244 | 237 | Packed | 50 mM pH | 128 ± 14 | 17.7 | — | LD |
| | | Vial* | 8.2 | | | | 244:237P |
| | | | phosphate | | | | |
| | | | w/ 200 mg/mL | | | | |
| | | | Proline | | | | |
| 229 | 229 | Packed Vial | 50 mM pH | 112 ± 11 | 18.3 | — | LD |
| | | | 8.2 | | | | 229:229P |
| | | | phospate | | | | |
| 288 | 288 | Packed Vial | 50 mM pH | 86 ± 13 | 13.6 | 41 | LD |
| | | | 5.5 succinate | | | | 288:288P |

Utilizing the LD pathway, dispersions formulated at ˜150 mg/mL mAb 1 exhibited low viscosities of ˜10 cP (Samples LD 158:158P and LD 146:146P, Table 3-3), compared with a value of 28 cP for the lowest viscosity solution control (Table 3-4). As the protein concentration increased to ˜250 mg/mL the dispersion viscosities were mostly >100 cP. Several different pH buffers (pH 5.5 50 mM succinate, pH 8.2 50 mM phosphate, pH 7.2 50 mM phosphate, and pH 9.3 50 mM carbonate) were used to try to reduce dispersion viscosity. In many cases the [q] was 16-19 ml/g for concentrations of ˜250 mg/mL, but only 12-14 ml/g for the lower protein concentrations. The change in [η], which reflects stronger interactions, may indicate onset of gelation. A particularly remarkable result was obtained for 288 mg/mL mAb 1, where the viscosity was only 86 cP with a low [η] of only 14 ml/g, despite the extremely high concentration. To further lower the viscosity and [η], other pathways were explored to attempt to attempt to minimize passing into metastable gel states.

TABLE 3-4
Protein solutions formed by centrifugal filtration as in Table 3-6
except with dilute trehalose as a stabilizer, without any proline.
The trehalose concentration was 30 mg/mL after buffer exchange (with
the exception of the DI water sample, which had no excipient). The
mAb solution formed in DI water equilibrated at pH 5.5.

| mAb | | | Inherent | Apparent |
|---|---|---|---|---|
| Conc. | Buffer | Viscosity | viscosity | hydrodynamic |
| (mg/mL) | Solution | (cP) ± s.d. | (ml/g) | diameter (nm) |
| 146 | DI Water | 26 ± 1 | 22.3 | 13 |
| 248 | 50 mM acetate, | 100 ± 13 | 18.5 | — |
| | pH 4.7 | | | |
| 244 | 50 mM acetate, | 243 ± 31 | 22.5 | — |
| | pH 5.5 | | | |
| 216 | 50 mM sodium | 230 ± 48 | 25.1 | — |
| | phosphate, | | | |
| | pH 7.2 | | | |
| 190 | 50 mM sodium | 247 ± 44 | 29.0 | — |
| | phosphate, | | | |
| | pH 8.2 | | | |
| 173 | 50 mM sodium | Could not be | — | — |
| | carbonate, | syringed | | |
| | pH 10 | | | |
| 224 | 50 mM sodium | 113 ± 14 | 21.1 | — |
| | carbonate, | | | |
| | pH 11.5 | | | |

Addition of a Dispersion Formed by Centrifugal Filtration Concentration Over Lyophilized Powder Composed of mAb 1 and Proline (C-D/P).

In the second pathway, a stock solution formed by concentrating mAb 1 to 150 mg/mL in DI water via centrifugation filtration was then added to lyophilized powder (1:1 mAb 1:proline) and concentrated inorganic buffer to form a dispersion (C-D/P, Table 3-5). The irreversible aggregate concentration was below 0.5% in the initial stock solution as determined by SEC (Table 3-2). In this pathway labeled as C-D/P in FIG. 2, the mAb 1 concentration increases without a change in the proline concentration during the centrifugation step, and then both mAb 1 and proline concentration increase with the addition of lyophilized powder.

TABLE 3-5
Nanocluster dispersions formed by adding mAb solution in DI
manufactured by centrifugation filtration at 150 mg/mL to 500 mM
phosphate buffer, solid proline powder, and 1:1 mass ratio mAb 1: proline
lyophilized powder (with the exception of row 1, where no lyophilized mAb
powder was added) to further concentrate the protein with crowder.

| | | % | | | | | |
|---|---|---|---|---|---|---|---|
| mAb | Proline | protein | | Viscosity | Inherent | Apparent | |
| Conc. | Conc | from | | (cP) ± | viscosity | hydrodynamic | Sample |
| (mg/mL) | (mg/mL) | solution | Buffer & pH | s.d. | (ml/g) | diameter (nm) | ID |
| 133 | 130 | 100 | DI Water | 10 ± 1 | 15.1 | 18 | C-D/P |
| | | | | | | | 133:130P |
| 156 | 150 | 76 | 50 mM pH | 13 ± 1 | 14.2 | 32 | C-D/P |
| | | | 7.2 phosphate | | | | 156:150P |
| 227 | 150 | 53 | 50 mM pH | 47 ± 2 | 15.5 | — | C-D/P |
| | | | 7.2 phosphate | | | | 227:150P |
| 252 | 250 | 39 | 50 mM pH | 129 ± 17 | 17.2 | 44 | C-D/P |
| | | | 7.2 phosphate | | | | 252:250P |
| 236 | 250 | 39 | 50 mM pH | 123 ± 10 | 17.7 | 45 | C-D/P |
| | | | 8.2 phosphate | | | | 236:250P |
| 238 | 250 | 39 | 50 mM pH | 122 ± 10 | 17.9 | 48 | C-D/P |
| | | | 9.3 carbonate | | | | 238:250P |

For dilute protein below 200 mg/mL, nanoclusters were formed with diameters below 40 nm. For protein concentrations from ˜230 to 250 mg/mL, the nanocluster diameters for similar conditions were again about 45 nm as seen in Table 3-3 and in FIG. 4 for various dispersion buffer pH values. With added protein, the pH shifted less than 0.5 units relative to the buffer. The viscosities of formulations produced by centrifugation filtration without added depletant are shown in Table 3-4. At pH values from 8.2 to 10, the viscosities were the highest when normalized for protein concentration, which may be expected near the isoelectric point where electrostatic repulsion is weak. At either lower or higher pH values as the protein became more charged, the viscosity (normalized by protein concentration) decreased. The dispersion viscosity was lower than that of comparable solutions at similar pH and protein concentration (as seen in Tables 3-3 and 3-4). Furthermore, as shown in FIG. 5, the dispersion viscosity was not sensitive to pH from pH 7.2 to 9.3 for the C-D/P pathway. Similarly to dispersions produced by the LD pathway, the stock dispersion over powder method produced very low viscosities (<15 cP) at ˜150 mg/mL mAb 1 concentration, but significantly higher viscosities at the higher 250 mg/mL mAb 1 concentration.

Centrifugation Filtration.

The third pathway tested was concentration of mAb 1 via centrifugal filtration (C), as shown via the C pathway in FIG. 2, from a solution with the desired final depletant concentration. Ideally, the depletant would permeate the filter non-selectively with the buffer, such that the depletant concentration would decrease about 15-20% from the increase in protein concentration and consequent increase in the protein volume fraction in the system. An SEC study of proline content in dispersions formed by centrifugation was undertaken to determine if proline was retained or excluded at a greater rate than mAb 1 (Table 3-S 1). The proline content of dispersions at pH 8 and 7 were seen to be on average 12% lower than the feed values, in line with the expected 15-20% decrease in depletant concentration due to the increase in protein volume fraction with increasing protein concentration. (In all other places in the paper, we report the feed proline value rather than the corrected value.) The SEC measurements suggest relatively non-selective permeation of proline across the membrane without significant complications of specific interactions with the protein, Donnan equilibrium across the membrane, or other effects.

TABLE 3-S1
SEC measured values of proline concentration from SEC peak
area, normalized by the mass of actual injected protein per
sample. Samples in rows 1-4 have fully defined compositions
(made by dispersion-over-powder technique). Samples in rows
5-9 were made by centrifugation concentration (C), and the
estimated proline concentrations were verified by SEC. On average,
measured proline concentrations were within 12% of those estimated
by quantification of mass of proline added (LD-D/P and C-D/P)
and the buffer proline concentration pre-buffer exchange (C),
even post-centrifugal filtration.

| | | | Proline | Proline | % Diff. |
|---|---|---|---|---|---|
| | | | Conc | Conc (by | btw |
| | Table | mAb | (Esti- | SEC) ± | SEC & |
| | Found | Conc. | mate, | s.d. | Esti- |
| Sample ID | In | (mg/mL) | mg/mL) | (mg/mL) | mate |
| LD-D/P 234:400P | 1 | 234 | 400 | 342 ± 7 | 14% |
| C-D/P 236:250P | 2 | 236 | 250 | 214 ± 4 | 14% |
| C-D/P 238:250P | 2 | 238 | 250 | 225 ± 5 | 10% |
| C-D/P 227:150P | 2 | 227 | 150 | 116 ± 1 | 22% |
| C-S 262:150P | 3 | 262 | 150 | 117 | 22% |
| C 213:150 mg/mL | — | 213 | 150 | 160 | 7% |
| mAb:Pro, 60 cP, | | | | | |
| intrinsic 11, | | | | | |
| cluster diameter | | | | | |
| 39 nm, in pH 8.2 | | | | | |
| phosphate buffer, | | | | | |
| dispersion pH 8 | | | | | |
| C 199:200 mg/mL | — | 199 | 200 | 215 | 8% |
| mAb:Pro, 57 cP, | | | | | |
| intrinsic 11, | | | | | |
| cluster diameter | | | | | |
| 33, in pH 7.2 | | | | | |
| phosphate buffer, | | | | | |
| dispersion pH 7 | | | | | |
| C 210:200 mg/mL | — | 210 | 200 | 225 | 13% |
| mAb:Pro, 47 cP, | | | | | |
| intrinsic 10, | | | | | |
| cluster diameter | | | | | |
| 38, in pH 7.2 | | | | | |
| phosphate buffer, | | | | | |
| dispersion pH 7 | | | | | |
| C 227:150 mg/mL | — | 227 | 150 | 151 | 1% |
| mAb:Pro, 60 cP, | | | | | |
| intrinsic 10, | | | | | |
| cluster diameter | | | | | |
| 41, in pH 8.2 | | | | | |
| phosphate buffer, | | | | | |
| dispersion pH 8 | | | | | |

For this method, the retentate was recovered under gravity (the “pour off” method), or by low-speed (1000 RCF) centrifugation (“spin off”). Our hypothesis was that the pour off method may leave behind protein that gelled and stuck to the filter, to enable recovery of a less viscous fraction that flowed out of the vessel. However, no consistent differences were seen in the viscosities between the two methods, despite lower yields in the pour off method (Table 3-6).

TABLE 3-6
Nanocluster dispersions formed by centrifugation filtration to
concentrate the protein after buffer exchange at a level of 4 mg/mL mAb 1.

| | Assumed | | | | | Apparent | | |
|---|---|---|---|---|---|---|---|---|
| mAb | Proline | | | Viscosity | Inherent | hydrodynamic | | |
| Conc. | Conc. | Buffer & | Recovery | (cP) ± | viscosity | diameter | % | Sample |
| (mg/mL) | (mg/mL) | pH | Method | s.d. | (ml/g) | (nm) | Yield | ID |
| 199 | 150 | 50 mM pH | Spin Off | 25 ± 2 | 14.1 | 22 | 59 | C- |
| | | 6.4 | | | | | | S199:150P |
| | | phosphate | | | | | | |
| 211 | 150 | 50 mM pH | Spin off | 20 ± 2 | 12.2 | 18 | 69 | C-S |
| | | 6.4 | | | | | | 211:150P |
| | | phosphate | | | | | | |
| 251 | 150 | 50 mM pH | Spin Off | 42 ± 2 | 13.3 | 25 | 94 | C-S |
| | | 6.4 | | | | | | 251:150P |
| | | phosphate | | | | | | |
| 243 | 150 | 50 mM pH | Spin Off | 47 ± 7.4 | 14.2 | — | 70 | C-S |
| | | 7.2 | | | | | | 243:150P |
| | | phosphate | | | | | | |
| 258 | 150 | 50 mM pH | Pour off | 35 ± 5 | 12.2 | 33 | 56 | C-P |
| | | 7.2 | | | | | | 258:150P |
| | | phosphate | | | | | | |
| 266 | 150 | 50 mM pH | Pour Off | 55 ± 2 | 13.5 | 37 | — | C-P |
| | | 7.2 | | | | | | 266:150P |
| | | phosphate | | | | | | |
| 225 | 150 | 50 mM pH | Spin off | 25 ± 2 | 12.5 | 44 | 91 | C-S |
| | | 8.2 | | | | | | 225:150P |
| | | phosphate | | | | | | |
| 228 | 150 | 50 mM pH | Spin Off | 40 ± 12 | 14.4 | 40 | 95 | C-S |
| | | 8.2 | | | | | | 228:150P |
| | | phosphate | | | | | | |
| 257 | 150 | 50 mM pH | Pour Off | 56 ± 3 | 14.1 | 41 | 46 | C-P |
| | | 8.2 | | | | | | 257:150P |
| | | phosphate | | | | | | |
| 262 | 150 | 50 mM pH | Spin Off | 80 ± 4 | 15.2 | 36 | 98 | C-S |
| | | 8.2 | | | | | | 262:150P |
| | | phosphate | | | | | | |

For a protein concentration of ˜250 mg/mL, the nanocluster size increased as the pH increased from 6.4 to 8.2 approaching the pI, as expected from the free energy model, due to lower charge repulsion between clusters and monomer (Table 3-6, FIG. 4). Even more remarkably, this centrifugation filtration process and depletant selection also produce these low viscosity dispersions at pH's of 6.4, 7.2 and 8.2. The charge suppressing effects of the proline that contribute to clustering also seem to lead to weak interactions and low viscosities. While the size of the nanoclusters decreased with a decrease in dispersion pH (as seen in FIG. 4), the nanoclusters were still formed a full 3 pH units away from the mAb 1 isoelectric point suggesting that the proline may decrease the electrostatic repulsion of the protein monomers, or that nanoclusters may still be formed at higher charges with sufficient depletion attraction. Nanocluster formation this far from the isoelectric point was not investigated in previous studies. [13,20]

The centrifugation filtration method produced moderately lower viscosities and inherent viscosities than the other two methods at a given composition. Unlike the lyophilization dilution and centrifugation dispersion over powder methods, the centrifugal filtration method produces repeatable dispersion viscosities in the 50 cP range with mAb 1 concentrations of ˜250 mg/mL. Despite these differences in viscosities, the nanocluster diameters were fairly similar for the various processes for similar compositions, suggesting equilibrium aspects are present in nanocluster formation. [20] Although the mechanism for the lower viscosities for this method is not well understood, a few factors may be considered. In the centrifugal filtration method, the large number of air bubbles formed in the other two processes is not present. Proteins may adsorb at air/solution interfaces and aggregate and nucleate gels of interacting clusters. Another possibility is that solid entities in the lyophilized powders do not fully dissociate into non-interacting clusters upon stirring but form gel networks of interacting clusters. Despite these complexities in the rheological behavior, it is interesting that the apparent nanocluster hydrodyanamic diameters were similar indicating a driving force toward aspects of equilibrium behavior.

Complications with the centrifugation technique include specific interactions of proline with the charged protein surface, Donnan equilibrium, exclusion, and preferential hydration effects, [46-48] all of which may cause unequal partitioning of depletant molecules across the filtration membranes. However, it has been demonstrated that with histidine, if diafiltration is done at the amino acid pI, unequal partitioning did not occur. [47] In addition, various previous studies on this topic utilized tangential-flow filtration (TFF) where the feed and permeate were in contact, whereby equilibrium processes may influence permeation. In centrifugal filtration the contact between the retentate and permeate was minimized, given the gap between the filter and the permeate at the bottom of the tube, and thus Donnan equilibrium may play a less significant role.

Experimental Section

mAb 1. The human monoclonal antibody used for this study was obtained from AbbVie. It was stored frozen in 10 mL aliquots at ˜100 mg/mL and −40° C., and thawed at 2-8° C. Each aliquot had been filtered through a 0.22 μm filter (Pall) prior to freezing. The protein concentration was verified via A280 prior to use. For extended refrigerated storage, mAb 1 was diluted to ˜4 mg/mL in pH 6.4 50 mM phosphate buffer and stored at 2-8° C.

Buffer Exchange.

After thawing and dilution, the ˜4 mg/mL solution of mAb 1 was buffer exchanged into 50 mM phosphate buffer with the desired amount of dissolved proline (Alfa Aesar), typically 150 mg/mL or none. The buffer exchange was carried out using centrifugal filter tubes (Millipore, Amicon Ultracel 30K centrifugal filters) with a molecular weight cutoff of 30 kDa and a capacity of 12 mL. A desired amount of the protein solution (typically 6-10 mL) was added to the filter tube, and the desired buffer for the dispersion was added to increase the solution volume to 12 mL. The buffer was forced through the membrane by centrifugal filtration at 4500 radial centrifugal force (rcf) for 12 minutes concentrating the protein solution in the retentate until the solution volume dropped to about 2 mL. The retentate protein solution was then diluted to 12 mL in the same buffer as before and concentrated down to 2 mL again. The dilution and centrifugation process was repeated 4 or more times until the permeate volume was 4-5 times the original solution volume, typically 40 mL. After buffer exchange, the solution was further concentrated to 50-80 mg/ml so that the final solution volume was about 0.5 mL.

Centrifugal Filtration of Protein Solution to Form a Dispersion of Nanoclusters Upon Concentration.

Tare weights were taken of a centrifugal filter assembly (Millipore Microcon, Ultracel YM-50 membrane, 50 kDa nominal molecular weight limit, diameter of filter, 0.25″). The desired volume (˜0.5 mL) of protein solution, after buffer exchange and concentration, was pipetted into the retentate chamber. The filter assembly was then centrifuged (Eppendorf Centrifuge 5415D) at 10,000 rcf—typically in 20-40 minute increments until the retentate reached the calculated volume needed to achieve the desired final protein concentration. The volume measurements were done using ImageJ image analysis to determine the height of the liquid column in the retentate chamber. Additionally, the protein concentration in the retentate dispersion was determined by measuring out 2 μL (±0.08 μL) of dispersion using an Eppendorf Research adjustable volume 0.5-10 μL pipette and diluting it into a receiving vessel containing 998 μL of 50 mM pH 6.4 phosphate buffer. For mixing, the solution was cycled 5 times into and back out of the pipette tip followed by light agitation with the pipette tip. The absorbance of the resulting solution at 280 nm was measured using a Cary 3E UV-visible spectrophotometer in a cuvette (Hellma cells) with a path length of 1 cm, and converted to concentration assuming an extinction coefficient of 1.42 mL mg⁻¹ cm⁻¹.

Once the desired concentration had been reached, the dispersion of protein nanoclusters in the retentate chamber was recovered either by inverting the filter assembly into a retentate recovery tube, and centrifuging it for 3-4 minutes at 1,000 RCF (“spin off”) or by inverting it into the retentate recovery tube and allowing the dispersion to flow into the tube under gravity (“pour off”). The resulting dispersion was transferred to a 0.1 mL conical vial (V-Vial, Wheaton), and the concentration was confirmed using 2 μL of the dispersion as described above.

Lyophilization Dilution.

Prior to lyophilization, the mAb 1 solution described above was buffer exchanged into DI water and concentrated to ˜60-80 mg/mL using a 50,000 molecular weight cutoff (MWCO) Centricon filter as described above in the buffer exchange step. Solid proline was then added to the mAb 1 solution as a cryoprotectant at a 1:1 protein: proline ratio by mass. The solution was filter-sterilized (0.22 μm), diluted to ˜50 mg/mL protein with DI water, and transferred to a glass vial. It was frozen over 6 hours at −40° C. and then lyophilized (VirTis Advantage Plus Benchtop Freeze Dryer) at 150 mTorr with 24 hours of primary drying at −40° C. followed by a 6 hour ramp to 25° C. and an additional 6 hours of secondary drying at 25° C. To prepare mAb 1 dispersions, typically 40 mg of lyophilized protein was compacted into a tared 0.1 mL conical vial (V-Vial, Wheaton). After addition of the desired buffer and/or additional proline, the resulting dispersion was stirred gently with the tip of a 25 gauge needle. The total volume and volume fractions of the components were calculated assuming ideal mixing based on known masses, and hypothetical pure liquid protein (1.35 g/cm³) and proline (1.40 g/cm³) densities, from their partial molar volumes at infinite dilution [40,41] and a known buffer volume. The final protein concentration was verified using light absorbance at 280 nm as described above.

Centrifugation Dispersion over Powder.

For C-D/P, lyophilized powder was made as described above. In addition, an intermediate dispersion of 150 mg/ml mAb 1 and proline was made via buffer exchange and centrifugation concentration as previously described. The required amount of lyophilized powder necessary to bring the dispersion to the desired final concentration, calculated assuming ideal mixing based on known masses, and hypothetical pure liquid protein (1.35 g/cm³) and proline (1.40 g/cm³) densities, was weighed into a tared 0.1 mL conical vial. The required volume of dispersion was then pipetted atop the powder, and the resulting mixture was stirred gently with the tip of a 25 gauge needle until the liquid dispersion was optically transparent.

Characterization of the Protein Nanocluster Dispersion

Zeta Potential.

Zeta potential analysis was conducted using a Brookhaven ZetaPlus zeta potential analyzer. Samples were formulated at 1 mg/mL mAb 1 concentration with the desired concentration of excipient in the specified buffer, and the averages for 30 single-cycle measurements are reported.

Hydrodynamic Diameters.

The effective short-time mutual diffusion coefficient Dₛ(q) of protein nanoclusters was extracted from intensity correlation functions measured using dynamic light scattering (DLS). Measurements were taken at an angle of 150° with a 632.8 nm laser (q=0.01918 nm⁻¹) and an avalanche photodiode at ˜23° C. using a custom apparatus (Brookhaven BI-9000AT and 60 μl Beckman Coulter sample cell). [13] The intensity autocorrelation function (ACF) was analyzed with the CONTIN algorithm (volume distribution), where the baseline was fit at the apparent base of the first exponential decay curve, corresponding to the primary peak in the diffusion coefficient distribution. A representative ACF and the corresponding fitted baseline for a concentrated mAb 1 dispersion were constructed. Apparent hydrodynamic cluster diameters D_{c} were estimated from the Dₛ(q) using Beenakker-Mazur theory [42] for Dₛ(q)/D₀, where D₀=kT/3πηD_{c} and η is the shear viscosity of the buffer solvent with added excipients. This approach assumes that the protein clusters act like suspended hard spheres occupying an effective packing fraction φ_{c}^{eff}=φ/φ_{c}^{int}, where φ_{c}^{int} is the protein packing fraction within a cluster. In this work, we assumed φc^{int}=0.60, which is consistent with light scattering data on protein nanoclusters reported previously. [13] We also verified that an alternative approximation, φ_{c}^{int} (D_{c}/2R)^{δ}^{_{f}}⁻³ (where δ_{f} is the fractal dimension, taken as 2.6), [13] resulted in similar cluster size estimates. The measured intensity correlation functions decayed on time scales between ˜10 to 50 consistent with short-time diffusion for clusters with the diameters and mobilities reported here.

Size Exclusion Chromatography.

For analysis of non-covalent aggregates, the sample was diluted to ˜1 mg/mL protein in 50 mM pH 6.4 sodium phosphate buffer. 20 μg of protein in the diluted dispersion was analyzed with a Waters Breeze HPLC, using TOSOH Biosciences TSKgel3000SW_{XL} and TSKgel2000SW columns in series and a mobile phase of 100 mM sodium phosphate, 300 mM sodium chloride, pH 7, with eluate monitored by absorbance at 214 nm. For analysis of proline concentration in retentate samples from ultrafiltration, samples were prepared identically to the non-covalent aggregate analysis, with the exception that final protein concentration was measured in order to determine dilution factor. The area under the curve for the proline peak was measured and compared to a standard curve that had been determined a priori in order to determine the proline concentration.

Viscosity.

The viscosities of the nanocluster dispersions were measured in triplicate using a 25 gauge (ID=0.1 mm) 1.5″ long needle (Becton Dickinson & Co. Precision Glide Needle) attached to a 1 mL syringe (Becton Dickinson & Co. 1 mL syringe with LUER-LOK™ tip), according to the Hagen-Poiseuille equation. [13] The flow rate of the dispersion through the needle was determined by correlating volume to the height of the liquid in the conical vial (using ImageJ software) and measuring the time taken for the dispersion column height to move between two points. The flow rate was correlated to viscosity from a calibration curve derived from a set of standards of known viscosities. [13]

pH Measurement.

The pH of the buffer solutions prepared with and without dissolved proline (for making dispersions by centrifugation and dispersion over powder, respectively) was measured using a pH meter (Mettler-Toledo) with an accuracy of ±0.01 pH units. Due to the small sample size (100 μL) of the final dispersion, the dispersion pH was measured using pH indicator strips (EMD Millipore) with an accuracy of ±0.5 pH units. pH measurements of the same sample by the two methods agreed within 0.5 pH units, which is at the maximum level of sensitivity of the pH indicator strips.

Conclusions.

Low viscosity dispersions of nanoclusters of concentrated protein were formed utilizing proline as a depletant, with viscosities an order of magnitude lower than those of solutions at comparable concentrations and pH. Despite the much smaller size of proline relative to previously used depletants, trehalose and polyethylene glycol, [13] depletion attraction was still sufficient to form nanoclusters. The high preferential exclusion of proline (more significant than trehalose) leads to increased solvophobic effects with any exposed protein backbone, increasing the tendency of proteins to stay folded in this environment. The relatively constant nanocluster size over a proline concentration range from 100 to 300 mg/mL may be contrasted with a significant increase in size for the sheep IgG as the depletant (trehalose) concentration was increased. In addition, proline self-assembles at concentrations greater than 200 mg/ml, [34] which may impede depletion at high concentrations and thus growth in cluster size. Furthermore, proline produces additional effects beyond depletion attraction, particularly the reduction of apparent surface charge for dilute mAb 1. If this electrostatic screening is present for concentrated mAb1, it may explain in part why nanoclusters could still be assembled 2-3 pH units away from the mAb pI, at a desirable pH for pharmaceutical use. The formation of these nanoclusters appeared to involve aspects of equilibrium behavior, [20] as similar apparent hydrodynamic diameters were produced via three different routes: centrifugation filtration, lyophilization dilution, and centrifugation filtration dispersion over lyophilized powder. Moreover, all routes produced similarly sized small clusters over a proline concentration range from 100 to 300 mg/mL. Upon dilution the nanoclusters dissociated back to monomer with less than 1% aggregates as shown by SEC. Dispersions produced via centrifugal filtration were modestly less viscous than those produced by the powder based techniques at similar compositions. Thus, the interactions between the nanoclusters depended upon the formation pathway. For the other cases, formation of air bubbles during mixing may have produced reversible protein adsorption and aggregation that raised the viscosity. In addition, proline was found to serve as a cryoprotectant for lyophilization even at levels as low as 1:0.1 mass of protein to proline, retaining monomeric protein post-lyophilization according to SEC.

### References (Example 3)

[1]. Laue T 2012. Proximity energies: a framework for understanding concentrated solutions. Journal of Molecular Recognition 25(3):165-173; [2]. 2011. Rituxan Dosing and Administration Guide. In Genentech, editor, ed; [3]. Sinko P J, Martin A. 2006. Martin's Physical Pharmacy and Pharmaceutical Sciences. 5 ed., Philadelphia: Lippincott Williams & Wilkins; [4]. Zhou H X, Rivas G, Minton AP 2008. Macromolecular Crowding and Confinement: Biochemical, Biophysical, and Potential Physiological Consequences Annual Review of Biophysics 37(1):375-397; [5]. Rosenberg E, Hepbildikler S, Kuhne W, Winter G 2009. Ultrafiltration concentration of monoclonal antibody solutions: Development of an optimized method minimizing aggregation. Journal of Membrane Science 342(1-2):50-59; [6]. Young™, Roberts C J 2009. Structure and Thermodynamics of Colloidal Protein Cluster Formation: Comparison of Square-Well and Simple Dipolar Models. The Journal of Chemical Physics 131(12):125104; [7]. Fields G B, Alonso D O V, Stigter D, Dill K A 1992. Theory for the Aggregation of Proteins and Copolymers. J Phys Chem 96(10):3974-3981; [8]. Shire S J, Shahrokh Z, Liu J 2004. Challenges in the Development of High Protein Concentration Formulations. J Pharm Sci 93(6):1390-1402; [9]. Rosenbaum D F, Zamora P C, Zukoski C F 1996. Phase Behavior of Small Attractive Colloidal Particles. Phys Rev Let 76(1):150-153; [10]. ten Wolde P R, Frenkel D 1997. Enhancement of Protein Crystal Nucleation by Critical Density Fluctuations. Science 277:1975-1978; [11]. Zaccarelli E 2007. Colloidal Gels: Equilibrium and Non-Equilibrium Routes. J Phys: Condens Matter 19:323101; [12]. Hartl F U, Hayer-Hartl M 2002. Protein Folding—Molecular Chaperones in the Cytosol: from Nascent Chain to Folded Protein. Science 295(5561):1852-1858; [13]. Johnston K P, Maynard J A, Truskett T M, Borwankar A, Miller M A, Wilson B, Dinin A K, Khan T A, Kaczorowski K J 2012. Concentrated Dispersions of Equilibrium Protein Nanoclusters That Reversibly Dissociate into Active Monomers. Acs Nano; [14]. Shen V K, Cheung J K, Errington J R, Truskett T M 2006. Coarse-Grained Strategy for Modeling Protein Stability in Concentrated Solutions II: Phase Behavior. Biophysical journal 90:1949-1960; [15]. Cheung J K, Truskett T M 2005. Coarse-Grained Strategy for Modeling Protein Stability in Concentrated Solutions. Biophysical journal 89(4):2372-2384; [16]. Murthy A K, Stover R J, Borwankar A U, Nie G D, Gourisankar S, Truskett T M, Sokolov K V, Johnston K P 2013. Equilibrium Gold Nanoclusters Quenched with Biodegradable Polymers. Acs Nano 7(1):239-251; [17]. Groenewold J, Kegel WK 2004. Colloidal Cluster Phases, Gelation and Nuclear Matter. J Phys-Condes Matter 16(42):54877-S4886; [18]. Groenewold J, Kegel WK 2001. Anomalously Large Equilibrium Clusters of Colloids. J Phys Chem B 105(47):11702-11709; [19]. Miller M A, Khan T A, Kaczorowski K J, Wilson B K, Dinin A K, Borwankar A U, Rodrigues M A, Truskett T M, Johnston K P, Maynard J A 2012. Antibody nanoparticle dispersions formed with mixtures of crowding molecules retain activity and In Vivo bioavailability. Journal of pharmaceutical sciences 101(10):3763-3778; [20]. Borwankar A U, Dinin A K, Laber J R, Twu A, Wilson B K, Maynard J A, Truskett T M, Johnston K P 2013. Tunable equilibrium nanocluster dispersions at high protein concentrations. Soft Matter 9(6):1766-1771; [21]. Srinivasan C, Weight A K, Bussemer T, Klibanov A M 2013. Non-Aqueous Suspensions of Antibodies are Much Less Viscous Than Equally Concentrated Aqueous Solutions. Pharm Res; [22]. Xia Y S, Nguyen T D, Yang M, Lee B, Santos A, Podsiadlo P, Tang Z Y, Glotzer S C, Kotov N A 2011. Self-assembly of self-limiting monodisperse supraparticles from polydisperse nanoparticles. Nat Nanotechnol 6(9):580-587; [23]. Zhang C, Pansare V J, Prud'homme R K, Priestley R D 2012. Flash nanoprecipitation of polystyrene nanoparticles. Soft Matter 8(1):86-93; [24]. Miller M A, Engstrom J D, Ludher B S, Johnston K P 2010. Low Viscosity Highly Concentrated Injectable Nonaqueous Suspensions of Lysozyme Microparticles. Langmuir: the ACS journal of surfaces and colloids 26(2):1067-1074; [25]. Hiemenz P C, Rajagopalan R. 1997. Principles of Colloid and Surface Chemistry. 3rd ed., New York: Marcel Dekker, Inc. p 650; [26]. Torquato S, Truskett T M, Debenedetti P G 2000. Is Random Close Packing of Spheres Well Defined? Phys Rev Lett 84(10):2064-2067; [27]. Yadav S, Liu J, Shire S J, Kalonia D S 2010. Specific Interactions in High Concentration Antibody Solutions Resulting in High Viscosity. Journal of pharmaceutical sciences 99(3):1152-1168; [28]. Civera M, Sironi M, Fornili S L 2005. Unusual properties of aqueous solutions of L-proline: A molecular dynamics study. Chem Phys Lett 415(4-6):274-278; [29]. Campbell M K, Farrell S O. 2003. Biochemistry. 4th ed., United States: Thomson-Brooks/Cole; [30]. Ignatova Z, Gierasch L M 2006 Inhibition of protein aggregation in vitro and in vivo by a natural osmoprotectant. Proc Natl Acad Sci USA 103(36):13357-13361; [31]. Troitzsch R Z, Vass H, Hossack W J, Martyna G J, Crain J 2008. Molecular mechanisms of cryoprotection in aqueous proline: light scattering and molecular dynamics simulations. J Phys Chem B 112(14):4290-4297; [32]. Shukla D, Schneider C P, Trout B L 2011. Molecular level insight into intra-solvent interaction effects on protein stability and aggregation. Adv Drug Deliv Rev 63(13):1074-1085; [33]. Bolli R, Woodtli K, Bartschi M, Hofferer L, Lerch P 2010. L-Proline reduces IgG dimer content and enhances the stability of intravenous immunoglobulin (IVIG) solutions. Biologicals 38(1):150-157; [34]. Samuel D, Kumar T K, Ganesh G, Jayaraman G, Yang P W, Chang M M, Trivedi V D, Wang S L, Hwang K C, Chang D K, Yu C 2000. Proline inhibits aggregation during protein refolding. Protein Sci 9(2):344-352; [35]. Auton M, Bolen D W 2005. Predicting the energetics of osmolyte-induced protein folding/unfolding. Proc Natl Acad Sci USA 102(42):15065-15068; [36]. Courtenay E S, Capp M W, Anderson C F, Record M T, Jr. 2000. Vapor pressure osmometry studies of osmolyte-protein interactions: implications for the action of osmoprotectants in vivo and for the interpretation of “osmotic stress” experiments in vitro. Biochemistry 39(15):4455-4471; [37]. Xie G, Timasheff S N 1997. The thermodynamic mechanism of protein stabilization by trehalose. Biophysical Chemistry 64:25-43; [38]. Cheung Miss., Klimov D, Thirumalai D 2005. Molecular Crowding Enhances Native State Stability and Refolding Rates of Globular Proteins. Proc Natl Acad Sci USA 102(13):4753-4758; [39]. Yadav S, Shire S J, Kalonia D S 2011. Viscosity Analysis of High Concentration Bovine Serum Albumin Aqueous Solutions. Pharm Res 28(8):1973-1983; [40]. Pilz I, Puchwein G, Kratky O, Herbst M, Haager O, Gall W E, Edelman G M 1970. Small Angle X-Ray Scattering of a Homogeneous GammaG1 Immunoglobin. Biochemistry 9(2):211-219; [41]. Miller D P, dePablo J J, Corti H 1997. Thermophysical properties of trehalose and its concentrated aqueous solutions. Pharm Res 14(5):578-590; [42]. Beenakker C W J, Mazur P 1984. DIFFUSION OF SPHERES IN A CONCENTRATED SUSPENSION 0.2. Physica A 126(3):349-370; [43]. Lekkerkerker H N W, Tuinier R. 2011. Colloids and the Depletion Interaction. 2011 ed.: Springer. p 268; [44]. Xin Y. 2007. Electrokinetic Modeling of Free Solution Electrophoresis. Department of Chemistry, ed.: Georgia State University; [45]. Kar K, Kishore N 2007. Enhancement of thermal stability and inhibition of protein aggregation by osmolytic effect of hydroxyproline. Biopolymers 87(5-6):339-351; [46]. Teeters M, Bezila D, Benner T, Alfonso P, Alred P 2011. Predicting diafiltration solution compositions for final ultrafiltration/diafiltration steps of monoclonal antibodies. Biotechnol Bioeng 108(6):1338-1346; [47]. Miao F, Velayudhan A, DiBella E, Shervin J, Felo M, Teeters M, Alred P 2009. Theoretical analysis of excipient concentrations during the final ultrafiltration/diafiltration step of therapeutic antibody. Biotechnol Prog 25(4):964-972; [48]. Stoner M R, Fischer N, Nixon L, Buckel S, Benke M, Austin F, Randolph T W, Kendrick BS 2004. Protein-solute interactions affect the outcome of ultrafiltration/diafiltration operations. Journal of pharmaceutical sciences 93(9):2332-2342.

### Example 4

### High Concentration Tangential Flow Ultrafiltration of Stable Monoclonal Antibody Solutions with Low Viscosities

Abstract.

Production of concentrated monoclonal antibody formulations by tangential flow ultrafiltration remains challenging due to high viscosities and aggregation that may cause extensive membrane fouling, flux decay and low product yields. The large wall shear stress τ_{w} (>100 Pa) stemming from large pressure drops present in typical TFF systems utilizing flat-sheet filter modules may promote aggregation. To minimize τ_{w}, 100-300 mM of the co-solutes trehalose and histidine or imidazole were added to weaken the protein interactions and reduce the viscosity by up to ten-fold to as low as 40 cP at 250 mg/mL. Hollow fiber filter modules with straight flow paths were used to minimize the axial ΔP and τ_{w}. The combination of the low solution viscosity and low ΔP and τ_{w} of 0.4 bar and 50 Pa, respectively, mitigated aggregation, membrane fouling, and consequently the degree of flux decay, even up to 280 mg/mL mAb. The concentrated mAb was stable by SEC before and after extended storage at 4° C.

Introduction.

For subcutaneous (subQ) delivery of monoclonal antibodies (mAb) and other protein therapeutics, the dosage often requires protein concentrations of 150 mg/mL or higher given the small injection volume of 1.5 mL. [1] At these concentrations, attractive short-ranged interactions between proteins may produce undesirable aggregation [1] and/or high viscosities [3,4] above the desired limit of 20-50 cP for subQ injection. [5] Even though tangential flow filtration (TFF) is the industry standard for manufacturing concentrated proteins, relatively few publications have reported results for concentrations above 150 mg/ml. Low fluxes in TFF and concentration polarization resulting from high viscosities [3,4,6] may result in protein gelation, membrane fouling and protein aggregation. [7] Thus, novel concepts would be highly beneficial for weakening protein interactions to reduce viscosity, both to improve TFF ultrafiltration and advance subcutaneous injection, in each case without the formation of aggregates.

Several studies of TFF have optimized the transmembrane pressure (TMP) and shear stress to reach concentrations of 150 to 200 mg/ml. [6,8-11] Single-pass TFF has been used to reach a final mAb concentration of 225 mg/mL by eliminating the recirculation loop to minimize mAb exposure to high shear stresses in the pump head. [12] These studies have attempted to minimize protein aggregation during filtration [6,8,12] and to avoid large viscosity increases and the associated decay in transmembrane flux [6,8,13-16] and large axial pressure drops. [3] The large pressure drops may exceed pump capacity [3,4] and increase axial variation in TMP with undesirably high TMP values at the influent. [14,15] The TMP is typically optimized based on a “knee-point” in the flux-TMP profile where the flux reaches an asymptote. At higher TMPs, proteins may aggregate and form gels near the membrane wall [6] as a consequence of concentration polarization. [17] As protein concentration increases, it was found that the optimal TMP decreases. [6] The low optimal TMP is problematic since high TMPs would be desirable to overcome losses in protein fluxes at high concentrations resulting from concentration polarization and fouling.

In TFF, fouling and concentration polarization may be mitigated by applying an appropriate wall shear stress τ_{w} or shear rate γ_{w} to sweep protein molecules near the wall back into the bulk flow. [13,18] If the shear stress is too large, it may cause protein denaturation and aggregation, [19] especially in the presence of air-solution and solution-solid interfaces. [20,21] In high mAb concentration TFF studies, emphasis has been placed on flat sheet cassettes (filter modules) due to facile scalability and high fluxes. [6,8-10,12,22,23] However, variations in flow patterns in the serpentine flow path, particularly the stagnation zones in the 90-degree bends, may require excessive wall shear stress to minimize fouling, which then may cause protein denaturation and aggregation. For example, an average τ_{w} of 200-300 Pa is recommended for ultrafiltration of concentrated mAb solutions at typical feed flow rates of 300-400 LMH (liters per square meter per hour) with the flat sheet geometry. [6,8] In contrast, more uniform flow in the hollow fiber geometry may offer the possibility of lower values of τ_{w} to prevent fouling and to minimize shear-induced protein aggregation, particularly at high concentrations. Improvement in the design of asymmetric hollow fiber membranes for higher fluxes makes them more attractive alternatives relative to flat sheets. [13,24]

The design of mAb formulations with lower viscosities at high concentrations would be highly beneficial for sub Q injection and for more efficient ultrafiltration in TFF. In TFF, lower viscosities would enable higher fluxes, lower axial ΔP, and better control of the TMP for lower values of τ_{w}. The viscosity may be lowered by modulation of a variety of types of interactions between the proteins. The exponential dependence of the solution viscosity on concentration is described by the Ross-Minton model [4,25] (Eq. 4-1),

$$η = {η}_{0} exp ( \frac{c [ η ]}{1 - \frac{k}{v} c [ η ]} ) \qquad ( 4 \text{-} 1 )$$

where η is the solution viscosity, η₀ is the solvent viscosity, c is the mAb concentration, [η] is the intrinsic viscosity, k is the crowding factor and v is the Simha shape factor. The exponential increase in viscosity arises from enhanced intermolecular interactions at higher concentrations. Even when protein-protein interactions are repulsive at dilute conditions, they may become attractive at high concentrations [2,4] due to attractive short-ranged polar and hydrophobic interactions which become dominant when protein separation distances become <5 nm. [26] The electrostatic interactions and charge-dipole interactions are anisotropic or “patchy” in nature due to the heterogeneous distribution of charged sites on the protein surface [27-29] and may be amplified at high protein concentration. [30] These interactions may result in the formation of protein networks or ordered structures, leading to viscous gels. [4,26,31,32] In some cases electrostatic interactions can be modulated by the addition of a chaotropic salt to screen the protein charge and minimize attractive electric dipole interactions. [4,11,32-35] Furthermore, hydrophobic [36] salts have been hypothesized to adsorb on hydrophobic sites and screen hydrophobic interactions, resulting in large viscosity reduction. Recently, we introduced a concept of adding a high concentration of a nonionic crowding agent, for example the disaccharide trehalose [37,38] to provide a depletion attraction force [39,40] to osmotically compress proteins to modulate their interactions to attempt to lower the viscosity. According to a free energy model, the compression generates nanoclusters of primary colloidal charged spheres which may weaken some of the attractive interactions, whereby the protein may adopt a more stable conformation through a self-crowding mechanism. [41,42\ Highly concentrated (250 mg/mL) mAb dispersions were prepared by reconstitution of lyophilized protein powder in buffer and by centrifugation filtration to concentrate the protein. To our knowledge, protein dispersions with elevated concentrations of co-solutes have not been formed by TFF.

Herein we utilize high concentrations of histidine and trehalose as co-solutes to form stable high-concentration mAb dispersions by TFF with viscosities as low as 70 and 40 cP at 280 and 255 mg/ml, respectively. To place these results in perspective, experiments were performed in a buffer with low co-solute concentrations, however, the solutions gelled and were ˜10 times more viscous. The viscosity reduction from the high levels of co-solutes is hypothesized to occur via disruption of aggregate-forming attractive interactions between mAb molecules, particularly by the concentrated charged histidine molecules, and by depletion attraction, which also alters the protein interactions. The design of the low viscosity dispersions and the choice of the hollow fiber geometry are shown to provide for low axial pressure drops, resulting in a relatively uniform and small wall shear stress and more uniform TMP. Because these factors mitigate membrane fouling from concentration polarization and protein aggregation and gelation, it became possible to achieve relatively low losses in membrane flux (low permeation resistance) even at the high protein concentrations. During concentration the decay in the transmembrane flux was found to depend more strongly on the dispersion viscosity rather than the mAb concentration. A secondary objective is to add a third process (in addition to lyophilization dilution and centrifugal filtration) for forming concentrated protein dispersions with high co-solute levels and low viscosities. [37,38] The mAb dispersions were diluted and studied by SEC before and after extended storage at 4° C. to show that the formation of irreversible aggregates was negligible.

Theory

In the Ross-Minton equation (Eq. 4-1), [η] represents the inherent contribution of the mAb to the dispersion viscosity and is equal to the effective specific volume of the mAb molecule at infinite dilution. [4,43] The inherent viscosity ηᵢₙₕ normalizes the dispersion viscosity η for the mAb concentration c and solvent viscosity η₀, as shown in Eq. 4-2, [44] allowing for a comparison of the effectiveness of the different buffer formulations and co-solutes at reducing the viscosity.

$${η}_{inh} = \frac{ln ( \frac{η}{{η}_{0}} )}{c} \qquad ( 4 \text{-} 2 )$$

The intrinsic viscosity is formally defined as ηᵢₙₕ at infinite dilution [44] (Eq. 4-3), but can also be determined from a two-parameter non-linear regression of the viscosity curve to the Ross-Minton equation, [43] where [η] and k/v are simultaneously fit.

[η]=lim_{c→0}ηᵢₙₕ (4-3)

The transmembrane flux J is proportional to the TMP and inversely proportional to the solvent viscosity η₀ and the total permeation resistance Rₜ (Eq. 4-4):

$$J = \frac{TMP}{{η}_{0} {R}_{t}} \qquad ( 4 \text{-} 4 )$$

The global TMP is given by Eq. 4-5,

$$TMP = \frac{( {P}_{i} + {P}_{0} )}{2} - {P}_{p} = {P}_{o} + \frac{Δ P}{2} - {P}_{p} , \qquad ( 4 \text{-} 5 )$$

where Pᵢ and Pₒ are the filter inlet and outlet pressures, respectively, and Pₚ is the permeate pressure. Eq. 4-5 suggests that the initial strategy for reducing the TMP to the optimal level requires decreasing Pₒ by opening a control valve regulating flow out of the filter module. Once the valve is fully opened and Pₒ is no longer adjustable, the TMP may be further reduced by decreasing the flow rate to decrease ΔP or restricting the permeate line to increase Pₚ, both of which tend to reduce the permeation rate.

The permeation resistance Rₜ in Eq. 4-4 is influenced by membrane fouling via pore blocking (Rₚₒᵣₑ), cake formation on the membrane (R_{cake}) and the buildup of a concentration polarization gel layer near the membrane (Rₚₒₗ). Additionally, each filter membrane inherently provides resistance (Rₘ) depending on the pore size and membrane thickness. The total resistance accounts for the combination of fouling mechanisms:

Rₜ=Rₘ+Rₚₒᵣₑ+R_{cake}Rₚₒₗ (4-6)

The mAb molecules experience a shear stress τ_{w} at the membrane wall during convective flow:

$${τ}_{w} = \frac{{d}_{H} Δ P}{4 L} , \qquad ( 4 \text{-} 7 )$$

where d_{H} is the hydrodynamic diameter of the flow channel, L is the axial length of the filter module and ΔP is the axial pressure drop.

The axial ΔP is the controlling parameter for tuning the TMP and τ_{w}, as is apparent in Eq. 4-5 and Eq. 4-7. A smaller ΔP allows for a lower TMP for a fixed Pₒ and Pₚ (such as when Pₒ is no longer adjustable, or when it is undesirable to restrict the permeate line further). The small ΔP also creates a more uniform local TMP along the length of the filter module, avoiding regions of ultrahigh TMP that are more prone to gelation. The hollow fiber geometry lends itself to smaller and more uniform TMP and wall shear stress profiles, as the lack of stagnation zones in the flow path leads to a small ΔP and a uniform pressure gradient ΔP/L. The straight flow path of the hollow fiber geometry also facilitates the mitigation of membrane fouling while maintaining gentle flow conditions to prevent protein aggregation.

Concentration polarization is particularly challenging at ultrahigh protein concentration as is apparent in the expression for the transmembrane flux J (Eq. 4-8):

$$J = \frac{{D}_{v}}{δ} ln ( \frac{{c}_{w}}{{c}_{b}} ) = {k}_{c} ln ( \frac{{c}_{w}}{{c}_{b}} ) \qquad ( 4 \text{-} 8 )$$

where k_{c} is the mass transfer coefficient, c_{w} is the protein concentration at the membrane wall (generally assumed to be a constant and equal to the solubility/gel limit c_{g}) [45] and c_{b} is the bulk protein concentration. Once c_{w} reaches the gel concentration c_{g}, further increases in the bulk concentration lead to an increase in the gel layer thickness [45] and a decrease in the concentration gradient driving force for back-diffusion of the polarized protein molecules. For ultrahigh c_{b} values, strategies to improve the membrane flux would be to increase k_{c} via lower viscosities or increasing c_{g} by designing the protein buffer formulation and excipients to improve solubility. Alternatively, it may be desired to keep c_{w} below the gel point by modifying the membrane charge to repel like-charged protein and lower fouling. [46,47]

Materials and Methods

Materials.

The IgG1 mAb used in this study was provided by Abbvie as a concentrated solution at 130 mg/mL in a proprietary buffer containing 10 mM histidine, and 0.1% Tween-80 at pH 5.8 (referred to as the “freezing buffer”). The mAb solution was aliquoted into 5 ml sub-samples into 5-mL cryogenic vials (Corning Incorporated, Corning, N.Y.) and frozen using a dry ice-ethanol freezing mixture for extended storage at −80° C. α-trehalose dihydrate (Tre) was purchased from Ferro Pfanstiehl Laboratories Inc., Waukegan, Ill. All other chemicals (L-histidine (His), imidazole (Im), citric acid monohydrate (CitrA), hydrochloric acid (HCl), o-phosphoric acid (PhosA)) were purchased from Thermo Fisher Scientific, Fair Lawn, N.J. Disposable 0.22 μm polyethersulfone (PES) bottle top and 13 mm syringe sterile filters were obtained from Celltreat Scientific Products, Shirley, Mass. (product codes 229717 and 229746). Disposable 50 kDa PES MidiKros hollow fiber filter modules with 0.5 mm ID (36 fibers and area 115 cm², part no. D02-E050-05-N) or 1.0 mm ID (12 fibers and area 75 cm², part no. D02-E050-10-N from Spectrum Labs, Rancho Dominguez, Calif.) were utilized for TFF. Amicon Ultra-15 Ultracel-30K centrifugal filters were purchased from Merck Millipore Ltd. Ireland.

Diafiltration and Ultrafiltration to 250 mg/mL by TFF.

Buffers were sterile filtered with the Celltreat bottle top PES filters and then degassed under vacuum for 30 minutes. The frozen mAb stock (25 mL in 5 vials) was thawed in a 4° C. water bath and diluted with an equal volume (25 mL) of the buffer, resulting in a mAb concentration of 65 mg/mL. The diluted mAb solution was gently mixed in a 50-mL centrifuge tube, which served as the retentate reservoir during the TFF experiments. In two of the experiments, the buffer exchange was done at a lower concentration of 20 mg/mL to attempt to form less turbid solutions. In one of these experiments, the diluted mAb solution was additionally sterile-filtered using the 0.22 μm bottle top filters and degassed prior to buffer exchange.

The diluted mAb solution was buffer-exchanged at constant concentration with permeation of six diavolumes (150 mL) of the desired formulation buffer with a KrosFlo Research II TFF system (Spectrum Labs, Rancho Dominguez, Calif.) operated in a constant-volume mode. The TMP was maintained at 0.80 bar using a KrosFlo automatic backpressure valve (Spectrum Labs, Rancho Dominguez, Calif.), which regulated the pressure by constricting the retentate line. The feed cross-flow rate was set at 100 mL/min for the 1.0 mm ID filter module and 220 mL/min for the 0.5 mm ID filter module, corresponding to calculated wall shear rates inside each fiber of 1415 and 8300 s⁻¹ respectively. The retentate reservoir was gently mixed throughout the process using a Vari Mix Platform Rocker (Thermo Fisher Scientific, Fair Lawn, N.J.) set at the maximum speed and rocking angle along with periodic hand swirling. The buffer-exchanged solution was recovered and sterile filtered, then stored overnight at 4° C., after which it was concentrated to ˜250 mg/mL the following day using the 1.0 mm ID filter module.

When the 1.0 mm ID filter module was used during diafiltration, the filter membrane was cleaned between diafiltration and ultrafiltration to recover membrane performance. When the 0.5 mm module was used for the diafiltration step, the ensuing ultrafiltration was performed with a new 1.0 mm ID module. The new filters were first flushed with 200 mL of DI water without recirculating or permeating any material in order to flush out residual glycerin impurities. Both the new and used membranes were then cleaned prior to filtration by permeating 4 mL DI water/cm² membrane area, circulating 0.5N NaOH for 30 minutes to decompose adsorbed protein, followed by permeating another 4 mL DI water/cm² membrane area to flush the residual NaOH and degraded protein out of the membrane pores. A normalized water permeability (NWP) test was then conducted to check the performance of the regenerated membrane prior to ultrafiltration. The NWP was also determined for the fouled membrane after both diafiltration and ultrafiltration.

The buffer-exchanged mAb solution was concentrated from 65 mg/mL to 250 mg/mL by ultrafiltration using the 1.0 mm ID hollow fiber module. The feed cross-flow rate was initially kept at 100 mL/min and the TMP was maintained at 0.80 bar. When the mAb solution became too viscous (>150 cP, the TMP could no longer be regulated. At this point, the TMP was maintained at 0.80 bar by partially restricting the permeate line (to increase the permeate back-pressure) and decreasing the feed cross-flow rate (to decrease the pressure drop across the filter module). The retentate was kept well-mixed using the rocker set at the maximum speed. The rocker angle for the Vari Mix Platform Rocker was gradually reduced over time as the fluid level in the retentate reservoir decreased in order to keep the feed and retentate return lines submerged. The retentate concentration at any given time was estimated based on the mass of the permeate which was monitored in real time assuming no permeation of mAb through the membrane, and was subsequently verified by UV-Vis spectroscopy via 150 μL aliquots. The final 250 mg/mL dispersion was recovered and mixed by gentle pipetting to homogenize the dispersion prior to characterization by DLS, UV-Vis spectroscopy and syringe capillary viscometry.

Low co-solute controls were also prepared by centrifugation filtration to place the results from TFF in perspective.

TFF Membrane Fouling Characterization.

The extent of membrane fouling at the end of ultrafiltration was assessed by measuring the percent normalized water permeability (NWP) reduction. The DI water flux of the cleaned membrane was measured at room temperature (20° C.) and a TMP of 0.25 bar, 0.50 bar and 0.8 bar before the start of ultrafiltration. The pre-ultrafiltration NWP in liters per square meter membrane area (LMH) per bar of applied TMP was calculated from the slope of the water flux vs TMP curve. The NWP of the fouled membrane after ultrafiltration was measured in the same way after the membrane was first washed by recirculating 200 mL DI at a cross-flow flux of 800 LMH for 10 minutes to recover adsorbed protein.

mAb Concentration Determination and Turbidity by UV-Vis Spectroscopy.

The mAb concentration in the intermediate and final dispersions was measured in duplicate at 500× dilution using a Cary 60 UV-Vis spectrophotometer (Agilent Technologies, Santa Clara, Calif.). The concentrated mAb dispersion was diluted in 50 mM pH 6.4 sodium phosphate buffer, and the absorbance at 280 nm was measured in a QS quartz cuvette (Hellma GmbH and Co, Mullheim, Germany) with a path length of 1 cm. The absorbance was converted to a mass concentration via the Beer-Lambert law assuming a mAb absorption coefficient of 1.42 mL/mg/cm. The turbidity of the buffer-exchanged 20 mg/mL mAb stock as well as the concentrated mAb dispersion was measured undiluted using a Cary 60 UV-Vis spectrophotometer (Agilent Technologies, Santa Clara, Calif.).

Syringe Capillary Viscometry.

The viscosity of the mAb dispersion was measured in triplicate using a custom syringe capillary viscometer as described previously. [37] Briefly, a 25G (ID=0.1 mm; L=1.5″) Precision Glide needle (Becton Dickinson & Co.) was attached to a 1.0 mL LUER-LOK™ syringe (Becton Dickinson & Co.). A 100-125 μL aliquot of the dispersion was placed in a 0.1 mL conical vial (catalog no. 03-341-15; Wheaton, Millville, N.J.). The dispersion was drawn into the syringe, and the flow rate of the dispersion through the needle was determined by ImageJ image analysis [48] of a video taken with a Kodak Z812 IS zoom digital camera. The change in the dispersion column height was tracked over time and correlated to the sample volume based on a previously-established calibration curve. [49] The flow rate was correlated to a viscosity based on the Hagen-Poiseuille equation, using a calibration curve determined from a set of viscosity standard solutions. The calibration curve was validated against an AR 2000ex cone-and-plate rheometer (TA Instruments, New Castle, Del.) over a shear range of 25-4000 s⁻¹ as described in earlier work. [49] The viscosities measured by the capillary viscometer correspond to an estimated shear rate of 400-1000 s⁻¹, assuming a Newtonian shear response to fluid flow (n=1 power-law dependence between viscosity and shear rate). [50]

Size Exclusion Chromatography.

For characterization of irreversible non-covalent aggregate levels in the final dispersions, the mAb dispersion was diluted to 2 mg/mL in the mobile phase (92 mM NaHPO₄, 211 mM Na₂SO₄, pH 7). The standard solution was prepared by diluting freshly-thawed mAb monomer stock (as provided in the original freezing buffer at 130 mg/mL) in the mobile phase. The diluted samples were sterile filtered through a 0.22 μm PES syringe filter (Celltreat Scientific Products, Shirley, Mass.). Duplicate 10 μL injections of each sample were analyzed with a Waters Breeze HPLC (Waters Corporation, Milford, Mass.) equipped with a Tosoh Biosciences TSKgel3000SW_{XL} column (Tosoh Corporation, Tokyo, Japan). The eluate was monitored by the UV absorbance at 214 nm and 280 nm. The aggregate level was quantified by determining the percent monomer by peak area for the diluted samples.

Sample Storage Stability Study.

Small 150 μL aliquots of the final mAb dispersion were stored in capped 0.1 mL conical vials (Wheaton, Millville, N.J.) sealed with Parafilm. The sealed vials were stored in a −40° C. freezer, in a refrigerator at 4° C. and in a water bath at 37° C. for up to 8 weeks. To minimize evaporative losses, the samples stored at 37° C. were additionally sealed with three additional alternating layers of aluminum foil and Parafilm. Individual aliquots were removed from storage at 4 days, 1 week, 2 weeks, 4 weeks and 8 weeks and characterized by UV-Vis spectroscopy, syringe capillary viscometry and DLS measurements at a scattering angle of 90°. The samples were discarded after characterization, so that a fresh aliquot was used for each time point.

Diafiltration and Ultrafiltration to 250 mg/ml by Centrifugation Filtration to Form Low Co-Solute Controls

Buffers were sterile filtered with the Celltreat bottle top PES filters and then degassed under vacuum for 30 minutes. Centrifugal filters were washed before use by permeating 2 mL DI water through the membrane at 4500 rcf using a Centrifuge 5810R (Eppendorf, Hamburg, Germany). The frozen mAb stock (130 mg/mL) was thawed in a 4° C. water bath, and 1.3 mL of the thawed stock was diluted to 13.5 mg/mL with 10.7 mL of the filtered and degassed buffer. The diluted mAb solution was then buffer exchanged by discontinuous diafiltration until the original buffer constituted less than 1% by volume of the total solution volume. Briefly, the mAb solution was filtered at 4500 rcf for 15 minutes using a Millipore Amicon centrifugal filter with a capacity of 12 mL and a regenerated cellulose membrane with a molecular weight cutoff of 30 kDa. The mAb solution was concentrated to a volume of ˜2 mL and a concentration of 80 mg/mL. Additional buffer was added to dilute the retained mAb solution to a volume of 12 mL and a concentration of 13 mg/mL, reducing the volume fraction of the initial buffer to 1.8% (10.8% of 2 mL out of 12 mL). The mAb solution was then concentrated again by centrifugal filtration to a volume of ˜2 mL, and the process was repeated 2-3 more times until the permeate volume was approximately 40 mL, corresponding to less than 1% by volume of the initial buffer in the final solution, assuming ideal mixing. The mAb solution was then concentrated to 80 mg/mL (˜2 mL) and transferred to a new Amicon centrifugal filter (with the same capacity and MWCO) for concentration to 250 mg/mL.

The tare weights of the individual filter assembly components (filter, permeate tube and retentate tube) of the second set of Amicon centrifugal filters were recorded before the start of concentration to 250 mg/mL mAb. The mass of mAb loaded into the second set of centrifugal filters was determined from the mass and exact concentration of the buffer-exchanged mAb solution (˜80 mg/mL). The desired retentate volume at 250 mg/mL and corresponding mass of the solution plus filter assembly was determined assuming a recovery of 80% of the mAb by mass. The filter assembly was then centrifuged at 4500 rcf in 10-15 minutes increments for 60-70 minutes. The mass of the retentate/filter assembly was monitored between increments, and filtration was stopped when the combined retentate/filter assembly mass reached the desired mass as calculated earlier. The concentrated mAb dispersion was then transferred to 0.1 mL conical V-vial (catalog no. 03-341-15; Wheaton, Millville, N.J.) for further characterization.

Turbidity Measurements by UV-Vis Spectroscopy.

The turbidity was measured as the absorbance of the mAb solution at 350 nm, where the chromophores in the mAb do not absorb. The absorbance was normalized for the path length, yielding a dimensional turbidity in cm⁻¹. Dilute mAb solutions (≦65 mg/mL) were loaded into a 1 cm path length QS quartz cuvette (Hellma GmbH and Co, Mullheim, Germany), while the concentrated (>150 mg/mL) dispersions were loaded into a 0.2 cm path length cuvette (Beckman Coulter, Indianapolis, Ind.). Turbidity measurements were made on both the unfiltered solutions as well as solutions sterile filtered with 0.22 μm PES bottle top (20 mg/mL stock) and 13-mm syringe filters (>150 mg/mL dispersion). Before sterile filtration, the syringe filters were washed by passing 100 μL DI water through the filter membrane to remove residual impurities in the membrane. Due to the ˜100 μL hold-up volume of the syringe filter, around 100 μL of DI was entrained in the filter, causing some dilution of the 250 mg/mL mAb dispersion during sterile filtration by syringe filters.

Dynamic Light Scattering of Concentrated mAb Dispersions.

An effective average diffusion coefficient of the species in the mAb dispersion was measured by dynamic light scattering (DLS) on a Brookhaven BI-9000AT (Brookhaven Instruments, Holtsville, N.Y.) at detector angles of 150° and 173° using a 632.8 nm laser. Additional samples were analyzed by DLS on a Brookhaven ZetaPALS at an angle of 90° using a 660 nm laser. The effective diffusion coefficient Dᵥ was determined from the decay time of the DLS autocorrelation function (ACF), which was fit using the CONTIN algorithm or quadratic cumulant (as specified in the relevant tables). The theoretical protein monomer diffusion coefficient D_{v,o} at infinite dilution in the same formulation buffer was calculated from the Stokes-Einstein equation using the solvent viscosity and assuming a hydrodynamic radius R_{H} of 5.5 nm. All reported D/D₀ values are the average of three replicate DLS measurements of 2 minutes each. Samples were not filtered prior to loading into the DLS cuvette (path length=0.2 cm; Beckman Coulter, Indianapolis, Ind.).

The diffusion coefficient of the high co-solute mAb dispersions will now be shown to be lower than the expected value for the monomer in the same buffer, indicating a change in the mAb interaction length scale. The autocorrelation function (ACF) from the DLS measurements was fit to a single average effective diffusion coefficient using the CONTIN algorithm, where the decay time for the first exponential decay was converted to a diffusion coefficient assuming Stokes-Einstein diffusion of the mAb through buffer. η was set equal to no in the Stokes-Einstein equation for the first decay, assuming that the first decay corresponds to a short-enough time scale that diffusing protein molecules do not encounter neighboring protein molecules. [53] In light of the possible polydisperse nature of the mAb dispersion, as well as the complexities involved in fitting the ACF, [83] the fitted Dᵥ only represents an effective average diffusivity. The theoretical diffusion coefficient of the monomer D_{v,0} in the given buffer was calculated from the Stokes-Einstein equation (Eq. 4-9) using η₀ for η and an average hydrodynamic radius R_{H} of 5.5 nm typical for an IgG mAb.

$${D}_{v} = \frac{{k}_{B} T}{6 π η {R}_{H}} \qquad ( 4 \text{-} 9 )$$

The ratio of the measured dispersion diffusion coefficient to the theoretical monomer diffusivity Dᵥ/D_{v,0} was 0.3-0.4 for the high-co-solute dispersions at 250 mg/mL mAb. In contrast, the values for the low co-solute dispersions were 0.8-0.85 at 250 mg/mL, closer to the value of 1.0 for a purely monomeric solution. Slightly smaller Dᵥ/D_{v,0} values of 0.5-0.7 were obtained for the low co-solute dispersions made by centrifugation filtration. However, the resolution of DLS is too low to be able to distinguish diffusion coefficient differences under a factor of 3-4 [84] and thus to discriminate between protein monomer and aggregates up to about 40 nm. The values of Dᵥ/D_{v,0} less than unity for the high co-solute dispersions may suggest restricted molecular motion relative to monomer diffusing through the solvent, or may indicate the diffusing species encounter other protein and a η greater than the solvent η. With the exception of the DI water mAb dispersion, the Dᵥ/D_{v,0} ratio also slowly decreased with increasing mAb concentration between 50 and ˜150 mg/mL before reaching a plateau. The gradual decrease in Dᵥ/D_{v,0} with mAb concentration could indicate the continuous growth of protein entities larger than monomer or an increase in attractive interparticle interactions leading to restricted motion at higher levels of depletion attraction (crowding). However, it is unclear how to choose a medium viscosity for converting the diffusion coefficient to a hydrodynamic diameter, given the diffusing species may contact multiple protein molecules during the long diffusion times of 100 μs. In contrast, self-diffusion of protein through solvent may be measured by neutron spin echo where the correlation time is only 50 ns. [32]

Results.

Low Viscosity 250 mg/mL mAb Dispersions with High-Co-Solute Formulations by TFF.

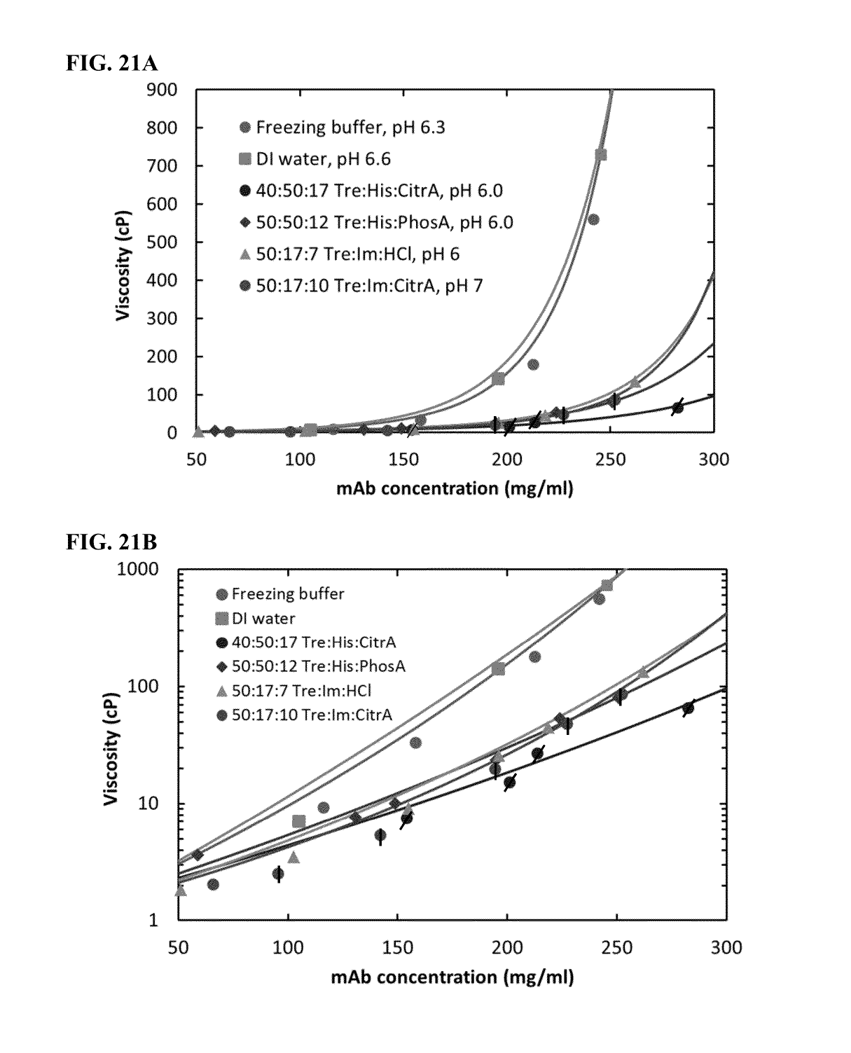

We begin by describing controls with low co-solute concentrations. As seen in Table 4-1, these controls manufactured by centrifugation filtration buffered with 10 mM histidine (freezing buffer) or 30 mM histidine were extremely viscous, exceeding 200 cP at 240 mg/mL and 400 cP at 270 mg/mL, corresponding to a high ηᵢₙₕ of ˜0.021 mL/mg. The addition of 150 mM NaCl lowered ηᵢₙₕ to 0.018 mL/mg, likely due to screening of the electrostatic interactions as observed for certain mAbs previously. [4,11,32] The same low-cosolute dispersion in the freezing buffer was significantly more viscous when manufactured by TFF, as seen in Table 4-2, increasing to 700 cP with a ηᵢₙₕ of 0.027 mL/mg at 250 mg/mL mAb. Similarly high viscosities were obtained for a cosolute-free control mAb dispersion in DI water (Table 4-2, FIGS. 21A-21B). The higher 11 in TFF likely resulted from prolonged shear (˜7 hours) resulting in more aggregation, compared to the relatively short (˜2 hours) and lower-shear CF process.

TABLE 4-1
Viscosity and filtration times for low co-solute control dispersions made by centrifugation filtration.
The diafiltration (DF) time corresponds to buffer exchange into 6 diavolumes and subsequent concentration to
~80 mg/mL mAb. The ultrafiltration (UF) time corresponds to concentration from ~80 mg/mL to 250 mg/mL.
No diafiltration step was required for the controls in the freezing buffer.

| mAb | | Exc 1 | | Exc 2 | | Exc 3 | | | | | | DF | UF |
|---|---|---|---|---|---|---|---|---|---|---|---|---|---|
| conc | | conc | | conc | | conc | | η | η₀ | η_{inch} | Turbidity | time | time |
| (mg/ml) | Exc 1 | (mM) | Exc 2 | (mM) | Exc 3 | (mM) | pH | (cP) | (cP) | (mL/mg) | (cm⁻¹) | (min) | (min) |
| 281 ± 8.7 | Freezing buffer (replicate 1) | | | | | | 6.1 | 424 | 1.1 | 0.0212 | 0.376 | 0 | 75 |
| 271 ± 0.5 | Freezing buffer (replicate 2) | | | | | | 6.1 | 460 | 1.1 | 0.0223 | 0.388 | 0 | 75 |
| 238 ± 3.3 | His | 30 | HCl | 19 | — | — | 6.2 | 188 | 1.0 | 0.0220 | 0.388 | 70 | 54 |
| 230 ± 2.8 | His | 30 | HCl | 19 | NaCl | 150 | 6.0 | 61 | 1.0 | 0.0179 | 0.581 | 85 | 69 |

TABLE 4-2
Viscosity and filtration times of low and high co-solute dispersions made by tangential flow filtration.
The inherent viscosities were evaluated at the final mAb concentration (~250 mg/mL). Alternating shaded rows
indicate different formulations. The 280 mg/mL replicate of the 40:50:17 mg/mL Tre:His:CitrA dispersion
corresponds to Replicate 4 and the 241 mg/mL replicate corresponds to Replicate 1.

| mAb | | | | | | | | | | | | | | | | |
|---|---|---|---|---|---|---|---|---|---|---|---|---|---|---|---|---|
| conc ± | | | | | | | | | | | | | | | DF | UF |
| stdev | Tre conc | | | Base conc | | | Acid conc | | | η | η₀ | η_{inch} | [η] | | time | time |
| (mg/ml) | mg/ml | mM | Base | mg/ml | mM | Acid | mg/ml | mM | pH | (cP) | (cP) | (ml/mg) | (ml/mg) | k/v | (min) | (min) |
| 242 ± 2.5 | Freezing buffer | | | | | | | | 6.4 | 559 | 1.1 | 0.0258 | 0.0163 | 0.0948 | 0^{a} | 99 |
| 245 ± 3.6 | DI water | | | | | | | | 6.7 | 729 | 0.98 | 0.0269 | 0.0206 | 0.0468 | 150 | 96 |
| 282 ± 3.7^{b,c} | 40 | 105 | His | 50 | 320 | CitrA | 17 | 80 | 6 | 66 | 1.27 | 0.0140 | 0.0121 | 0.0406 | 68^{d} | 373^{e} |
| 241 ± 0.3^{b} | 40 | 105 | His | 50 | 320 | CitrA | 17 | 80 | 6 | 50 | 1.27 | 0.0153 | 0.0062 | 0.4030 | 253 | 136 |
| 251 ± 18.5 | 50 | 130 | His | 50 | 320 | PhosA | 12 | 120 | 6 | 80 | 1.25 | 0.0166 | 0.0135 | 0.0556 | 270 | 100 |
| 252 ± 0.5 | 50 | 130 | Im | 17 | 250 | CitrA | 10 | 50 | 7 | 86 | 1.16 | 0.0171 | 0.0111 | 0.1318 | 284 | 145 |
| 262 ± 4.5 | 50 | 130 | Im | 17 | 250 | HCl | 7 | 190 | 6.4 | 133 | 1.11 | 0.0182 | 0.0119 | 0.112 | 243 | 147 |

^{a}No diafiltration step was needed for the freezing buffer dispersion
^{b}The mAb solution was buffer exchanged at 20 mg/ml instead of 60 mg/ml
^{c}The mAb solution was additionally degassed before diafiltration and ultrafiltration (optimized TFF procedure)
^{d}Buffer exchanged using a 115 cm² area membrane (all other rows buffer exchanged with 75 cm² membrane), leading to faster permeation rate
^{e}Larger sample volume (250 mL at start of ultrafiltration) compared to other rows (50 mL at start of ultrafiltration)

We now show that the addition of high concentrations of histidine (50 mg/mL=320 mM) together with trehalose (40 to 50 mg/mL) to the mAb dispersion buffer formulation resulted in a marked reduction of the dispersion η, particularly at the highest mAb concentrations. Remarkably, the high co-solute dispersions manufactured by TFF were significantly less viscous than the low co-solute controls made by both TFF and CF. The high-cosolute dispersions remained below 50 cP with phosphoric acid (PhosA) up to 220 mg/mL and with citric acid (CitrA) all the way to ˜260 mg/mL, corresponding to ηᵢₙₕ of 0.017 and 0.014 mL/mg respectively (FIGS. 21A-21B, Table 4-2). On a semi-log plot (FIG. 21B), the slope gives a qualitative measure of [η] of the mAb in the given buffer, as described by the Ross-Minton model. Two-parameter fits of the viscosity profiles to the Ross-Minton model showed significant differences between the low and high co-solute dispersions in the strength of the protein-protein interactions, as indicated by [η] and ηᵢₙₕ. A larger [η] (and therefore slope in FIG. 21B) suggests stronger attractive protein-protein interactions that lead to a greater η increase with concentration. The smaller slope of the η curve for the high co-solute dispersions suggests that the addition of high concentrations of His and Tre somehow weakened the mAb-mAb interactions. Although both His-containing high co-solute formulations resulted in low η at 250 mg/mL, the dispersion with CitrA as the counteracid was slightly less viscous, as shown in FIGS. 21A-21B. Similarly low viscosities were also observed when a structurally-analogous base, imidazole (Im), was used instead of His as the crowding agent, as shown in FIGS. 21A-21B.

Large differences in the regressed parameters [η] and k/v (Table 4-2) were observed between the low and high co-solute dispersions. With the exception of the 240 mg/mL replicate of the Tre:His:CitrA dispersion, which was fit only from 3 data points, the regressed [η] values for all of the high-co-solute dispersions were 0.014 mL/mg or lower, whereas they were much higher at ˜0.02 mL/mg for the low co-solute dispersions. The corresponding fitted k/v was comparable between the freezing buffer (10 mM His) and the two histidine high co-solute dispersions with a value of ˜0.06, whereas the DI water dispersion had a much smaller k/v of 0.026 and the two imidazole dispersions had a higher k/v of 0.09 and 0.13. The combined parameter k/v accounts for the effect of both self-crowding (k=1/Φₘₐₓ [43] where Φₘₐₓ is the maximum packing fraction) as well as the particle shape (where a sphere corresponds to v=2.5 and elongated shapes have v>2.5) on η. [43,51] Since k/v is fit as a single parameter, the two effects cannot be easily deconvoluted from a single value for k/v, making quantitative interpretation difficult. [4] However, the crowding factor k is not expected to change significantly between the low and high co-solute dispersions at the same mAb concentration/volume fraction. The modestly higher fitted values of k/v for the high co-solute dispersions therefore suggest a small decrease in v approaching a more spherical mAb molecule. A decrease in v suggests the adoption of a more compact and folded conformation which would be beneficial for stability and lower viscosities. A smaller value of Dᵥ/D_{v,0} below unity was also observed for the high co-solute dispersions as well as the low co-solute dispersions with the extremely high protein concentrations. Although the mechanism of protein diffusion at high concentrations is highly complex, these results may indicate an influence of depletion attraction on the configuration of reversible protein aggregates or clusters [37,38,52-56].

A gradual increase in ηᵢₙₕ with concentration was also observed for all of the mAb dispersions. However, the high co-solute dispersions consistently had lower ηᵢₙₕ than the low co-solute dispersions. The nearly 2-fold lower ηᵢₙₕ of the high co-solute dispersions at ˜250 mg/mL (0.011-0.0135 mL/mg) compared to the low co-solute dispersions (˜0.027 mL/mg) as shown in Table 4-2 indicate a significant reduction in attractive protein interactions, leading to the lower viscosities. A decrease in shear stress-induced aggregation, such as by manufacture of the mAb dispersions by CF instead of TFF, also significantly reduced ηᵢₙₕ, as can be seen from comparing ηᵢₙₕ of the freezing buffer dispersion manufactured by TFF (0.027 mL/mg, Table 4-2) and CF (0.021-0.022 mL/mg, Table 4-1). While mAb exposure to significant shear stress is inherent to the TFF process, the use of high concentrations of interacting co-solutes to reduce η by modifying protein-protein interactions leads to smaller wall shear stresses, as will be discussed in a later section. The addition of interacting co-solutes therefore reduces ηᵢₙₕ for TFF by simultaneously weakening attractive interactions and minimizing the extent of shear-induced aggregation during filtration.

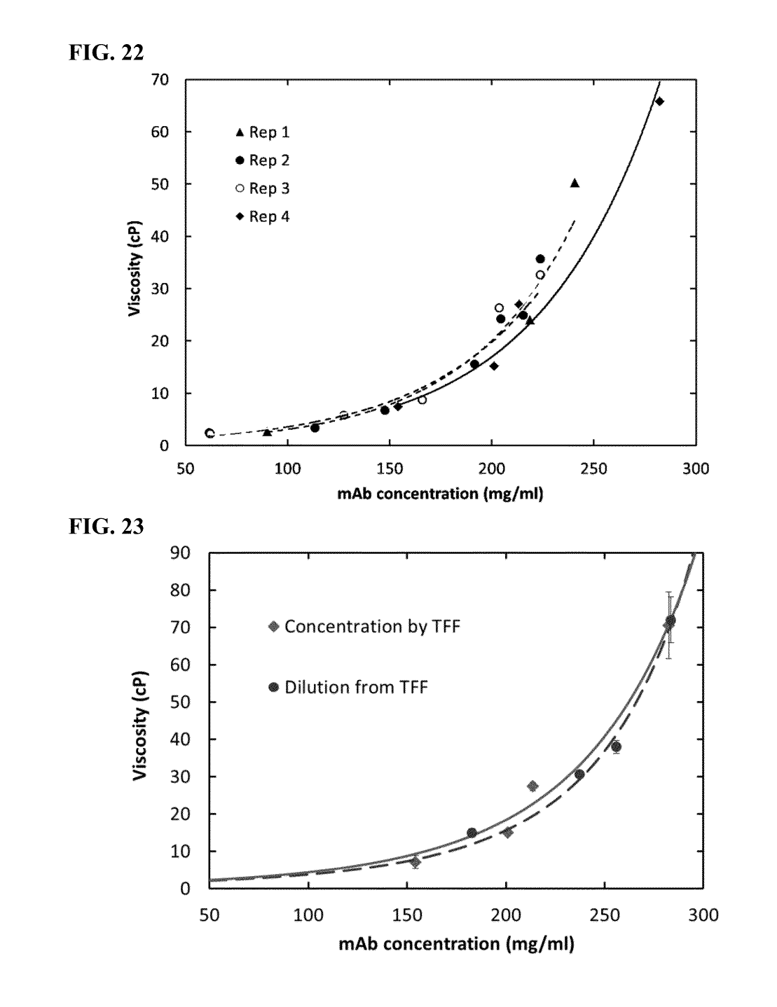

Low viscosities were obtained by ultrafiltration with four different TFF diafiltration conditions for the high co-solute Tre:His:CitrA formulation as shown in Table 4-3 and FIG. 22. In Replicates 2 and 3, buffer exchange was done at 65 mg/mL mAb with a shear rate of 1405 (1.0 mm fiber) or 8200 s⁻¹ (0.5 mm fiber), as indicated in Table 4-3. The much higher water base flux for the latter may explain the higher permeate flux. However the turbidity increased with the higher shear rate suggesting greater protein aggregation. These results suggest that the greater aggregation at the higher shear rate outweighed the potentially greater hydrodynamic removal of protein from the membrane surface. When the mAb solution was buffer exchanged at only 20 mg/mL in Replicates 1 and 4, the opposite trend was observed where the turbidity decreased with shear rate, suggesting a smaller role of protein aggregation. In this experiment, the degassing and filtration prior to buffer exchange may have lowered the propensity towards aggregation. The lower turbidity and aggregation were key factors that led to the lowest viscosity for Replicate 4 as shown in FIG. 22. There was also little hysteresis in the viscosity of Replicate 4 upon dilution, as seen in FIG. 23, again suggesting very little gelation and irreversible aggregation during ultrafiltration, consistent with the lower turbidity. [8] The greater interparticle separation at low mAb concentration may partially mitigate the risk of aggregation. In addition, the sterile filtration of the buffer-exchanged stock before ultrafiltration may not completely remove soluble low-MW aggregates, which could grow into larger aggregates during ultrafiltration and contribute to higher viscosities. In summary, the lowest viscosity and turbidity in Replicate 4 was achieved by various factors: degassing and sterile filtration before buffer exchange, and buffer exchange with a low protein concentration.

TABLE 4-3
Different TFF buffer exchange conditions for Replicates 1-4 of
the 40:50:17 mg/ml Tre:His:CitrA dispersion and resulting turbidity. The 0.5 mm
fiber ID modules have a higher water flux than the 1.0 mm fiber ID
modules. The turbidity of the buffer-exchanged solution was measured as the
absorbance at 350 nm for a 1 cm path length.

| | mAb | | | | | | | |
|---|---|---|---|---|---|---|---|---|
| | conc | | | | | | | Turbidity |
| | during | | Filter | Hollow | Membrane | | Turbidity | post- |
| | buffer | | shear | fiber | mAb | Permeate | pre-sterile | sterile |
| | exchange | TMP | rate | ID | loading | flux | filtration | filtration |
| Sample | (mg/ml) | (bar) | (s⁻¹) | (mm) | (g/m²) | (LMH) | (cm⁻¹) | (cm⁻¹) |
| Replicate 1 | 22 | 0.8 | 1405 | 1.0 | 390 | 9 | 0.389 | — |
| Replicate 4 | 20 | 0.8 | 8200 | 0.5 | 407 | 20 | 0.140 | 0.061 |
| Replicate 2 | 65 | 0.8 | 1405 | 1.0 | 390 | 5 | 0.962 | 0.153 |
| Replicate 3 | 66 | 0.8 | 8250 | 0.5 | 254 | 9 | 1.780 | 0.150 |

TFF Membrane Flux Decay at High Concentration.

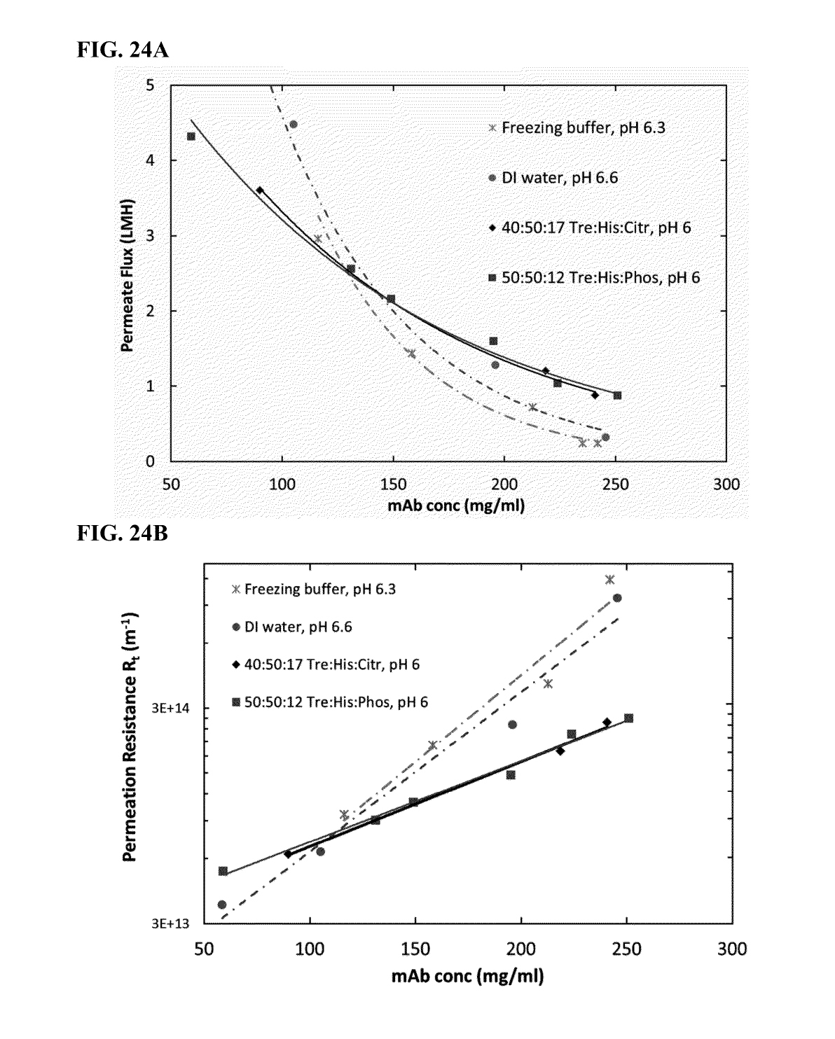

The high co-solute dispersions will now be shown to exhibit significantly slower membrane flux decay with increasing mAb concentration than the low-co-solute dispersions. The instantaneous flux was measured at the operating TMP of 0.80 bar. The permeate flux J of the low co-solute and His high co-solute dispersions is plotted against the mAb concentration in FIG. 24A. The low co-solute dispersions started with a higher membrane flux than the high co-solute dispersions, likely due to the greater no. However, a crossover in the flux between the low and high co-solute formulations was seen at 130 mg/mL due to the slower flux decay of the high co-solute systems. As a result, ultrafiltration of the high co-solute dispersions ended at a membrane flux three times higher than that of the low co-solute dispersions at 250 mg/mL. Overall, the high co-solute dispersions exhibited a 5-fold flux reduction from 4 LMH (L/m² membrane area/hr) to 0.8 LMH over a concentration range of 60 to 250 mg/mL, whereas the low co-solute dispersions saw a 32-fold flux reduction from 8 to 0.25 LMH over the same concentration range. The membrane flux was also normalized for differences in η₀ and plotted as the overall permeation resistance Rₜ. The resistance was found to increase exponentially with the mAb concentration, as seen in FIG. 24B. The crossover in Rₜ occurred at an earlier mAb concentration of ˜110 mg/mL than the flux crossover, and Rₜ was found to increase at a significantly slower rate for the high co-solute dispersions.

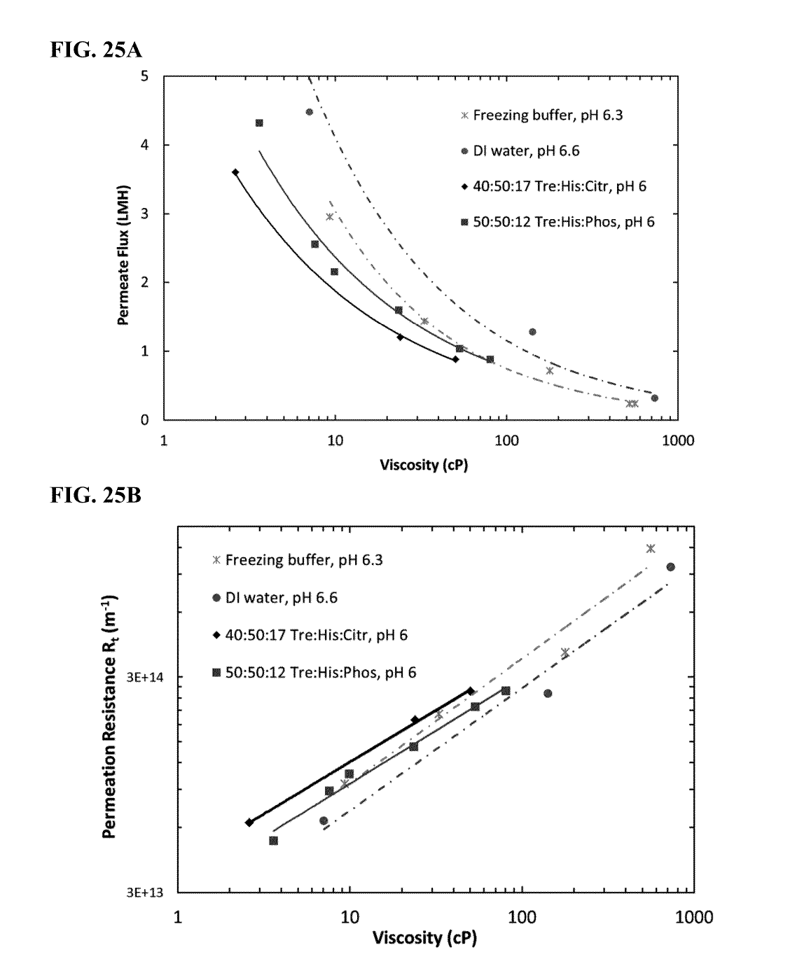

For a plot of the membrane flux as a function of the dispersion viscosity instead of the mAb concentration, the low and high co-solute dispersions were found to give similar fluxes, as shown in FIG. 25A. The low co-solute dispersions again start at a higher flux at low viscosity, but no flux crossover is observed. Instead, the low and high co-solute flux profiles seem to converge at high viscosities. When the flux was normalized for η₀ via the permeation resistance, the resistance curves seemingly collapsed into a relatively universal curve, as shown in FIG. 25B. This result seems somewhat surprising since a given dispersion η corresponded to greatly different mAb concentrations for the low and high co-solute dispersions as discussed in detail below.

The membrane fouling between the low and high co-solute dispersions was examined in terms of the post-ultrafiltration reduction in the membrane normalized water permeability (NWP). The fouled membrane was first flushed with DI water for ten minutes to wash off reversibly adsorbed mAb and remove the concentration polarization gel layer before the NWP measurement. The NWP reduction due to fouling by irreversibly adsorbed mAb was comparable between the low and high co-solute dispersions at ˜60%, as seen in Table 4-4, with the exception of the Tre:His:CitrA dispersions. The Tre:His:CitrA formulation, which gave the lowest viscosities among the dispersions, consistently exhibited a smaller NWP reduction of ˜30%. However, no clear trend was found between the mass of mAb lost to irreversible adsorption on the membrane (which varied between 100 and 300 mg mAb, based on the mass of mAb unaccounted for after recovery of the mAb in the dispersion and filter wash water) and the NWP reduction. Since the NWP only accounts for irreversibly adsorbed mAb, the NWP reduction could be interpreted as the contribution of membrane fouling by pore blocking to flux decay. The comparable NWP reductions suggest that the TFF filter membrane underwent the same degree of fouling by pore blocking under both low and high co-solute conditions despite large differences in the flux decline of the two types of dispersions. The deposition of a significant mass of mAb (100-300 mg) on the membrane and inside the pores likely caused a large initial drop in the membrane flux from the pure water membrane flux during the first few seconds of filtration. Due to the likely near-instantaneous nature of the drop in membrane flux from pore blocking, the initial drop was not captured by the flux curves (FIG. 5 and FIG. 6). The initial flux drop occurs at low concentration when the low and high co-solute dispersions have comparable viscosities, when concentration polarization is negligible. The initial drop in membrane flux is therefore expected to depend only on the extent of pore blocking and will be similar in magnitude between the two types of dispersions. After the initial flux drop, however, the low and high co-solute dispersions exhibit large differences in the flux decay with mAb concentration, which cannot be explained through membrane fouling by pore blocking. As such, pore blocking likely has a much smaller effect on the membrane flux decay at high concentration compared to concentration polarization.

TABLE 4-4
Membrane fouling and protein adsorption after ultrafiltration by
TFF. The membrane normalized water permeability (NWP) was measured at
room temperature (20° C.). The mass of mAb recovered during the filter
wash was determined from the mAb concentration in the wash water after
recirculation. The small calculated negative mAb mass losses for some
formulations may be due to minor experimental error in concentration
measurements.

| | | | | | | | | |
|---|---|---|---|---|---|---|---|---|
| | | | | mAb | | | | |
| | | Post- | | recovered | mAb | mAb | mAb | % mAb |
| | Pre-UF | UF | % NWP | from | recovered | in UF | lost to | lost to |
| | NWP | NWP | reduction | filter | in | feed | adsorpt. | irrevers. |
| | (LMH/ | (LMH/ | post- | wash | dispersion | stock | on filter | adsorption |
| Formulation | bar) | bar) | fouling | (mg) | (mg) | (mg) | (mg) | on filter |

Low co-solute dispersions

| | | | | | | | | |
|---|---|---|---|---|---|---|---|---|
| Freezing | 298 | 80 | 62 | 758 | 1565 | 2624 | 301 | 11.5 |
| buffer | | | | | | | | |
| DI water | 213 | 78 | 63 | 775 | 1480 | 2232 | −23 | −1.0 |

High co-solute dispersions

| | | | | | | | | |
|---|---|---|---|---|---|---|---|---|
| 40:50:17 mg/ml | 232 | 166 | 29 | 674 | 3080 | 4121 | 367 | 8.9 |
| Tre:His:CitrA | | | | | | | | |
| (Replicate | | | | | | | | |
| 4) | | | | | | | | |
| 40:50:17 mg/ml | 221 | 141 | 36 | 522 | 1445 | 2131 | 164 | 7.7 |
| Tre:His:CitrA | | | | | | | | |
| (Replicate | | | | | | | | |
| 1) | | | | | | | | |
| 50:50:12 mg/ml | 289 | 107 | 63 | 600 | 1745 | 2327 | −18 | −0.8 |
| Tre:His:PhosA | | | | | | | | |
| 50:17:7 mg/ml | 277 | 138 | 50 | 707 | 1503 | 2284 | 74 | 3.2 |
| Tre:Im:HCl | | | | | | | | |
| 50:17:10 mg/ml | 267 | 57 | 79 | 573 | 1769 | 2606 | 264 | 10.1 |
| Tre:Im:CitrA | | | | | | | | |

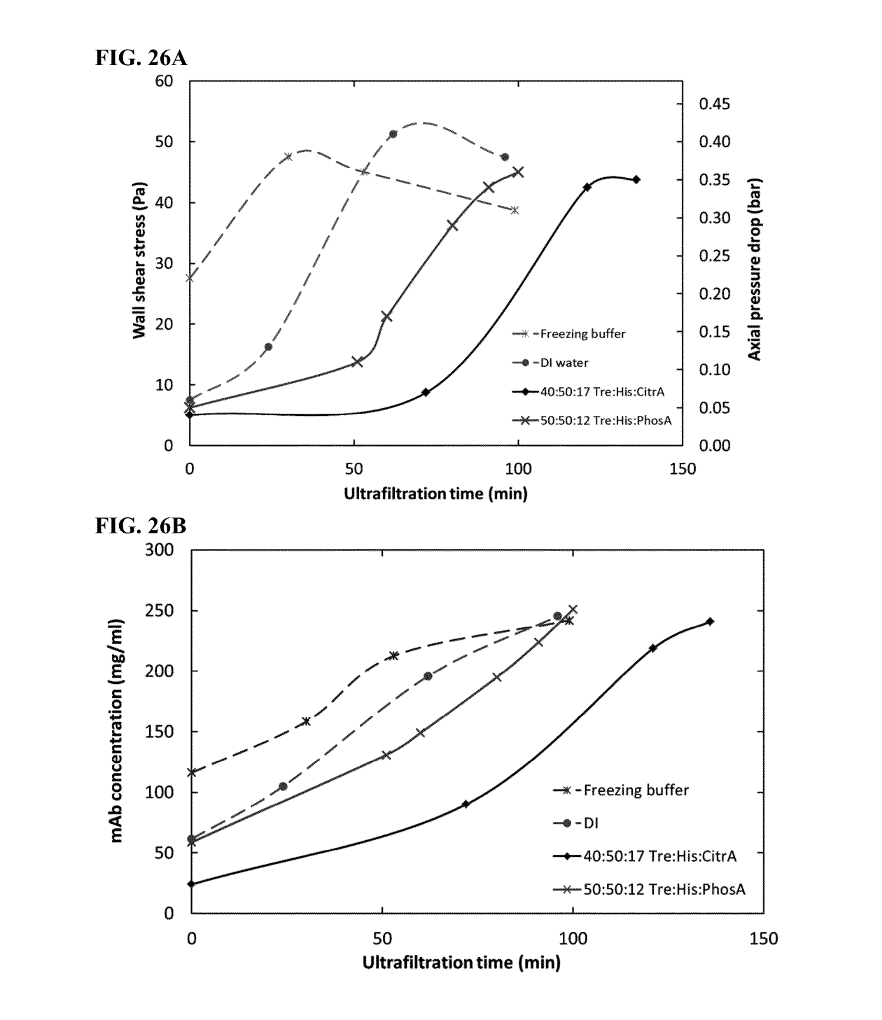

The apparent universal dependence of the membrane flux on η (FIG. 26) and negligible differences in pore blocking suggests that the observed flux decay was dominated by the concentration polarization effect, which in turn was mitigated by the low η of the high co-solute dispersions. Early membrane fouling models suggest that the membrane resistance evolves in two stages—initial development of concentration polarization, followed by pore blocking at a constant level of polarization. [14] A later model from Kanani and Ghosh proposes that concentration polarization only has a small impact on resistance compared to pore blocking and cake formation, but the associated study was done at a low TMP below 0.25 bar and at a dilute protein concentration of 0.32 mg/ml. [16] In the current study, significant differences were seen in the membrane flux and permeation resistance between the low and high co-solute dispersions at concentration greater than 150 mg/mL (FIG. 25). However, no significant difference was found in the NWP reduction and protein adsorption after removal of the polarization effect (Table 4-4). This discrepancy suggests that the differences in flux decay were due to concentration polarization, contrary to the observations of Kanani and in better agreement with the earlier model. The effect of concentration polarization on the flux can be reduced by increasing the mass transfer coefficient k_{c} of the protein (Eq. 4-1) for enhanced removal of the foulant from the membrane surface. [6,57] The mass transfer coefficient is proportional to the mAb diffusivity Dᵥ in the bulk solution, which in turn is inversely proportional to the dispersion η. The strong agreement between the low and high co-solute dispersions in the flux decay as a function of η, despite differences in mAb concentration for different formulations at the same η, suggests that k_{c} has a larger effect than the concentration gradient ln(c_{wall}/c_{bulk}) on the concentration polarization transmembrane flux (Eq. 4-1). As a result, the low η of the high co-solute dispersions facilitated the retention of high transmembrane flux, even at the ultrahigh mAb concentrations of 250-280 mg/mL.

We now demonstrate that the low-n, high co-solute dispersions resulted in better regulation of the membrane fouling from improved control and reduction of τ_{w} during ultrafiltration. The τ_{w} was found to increase slowly from 5 to 15 Pa during the first 60 minutes of ultrafiltration, as seen in FIG. 26A, corresponding to a concentration range of 60 to 100 mg/mL as seen in FIG. 26B. The τ_{w} then rapidly rose to a maximum value of 40 Pa before leveling off. In contrast, the τ_{w} of the low-co-solute dispersions quickly increased to a maximum of 50 Pa at 150-160 mg/mL before decreasing with further increases in the mAb concentration. The unexpected decrease in τ_{w} arose from the decrease in the axial pressure drop, as seen in FIG. 26A. The pressure drop is expected to increase with the dispersion η and feed cross-flow rate, as described by the Hagen-Poiseuille equation for flow through a cylindrical geometry. Since η increases monotonically with the mAb concentration, the reduced pressure drop and τ_{w} must have arisen from a decrease in the cross-flow rate. The settings on the TFF pump were unchanged throughout the ultrafiltration experiment, so the theoretical cross-flow rate remained constant. Instead, the actual cross-flow rate decreased when the ultraviscous low co-solute dispersions exceeded the capacity of the TFF pump to accurately regulate the dispersion flow-rate. At lower mAb concentration/η, the τ_{w} profiles of the low co-solute dispersions resembled those of the high co-solute dispersions. The shear and concentration profiles of the Im systems were observed.

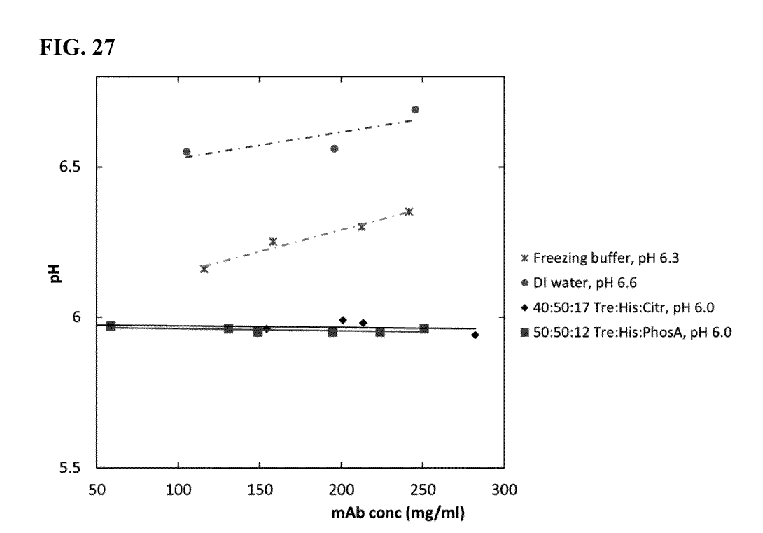

The hollow fiber filter geometry will now be shown to enable a relatively small τ_{w} for maintaining a high cross-flow and transmembrane flux while avoiding protein aggregation. A low τω of 5-50 Pa was maintained throughout ultrafiltration to 250 mg/mL with the hollow fiber module (FIG. 27B), and was sufficient to prevent rapid flux decline from significant membrane fouling. In contrast, in TFF studies from Rosenberg and Ahrer with flat sheet cassettes, a higher τ_{w} of 200-400 Pa was applied to minimize fouling and maximize membrane flux at mAb concentration greater than 50 mg/mL. [6,8] At the same time, the aggregation population was found to increase with the τ_{w} between 100 and 300 Pa. [6,8] Due to large pressure drops concentrated at the 90° bends in the serpentine flow path of the flat sheet geometry, maintaining a high TMP may have required application of a large wall shear. The non-uniformity of the pressure drop across the module could additionally create local areas of high TMP and τ_{w} which may increase the potential for aggregation. In order to minimize protein aggregation from the applied shear and avoid regions of ultrahigh shear, Rosenberg developed a method for ‘programming’ the TMP throughout ultrafiltration to maintain the TMP at its optimal value (lowest TMP needed to achieve maximum flux) at any given mAb concentration. The optimal TMP was found to decrease with the mAb concentration, and consequently the TMP was programmed from 1.25 bar to 0.85 bar with increasing concentration above 50 mg/mL. [6] Concurrently, the cross-flow flux was adjusted between 290 and 450 LMH over the concentration range 5-90 mg/mL to maintain the appropriate low TMP and τ_{w}. [6] At high mAb concentrations, it becomes difficult to maintain the relatively low optimal TMP given the large ΔP as shown in Eq. 4-5.

In our case, the low viscosities of our concentrated dispersions as well as our choice of the hollow fiber geometry resulted in a small axial pressure drop and τ_{w}, which appeared to lower the risk of protein aggregation. With a small pressure drop and straight flow path of the hollow fiber geometry, the TMP and τ_{w} profiles were relatively uniform along the length of the filter module. TMP optimization was therefore not as important for preventing protein aggregation during ultrafiltration. We were able to maintain a low AP at our chosen TMP of 0.8 bar without adjusting the permeate pressure while maintaining a constant high cross-flow flux of 800 LMH. The low τ_{w} made possible by the low η and hollow fiber geometry therefore allowed us to maintain a high membrane flux while avoiding significant aggregation and gelation of the mAb at high concentration.

Turbidity of High Concentration mAb Dispersion.

The dispersion turbidity was measured after 3 months of storage at 4° C., during which mild evaporative losses caused an increase in the mAb concentration to 300 mg/mL. For a path length of 0.2 cm, the absorbance at 300 mg/mL at 350 nm was 0.165, corresponding to a turbidity of 0.826 cm⁻¹ and a concentration-normalized turbidity of 0.0027 mL*mg⁻¹cm⁻¹, as reported in Table 4-5. In an earlier study, the absorbance decreased by 0.05-0.15 units for three different mAbs at 90 mg/mL (from 0.72 to 0.57; 0.52 to 0.41; 0.45 to 0.39 respectively) [6] upon optimizing a program for multiple TMPs. Assuming a path length of 1 cm, the corresponding turbidity decreased from 0.5-0.7 cm⁻¹ to 0.4-0.5 cm⁻¹ for a mAb concentration of 90 mg/mL The comparable (if slightly higher) turbidity between the ultraconcentrated mAb dispersion in this study and the mAb solutions obtained in the previous study under optimized conditions is surprising, given the significantly higher mAb concentration in the current study (300 mg/mL versus 90 mg/mL). Extrapolation of the apparent linear increase in turbidity with concentration in the earlier study [6] suggests that the turbidity would have increased to 1.5 cm⁻¹ at 250 mg/mL, even under the optimized TMP program. The low relative turbidity at 300 mg/mL in the current study likely resulted from the low dispersion η, axial ΔP and τ_{w}, which minimized the tendency for protein aggregation. The low levels of aggregation is also favorable for reducing pore blocking and cake formation. [58].

TABLE 4-5
Turbidity (0.2 cm path length), turbidity/concentration (c) and
viscosity before and after syringe sterile filtration of Replicate 4 of the
40:50:17 mg/ml Tre:His:CitrA dispersion.

| | mAb | | | | Turbidity at | |
|---|---|---|---|---|---|---|
| Sample | conc | η | ηᵢₙₕ | Absorbance | 350 nm | Turbidity/c |
| ID | (mg/mL) | (cP) | (mL/mg) | at 350 nm | (cm⁻¹) | (mL * mg⁻¹cm⁻¹) |
| Before | 307 | 138 | 0.0153 | 0.1652 | 0.8260 | 0.0027 |
| filtration | | | | | | |
| After | 223 | 22 | 0.0128 | 0.0743 | 0.3715 | 0.0017 |
| filtration | | | | | | |

Sterile filtration of the 300 mg/mL mAb dispersion resulted in a decrease in concentration to 223 mg/mL, due to the presence of entrained water in the washed syringe filter membrane. The resulting absorbance at 350 nm was 0.0743, corresponding to a turbidity of 0.372 cm⁻¹ [1] and a concentration-normalized turbidity of 0.0017 mL*mg⁻¹cm⁻¹ (Table 4-5). The two-fold reduction in turbidity lowered ηᵢₙₕ significantly from 0.015 to 0.013 mL/mg. A normalized effective Dᵥ from DLS increased from 0.36 to 0.43 after sterile filtration, which may suggest removal of larger aggregates, consistent with the smaller contribution to the ACF at long times.

mAb Stability in the Ultraconcentrated Dispersions.

The high co-solute dispersions underwent a negligible pH shift during ultrafiltration compared to the low co-solute dispersions, lending to improved mAb stability at high concentration due to the minimal change in the solvent environment of the mAb molecules. The pH of the high co-solute dispersions remained essentially constant during ultrafiltration from 60 to 250 mg/mL as shown in FIG. 27. Additionally, intermediate pH measurements during ultrafiltration of a representative high co-solute dispersion (50:17:10 mg/mL Tre:Im:CitrA) revealed a negligible difference in the pH of the retentate and permeate streams (shift of ˜0.02 units), both of which remained constant at 7.0. The matching, unchanging pH in the permeate and retentate streams over the entire mAb concentration range suggests that the charged buffer species (His, Im, counteracids) did not partition significantly across the membrane during filtration. [9] The dispersion pH was in good agreement with the initial pH of the protein-free buffer, although a small shift of <0.1 units was observed for the buffers prepared at pH 6.5 and 7 due to the self-buffering capacity of the mAb, [59] which was found to buffer at pH 6.0. In contrast, a noticeable pH shift of 0.2 units from 60 to 240 mg/mL was observed during filtration of the low co-solute dispersions, suggesting the pardoning of the buffer species across the membrane due to the Donnan effect [9] or electrical double-layer volume exclusion. [10,60] The pH shift presents a change in the mAb solvent environment and could lead to changes in the mAb stability. Size exclusion chromatography (SEC) of the final 250 mg/mL dispersions after 2-4 weeks of storage at 4° C. show that the high co-solute dispersions were stable against irreversible aggregation and had less than 1% aggregates upon dilution to 2 mg/mL, as seen in Table 4-6. The only exception was the Tre:Im:CitrA dispersion, which showed 2.6% aggregates when measured after 7 months of storage at 4° C. While SEC measurements of the low co-solute dispersions also showed less than 1% aggregates at 2 mg/mL, a significant amount of visible gel-like aggregates were observed during dilution of the concentrated low-co-solute dispersions. The visible aggregates were filtered out during sample preparation and were therefore not detected by SEC, but their presence during dilution indicates the instability of the mAb in the low co-solute formulations at high concentration.

TABLE 4-6
Stability of mAb dispersions by SEC. Samples were analyzed
by SEC within two weeks of when the samples were manufactured
except for the Trehalose-Imidazole-Citric Acid dispersion,
which was measured seven months after the dispersion was
manufactured. All samples were stored at 4° C. between
manufacture and SEC measurements.

| | |
|---|---|
| Formulation | % Monomer |

Concentrated dispersion from TFF

| | |
|---|---|
| mAb standard (130 mg/ml) | 99.74 |
| 40:50:17 mg/ml Tre:His:CitrA, pH 6 (280 mg/ml) | 99.47 |
| 40:50:17 mg/ml Tre:His:CitrA, pH 6 (240 mg/ml) | 99.90 |
| 50:50:12 mg/ml Tre:His:PhosA, pH 6 (250 mg/ml) | 99.93 |
| 50:17:7 mg/ml Tre:Im:HCl, pH 6.3 (260 mg/ml) | 99.02 |
| 50:17:10 mg/ml Tre:Im:CitrA, pH 7 (250 mg/ml) | 97.38^{a} |
| Freezing buffer (235 mg/ml) | 99.46^{b} |
| DI water (245 mg/ml) | 99.39^{b} |

Dilution of 40:50:17 mg/ml Tre:His:CitrA dispersion (original 280 mg/m)

| | |
|---|---|
| 256 mg/ml dilution | 99.51 |
| 237 mg/ml dilution | 99.50 |
| 183 mg/ml dilution | 99.52 |

^{a}after 7 months storage at 4° C.
^{b}Visible ~1 mm gel-like clumps (aggregates) observed upon dilution to 2 mg/mL in mobile phase; removed by sterile filtration prior to SEC analysis

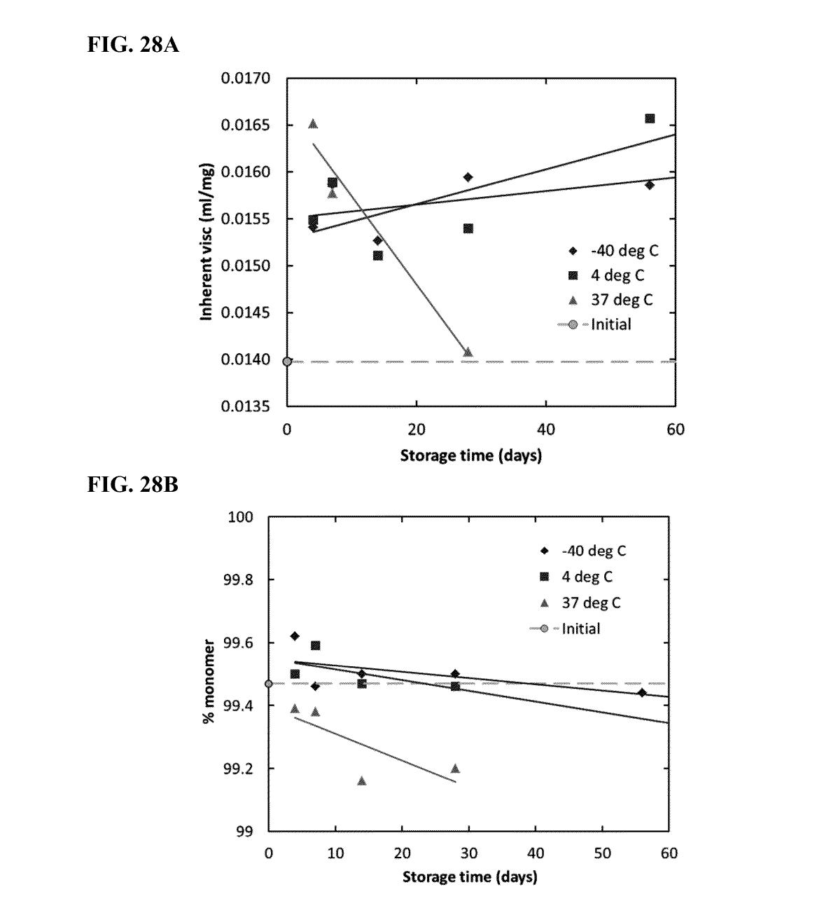

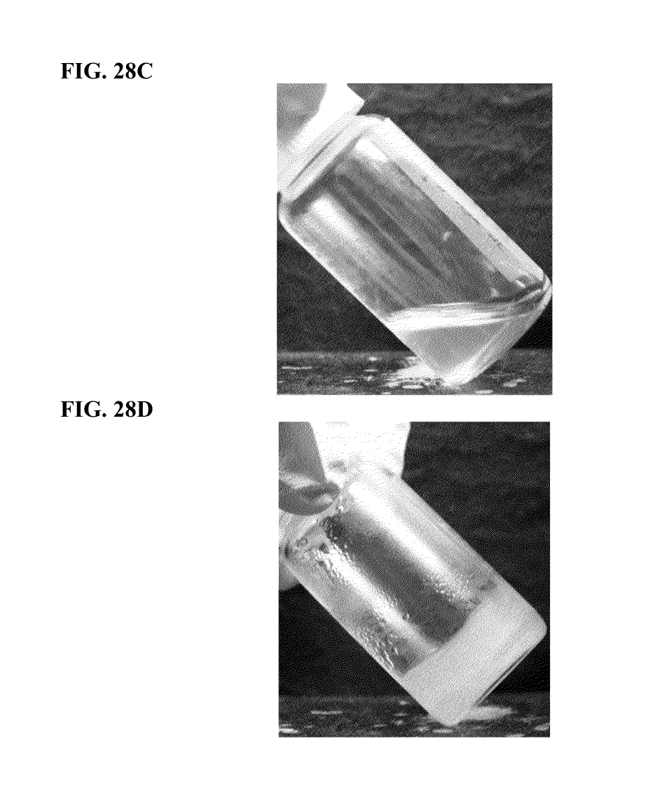

To test storage stability, Replicate 4 of the Tre:His:CitrA dispersion was stored over 8 weeks at −40° C., 4° C. and 37° C. See Table 4-7. Due to evaporative losses during storage, especially at 37° C., the concentrations increased in some cases. Despite the high mAb concentrations, η remained below 100 cP for a majority of the stored samples and changes in Dᵥ/Dv_{,o} were negligible. The ηᵢₙₕ of the samples stored at −40° C. and 4° C. was relatively constant as seen in FIG. 28A, whereas ηᵢₙₕ at 37° C. decreased from 0.0164 mL/mg at 4 days to 0.014 mL/mg at 28 days. At each temperature, the monomer content was >99.2% even after 8 weeks (FIG. 28B). Visible aggregates were not present in the samples. If they were present, they would have been removed by sterile filtration during sample preparation for SEC. The concentrated high co-solute dispersions remained clear, even after up to four months of storage at 4° C. as shown in FIG. 28C. In contrast, the low co-solute dispersions became cloudy and formed a rigid, opaque gel within minutes of exposure to the 4° C. environment, as shown in FIG. 28D. Although the gelled dispersion returns to a liquid state upon being warmed to room temperature, irreversible phase separation was evident. It is likely that the concentrated co-solute capped various charged patches and hydrophobic sites on the protein to inhibit the formation of aggregates and gelation during storage.

TABLE 4-7
Viscosity and SEC stability of Replicate 4 of the 40:50:17 mg/mL
Tre:His:CitrA dispersion after up to 4-weeks of storage at −40° C., 4° C.
and 37° C. No 56-day sample at 37° C. was available due to evaporative
losses which rendered the sample too concentrated and viscous even for
concentration measurements.

| | Storage | | | | | % mon- |
|---|---|---|---|---|---|---|
| Time | temp | mAb conc | η | ηᵢₙₕ | | omer |
| (days) | (° C.) | (mg/ml) | (cP) | (ml/mg) | pH | by SEC |
| 4 | −40 | 278 ± 5.3 | 92 ± 3.7 | 0.0154 | 5.99 | 99.62 |
| 4 | 4 | 274 ± 6.6 | 88 ± 5.5 | 0.0155 | 5.96 | 99.50 |
| 4 | 37 | 330 ± 2.7 | 297 ± 3.9 | 0.0165 | 5.95 | 99.39 |
| 7 | −40 | 263 ± 2.7 | 82 ± 3.7 | 0.0159 | 5.98 | 99.46 |
| 7 | 4 | 266 ± 10.0 | 88 ± 2.3 | 0.0159 | 5.97 | 99.59 |
| 7 | 37 | 260 ± 10.9 | 77 ± 0.6 | 0.0158 | 5.97 | 99.38 |
| 14 | −40 | 287 ± 5.1 | 101 ± 0.9 | 0.0153 | 5.95 | 99.50 |
| 14 | 4 | 288 ± 3.4 | 98 ± 5.7 | 0.0151 | 5.98 | 99.47 |
| 14 | 37 | 454 ± 34.8 | —^{b} | —^{b} | —^{b} | 99.16 |
| 28 | −40 | 291 ± 3.5 | 132 ± 0.8 | 0.0159 | 5.92 | 99.50 |
| 28 | 4 | 302 ± 6.4 | 133 ± 1.9 | 0.0154 | 5.94 | 99.46 |
| 28 | 37 | 299 ± 4.2 | 86 ± 2.4 | 0.0141 | 5.99 | 99.20 |
| 56 | −40 | 279 ± 1.4 | 106 ± 2.6 | 0.0159 | 5.95 | 99.44 |
| 56 | 4 | 272 ± 0.5 | 114 ± 6.1 | 0.0166 | 5.96 | —^{c} |

^{a}Remaining sample volume after viscometry and concentration measurements was insufficient for DLS measurements.
^{b}Sample became too concentrated and viscous to characterize by viscosity and DLS.
^{c}Remaining sample volume after viscometry and concentration measurements was insufficient for SEC measurements.

### Discussion

At concentrations of 250 mg/ml (˜20% volume fraction) when the spacing between proteins is on the order of the molecular diameter, [61] the relationship between viscosity and protein interactions is highly complex and not very well understood. Even with a range of techniques including SANS [31,32,56,62-64] SAXS, [32] SLS, [2] DLS, [52,53,65] simulation, [66-69] and rheological studies [4,11,26, 27,30,64,70-74] and combinations thereof, it is highly challenging to determine the interactions and structure of protein dispersions. From a rheological point of view, [η] and ηᵢₙₕ of the concentrated mAb offer useful insight into the extent of attractive protein interactions that influence the dispersion viscosity. The marked reduction in [η] and ηᵢₙₕ, up to two-fold for the highly concentrated dispersions as seen in Table 4-2, by addition of high concentrations of histidine and imidazole will now be shown to be consistent with a weakening of attractive protein interactions.

An early study by Liu et al. examined two ‘well-behaved’ low-viscosity mAbs with weak intermolecular interactions and an ‘ill-behaved’ viscous mAb with strong interactions, as seen in a sharp deviation from the Ross-Minton model. [4] In each case the value for the “well behaved” mAbs of 0.0063 mL/mg was used for [η]. In contrast, when both [η] and k/v were fit by non-linear regression, [η] was found to be approximately 0.009 mL/mg for both well-behaved antibodies and 0.03 mL/mg for the ill-behaved antibody. [4] Other antibody studies found a [η] of 0.0084 mL/mg for an inherently non-viscous human IgG antibody [75] and a [η] of 0.0086 mL/mg for a polyclonal antibody. [43] Polyclonal antibodies are known to exhibit weaker interactions and lower viscosities in some cases than monoclonal antibodies. [11] On the basis of these studies, the reduction in the mAb intrinsic viscosity from ˜0.02 to as low as 0.0058 mL/mg in Table 4-2 with the addition of the co-solutes His/Im with Tre suggests that the attractive protein interactions became much weaker.

The weakening of the attractive protein interactions with high levels of His or Im may be anticipated to result from disruption of the local anisotropic electrostatic, [31,32] dipolar and hydrophobic [36] interactions through capping of charged and hydrophobic patches on the protein surface. The viscosity of concentrated antibody solutions has been shown to increase with the formation of small low-density clusters of interacting [26,51] or physically entangled [76] mAb molecules. In some cases, weakening of the attractive anisotropic electrostatic interactions through charge screening by the addition of salts was observed in neutron spin echo measurements of self-diffusion at high protein concentrations, where the diffusion coefficient increased. With this decrease in the intermolecular protein interactions, the viscosity decreased. [27,30,32] However, the addition of 100-300 mM of salts such as NaCl has produced a wide variety of protein-specific effects on the viscosity; salts such as NaCl may reduce, p4,11,32] increase [77] or not change the viscosity. [36] The complex effects of salt on the viscosity depend partially on the salt concentration via salting in versus salting out effects, [77] but also on the local heterogeneous distribution of charged sites on the protein surface. [28,29] The anisotropy in the charge distribution of interacting patches on neighboring mAb molecules will influence the mAb viscosity beyond what may be expected merely from the net protein charge. [28] In addition, a recent study has found a stronger dependence of η on hydrophobic interactions rather than electrostatic interactions at high mAb concentration. [36] These interactions were weakened upon the addition of hydrophobic salts, resulting in significant reduction in the viscosity.

We chose to study the basic co-solute histidine (side chain pKa=6.04) since it is a common pharmaceutical buffer component, positively charged below pH 6 and slightly hydrophobic with a Hopp-Woods hydrophobicity index of −0.5, where a negative index indicates an apolar residue in a protein sequence. [78] Below pH 6 the cationic imidazole (pKa=7.05) functional group on His is the primary site that interacts with mAbs. Thus, either His or Im will modulate both net and local electrostatic and dipolar interactions as well as hydrophobic interactions. What sets His and Im apart from inorganic salts and many co-solutes are their strong and selective binding affinities for the arginine (Arg) and His residues on the protein surface. [79] Imidazole has been found to competitively bind to His residues at pH 7.5 or lower, leading to enhanced protein solubility by preventing His-His binding between neighboring proteins and subsequent precipitation. [80] The imidazole group on His may be expected to show similar behavior. By selectively binding to the His residues, which contributes significantly to attractive protein interactions and aggregation, [80] His and Im may mitigate the anisotropic, site-specific protein interactions and lower the viscosity. Additionally, molecular dynamics simulations have suggested that positively-charged His is capable of pairing with both neutral and positively-charged His and Arg residues on the protein surface despite the Coulombic repulsion between like-charged pairs. [79] The ability of His to form like-charged pairs with itself and Arg is thought to be due to stacking of the conjugated imidazole and guanidyl side chains respectively. [79] As a result, His is capable of neutralizing negatively-charged residues through Coulombic attraction while blocking neutral and positively-charged His and Arg residues through stacking of the conjugated side chains, leading to its efficacy in screening both the anisotropic attractive electrostatic and hydrophobic interactions. His and Im therefore weaken these interactions more efficiently than inorganic salts such as NaCl that do not undergo specific binding with the protein. The ability of His and Im at a high concentration of 50 and 17 mg/mL respectively (300 and 250 mM) to modulate the anisotropic electrostatic, dipolar and hydrophobic interactions may be expected to inhibit aggregate formation through competitive binding for the protein interaction sites. The prevention of aggregation, as suggested by lower turbidities as observed in Table 4-3, may contribute to the much lower viscosities than in the low co-solute systems. The very high concentrations of these bases along with Tre further perturbs all of the electrostatic, dipolar and hydrophobic interactions by the high degree of depletion attraction. In addition the preferential exclusion of Tre from the surface favors folding and enhanced protein stability. [81,82]

Conclusion.

For highly concentrated 250 to 280 mg/mL mAb dispersions formed by TFF, the addition of high concentrations of histidine or imidazole resulted in low viscosities from 40-80 cP, compared to values >300 cP under low co-solute conditions. Furthermore, the transmembrane flux was two to three-fold higher at 250 mg/mL mAb and depended strongly on the mAb dispersion viscosity, and at a given viscosity, was independent of the mAb concentration. As a consequence of the low viscosities and hollow fiber membrane geometry, the axial JP and wall shear stress were reduced to 0.4 bar and 50 Pa, respectively, nearly two-fold lower than typical values applied to flat sheet cassette TFF filters. The reduced JP and wall shear likely lowered protein aggregation as indicated by the turbidity. Furthermore, increased mass transfer rates at lower viscosities reduce concentration polarization and membrane fouling. The lowest viscosity of 70 cP at 282 mg/mL (40 cP upon dilution to 255 mg/mL) was obtained when the solution was degassed and filtered prior to buffer exchange and the exchange was done for a low mAb concentration of 20 mg/ml. The addition of high concentrations of His and Im is thought to reduce the viscosity through disruption of attractive local anisoptropic electrostatic, dipolar and hydrophobic interactions between mAb molecules via passivation of charged and hydrophobic sites on the protein surface. Furthermore, the osmotic compression generated by the high co-solute concentrations modifies all of these interactions and may possibly reconfigure the morphology of mAb reversible aggregates on the basis of the DLS results. According to SEC, the addition of either of the two basic co-solutes was not found to adversely affect the stability of the mAb against irreversible aggregation. The high co-solute formulations were found to stabilize the mAb during extended storage of the 250 mg/mL dispersions at −40° C., 4° C. and 37° C. and prevent gelation and precipitation even at concentrations of 300 mg/mL mAb and higher. In contrast, the onset of gelation and precipitation was almost immediate for the concentrated low co-solute dispersions upon exposure to the 4° C. storage environment.

### Example 5

### Further Studies on Viscosities of mAb1 with Co-Solutes

Introduction.

Hundreds of monoclonal antibodies (mAbs) are under development for treating many diseases, including cancer, rheumatoid arthritis, autoimmune diseases, and asthma. (Carter 2006; Reichert 2014) To avoid the need for IV delivery of dilute solutions in the clinic, it would be desirable to deliver a typical ˜250 mg dose in a 1 ml injection subcutaneously. (Shire, Shahrokh et al. 2004; Srinivasan, Weight et al. 2013; Severson 2015) At these high concentrations, the interactions between closely spaced proteins often result in high viscosities, often above the 15-50 cP limit for SC injection. (Shire, Shahrokh et al. 2004; Miller, Engstrom et al. 2010; Buck, Chaudhri et al. 2015; Wang 2015) Furthermore, concentrated proteins are prone to reversible and irreversible aggregation, which has the potential to produce immunogenic responses. (Shire, Shahrokh et al. 2004; Saluja and Kalonia 2008; Carpenter, Randolph et al. 2009; Roberts 2014; Fathallah, Chiang et al. 2015) In some cases the mAb sequence in the CDR may be modified to reduce aggregation and viscosity, but this approach is time consuming and may impact the therapeutic efficacy of the mAb. (Connolly, Petry et al. 2012; Ladiwala, Bhattacharya et al. 2012; Perchiacca, Ladiwala et al. 2012; Chaudhri, Zarraga et al. 2013; Zarraga, Taing et al. 2013) An alternative would be to modify the viscosity with an electrolyte (Liu, Nguyen et al. 2005; Kanai, Liu et al. 2008; Yearley, Godfrin et al. 2014) or a co-solute (Connolly, Petry et al. 2012; Inoue, Takai et al. 2014). While the effects of cosolutes on protein stabilization have been studied extensively for disaccharide and amino acids, (Izutsu, Yoshioka et al. 1991; Webb, Rule et al. 1997; Kreilgaard, Frokjaer et al. 1998; Kreilgaard, Frokjaer et al. 1999; Samuel, Kumar et al. 2000; Chen, Bautista et al. 2003; Ignatova and Gierasch 2006; Arakawa, Ejima et al. 2007) relatively few studies have examined their effects on viscosity, (Du and Klibanov 2011; He, Woods et al. 2011; Inoue, Takai et al. 2014) particularly for concentrations above 200 mg/ml.

The complex relationship between the viscosity of a concentrated mAb solution and the nature of the protein-protein interactions (PPI), including electrostatic, charge-dipole, H-bonding and hydrophobic interactions, is not well understood. (Kanai, Liu et al. 2008; Chaudhri, Zarraga et al. 2012; Chaudhri, Zarraga et al. 2013; Sarangapani, Hudson et al. 2013; Li, Kumar et al. 2014; Buck, Chaudhri et al. 2015; Sarangapani, Hudson et al. 2015) Attractive interactions between the fAb regions of the antibody (fAb-fAb or fAb-fc)—particularly the complementary determining regions (CDRs) often play a large role in raising the viscosity. (Liu, Nguyen et al. 2005; Kanai, Liu et al. 2008; Saluja and Kalonia 2008; Chaudhri, Zarraga et al. 2012; Li, Kumar et al. 2014; Schmit, He et al. 2014; Buck, Chaudhri et al. 2015) For a variety of mAbs a decrease in viscosity was seen with mutations that changed the PPI from attractive to repulsive, as determined by second virial coefficients from dynamic light scattering. (Connolly, Petry et al. 2012) For IgG1 mAbs, high protein viscosities have been correlated with negatively charged fAb regions due to their attractive electrostatic interactions with the positively charged fc region. (Li, Kumar et al. 2014) A recent study has shown that the viscosity increases with an increase in attractive PPI, as characterized by the apparent radius of gyration and apparent maximum dimension as determined by small angle x-ray scattering (SAXS). (Fukuda, Moriyama et al. 2015) In some cases, strong attractive interactions have been known to cause mAb oligomerization associated with increases in viscosity; (Yadav, Shire et al. 2010; Chaudhri, Zarraga et al. 2012; Zarraga, Taing et al. 2013; Schmit, He et al. 2014; Yearley, Godfrin et al. 2014) where in one case the viscosity increased from 20 to 450 cP as the aggregation number increased from 1 to 9. (Lilyestrom, Yadav et al. 2013)

The anisotropic charge distribution on protein surfaces can cause attraction due to alignment of local patches on two protein molecules. (Li, Kumar et al. 2014; Roberts and Blanco 2014; Buck, Chaudhri et al. 2015) Consequently, mAb solution viscosities may in some cases be lowered by adding salts to screen these attractive local anisotropic electrostatic and charge-dipole PPI. (Liu, Nguyen et al. 2005; Kanai, Liu et al. 2008; Yearley, Godfrin et al. 2014) Typically, chaotropic salts have been shown to reduce viscosities to a greater extent than kosmotropic salts by disrupting the water structure near the protein surface. (Liu, Nguyen et al. 2005; Kanai, Liu et al. 2008; Scherer 2013) The extent of viscosity reduction however has not been found to directly follow a predictable trend such as the Hofmeiser series. (Liu, Nguyen et al. 2005; Lo Nostro and Ninham 2012) For other proteins, the addition of salt has been shown to have little effect or even to increase the solution viscosity. (Du and Klibanov 2011; Lilyestrom, Yadav et al. 2013; Inoue, Takai et al. 2014; Yearley, Godfrin et al. 2014) Another strategy to achieve low viscosity is to disperse insoluble micron-sized protein particles in hydrophobic organic solvents such as ethanol and benzyl benzoate that do not dissolve the protein. Here, the low viscosities may be attributed to the relatively weak interactions between the colloidal protein particles. (Miller, Engstrom et al. 2010; Johnson and Lenhoff 2013; Srinivasan, Weight et al. 2013) In contrast, for aqueous media, the soluble protein has a large influence on the viscosity behavior.

Relative to inorganic salts, less is known about how organic co-solutes, such as saccharides or amino acids lower the viscosity of protein dispersions. These co-solutes can be divided into two groups: preferentially excluded co-solutes, which are typically neutral molecules, such as disaccharides, and are excluded from the protein surface; and preferentially interacting co-solutes, that are often charged and have favorable interactions with the protein surface. (Arakawa, Ejima et al. 2007) Low viscosities have been observed for highly concentrated mAb solutions with the preferentially excluded, neutral co-solute trehalose, which at high concentration causes depletion attraction between the proteins. (Asakura and Oosawa 1958; Vrij 1976; Johnston, Maynard et al. 2012; Borwankar, Dinin et al. 2013) The mAbs in these systems have been shown to remain stable upon dilution both in vitro and in vivo; however, the degree to which the co-solute lowered the viscosity was not reported. For other mAbs, these co-solutes increase the viscosity. (He, Woods et al. 2011) In most cases the formation of protein oligomers (also referred to as aggregates or nanoclusters) has been correlated with elevated viscosities. (Chaudhri, Zarraga et al. 2012; Lilyestrom, Yadav et al. 2013; Pathak, Sologuren et al. 2013; Zarraga, Taing et al. 2013; Castellanos, Pathak et al. 2014; Yearley, Godfrin et al. 2014) The amino acid arginine, a preferentially interacting co-solute, has been shown to reduce the viscosity of several different proteins, including mAbs. (Liu, Nguyen et al. 2005; Connolly, Petry et al. 2012; Inoue, Takai et al. 2014) Like inorganic salts, arginine can reduce solution viscosities through charge screening of local anisotropic attractive electrostatic interactions. However, arginine additionally interacts with the protein surface via its guanidyl group, such that arginineH+ increases the balance of positive charges on the protein surface and thus weakens the local attractive electrostatic PPI. (Arakawa, Ejima et al. 2007; Schneider and Trout 2009; Vondrasek, Mason et al. 2009; Schneider, Shukla et al. 2011; Shukla, Schneider et al. 2011; Shukla and Trout 2011; Shih, England et al. 2013; Fukuda, Kameoka et al. 2014) Below the protein pI, the increase in the net positive charge of the protein surface makes the mAb interactions more repulsive. Although the effectiveness of arginine for viscosity reduction varies with mAb sequence (Connolly, Petry et al. 2012) the extent of the reduction tends to increase with arginine concentration. (Inoue, Takai et al. 2014) In addition to arginine, several other organic charged molecules have been shown to reduce protein viscosity by modulating the electrostatic and hydrophobic attractive PPI. (Du and Klibanov 2011; Guo, Chen et al. 2012) However, the effects of these co-solutes have not been correlated with solution pH and ionic strength, which would shed further insight on the effects of PPI on viscosity. Even more importantly, relatively few studies of co-solute effects on viscosity at high concentration have also examined the protein aggregation, which is not only important scientifically, but essential for applying this technology to subcutaneous delivery.

Herein, we demonstrate reductions in the viscosity and increases in the stability of ˜200-250 mg/ml mAb solutions by manipulation of the PPI upon adding elevated concentrations of charged organic co-solutes, including three bases histidine (His), arginine (Arg), imidazole (Imid) and also the hydrophobic anion camphorsulfonate (CSA) over a range of pH values. For each of these co-solutes, we achieved the goal of viscosities of 20 cP and less at mAb concentrations greater than 200 mg/ml with monomer retention of >98% after four weeks of 40° C. storage. The organic bases were chosen to interact with charged and polar regions on the protein via electrostatic, hydrogen bonding and charge-dipole interactions, as well as with aromatic residues via it electrons in their imidazole or guanidyl groups. The interaction of the cosolutes with the aromatic residues will lower the attractive hydrophobic PPI. Our hypothesis is that the bases in the protonated cationic increase the effective positive charge of the protein surface by neutralizing negatively charged sites, making neutral sites positive and increasing the magnitude of positively charged sites to produce two beneficial effects: (1) increasing net repulsion in the PPI and (2) weakening local anisotropic electrocstatic and charge dipole attraction. At pH 5 and 6 arg and im are fully protonated, but his is only partially protonated; this difference is shown to have an influence on the degree of viscosity reduction.

Interaction of uncharged his with aromatic residues covers hydrophobic regions with zwitterions, which are known to repel proteins. (Chen, Cao et al. 2009; Jiang and Cao 2010) Imidazole behaves similarly to his, but is more strongly cationic for a given pH and lacks the zwitterionic moiety. Viscosities with these the three organic bases are shown to decrease with pH as local electrostatic attractive PPI become weaker as the effective positive charge on the protein increases. In contrast, interactions with the anionic CSA increase the effective negative charge on the protein; therefore, CSA yields lower viscosities with an increase in pH. Other co-solutes, including inorganic electrolytes and weakly interacting amino acids are shown to have only minimal effects on the mAb viscosity and stability.

In addition, we test two distinct pathways for forming concentrated mAb dispersions, centrifugal filtration and dissolution of lyophilized powder and show that, for the cases tested, the viscosity is similar for both pathways.

### Results and Discussion

Effect of Histidine Concentration and pH on Viscosity

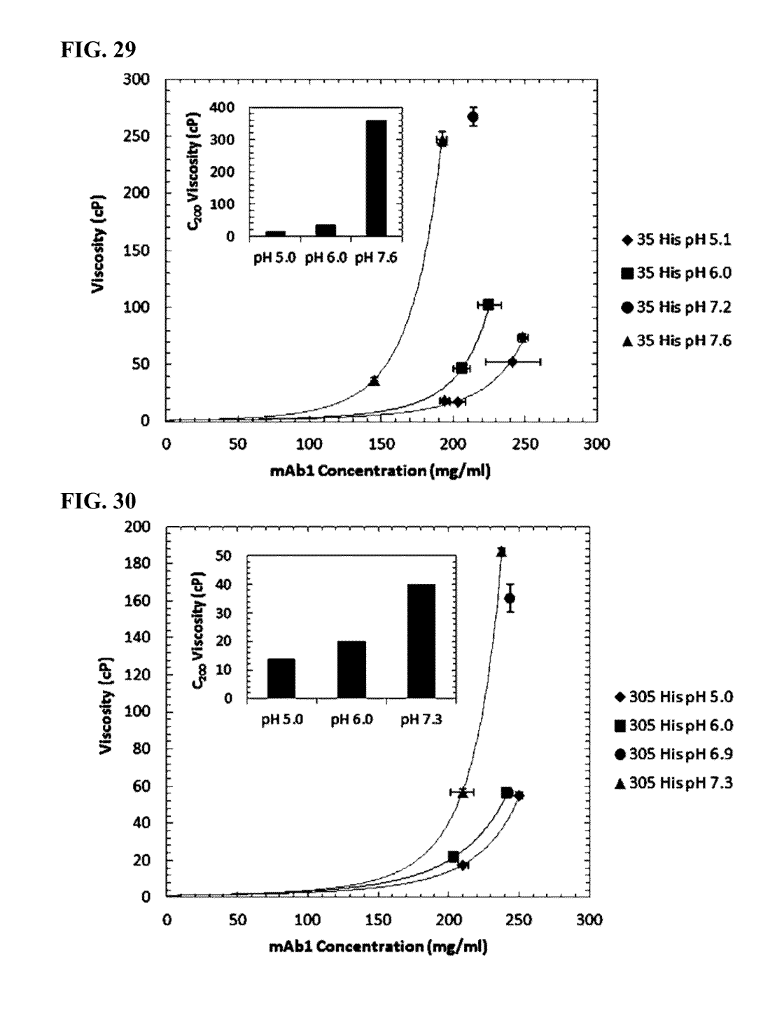

The viscosities of highly concentrated dispersions of mAb1 were examined for histidine (His) at two concentrations, 35 and 305 mM, with the centrifugation filtration (CF) process. To provide a control with low co-solute concentration, the protein was concentrated by CF up to 250 mg/ml in aqueous solvents containing 35 mM His with HCl at pH values ranging from ˜5 to 7.6. As shown in FIG. 29, the viscosity of 150-250 mg/ml mAb1 dispersions decreases markedly with continual reductions in pH from 7.6 all the way to 5 at each mAb concentration tested. The final concentration of co-solute in the retentate, C_{c,R}, will likely be lowered because of the increase in volume occupied by the protein during ultrafiltration. Assuming protein-co-solute interactions are only from volume-exclusion effects, C_{c,R} will depend on the concentration of the co-solute in the solvent feed, C_{c,F}, and the final protein concentration, cₚ, as follows: (Teeters, Bezila et al. 2011)

C_{c,R}=C_{c,F}*(1− v̅ₚ*Cₚ) (5-5)

where v̅ₚ is the partial molar volume of the protein, assumed for mAb1 to be 0.7407 ml/g. (Aragon and Hahn 2006; Zhao, Brown et al. 2011) Thus, a solvent feed His concentration of 35 mM will result in a Cc,F of 30 mM at 250 mg/ml protein, which is a commonly used buffer for pH ˜6 protein formulations. (Liu, Nguyen et al. 2005; Connolly, Petry et al. 2012; Karow, Bahrenburg et al. 2013) As shown in FIG. 29 and Table 5-S1, the viscosity of these systems is markedly smaller at the lower pH values. The results were fit to the Ross-Minton equation (Eq. 3) with the fit parameters given in Table 5-S2. From the fits of Eq. 3, the predicted viscosities at 200 mg/ml are 17 cP at pH 5, 37 cP at pH 6 and 361 cP at pH 7.6 as shown in the inset. It should be noted that for all of these samples the pH drifted upward during diafiltration by ˜0.1 units at pH 5 and 6 and ˜0.4 units near pH 7. This likely indicates that the positively charged protein preferentially repels the fraction of positively charged HisH+ through the ultrafiltration membrane more than neutral His. This may indicate that the final solvent concentrations could be somewhat different than predicted by equation 5. (Teeters, Bezila et al. 2011)

TABLE 5-S1
Viscosity and stability of mAb1 solutions concentrated by CF in
solvent containing HCl and 35 mM histidine. Inherent viscosities are
calculated using a solvent viscosity of 1 cP. Properties that were measured
multiple times are represented as the average measurement plus or minus
the standard deviation of the measurements. Viscosity at C₂₀₀ represents the
calculated viscosity from the best fit of the data at 200 mg/ml mAb1.

| | | mAb1 | | Inherent | mAb1 | | Inherent | Viscosity | 4 Week |
|---|---|---|---|---|---|---|---|---|---|
| Solvent | Solution | Conc. | Viscosity | Viscosity | Conc. | Viscosity | Viscosity | at C₂₀₀ | SEC % |
| pH | pH | 1 (mg/ml) | 1 (cP) | 1 (ml/g) | 2 (mg/ml) | 2 (cP) | 2 (ml/g) | (cP) | Monomer |
| 4.94 | 5.07 | 242 ± 19 | 53 ± 1 | 16.4 | 194 ± 3 | 18 ± 0.3 | 14.9 | 17 | 95.8 |
| 4.86 | 5.10 | 249 ± 4 | 73 ± 4 | 17.3 | 203 ± 5 | 17 ± 0.4 | 14.1 | 17 | 92.3 |
| 5.92 | 6.04 | 225 ± 8 | 102 ± 1 | 20.5 | 206 ± 6 | 46 ± 1 | 18.6 | 37 | 86.5 |
| 6.77 | 7.19 | 214 ± 2 | 267 ± 8 | 26.1 | — | — | — | — | TBD |
| 7.14 | 7.61 | 192 ± 4 | 248 ± 6 | 28.7 | 145 ± 1 | 37 ± 1 | 24.9 | 361 | 86.0 |

TABLE 5-S2
Fit parameters for the Ross-Minton equation for CF data.

| | Intrinsic | Ratio of crowding |
|---|---|---|
| | Viscosity, | factor to Simha |
| Sample | [η], (ml/mg) | parameter, k/v, |
| 35 mM His pH 5.1 | 0.0081 | 0.2632 |
| 35 mM His pH 6.0 | 0.0093 | 0.2626 |
| 35 mM His pH 7.6 | 0.0178 | 0.1115 |
| 305 mM His pH 5.0 | 0.0075 | 0.2780 |
| 305 mM His pH 6.0 | 0.0098 | 0.1718 |
| 305 mM His pH 7.3 | 0.0098 | 0.2355 |
| 305 mM Arg pH 5.3 | 0.0072 | 0.2856 |
| 305 mM Arg pH 5.9 | 0.0078 | 0.2513 |
| 305 mM Arg pH 6.9 | 0.0114 | 0.0916 |
| 305 mM Lys pH 6.0 | 0.0150 | 0.0476 |
| 270 mM NaCl pH 6.0 | 0.0176 | 0.0150 |
| 305 mM Imid pH 5.9 | 0.0066 | 0.4011 |
| 305 mM Imid pH 6.9 | 0.0066 | 0.4350 |
| 270 mM Ala pH 6.0 | 0.0050 | 0.7208 |
| 270 mM NaCSA pH 7.0 | 0.0069 | 0.3567 |
| 305 mM ArgCSA pH 5.9 | 0.0113 | 0.0895 |
| 305 mM ArgCSA pH 6.8 | 0.0095 | 0.1453 |

To determine the effects of His-protein interactions on viscosity, the above experiments were conducted using a large co-solute concentration of 305 mM His, (targeting 250 mM at 250 mg/ml mAb1 according to Eq. 5). As shown in FIG. 30 and Table 5-S3, the viscosity decreases with decreasing pH at all mAb1 concentrations, which was also seen for the samples mentioned above in FIG. 29. Furthermore, increasing the His concentration to 305 mM decreases the viscosity relative to 35 mM His at each pH tested (5 to 7.5). At 200 mg/ml mAb1, the predicted viscosity reductions with the increase in His concentration were ˜20%, ˜45%, and ˜90% for pH values of ˜5, ˜6, and ˜7.5 respectively. Despite the greater reduction at the higher pH, the actual viscosity was lowest at pH 5 as the predicted viscosity with Eq. 3 at 200 mg/ml is only 14 cP. As with the 35 mM His samples, the pH drifted upward during ultrafiltration for these samples; however, the magnitude of the drift was consistently smaller than it was at 35 mM, (<0.1 unit at pH 5 and 6, ˜0.2 units near pH 7), possibly due to ionic screening.

TABLE 5-S3
Viscosity and stability of mAb1 solutions concentrated by CF in
solvent containing HCl and 305 mM histidine. Inherent viscosities are
calculated using a solvent viscosity of 1.05 cP. Properties that were measured
multiple times are represented as the average measurement plus or minus
the standard deviation of the measurements. Viscosity at C200 represents the
calculated viscosity from the best fit of the data at 200 mg/ml mAb1.

| | | mAb1 | | Inherent | mAb1 | | Inherent | Viscosity | 4 Week |
|---|---|---|---|---|---|---|---|---|---|
| Solvent | Solution | Conc. | Viscosity | Viscosity | Conc. | Viscosity | Viscosity | at C₂₀₀ | SEC % |
| pH | pH | 1 (mg/ml) | 1 (cP) | 1 (ml/g) | 2 (mg/ml) | 2 (cP) | 2 (ml/g) | (cP) | Monomer |
| 4.94 | 5.01 | 250 ± 2 | 55 ± 1 | 15.8 | 210 ± 4 | 18 ± 0.4 | 13.5 | 14 | 99.7 |
| 5.97 | 5.98 | 242 ± 4 | 56 ± 1 | 16.5 | 204 ± 1 | 22 ± 1 | 14.9 | 20 | 98.6 |
| 6.75 | 6.90 | 244 ± 1 | 161 ± 8 | 20.6 | — | — | — | — | TBD |
| 7.11 | 7.34 | 238 ± 2 | 187 ± 2 | 21.8 | 210 ± 8 | 57 ± 2 | 19.0 | 40 | 92.7 |

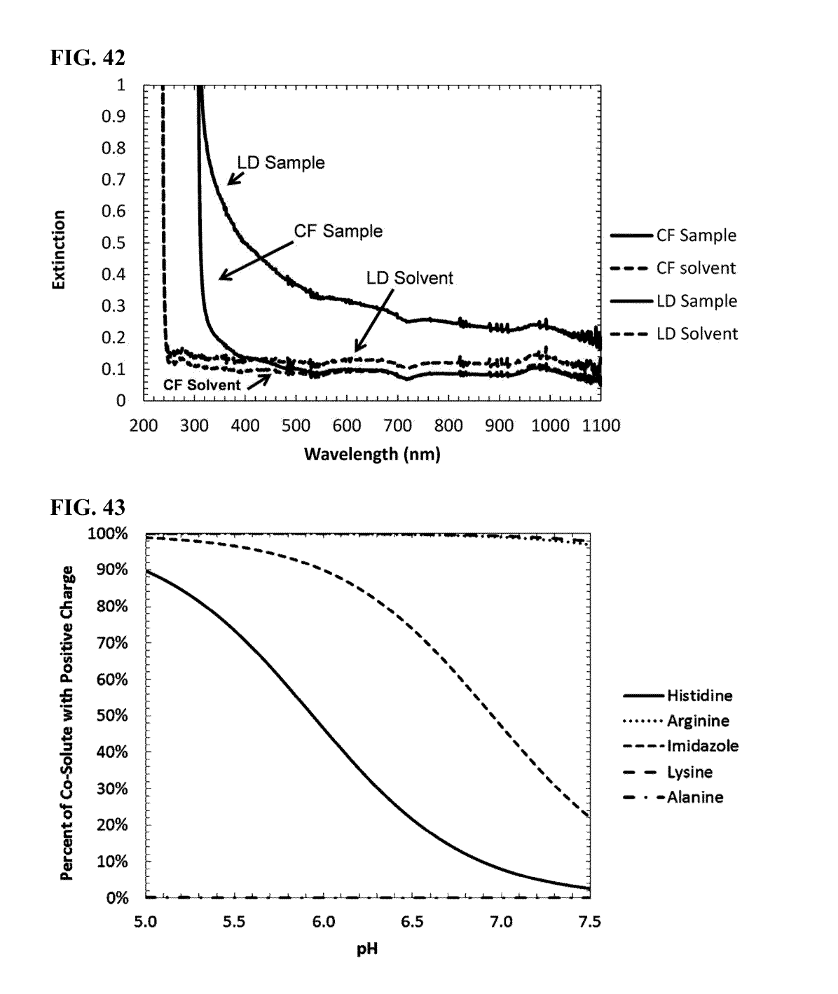

To complement these results, protein dispersions with similar formulations were made by the lyophilization dilution (LD) technique. An advantage of this technique is that the final concentration of the co-solute is known, because all of the components are measured gravimetrically and mixed into the sample. For these LD samples, the mAb1 was concentrated to ˜250 mg/ml with both 30 mM and 250 mM His concentrations over the same 5-7.5 pH value range. Due to the strong buffer capacity of the lyophilized protein powder, the pH of the 30 mM His samples only ranged from 5.5-7. (Karow, Bahrenburg et al. 2013) Unlike the CF samples, these samples also contained 130 mM trehalose in addition to the His and HCl, as the trehalose was used as a cryoprotectant/lyoprotectant during formation of the lyophilized powder. (Kreilgaard, Frokjaer et al. 1998; Kreilgaard, Frokjaer et al. 1999) The LD samples were slightly turbid by eye unlike the samples made by CF, which were all transparent. The turbidity measured for select samples clearly showed these elevated turbidities for LD samples, presumably from aggregates formed at the air-water interfaces of small air bubbles produced during dissolution of the powder. Representative spectra are given in FIG. 42 for concentrated mAb1 dispersions (˜250 mg/ml) formed by LD and CF. Additionally, the turbidity was also observable in the 500× dilutions of the samples used for measuring the protein concentration by A280. In addition to measuring A280, the absorbance at 350 nm (A350) was also measured, as is often done to characterize the turbidity. (Hawe, Romeijn et al. 2012) The measured A350s of the LD samples were ˜5.5±0.6% of the measured A280s. In contrast, it was <1% of A280 for all samples made by CF, as shown for representative samples in FIG. 41. Despite the differences in turbidity, as well as trehalose concentrations, between the CF and LD samples, the final viscosities were remarkably similar for a given His concentration, pH and mAb1 concentration.

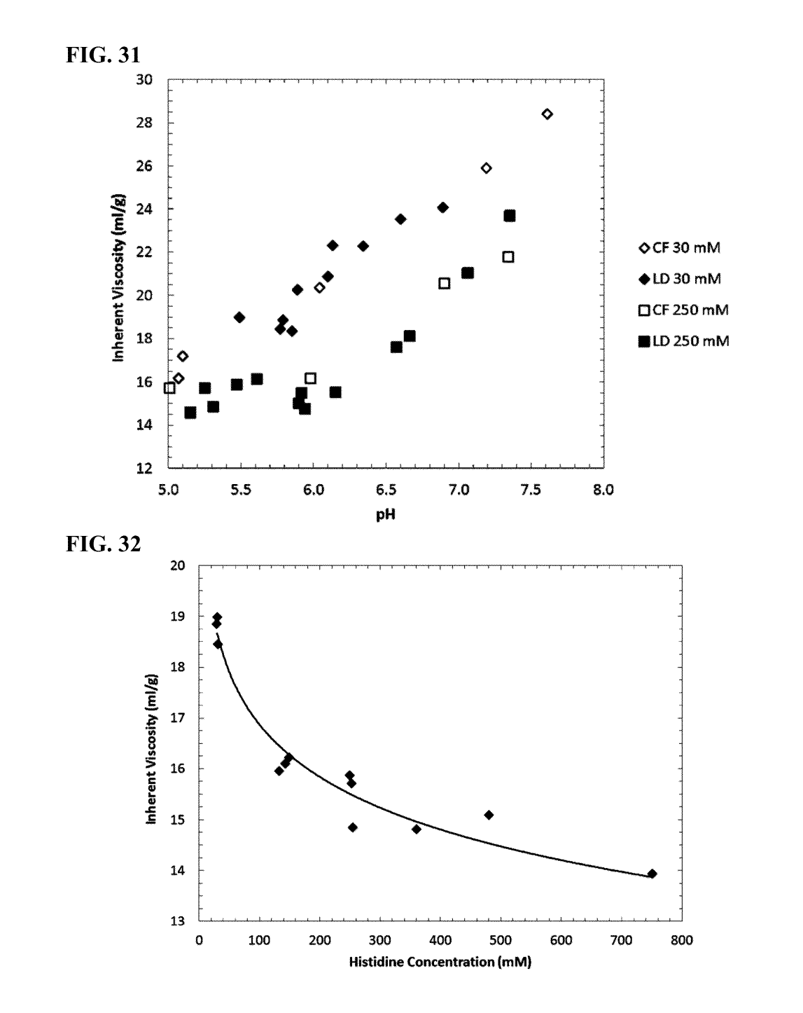

FIG. 31 depicts the inherent viscosities, ηᵢₙₕ=ln(η/η₀)/c, as a function of pH for CF and LD samples at 30 mM and 250 mM His for ˜250 mg/ml mAb1. The mAb1 concentration ranged from 222 to 260 mg/ml, with an average value of 245 mg/ml and standard deviation of 8 mg/ml. The ηᵢₙₕ is used instead of absolute viscosity for comparison between formulations to mitigate the dependence on protein concentration, given the exponential dependence of viscosity on concentration. (Liu, Nguyen et al. 2005; Yadav, Shire et al. 2010) Therefore at ˜250 mg/ml, even slight differences in concentration can cause drastic differences in viscosity for the same formulation. On the other hand, the change in ηᵢₙₕ with concentration is much more gradual, as shown by the following relationship achieved by simultaneously solving Eqn. 5-3 with the definition of inherent viscosity.

$${η}_{inh} ≡ \frac{ln ( η / {η}_{0} )}{c} = \frac{[ η ]}{1 - [ η ] \frac{k}{v} c} \qquad ( 5 \text{-} 6 )$$

Thus, ηᵢₙₕ is useful in comparing formulations at similar, but not identical concentrations.

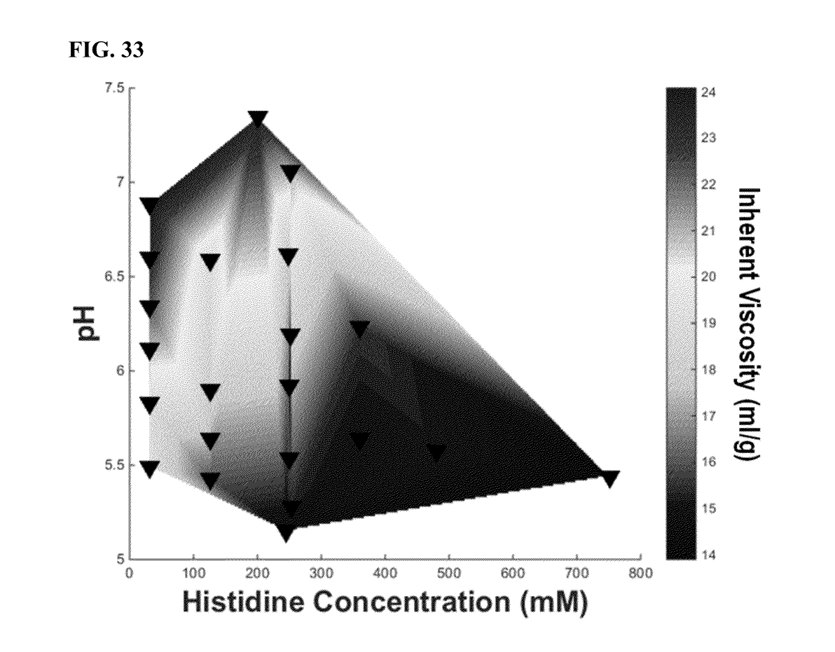

As seen in FIG. 31 and Tables 5-S4 and 5-S5, ηᵢₙₕ at ˜250 mg/ml mAb1 with 250 mM His decreases sharply with pH from 7.5 to 6, then remains relatively steady down to pH 5. For similar systems with 30 mM His the ηᵢₙₕ decreases fairly linearly with pH over the entire range tested and approaches the value of ηᵢₙₕ for 250 mM His at pH 5. The reduction in ηᵢₙₕ with increasing His concentration is further characterized in FIG. 32 for LD samples over a large number of His concentrations with ˜250 mg/ml mAb 1. At pH ˜5.5 ηᵢₙₕ continually decreases all the way up to 750 mM; however, the rate of reduction steadily decreases. FIG. 33 and Tables 5-S4-5-S7 show the combined effects of His concentration and pH for these LD samples, showing that high concentrations of His and low pH both reduce ηᵢₙₕ across the spectrum tested. The upper right-hand region of FIG. 33 (corresponding to high concentration His and high pH) is not filled in because His is not soluble in this region.

TABLE 5-S4
Viscosity of mAb1 dispersions at ~250 mg/ml with 130
mM trehalose and 30 mM histidine. Inherent viscosity is
calculated using a solvent viscosity of 1.1 cP. Rows are
listed by increasing pH. The plus or minus represents the
standard deviation between replicate measurements.

| mAb1 | HCl | | | Inherent |
|---|---|---|---|---|
| Concentration | Concentration | | Viscosity | Viscosity |
| (mg/ml) | (mM) | pH | (cP) | (ml/g) |
| 254 ± 8 | 47 | 5.49 | 138 ± 6 | 19.0 |
| 255 ± 2 | 32 | 5.77 | 122 ± 4 | 18.4 |
| 255 ± 6 | 30 | 5.79 | 135 ± 5 | 18.8 |
| 235 ± 12 | 32 | 5.85 | 81 ± 11 | 18.3 |
| 251 ± 6 | 32 | 5.89 | 180 ± 14 | 20.3 |
| 243 ± 2 | 20 | 6.10 | 176 ± 2 | 20.9 |
| 248 ± 4 | 29 | 6.13 | 277 ± 43 | 22.3 |
| 254 ± 13 | 17 | 6.34 | 313 ± 35 | 22.3 |
| 254 ± 7 | 8 | 6.60 | 428 ± 12 | 23.5 |
| 222 ± 8 | 0 | 6.89 | 232 ± 25 | 24.1 |

TABLE 5-S5
Viscosity of mAb1 dispersions at ~250 mg/ml with 130 mM trehalose
and 250 mM histidine or arginine. The final row only has 200 mM
histidine due to low histidine solubility at this pH. Inherent
viscosity is calculated using a solvent viscosity of 1.2 cP. Rows
are listed by increasing pH. The plus or minus represents the
standard deviation between replicate measurements

| mAb1 | Main | HCl | | | Inherent |
|---|---|---|---|---|---|
| Concentra- | Buffer | Concentra- | | Viscosity | Viscosity |
| tion (mg/ml) | Component | tion (mM) | pH | (cP) | (ml/g) |
| 244 ± 3 | His | 248 | 5.15 | 42 ± 1 | 14.6 |
| 257 ± 3 | His | 241 | 5.15 | 51 ± 1 | 14.6 |
| 260 ± 2 | His | 253 | 5.25 | 72 ± 10 | 15.7 |
| 239 ± 7 | His | 255 | 5.31 | 41 ± 7 | 14.8 |
| 241 ± 8 | His | 229 | 5.47 | 55 ± 2 | 15.9 |
| 250 ± 1 | His | 176 | 5.90 | 51 ± 4 | 15.0 |
| 240 ± 10 | His | 166 | 5.92 | 50 ± 2 | 15.5 |
| 249 ± 10 | His | 166 | 5.94 | 47 ± 3 | 14.7 |
| 236 ± 2 | His | 132 | 6.15 | 47 ± 3 | 15.5 |
| 248 ± 3 | His | 131 | 6.22 | 61 ± 6 | 15.8 |
| 241 ± 8 | His | 63 | 6.57 | 84 ± 7 | 17.6 |
| 238 ± 2 | His | 54 | 6.66 | 90 ± 5 | 18.1 |
| 235 ± 15 | His | 19 | 7.06 | 167 ± 14 | 21.0 |
| 242 ± 9 | His | 0 | 7.35 | 373 ± 3 | 23.7 |
| 245 ± 7 | Arg | 250 | 6.36 | 46 ± 4 | 14.9 |

TABLE 5-S6
Viscosity of mAb1 dispersions at ~250 mg/ml with 130
mM trehalose and 125 mM histidine. Inherent viscosity is
calculated using a solvent viscosity of 1.12 cP for row
1 and 1.15 cP other rows. The plus or minus represents the
standard deviation between replicate measurements (if no
plus or minus is present only one measurement was taken).

| mAb1 | HCl | | | Inherent |
|---|---|---|---|---|
| Concentration | Concentration | | Viscosity | Viscosity |
| (mg/ml) | (mM) | pH | (cP) | (ml/g) |
| 262 ± 4 | 126 | 5.34 | 78 ± 9 | 16.1 |
| 276 ± 6 | 126 | 5.44 | 101 ± 36 | 16.2 |
| 239 ± 1 | 127 | 5.50 | 52 ± 2 | 15.9 |
| 243 ± 7 | 115 | 5.64 | 80 ± 5 | 17.5 |
| 238 ± 11 | 85 | 5.90 | 95 ± 10 | 18.5 |
| 244 ± 0.02 | 66 | 6.57 | 125 ± 13 | 19.2 |
| 240 ± 8 | 33 | 6.61 | 111 ± 9 | 19.0 |

TABLE 5-S7
Viscosity of mAb1 dispersions at ~250 mg/ml with 130 mM trehalose
and >250 mM histidine. Inherent viscosity is calculated using
a solvent viscosity of 1.25 cP for rows 1 and 2, 1.30 cP for
row 3 and 1.45 cP for row 4. The plus or minus represents the
standard deviation between replicate measurements.

| mAb1 | Histidine | HCl | | | Inherent |
|---|---|---|---|---|---|
| Concentra- | Concentra- | Concentra- | | Viscosity | Viscosity |
| tion (mg/ml) | tion (mM) | tion (mM) | pH | (cP) | (ml/g) |
| 213 ± 2 | 360 | 383 | 5.64 | 29 ± 2 | 14.8 |
| 230 ± 3 | 360 | 232 | 6.23 | 44 ± 1 | 15.5 |
| 239 ± 2 | 480 | 414 | 5.58 | 48 ± 3 | 15.1 |
| 238 ± 5 | 750 | 644 | 5.44 | 40 ± 3 | 13.9 |

For IgG1 antibodies, the fc region is positively charged near physiological pH values, whereas the fAb region is often negatively charged. (Li, Kumar et al. 2014; Buck, Chaudhri et al. 2015) Thus, a decrease in pH reduces the negative charges in the fAb region and increases the positive charges in the fc region and thus weakens local anisotropic attractive fAb-fc electrostatic and charge-dipole interactions. Simultaneously, lowering the pH increases the global net positive charge of the mAb, increasing long-ranged charge repulsion. Therefore, as the pH is lowered starting at the pI of 9.3, the local attractive protein-protein interactions (PPI) become weaker and the repulsive PPI become stronger, both of which would be expected to reduce the viscosity, as is seen for 35 mM His in FIG. 33. (Connolly, Petry et al. 2012; Li, Kumar et al. 2014; Fukuda, Moriyama et al. 2015) The viscosity also decreased with 250 mM His down to pH 6 (FIGS. 31 and 33). However, at pH values below 6 the addition of His at 250 mM or higher had a relatively small effect on the viscosity. The HisH+ and HCl ions in the double layer may screen the charges on the protein surface and lower the local attractive electrostatic and charge-dipole PPI. In addition to charge screening, the carboxylate, amino and imidazole groups of the His can interact with the charged sites on the protein via hydrogen bonding, electrostatic and charge-dipole interactions. (Chen, Bautista et al. 2003; Hou and Cramer 2011; Kamerzell, Esfandiary et al. 2011; Holstein, Parimal et al. 2012) Through these preferential interactions, the positively charged HisH+increases the effective positive charge of the protein by neutralizing negatively charged sites, adding a positive charge to neutral sites, and even making positively charged sites more strongly positive. Thus His can strengthen long-range repulsive PPI and weaken short-range attractive PPI, analogously to the aforementioned behavior with reductions in pH without co-solute. Since the protein charge is already relatively high at a low pH, for example at pH 5, the impact of His on the charges on the protein surface may be expected to be less significant than at higher pH values. This behavior would be consistent with the observation of larger viscosity reductions with added His at elevated pH. However, as the concentration of His is continually increased, saturation of charged protein surface sites available to interact with His could explain why the viscosity reduction increases with His concentration at a diminishing rate, as seen in FIG. 32.

In addition to modulating the local and global electrostatic PPIs, His can block attractive hydrophobic patch interactions, which have been hypothesized to play a large role in raising protein solution viscosities. (Du and Klibanov 2011; Guo, Chen et al. 2012). His is known to preferentially interact with hydrophobic aromatic residues on the protein surface (tryptophan, tyrosine, phenylalanine, and other His) via its imidazole ring through cation-π interactions, and π-π stacking, (Loewenthal, Sancho et al. 1992; Fersht and Serrano 1993; Heyda, Mason et al. 2010; Du, Meng et al. 2012; Du, Wang et al. 2013; Liao, Du et al. 2013) which have interaction strengths on the same order as hydrogen bonds. (Vondrasek, Mason et al. 2009; Heyda, Mason et al. 2010; Du, Wang et al. 2013; Liao, Du et al. 2013) The preferential interaction of neutral/zwitterionic His or cationic HisH+ with an aromatic residue will effectively replace a hydrophobic patch with either a zwitterion, or a formal positive charge, respectively. Several studies have shown that proteins interact weakly with zwitterions, as they are highly hydrated. (Chen, Cao et al. 2009; Jiang and Cao 2010) Therefore, either of these substitutions should weaken hydrophobic attraction and strengthen electrostatic repulsion between proteins. At pH 7, the fraction of protonated His is only 8% (see FIG. 43), whereas it increases to 90% at pH 5. Thus, we now describe the effects of stronger bases that are fully protonated at pH 7 on the viscosity behavior.

Effect of Arginine Concentration and pH on Viscosity at ˜200-250 mg/Ml.

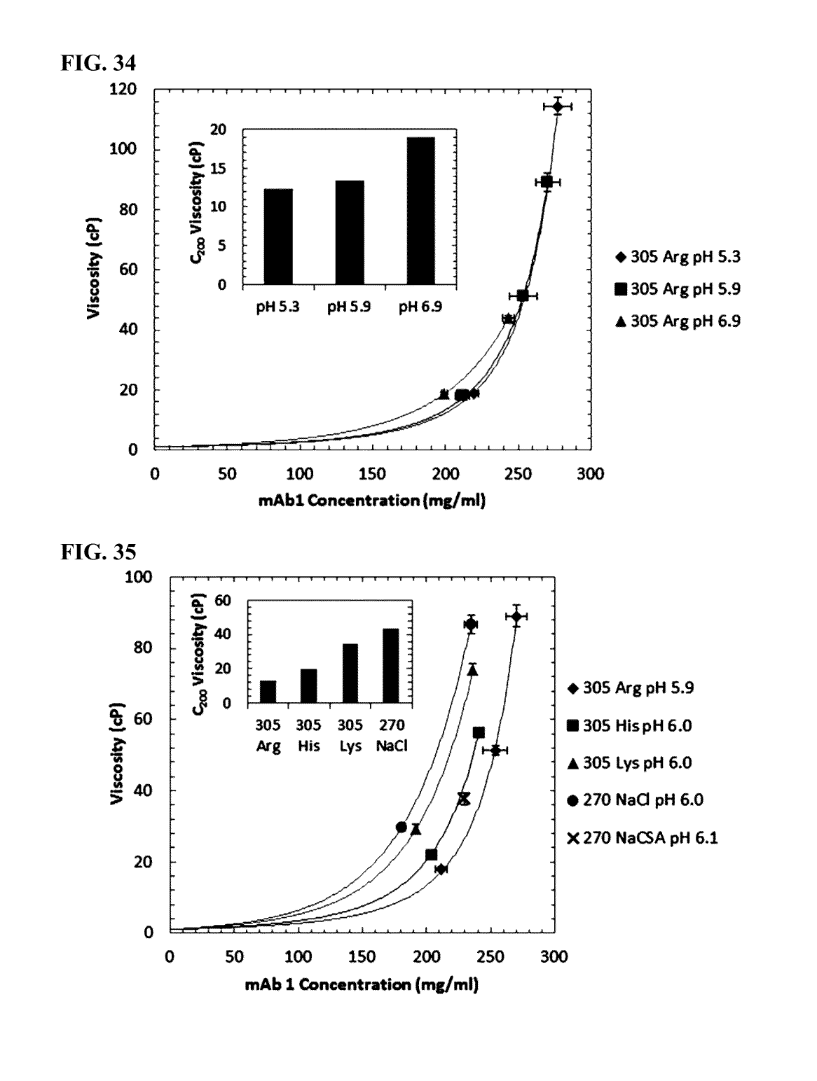

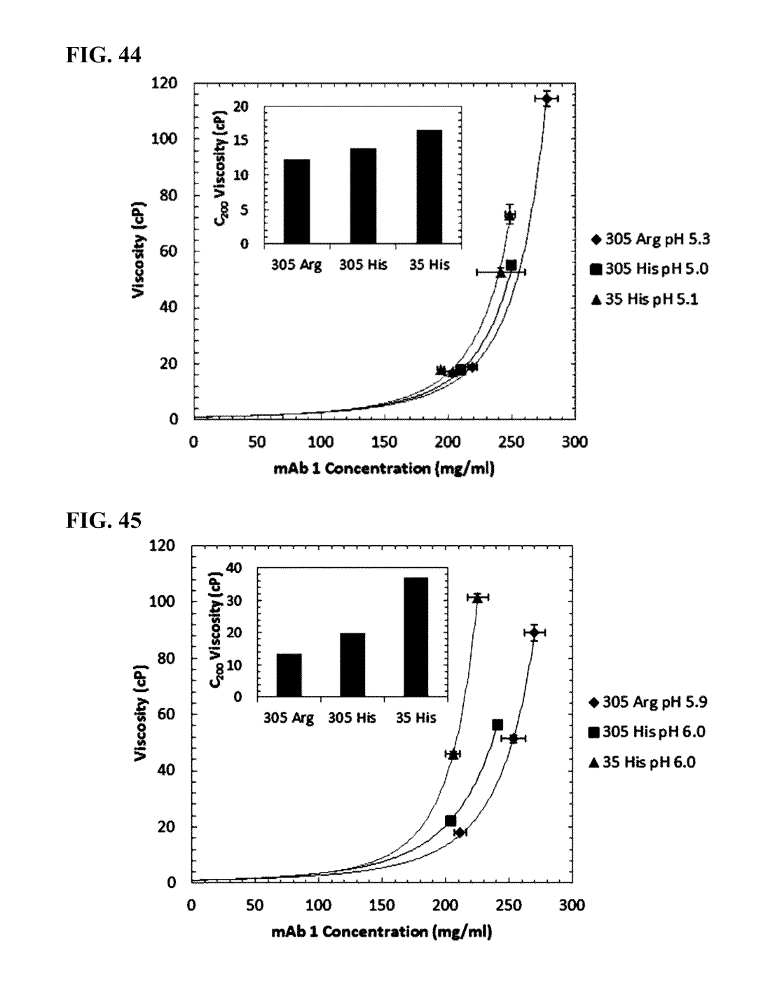

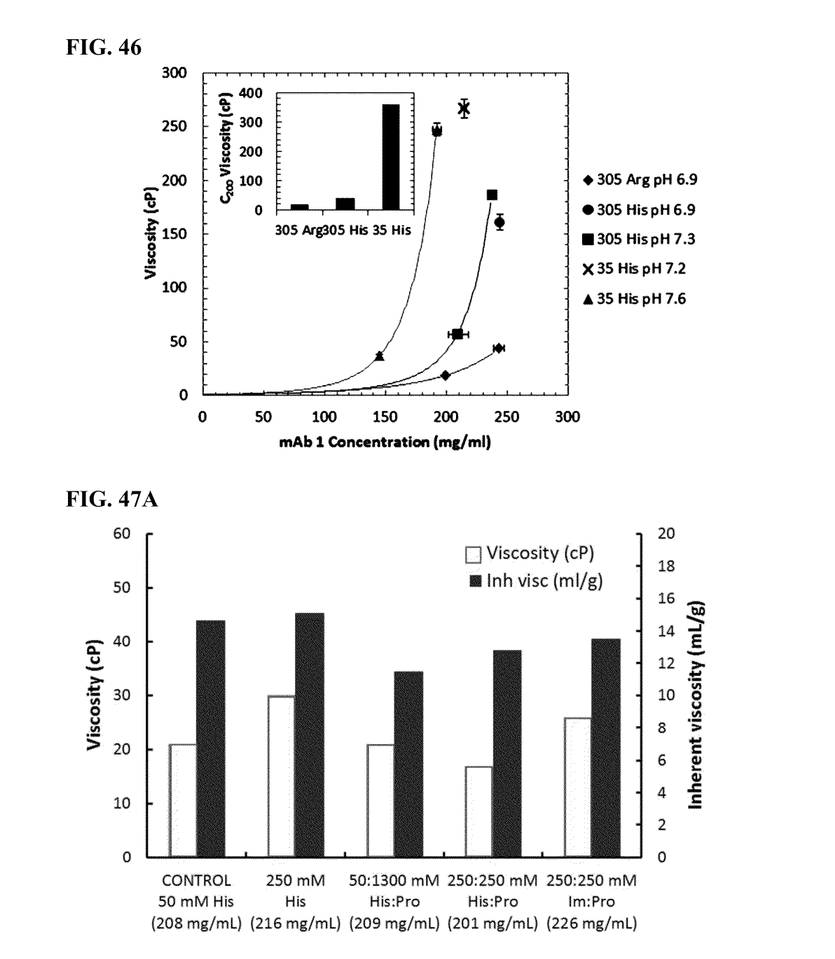

To examine the effects of arginine (Arg) which has a highly basic guanidyl functionality, mAb1 dispersions were concentrated to ˜250 mg/ml by CF with 305 mM Arg at pH values ranging from 5 to 7. As shown in FIG. 34 and Table 5-S8, the viscosity is essentially identical at pH 5 and 6 and is only slightly higher at pH 7, in contrast with the aforementioned strong pH dependence for 305 mM His. FIGS. 44-46, compare the buffer control with 35 mM His to dispersions with 305 mM His or Arg at pH values of ˜5, ˜6 and ˜7, respectively. At each pH value, the viscosity decrease, relative to the control, was larger for Arg than for His. For 305 mM Arg, the predicted (interpolated) viscosities from the Ross-Minton equation were 12, 13 and 19 cP at 200 mg/ml at pH values of 5, 6 and 7, respectively. A summary of the inherent viscosities at ˜200 mg/ml of all the systems tested by CF is given in Table 5-1. Although high concentrations of Arg and His lowered the viscosities at all pH values tested (relative to 35 mM His), the amount of the decrease became smaller as the pH decreased. At pH 7 the predicted viscosity reductions at 200 mg/ml were 90% and 95% for 305 mM His and Arg, respectively, while at pH 6 the reductions were 45% and 65%, and at pH 5, 20% and 30%.

TABLE 5-S8
Viscosity and stability of mAb1 solutions concentrated by CF in
solvent containing HCl and 305 mM arginine. Inherent viscosities are
calculated using a solvent viscosity of 1.05 cP. Properties that were measured
multiple times are represented as the average measurement plus or minus
the standard deviation of the measurements. Viscosity at C200 represents the
calculated viscosity from the best fit of the data at 200 mg/ml mAb1.

| | | mAb1 | | Inherent | mAb1 | | Inherent | | |
|---|---|---|---|---|---|---|---|---|---|
| Solvent | Solution | Conc. 1 | Viscosity | Viscosity | Conc. 2 | Viscosity 2 | Viscosity | Viscosity at | 4 Week SEC |
| pH | pH | (mg/ml) | 1 (cP) | 1 (ml/g) | (mg/ml) | (cP) | 2 (ml/g) | C₂₀₀ (cP) | % Monomer |
| 5.08 | 5.32 | 277 ± 9 | 115 ± 3 | 16.9 | 219 ± 4 | 19 ± 1 | 13.2 | 12 | 98.0 |
| 5.88 | 5.91 | 270 ± 9 | 89 ± 3 | 16.4 | 211 ± 5 | 18 ± 0.4 | 13.5 | 13 | — |
| 6.02 | 6.22 | 254 ± 10 | 51 ± 1 | 15.3 | — | — | — | — | TBD |
| 6.90 | 6.92 | 243 ± 4 | 44 ± 1 | 15.4 | 199 ± 2 | 19 ± 0.5 | 14.5 | 19 | 97.9 |

Arg is known to reduce the viscosity of various proteins (Liu, Nguyen et al. 2005; Connolly, Petry et al. 2012; Inoue, Takai et al. 2014) by preferentially interacting with the protein surface. (Arakawa, Ejima et al. 2007; Schneider and Trout 2009; Schneider, Shukla et al. 2011; Shukla, Schneider et al. 2011; Shukla and Trout 2011) These surface interactions are often thought to occur via the protonated guanidyl group, which interacts preferentially with aromatic groups and with other guanidyl and imidazole groups, even when both are cationic. (Arakawa, Ejima et al. 2007; Vondrasek, Mason et al. 2009; Heyda, Mason et al. 2010; Shih, England et al. 2013) Like His, Arg is thought to interact preferentially with charged groups through hydrogen bonding and charge-dipole interactions, as well as with hydrophobic groups through π interactions via its guanidyl group. (Arakawa, Ejima et al. 2007; Hou and Cramer 2011; Holstein, Parimal et al. 2012) In this respect, the guanidyl group functions analogously to the imidazole group of His. One major difference, however, is that the guanidyl group is essentially completely protonated up to pH 7.6; whereas, the imidazole group in His is only approximately 50% protonated at pH 6 and <10% protonated at pH 7 (FIG. 43). Since a cationic molecule would likely be expected to produce a larger increase in repulsion relative to a zwitterionic molecule and a larger decrease in local electrostatic attraction, Arg may be expected to produce a larger viscosity reduction at pH 7 than His. As expected, the predicted viscosity at 200 mg/ml was 19 cP for Arg versus 40 cP for His.

Effect of NaCl on Viscosity.

To distinguish between the effects of preferential interactions of co-solutes on the protein surface from those of simple screening of the electrical double layer, experiments were performed with the inorganic electrolyte NaCl. MAb1 was concentrated by CF at pH 6 with 35 mM His buffer and 270 mM NaCl to match the ionic strength of the 305 mM Arg solvents. As shown in FIG. 44 and Table 5-S9, this sample was much more viscous than those containing 305 mM His or Arg. In fact, the addition of 270 mM NaCl did not cause any noticeable viscosity reduction relative to 35 mM His at pH 6; therefore, the large effects of Arg and His on viscosity are not caused by simple charge screening.

TABLE 5-S9
Viscosity and stability of mAb1 solutions concentrated by CF in
solvent containing 35 mM histidine buffer with HCl and 270 mM of an
additional additive. Inherent viscosities are calculated using a solvent
viscosity of 1.05 cP for NaCl and Ala and 1.30 cP for NaCSA. Properties that
were measured multiple times are represented as the average measurement
plus or minus the standard deviation of the measurements. Viscosity at C200
represents the calculated viscosity from the best fit of the data at 200 mg/ml
mAb1.

| | | | | | Inherent | | | Inherent | Viscosity | 4 Week |
|---|---|---|---|---|---|---|---|---|---|---|
| | Solvent | Solution | mAb1 Conc. | Viscosity | Viscosity | mAb1 Conc. | Viscosity | Viscosity | at C₂₀₀ | SEC % |
| Additive | pH | pH | 1 (mg/ml) | 1 (cP) | 1 (ml/g) | 2 (mg/ml) | 2 (cP) | 2 (ml/g) | (cP) | Monomer |
| NaCl | 5.97 | 5.99 | 235 ± 5 | 87 ± 3 | 18.8 | 180 ± 0.1 | 30 ± 1 | 18.5 | 44 | TBD |
| NaCSA | 6.01 | 6.05 | 230 ± 0.4 | 38 ± 2 | 14.7 | — | — | — | — | TBD |
| NaCSA | 7.00 | 6.91 | 229 ± 3 | 51 ± 2 | 16.0 | 192 ± 3 | 16 ± 1 | 13.2 | 20 | TBD |
| Ala | 5.98 | 6.04 | 220 ± 7 | 210 ± 6 | 24.1 | 205 ± 5 | 52 ± 2 | 19.0 | 37 | TBD |

Effect of Lysine and Imidazole on Viscosity.

Additionally, mAb1 was concentrated in 305 mM lysine (Lys) at pH 6. Like His and Arg, Lys is a positively charged amino acid and is thus much more similar to His/Arg than a sodium ion. Like Arg, (and unlike His) Lys is essentially entirely charged at pH 6 (see FIG. 43). However, its charged R group is a primary amine, which according to molecular dynamics models does not preferentially interact with the protein surface to the extent observed for a guanidyl or imidazole group, as it does not contain any it electrons. (Heyda, Mason et al. 2010) As seen in FIG. 44 and Table 5-S10, the viscosity reduction caused by Lys is larger than that of NaCl, but is much smaller than observed for either His or Arg. This result further supports the hypothesis that the large viscosity reductions from His and Arg are favored by the preferential interactions of the imidazole and guanidyl groups with the protein surface to modulate the PPI. As shown in Tables 5-1 and 5-S10, the viscosity reduction for imidazole (Imid) at 305 mM was similar as for His at pH 6, but was larger at pH 7. However it was not quite as large as for Arg. At pH 7, Imid is more protonated than His (shown in FIG. 43), which may explain why it reduced the viscosity more than did His. The zwitterionic group of His in the mostly neutral state (small HisH⁺ concentration) was not as effective in reducing the viscosity, compared to cationic Imid which does not contain a zwitterionic moiety.

TABLE 5-S10
Viscosity and stability of mAb1 solutions concentrated by CF in
solvent 305 mM buffer. Inherent viscosities are calculated using a solvent
viscosity of 1.05 cP for Lys(HCl) and 1.30 cP for Arg(CSA). Properties that
were measured multiple times are represented as the average measurement
plus or minus the standard deviation of the measurements. Viscosity at C200
represents the calculated viscosity from the best fit of the data at 200 mg/ml
mAb1.

| | | | mAb1 | | Inherent | mAb1 | | Inherent | Viscosity | 4 Week |
|---|---|---|---|---|---|---|---|---|---|---|
| Buffer | Solvent | Solution | Conc. 1 | Viscosity | Viscosity | Conc. 2 | Viscosity | Viscosity | at C₂₀₀ | SEC % |
| Components | pH | pH | (mg/ml) | 1 (cP) | 1 (ml/g) | (mg/ml) | 2 (cP) | 2 (ml/g) | (cP) | Monomer |
| Lys(HCl) | 5.81 | 5.98 | 236 ± 1 | 74 ± 2 | 18.0 | 192 ± 1 | 29 ± 1 | 17.3 | 35 | TBD |
| Imid(HCl) | 5.90 | 5.96 | 231 ± 3 | 53 ± 2 | 16.9 | 200 ± 2 | 17 ± 1 | 14.0 | 17 | TBD |
| Imid(HCl) | 6.89 | 6.98 | 221 ± 1 | 56 ± 1 | 18.0 | 203 ± 3 | 25 ± 1 | 15.7 | 23 | TBD |
| Arg(CSA) | 5.75 | 5.86 | 252 ± 6 | 60 ± 2 | 15.2 | 187 ± 4 | 18 ± 1 | 13.9 | 22 | TBD |
| Arg(CSA) | 6.85 | 6.82 | 245 ± 1 | 45 ± 1 | 14.4 | 184 ± 11 | 14 ± 1 | 12.8 | 18 | TBD |

To further investigate the effect of neutral amino acids, an experiment was performed with alanine, with a CH₃ group that does not interact preferentially with proteins. As shown in Tables 5-1 and 5-S9 it had almost no effect on mAb1 viscosity. Here, the driving force to add zwitterionic groups to charged or hydrophobic sites on the protein is small given the weak interactions of alanine with proteins.

Effect of an anionic hydrophobic salt on viscosity. Previous studies have shown significant viscosity reductions for protein systems, including mAbs, using hydrophobic organic anions, including camphorsulfonic acid (CSA). (Du and Klibanov 2011; Guo, Chen et al. 2012) Therefore, experiments were performed with NaCSA and compared to those with NaCl. As shown in FIG. 35, NaCSA reduces the viscosity similarly to HisH⁺ at pH 6, whereas NaCl had no effect. These results agree with earlier studies (Du and Klibanov 2011; Guo, Chen et al. 2012) showing that blocking hydrophobic sites with hydrophobic anions as well as cations can reduce the viscosity of protein solutions. As shown in Table 5-S9 at pH 7, CSA⁻ causes a similar viscosity reduction as Arg. The removal of a protein surface hydrophobic site with either a cation or anion can weaken hydrophobic PPI and thus reduce viscosity. According to this scenario, blocking hydrophobic interactions is more important than whether the charge on the surface of the protein is increased or decreased. However, from the viewpoint of anisotropic local electrostatic and charge dipole interactions, it would seem that adding negative charges with CSA⁻ could produce the opposite effect of either lowering pH (without added co-solute) or adding cationic protonated bases. For CSA⁻, the inherent viscosity decreased as the pH was increased (Table 5-1), opposite to the behavior for the cationic co-solutes. It is conceivable for an anionic co-solute the strategy would be to shift the balance of protein charge towards negative charges to minimize local anisotropic attractive interactions with positive sites and to increase electrostatic repulsion. Thus, an increase in pH would favor this scenario consistent with the observed decrease in the inherent viscosity.

To further understand the behavior for ArgH⁺ and CSA⁻, the two were combined with 305 mM of each without the need for counter-ions such as Na⁺ and Cl⁻. As shown in Table 5-S10, at both pH 6 and pH 7 the viscosity reduction remained comparable to what was seen for either Arg or CSA⁻ alone. In this case the total concentration of hydrophobic salts was twice as high, 610 mM or 500 mM nominally (after correction in eq. 5). Thus, it appeared that 250 mM of either Arg or CSA may have been enough to saturate the hydrophobic sites of mAb1. However, this simplified analysis does not address the question of the change in charges with added cations or anions.

Effects of Co-Solutes and pH on Turbidity and Stability.

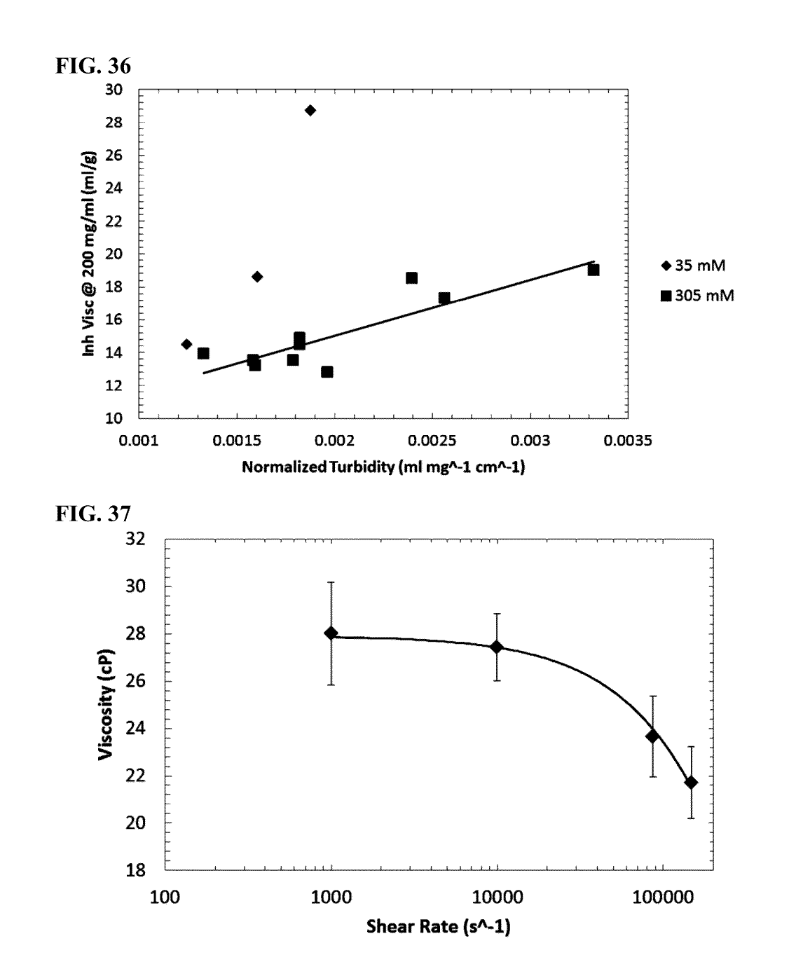

For pharmaceutical formulations, it is also essential to determine the effects of these co-solutes on the stability of mAb1 as well as viscosity. Therefore, the initial (pre-storage) stability of many of the CF samples was measured via turbidity at 350 nm without diluting the samples. As all samples were visually transparent, the measured turbidities were all relatively low, as shown in Table 5-2; however, for a given co-solute type and concentration, the turbidity tended to increase with pH. This trend is expected as the higher pH is closer to the pI of the mAb, whereas most attractive PPI promote more aggregation as characterized by higher turbidity. (Chari, Jerath et al. 2009; Hawe, Romeijn et al. 2012) As shown in FIG. 36, the turbidity of samples containing 305 mM co-solute was correlated with the viscosity. The samples with the highest viscosities (305 mM his at pH 7.3, 305 mM lys at pH 6, and 35 mM his with 270 mM NaCl at pH 6) had the highest turbidities, while the Arg, ArgCSA, and lower pH his samples all had lower turbidities. The samples made in 35 mM his also showed larger increases viscosity with increasing turbidity.

In addition to initial stability, storage stability is essential for the application of proteins. Therefore the monomer retention for CF samples was measured by SE-HPLC after accelerated storage at 40° C. for four weeks. As shown in Table 5-3, the stability of the His and Arg samples showed similar trends as for viscosity reduction, as the least viscous samples tended to have the highest stability and lowest turbidity and vice versa. The systems with low viscosities, pH ˜5 and ˜6 His and pH ˜5, ˜6 and ˜7 Arg all had monomer retention of ˜98% and greater. This correlation seems reasonable as weakening attraction should simultaneously improve viscosity and stability. (Connolly, Petry et al. 2012; Buck, Chaudhri et al. 2015) However, the data suggest that for similarly viscous systems, such as 35 mM His at pH 6 and 305 mM His at pH 7.3 the sample with more His is more stable. One possible explanation for this is that the greater amount of unbound His in solution for the 305 mM concentration is interacting with the protein via depletion attraction, promoting conformational stability. (Shen, Cheung et al. 2006; Johnston, Maynard et al. 2012)

Effect of Shear Rate on Viscosity.

So far, all of the viscosities reported have been at a shear rate of 1000 s-1 for CF samples and less than 1000 s-1 for LD samples. However, several studies have shown that mAbs may undergo shear thinning at very high shear rates. (Liu, Nguyen et al. 2005; Jezek, Rides et al. 2011; Rathore, Pranay et al. 2012; Zarraga, Taing et al. 2013; Castellanos, Pathak et al. 2014) The applied shear rate for subcutaneous injection, often greater than 100,000 s-1, may be beneficial for lowering the viscosity if the shear thinning breaks protein interactions. (Burckbuchler, Mekhloufi et al. 2010; Rathore, Pranay et al. 2012; Allmendinger, Fischer et al. 2014) Unfortunately, we only report one measurement at high shear rate given the large sample volume of several mL's needed for a reliable measurement with the Rheosense m-VROC. Here, a large sample of mAb1 was concentrated by CF to 215 mg/ml in a solvent containing 320 mM His, 80 mM citric acid and 105 mM trehalose at pH 5.92. As shown in FIG. 37 the measured viscosity of this sample was 28 cP at 1000 s-1 and remained relatively constant up to 10,000 s-1. However, the viscosity dropped to 22 cP at 150,000 s-1, a reduction of over 20%. This level of reduction in viscosity at shear rates relevant to subcutaneous injection would be beneficial to reach a commonly indicated target of 20 cP.

Experimental Section

Materials

The monoclonal antibody used in this study (mAb1) is an IgG1 type antibody with an isoelectric point (pI) of 9.3. MAb1 was supplied at −120 mg/ml in a proprietary buffer composition. Alanine, arginine, arginine hydrochloride, camphorsulfonic acid (CSA), histidine, histidine hydrochloride monohydrate, imidazole, lysine, lysine hydrochloride, sodium chloride, and hydrochloric acid were purchased from Fisher Scientific, Fairlawn, N.J. Sodium camphorsulfonate was purchased from TCI America, Portland, Oreg. Trehalose dihydrate was obtained from Ferro Pfanstiehl Laboratories Inc., Waukegan, Ill. Amicon Ultra-15 Ultracel-30K and Amicon Ultra 0.5 Ultracel-50K centrifugal filters were purchased from Merck Millipore Ltd. Ireland.

Sample Preparation

Centrifugal Diafiltration.

1 mL of the 120 mg/mL mAb1 solution was initially diluted in 11 mL of a buffer containing desired concentrations of co-solutes (12 mL total, initial buffer volumetric fraction is 8.3% (1 mL out of 12 mL)). The resulting solution was then filtered in a centrifuge (Eppendorf Centrifuge 5415D) using a Millipore Amicon Ultra 15 centrifugal filter unit with a molecular weight cutoff of 30 kDa and a capacity of 12 mL at a spin speed of 4500 rcf, and a controlled temperature of 20° C. for 15 minutes. The protein solution was concentrated to a solution volume of about 2 mL and a protein concentration of ˜60 mg/mL. The retained protein solution was then again diluted using the desired dispersion buffer to make up the volume to 12 ml (initial buffer volume fraction reduced to 1.4% (8.3% out of 2 mL in 12 mL)) and then centrifuged again. This process was repeated 2 or more times until the volume of flow through was about 40 mL and the volumetric fraction of initial buffer was less than 1% assuming ideal mixing. After this the solution was further concentrated by continuing the centrifugation so that the final volume was about 2 mL at about 60 mg/mL; the protein concentration was then measured via UV-Vis absorbance (below).

Centrifugal Ultrafiltration (CF).

Some of the protein samples were concentrated by centrifugal filtration (CF). These samples were made directly from the final retentate at the end of centrifugal diafiltration. These samples were pipetted into a new filter and weighed after tare weights were taken of the individual components (filter, permeate tube and retentate tube) of the centrifugal filter assembly (Millipore Amicon Ultra 15). The filter assembly was then centrifuged (Eppendorf Centrifuge 5415D) at 4500 rcf and 20° C. for about 60 minutes in 5-20 minute increments, with the retentate volume monitored gravimetrically at every stop until the calculated final volume for the desired final concentration was reached. The target volume was concentrated assuming a density of 1350 mg/mL for solvated mAb1, 2200 mg/mL for solvated sodium and chloride ions, 1500 and 1400 mg/mL for solvated imidazole and CSA, respectively and amino acid densities as provided by Zhao, et. al. (Zhao, Brown et al. 2011) Once the desired concentration had been reached, the protein dispersion in the retentate was mixed and then recovered into an Axygen Microtube (MCT 150-C) via pipette.

Many of these samples have viscosities reported at two concentrations. For those samples the system was concentrated to the highest concentration first, followed by measurement of the concentration, viscosity and pH. Then the sample was diluted to a lower concentration in the same solvent that it had been diafiltered into, before re-measuring the concentration, viscosity and pH. The pH reported is that at the highest concentration, but the pH change upon dilution was always <0.1 pH units.

Lyophilization Dilution (LD).

Other mAb1 samples were made by lyophilization dilution (LD), in which we mixed lyophilized protein powder into an aqueous buffer to achieve 100150 μl of a ˜250 mg/ml mAb1 dispersion. A 5% excess by mass of the lyophilized powder was used relative to the calculated mass of powder to produce 100 μl of a 250 mg/mL mAb1 dispersion. The 5% excess was added in case the lyophilized powder was not 100% dry. The lyophilized powder contained a 0.2:1 mass ratio of trehalose to mAb1 without any other solids. Consequently, all additional co-solutes are added via the solvent. The lyophilized powder was made by centrifugal diafiltration of mAb1 into DI water and then adding a calculated amount of 300 mg/ml trehalose solution to achieve the 0.2:1 mass ratio. The solution was then frozen in a −40° C. freezer, and lyophilized in a tray lyophilizer at −40° C. for 1250 min and then −25° C. for 4250 min. Therefore all mAb solutions made by LD contained ˜130 mM trehalose.

Sample Characterization

Viscosity Measurement.

The viscosity of the samples made by CF was measured using a microfluidic Viscometer/Rheometer-on-Chip (m-VROC, Rheosense Inc. San Ramon, Calif.) with a C05 chip, for shear rates ranging from 500 to 10,000 s⁻¹, and an E04 chip for higher shear rates. The sample was loaded into either a 500 μL glass syringe, or a 10 mL glass syringe for >10,000 s⁻¹ measurements (#1750 and #1010, Hamilton Co., Reno, Nev.) which was loaded into the m-VROC. Viscosity measurements were taken at shear rates of 500 s⁻¹ and 1000 s⁻¹ twice to make sure the measured viscosity had stabilized. The viscosity and standard deviation reported is from the second measurement at 1000 s⁻¹, though Newtonian behavior was observed for all samples in this shear range. All viscosities were measured at 25° C.

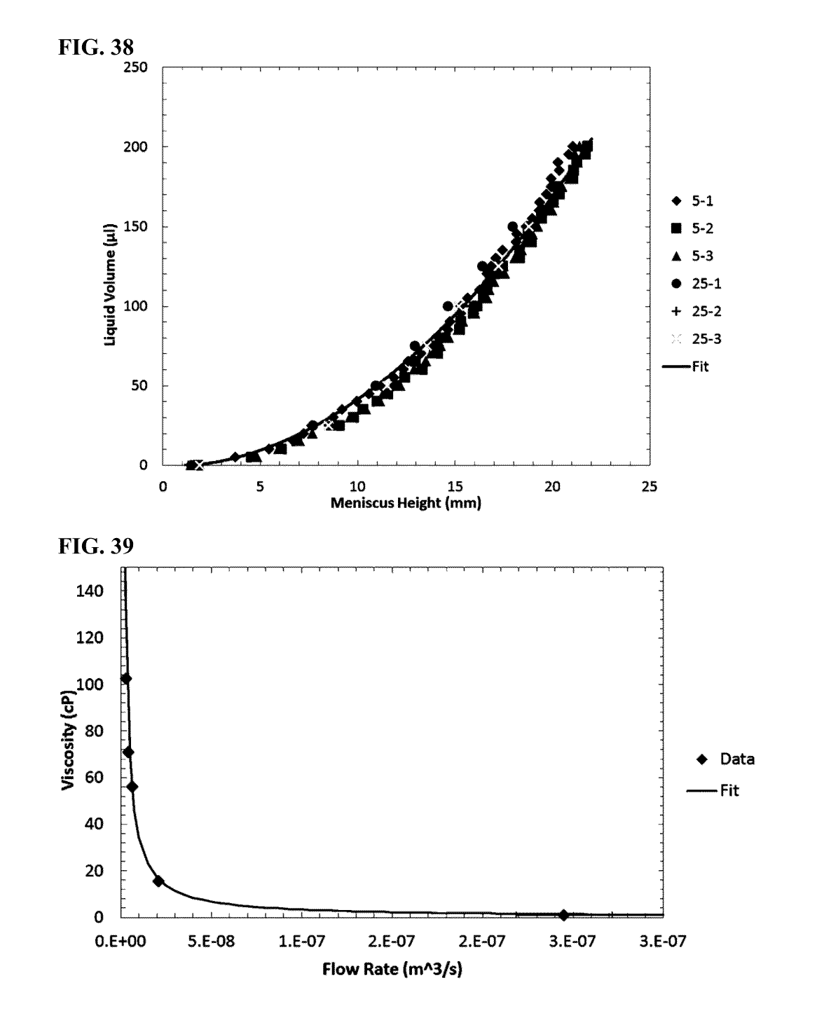

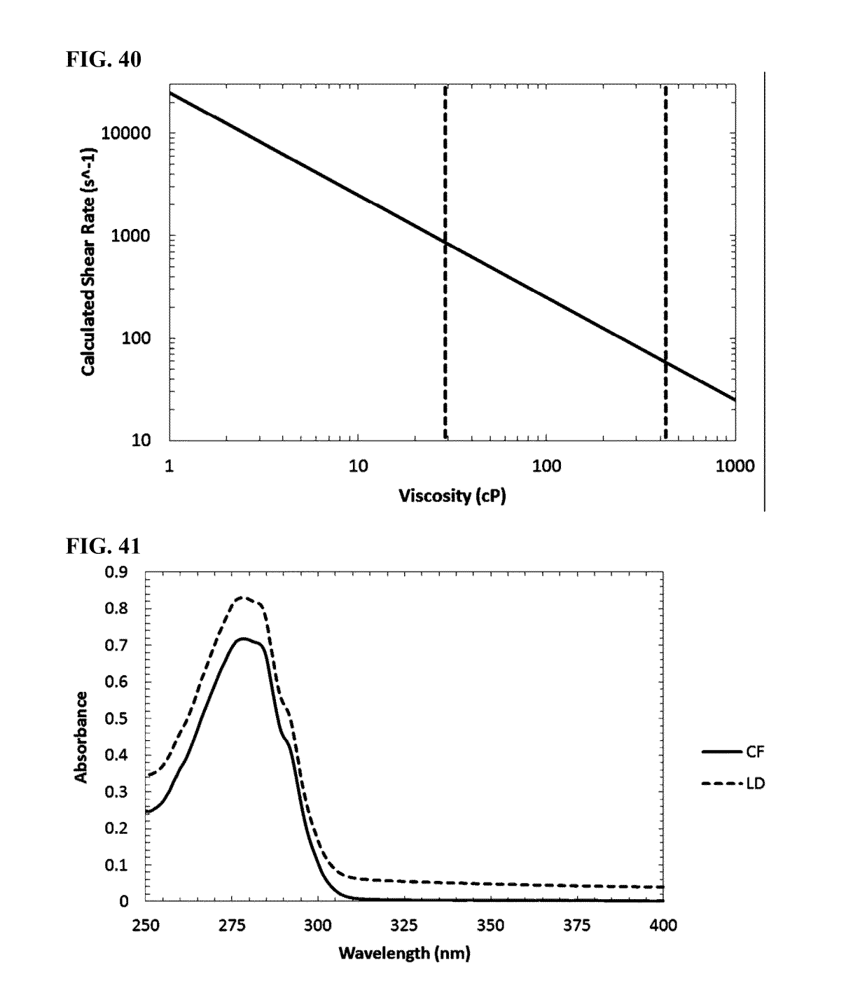

Due to the small size of the samples made by LD, the viscosity of these samples was measured with a syringe viscometer calibrated using viscosity standards S60, N44, N35 and N10 (Cannon Instrument Company, State College, Pa.) and DI water. All viscosities were measured in triplicate after equilibrating the sample temperature to 25° C. on a heating block. Reported viscosities are the average of the three measurements plus or minus the standard deviation of these measurements. The syringe viscometer measures the flow rate of liquid through a needle, and the viscosity is calculated using the Hagen-Poiseuille equation (Eqn. 5-1)

$$η = \frac{π {r}^{4} Δ P}{8 LQ} \qquad ( 5 \text{-} 1 )$$

where η is the viscosity, r is the inner diameter of the needle, L is the length of the needle, Q is the liquid flow rate through the needle, and ΔP is the pressure drop across the needle (which has been previously determined for this system using the viscosity standards, shown in supplemental). To measure the viscosity, ˜100 uL of the protein sample was pipetted into a 0.1 ml conical vial (catalog no. 03-341-15; Wheaton, Millville, N.J.). A 25-gauge (0.26 mm diameter), 1.5″ (4 cm) Precision Glide needle (Becton Dickinson & Co.) attached to a 1 ml Luer-Lok™ syringe (Becton Dickinson & Co.) is inserted into the bottom of the vial. The syringe stopper is pulled up to the 1 ml mark in one motion taking approximately one second, and the vial is filmed by a Kodak Z812 IS zoom digital camera at a frame rate, x, of 30 frames per second. The videos are analyzed using ImageJ image analysis (Schneider, Rasband et al. 2012) to determine the height of the liquid column in the vial (which has been correlated with the volume of liquid inside the vial, shown in FIG. 38 of the supplemental section) as a function of time. The resulting liquid flow rate, Q, is determined from Eqn. 5-2:

$$Q = \frac{{V}_{1} - {V}_{2}}{t} , t = \frac{{f}_{2} - {f}_{1}}{x} \qquad ( 5 \text{-} 2 )$$

where t is the time between time points, V₁ is the liquid volume in the vial when the liquid height has reached approximately 80% of its original height, and V₂ is the liquid volume at a second time point before the liquid volume reaches zero, f₁ is the frame number of the video at the first time point, f₂ is the frame number at the second time point, and x is the frame rate of the camera (30 frames per second). The methods of determining both the correlation between the liquid volume and height in the vial as well as the pressure drop across the needle can be found in FIG. 39 of the supplemental section. It should be noted that the applied shear rate from this viscometer depends on the viscosity of the sample. Shear rate predictions are shown in FIG. 40 of the supplemental section. The equation for shear rate prediction is calculated assuming Newtonian flow from equation 5-S1 following:

$$γ = \frac{r Δ P}{2 η L} ( \frac{3 n + 1}{4 n} ) \qquad ( 5 \text{-} S1 )$$

where γ is shear rate, ΔP is pressure drop across the needle, η is viscosity, r is the needle radius and L is the needle length, for Newtonian fluids (as is assumed in this case) n=1.

Correlation Between Vial Height and Volume for Viscosity Measurements of LD Samples.

To determine the volume of liquid in the vial, the meniscus height was correlated with the liquid volume for eight different vials, where a known volume of liquid was pipetted into the vial using an Eppendorf Research Plus adjustable volume pipette (Eppendorf AG, Hamburg, Germany). The meniscus height was measured by ImageJ analysis of a photo of the liquid column inside the vial, measuring from the bottom of the vial to the bottom of the liquid meniscus. The meniscus height was measured for volumes between 0 and 200 uL over eight vials in increments of 5 μl for three of the vials and 25 μl for three of the vials. The liquid volume as a function of the liquid height was determined by a quadratic regression of the data, as shown in FIG. 38.

Needle Pressure Drop Calculation for Viscosity Measurements of LD Samples.

The pressure drop across the needle was determined using the viscosity standards S60, N44, N35, and N10 (Cannon Instrument Company, State College, Pa.) and DI water, which at room temperature (25° C.) have viscosities of 102.4, 70.92, 55.75, 15.39, and 0.986 cP, respectively. The flow rate was measured three times using 100 μl of each standard in five different vials, totaling 15 measurements per sample. These average flow rates were plotted against the known viscosity of the liquids, and the pressure drop was fit to these points using the Hagen-Poiseuille equation, giving a pressure drop across the needle of 7942 Pa. The viscosity calibration and fit can be seen in FIG. 39.

Samples with viscosities measured at two or more concentrations were fit to the modified version of the Mooney equation, also referred to as the Ross-Minton equation

$$\frac{η}{{η}_{0}} = exp ( \frac{[ η ] c}{1 - [ η ] \frac{k}{v} c} ) \qquad ( 5 \text{-} 3 )$$

where η₀ is the solvent viscosity, c is the mAb1 concentration, [η] is the intrinsic viscosity, k is the crowding factor, and v is the Simha parameter (shape-determining factor). (Mehl, Oncley et al. 1940; Kanai, Liu et al. 2008) Both [η] and the ratio k/v were used as fit parameters. The results of these fit parameters are reported in Table 5-S7. Equation 3 is used to determine the predicted viscosity at 200 mg/ml.

Protein Concentration and pH Measurement.

The concentration of the mAb1 dispersions were measured in duplicate, where 2 μl of solution was diluted into 998 μl of 50 mM pH 6.4 phosphate buffer and mixed well. The background-corrected absorbance of the resulting solution was measured over a spectrum of 400 to 250 nm using a Cary 3E UV-visible spectrophotometer in a quartz cuvette (Hellma GmbH and Co, Mullheim, Germany) with a path length of 1 cm. The concentration was determined from the absorbance using a modified Beer's law (Eqn. 5-4)

A280-A350=ε*b*c (5-4)

where A280 is the absorbance at 280 nm and A350 is the absorbance at 350 nm, c is the extinction coefficient (1.42 ml mg⁻¹ cm⁻¹ for mAb1) and b is the cell path length (1 cm). Representative absorbance spectra can be seen in FIG. 41. The solution pH was measured by a pH meter (Mettler Toledo InLab Micro) calibrated using standards of pH 4 and 7 (Lab Safety Supply).

Protein Turbidity.

The protein turbidity was measured using a Cary 3E UV-visible spectrophotometer in a Micro Volume Size Cell (A54094 Beckman Coulter) with a path length of 0.2 cm over a spectrum of 1100 to 200 nm. The turbidity was measured on both the sample without any dilution and the protein-free solvent. Representative turbidity spectra can be seen in FIG. 42. The reported values are the measured A350 of the sample minus that of the solvent divided by the protein concentration in ml/mg and the sample path length in cm.

Size Exclusion High Performance Liquid Chromatography (SE-HPLC).

For characterization of irreversible non-covalent aggregate levels in the final dispersions, the mAb dispersion was diluted to 2 mg/mL in the mobile phase (92 mM sodium phosphate dibasic, 211 mM sodium sulfate, pH 7). The diluted samples were sterile filtered through a 0.22 μm PES syringe filter (Celltreat Scientific Products, Shirley, Mass.). Duplicate 10 μL injections of each sample were analyzed with a Waters Breeze HPLC (Waters Corporation, Milford, Mass.) equipped with a Tosoh Biosciences TSKge13000SW_{XL} column (Tosoh Corporation, Tokyo, Japan). The eluate was monitored by the UV absorbance at 214 nm and 280 nm.

Accelerated Storage Stability.

Small aliquots (50 μL) of the final mAb1 dispersions were stored in 300 μL HPLC vial inserts (Thermo Scientific) inside 1 mL HPLC vials (Fisher Scientific) sealed with two layers of Parafilm surrounding a layer of aluminum foil. The mAb1 concentration of each sample stored can be found in Tables 5-S1-5-S5 under the column title “mAb1 Concentration 2 (mg/ml)”. For the samples that do not have value in this column, the mAb1 concentration was diluted to ˜200 mg/ml for storage. The sealed vials were stored in a 40° C. incubator for 4 weeks. After 4 weeks of storage the samples were analyzed by SE-HPLC.

Conclusion.

Viscosities below 20 cP at 200 mg/ml mAb1 were achieved while maintaining 98% monomer or more after accelerated storage with the basic co-solutes Arg, and His at pH values ranging from 5 to 7. Low viscosities were achieved for the basic co-solute Imid and for the hydrophobic anion camphorsulfonate; however, the storage stability of these samples is yet to be determined. In contrast, addition of inorganic electrolytes to screen charges in the double layer did not influence the viscosity. The addition of interacting, cationic, organic co-solute bases, Arg, His and Imid at high concentrations adds positive charges to mAb1, thus weakening local anisotropic attractive electrostatic PPI and strengthening electrostatic repulsion. With less attraction between proteins, the viscosity decreased and the protein stability against aggregation increased. For all of the co-solutes studied, the turbidity decreased as the viscosity decreased. This behavior may indicate a decrease in the concentration of aggregates that raise viscosity. The lowest absolute viscosities were observed at pH 5 to 5.5 with viscosities as low as 44 cP at 250 mg/ml and 12 cp at 200 mg/ml (interpolation with the Ross-Minton equation). However, the degree of viscosity reduction was much smaller at lower pH values, further below the pI, where even without added co-solute, the local anisotropic electrostatic PPI are less attractive, given the larger number of positive charges. (Chari, Jerath et al. 2009) All of the strong organic bases produced large viscosity reductions at pH 7 by adding positive charges to the protein surface, whereas His had a weaker effect, as it is mostly neutral. Since the addition of inorganic electrolytes did not improve the viscosity or the stability, it appears that preferential interactions of co-solutes with the protein, and not merely double layer screening, are required. These interactions mitigate local attractive electrostatic PPI and increase long ranged electrostatic repulsion between the proteins. Because His, Arg and Imid cations as well as CSA anions interact with the aromatic sites on the protein surface, they may also screen hydrophobic interactions. While the effect of co-solutes on the viscosities were similar for protein dispersions produced by the lyophilization dilution and centrifugation filtration methods, despite the highly different pathways, future work would be warranted to examine how these process influence the protein morphology and stability.

### References (Example 5)

Allmendinger, A., S. Fischer, et al. (2014), European journal of pharmaceutics and biopharmaceutics: official journal of Arbeitsgemeinschaft fur Pharmazeutische Verfahrenstechnik e.V 87(2): 318-328; Aragon, S. and D. K. Hahn (2006), Biophysical Journal 91(5): 1591-1603; Arakawa, T., D. Ejima, et al. (2007), Biophysical chemistry 127(1-2): 1-8; Asakura, S. and F. Oosawa (1958), Journal of Polymer Science 33(126): 183-192; Borwankar, A. U., A. K. Dinin, et al. (2013), Soft Matter 9(6): 1766-1771; Buck, P. M., A. Chaudhri, et al. (2015), Molecular pharmaceutics 12(1): 127-139; Burckbuchler, V., G. Mekhloufi, et al. (2010), Eur. J. Pharm. Biopharm. 76(3): 351-356; Carpenter, J. F., T. W. Randolph, et al. (2009), Journal of Pharmaceutical Sciences 98(4): 1201-1205; Carter, P. J. (2006), Nature Reviews Immunology 6(5): 343-357; Castellanos, M. M., J. A. Pathak, et al. (2014), Soft Matter 10(1): 122-131; Castellanos, Maria M., Jai A. Pathak, et al. (2014), Biophysical Journal 107(2): 469-476; Chari, R., K. Jerath, et al. (2009), Pharmaceutical Research 26(12): 2607-2618; Chaudhri, A., I. E. Zarraga, et al. (2012), The journal of physical chemistry. B 116(28): 8045-8057; Chaudhri, A., I. E. Zarraga, et al. (2013), The journal of physical chemistry. B 117(5): 1269-1279; Chen, B., R. Bautista, et al. (2003), Pharmaceutical Research 20(12): 1952-1960; Chen, S., Z. Cao, et al. (2009), Biomaterials 30(29): 5892-5896; Connolly, B. D., C. Petry, et al. (2012), Biophysical Journal 103(1): 69-78; Du, Q. S., J. Z. Meng, et al. (2012), Journal of molecular graphics & modelling 34: 38-45; Du, Q. S., Q. Y. Wang, et al. (2013), Chemistry Central journal 7: 92; Du, W. and A. M. Klibanov (2011), Biotechnology and bioengineering 108(3): 632-636; Fathallah, A. M., M. Chiang, et al. (2015), Journal of Pharmaceutical Sciences; Fersht, A. R. and L. Serrano (1993), Current Opinion in Structural Biology 3(1): 75-83; Fukuda, M., D. Kameoka, et al. (2014), Pharmaceutical Research 31(4): 992-1001; Fukuda, M., C. Moriyama, et al. (2015), Pharmaceutical Research; Guo, Z., A. Chen, et al. (2012), Pharmaceutical Research 29(11): 3102-3109; Hawe, A., S. Romeijn, et al. (2012), Journal of Pharmaceutical Sciences 101(11): 4129-4139; He, F., C. Woods, et al. (2011), Pharmaceutical Research 28(7): 1552-1560; Heyda, J., P. E. Mason, et al. (2010), Journal of Physical Chemistry B 114(26): 8744-8749; Holstein, M. A., S. Parimal, et al. (2012), Biotechnology and bioengineering 109(1): 176-186; Hou, Y. and S. M. Cramer (2011), Journal of chromatography. A 1218(43): 7813-7820; Ignatova, Z. and L. M. Gierasch (2006), Proceedings of the National Academy of Sciences of the United States of America 103(36): 13357-13361; Inoue, N., E. Takai, et al. (2014), Molecular pharmaceutics 11(6): 1889-1896; Izutsu, K., S. Yoshioka, et al. (1991), International Journal of Pharmaceutics 71(1-2): 137-146; Jezek, J., M. Rides, et al. (2011), Advanced drug delivery reviews 63(13): 1107-1117; Jiang, S. and Z. Cao (2010), Advanced materials 22(9): 920-932; Johnson, H. R. and A. M. Lenhoff (2013), Molecular pharmaceutics 10(10): 3582-3591; Johnston, K. P., J. A. Maynard, et al. (2012), ACS Nano; Kamerzell, T. J., R. Esfandiary, et al. (2011), Advanced drug delivery reviews 63(13): 1118-1159; Kanai, S., J. Liu, et al. (2008), J Pharm Sci 97(10): 4219-4227; Karow, A. R., S. Bahrenburg, et al. (2013), Biotechnology progress 29(2): 480-492; Kreilgaard, L., S. Frokjaer, et al. (1998), Archives of Biochemistry and Biophysics 360(1): 121-134; Kreilgaard, L., S. Frokjaer, et al. (1999), Journal of Pharmaceutical Sciences 88(3): 281-290; Ladiwala, A. R. A., M. Bhattacharya, et al. (2012), Proceedings of the National Academy of Sciences of the United States of America 109(49): 19965-19970; Li, L., S. Kumar, et al. (2014), Pharmaceutical Research 31(11): 3161-3178; Liao, S. M., Q. S. Du, et al. (2013), Chemistry Central journal 7(1): 44; Lilyestrom, W. G., S. Yadav, et al. (2013), The journal of physical chemistry. B 117(21): 6373-6384; Liu, J., M. D. Nguyen, et al. (2005), Journal of Pharmaceutical Sciences 94(9): 1928-1940; Lo Nostro, P. and B. W. Ninham (2012), Chemical Reviews 112(4): 2286-2322; Loewenthal, R., J. Sancho, et al. (1992), Journal of Molecular Biology 224(3): 759-770; Mehl, J. W., J. L. Oncley, et al. (1940), Science (New York, N.Y.) 92(2380): 132-133; Miller, M. A., J. D. Engstrom, et al. (2010), Langmuir 26(2): 1067-1074; Pathak, Jai A., Rumi R. Sologuren, et al. (2013), Biophysical journal 104(4): 913-923; Perchiacca, J. M., A. R. A. Ladiwala, et al. (2012), Proceedings of the National Academy of Sciences of the United States of America 109(1): 84-89; Rathore, N., P. Pranay, et al. (2012), Journal of Pharmaceutical Sciences 101(12): 4472-4480; Reichert, J. M. (2014), Retrieved Jan. 15, 2015, from website: www.antibodysociety.org/news/approved_mabs.php; Roberts, C. J. (2014), Current Opinion in Biotechnology 30: 211-217; Roberts, C. J. and M. A. Blanco (2014), The journal of physical chemistry. B 118(44): 12599-12611; Saluja, A. and D. S. Kalonia (2008), International Journal of Pharmaceutics 358(1-2): 1-15; Samuel, D., T. K. S. Kumar, et al. (2000), Protein Science 9(2): 344-352; Sarangapani, P. S., S. D. Hudson, et al. (2015), Biophysical Journal 108(3): 724-737; Sarangapani, P. S., S. D. Hudson, et al. (2013), Biophysical Journal 105(10): 2418-2426; Scherer, T. M. (2013), The journal of physical chemistry. B 117(8): 2254-2266; Schmit, J. D., F. He, et al. (2014), The journal of physical chemistry. B 118(19): 5044-5049; Schneider, C. A., W. S. Rasband, et al. (2012), Nature Methods 9(7): 671-675; Schneider, C. P., D. Shukla, et al. (2011), Journal of Physical Chemistry B 115(22): 7447-7458; Schneider, C. P. and B. L. Trout (2009), Journal of Physical Chemistry B 113(7): 2050-2058; Severson, C. C. (2015), Canadian Oncology Nursing Journal 25(3): 341-346; Shen, V. K., J. K. Cheung, et al. (2006), Biophysical Journal 90(6): 1949-1960; Shih, O., A. H. England, et al. (2013), The Journal of Chemical Physics 139(3): 035104; Shire, S. J., Z. Shahrokh, et al. (2004), J. Pharm. Sci. 93(6): 1390-1402; Shukla, D., C. P. Schneider, et al. (2011), Advanced drug delivery reviews 63(13): 1074-1085; Shukla, D. and B. L. Trout (2011), The journal of physical chemistry. B 115(5): 1243-1253; Srinivasan, C., A. K. Weight, et al. (2013), Pharmaceutical Research 30(7): 1749-1757; Teeters, M., D. Bezila, et al. (2011), Biotechnology and bioengineering 108(6): 1338-1346; Vondrasek, J., P. E. Mason, et al. (2009), Journal of Physical Chemistry B 113(27): 9041-9045; Vrij, A. (1976), Pure and Applied Chemistry 48(4): 471-483; Wang, W. (2015), Protein science: a publication of the Protein Society 24(7): 1031-1039; Webb, S., S. Rule, et al. (1997), Pharmaceutical Research (New York) 14(11 SUPPL.): S159; Yadav, S., S. J. Shire, et al. (2010), J. Pharm. Sci. 99(12): 4812-4829; Yearley, E. J., P. D. Godfrin, et al. (2014), Biophysical Journal 106(8): 1763-1770; Zarraga, I. E., R. Taing, et al. (2013), Journal of Pharmaceutical Sciences 102(8): 2538-2549; Zhao, H., P. H. Brown, et al. (2011), Biophysical Journal 100(9): 2309-2317.

### Example 6

### Further Studies on Viscosity with Proline Addition

Materials and Methods

The monoclonal antibody used in this study (mAb1) is an IgG1 type antibody with an isoelectric point (pI) of 9.3, was supplied at ˜120 mg/ml in a proprietary buffer. Concentrated mAb dispersions were prepared by the centrifugal filtration (CF) technique as described in earlier examples. In order to account for the effects of volume exclusion on the final buffer composition (cosolute concentrations) after ultrafiltration, buffers were prepared at a higher cosolute concentration (C_{co,prep}) than the target final concentration (C_{co,target}) according to Eqn. 6-1,

$${C}_{co , prep} = \frac{{c}_{co , target}}{( 1 - \frac{{c}_{protein}}{{ρ}_{prot}} )} \qquad ( 6 \text{-} 1 )$$

where Cₚᵣₒₜₑᵢₙ was the targeted mAb concentration (set at 230 mg/mL) and ρₚᵣₒₜ is the assumed protein density (1350 mg/mL [1]). The initial mAb viscosity and stability after 4 weeks of accelerated storage at 40° C. was measured by HP-SEC as described in earlier examples.

Results

Increasing Viscosity Reduction with Proline Addition.

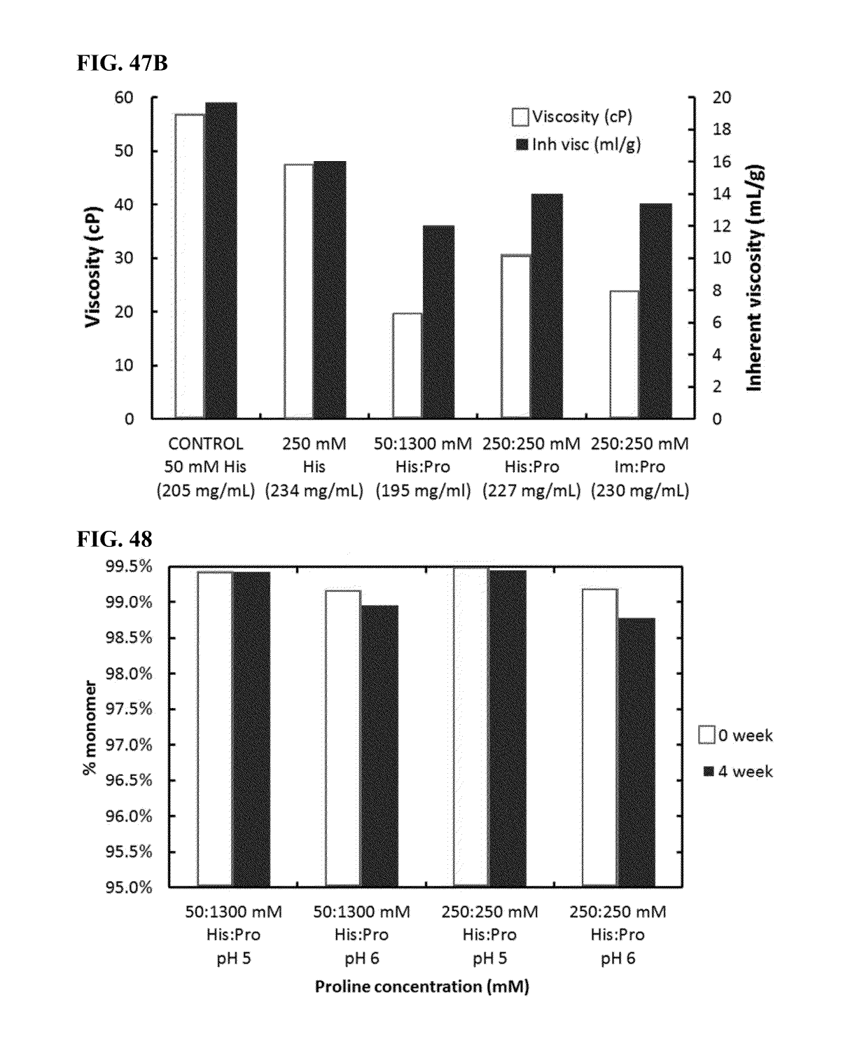

An ultrahigh concentration of proline (1300 mM) in 50 mM histidine was added to the mAb dispersion to reduce the viscosity at pH 6 to low levels for subcutaneous injection, close to the threshold concentration of 1.5 M, above which proline has been shown to solubilize poorly-soluble proteins and suppress aggregation during protein refolding. [1,2] The proline addition yielded a 3× viscosity reduction down to 20 cP at 200 mg/mL (FIG. 47B; Table 6-1). The proline caused a smaller reduction at pH 5, down to 21 cP at 210 mg/mL (FIG. 47A; Table 601).

To attempt to further modulate the electrostatic and hydrophobic PPI to reduce the viscosity, we examined mixtures of proline with a charged cosolute, either protonated histidine or imidazole. As shown in FIGS. 47A-47B, the combination of 250 mM proline in 250 mM histidine or imidazole led to a large reduction in viscosity and inherent viscosity (Table 6-2) comparable to 1300 mM proline. The reduction in inherent viscosity was larger at pH 6 than at pH 5, similar to the trends observed earlier for the mAb dispersions buffered with only 50 mM histidine.

Increasing mAb Stability with Increasing Proline Concentration.

In order for the concentrated mAb dispersions to be suitable for use in subcutaneous injection, in addition to having a low viscosity, the dispersion must remain stable with regard to irreversible aggregation. As seen in FIG. 48, mAb1 showed a high initial stability at both pH 5 and 6 with 99% monomer. The addition of ultrahigh proline was found to greatly improve the mAb stability after 4-week accelerated storage at 40° C., reducing the monomer loss down to 0.2% at the highest proline concentration of 1300 mM. In contrast, the mixed proline-histidine dispersions showed slightly lower stabilities, especially at pH 6, but still maintained a relatively high monomer content of >98.5%.

In summary, the mixed-systems afforded the second lowest inherent viscosities in the study, with viscosities in the 20-30 cP range for both pH 5 (FIG. 47A) and pH 6 (FIG. 47B). The lowest inherent viscosities of ˜12 mL/g at both pH were obtained with 50 mM histidine and 1300 mM proline. However, the lowest viscosity of 17 cP was obtained for 201 mg/ml mAb at pH 6 for 250 mM each of histidine and proline. Although the addition of 1300 mM proline yielded the greatest viscosity reduction and protein stabilization, the cosolute concentration required is too high to be practical. The similarly low viscosities and high stabilities obtained with the mixed proline and histidine systems at half the cosolute concentration therefore offer a more practical alternative for reducing viscosity and enhancing stability.

### Tables (Example 6)

TABLE 6-1
Dependence of mAb viscosity on proline concentration
at pH 5 and 6 with 50 mM His(HCl) for mAb dispersions
formed by centrifugation filtration.

| | | | | | Sol- | | |
|---|---|---|---|---|---|---|---|
| mAb | | | | | vent | Inh | |
| conc | | Proline conc | | Viscosity | visc. | visc | A₃₅₀ |
| (mg/ml) | pH | mg/ml | mM | (cP) | (cP) | (ml/g) | (cm⁻¹) |

pH 5

| | | | | | | | |
|---|---|---|---|---|---|---|---|
| 208 ± 4.3 | 4.93 | 0 | 0 | 20.8 ± 2.1 | 1 | 14.6 | 0.293 |
| 225 ± 2.2 | 5.12 | 29 | 250 | 26.0 ± 3.2 | 1.1 | 14.1 | 0.272 |
| 239 ± 1.0 | 5.15 | 86 | 750 | 29.2 ± 1.0 | 1.8 | 11.7 | 0.427 |
| 209 ± 0.9 | 5.27 | 150 | 1300 | 20.6 ± 0.9 | 1.9 | 11.5 | 0.431 |

pH 6

| | | | | | | | |
|---|---|---|---|---|---|---|---|
| 205 ± 9.6 | 5.96 | 0 | 0 | 56.5 ± 2.4 | 1 | 19.7 | 0.327 |
| 205 ± 0.4 | 5.94 | 29 | 250 | 45.2 ± 1.9 | 1.2 | 17.6 | 0.380 |
| 205 ± 2.9 | 6.05 | 86 | 750 | 32.5 ± 2.7 | 1.8 | 14.3 | 0.511 |
| 195 ± 1.3 | 6.07 | 150 | 1300 | 19.6 ± 1.6 | 1.9 | 12.0 | 0.525 |

TABLE 6-2
Reduction of mAb viscosity at pH 5-6 by addition of 250 mM proline
to 50-250 mM interacting buffer species (His(HCl) or Im(HCl))
for mAb dispersions formed by centrifugation filtration.

| | | | | | | | Sol- | | |
|---|---|---|---|---|---|---|---|---|---|
| mAb | | | Buffer | | | | vent | Inh | |
| conc | | | conc | Proline conc | | Viscosity | visc | visc | A₃₅₀ |
| (mg/ml) | pH | Buffer | (mM) | mg/ml | mM | (cP) | (cP) | (ml/g) | (cm⁻¹) |

pH 5

| | | | | | | | | | |
|---|---|---|---|---|---|---|---|---|---|
| 225 ± 2.2 | 5.12 | His | 50 | 29 | 250 | 26 ± 3.2 | 1.1 | 14.1 | 0.272 |
| 201 ± 1.0 | 4.96 | His | 250 | 29 | 250 | 16.8 ± 2.1 | 1.3 | 12.8 | 0.338 |
| 226 ± 2.6 | 5.4 | Im | 250 | 29 | 250 | 26.5 ± 0.7 | 1.25 | 13.5 | 0.303 |

pH 6

| | | | | | | | | | |
|---|---|---|---|---|---|---|---|---|---|
| 205 ± 0.4 | 5.94 | His | 50 | 29 | 250 | 45.2 ± 1.9 | 1.2 | 17.6 | 0.380 |
| 227 ± 0.8 | 5.92 | His | 250 | 29 | 250 | 30.8 ± 0.6 | 1.3 | 14.0 | 0.342 |
| 223 ± 0.3 | 6.11 | Im | 250 | 29 | 250 | 24 ± 0.4 | 1.2 | 13.4 | 0.393 |

### References (Example 6)

[1]. Zhao, H., P. H. Brown, et al. (2011), Biophysical Journal 100(9): 2309-2317; [2]. Samuel D, et al. 2000. Protein Sci 9(2):344-352

### EMBODIMENTS

### Embodiment 1

A low viscosity dispersion comprising a protein and a viscosity lowering agent, wherein the viscosity lowering agent is present at a concentration from about 10 mg/mL to about 300 mg/mL; and the protein is present at a concentration of greater than about 200 mg/mL and within a plurality of protein nanoclusters; wherein the viscosity lowering agent is proline, histidine, lysine, arginine, glutamic acid, betaine, glutamine, asparagine, or imidazole.

### Embodiment 2

The dispersion of Embodiment 1, wherein the viscosity is 25 cP or less.

### Embodiment 3

The dispersion of Embodiment 1, wherein the viscosity is 15 cP or less.

### Embodiment 4

The dispersion of Embodiment 1 to 3, wherein said viscosity is with a shear rate of 100 sec⁻¹ to 10,000 sec⁻¹.

### Embodiment 5

The dispersion of Embodiment 1 to 3, wherein said viscosity is with a shear rate of 500 sec⁻¹ to 5,000 sec⁻¹.

### Embodiment 6

The dispersion of Embodiment 1 to 3, wherein said viscosity is with a shear rate of 1000 sec⁻¹ to 5,000 sec⁻¹.

### Embodiment 7

The dispersion of Embodiment 1 to 3, wherein said viscosity is with a shear rate of 2000 sec⁻¹ to 4,000 sec⁻¹.

### Embodiment 8

The dispersion of Embodiment 1 to 7, further comprising a saccharide.

### Embodiment 9

The dispersion of Embodiment 8, wherein said saccharide is trehalose.

### Embodiment 10

The dispersion of Embodiment 8, wherein said saccharide is sucrose.

### Embodiment 11

The dispersion of Embodiment 8 or 10, wherein said saccharide is present at a concentration from about 10 mg/mL and about 200 mg/mL.

### Embodiment 12

The dispersion of Embodiment 1 to 11, comprising between about 200 mg/mL and about 400 mg/mL of the protein.

### Embodiment 13

The dispersion of Embodiment 1 to 12, wherein said dispersion is translucent or transparent.

### Embodiment 14

The dispersion of Embodiment 1 to 13, wherein the said nanoclusters are nanoparticles.

### Embodiment 15

The dispersion of Embodiment 1 to 14, wherein the tonicity of said dispersion is between about isotonic with human blood and about 3 times the tonicity of human blood.

### Embodiment 16

The dispersion of Embodiment 1 to 14, wherein the tonicity of said dispersion is between about isotonic with human blood and about 2 times the tonicity of human blood.

### Embodiment 17

The dispersion of Embodiment 1 to 14, wherein the tonicity of said dispersion is between about isotonic with human blood and about 1.5 times the tonicity of human blood.

### Embodiment 18

The dispersion of Embodiment 1 to 14, wherein said dispersion is approximately isotonic with human blood.

### Embodiment 19

The dispersion of Embodiment 1 to 18, having pH from about 4.0 to about 10.0.

### Embodiment 20

The dispersion of Embodiment 1 to 18, having pH from about 5.5 to about 10.0.

### Embodiment 21

The dispersion of Embodiment 1 to 18, having pH from about 5.5 to about 6.5.

### Embodiment 22

The dispersion of Embodiment 1 to 21, wherein said viscosity lowering agent is histidine.

### Embodiment 23

The dispersion of Embodiment 1 to 22, wherein said viscosity lowering agent is present at a concentration from about 20 mg/mL to about 150 mg/mL and said saccharide is present at a concentration from about 40 mg/mL to about 100 mg/mL.

### Embodiment 24

The dispersion of Embodiment 1 to 22, wherein said viscosity lowering agent is present at a concentration from about 20 mg/mL to about 150 mg/mL and said saccharide is present at a concentration from about 40 mg/mL to about 60 mg/mL.

### Embodiment 25

The dispersion of Embodiment 1 to 22, wherein said viscosity lowering agent is present at a concentration from about 30 mg/mL to about 60 mg/mL and said saccharide is present at a concentration from about 40 mg/mL to about 60 mg/mL.

### Embodiment 26

The dispersion of Embodiment 1 to 22, wherein said viscosity lowering agent is present at a concentration from about 30 mg/mL to about 60 mg/mL and said saccharide is present at a concentration from about 40 mg/mL to about 60 mg/mL.

### Embodiment 27

The dispersion of Embodiment 1 to 21, wherein said viscosity lowering agent is proline.

### Embodiment 28

The dispersion of Embodiment 27, wherein said proline is present at a concentration between about 10 mg/mL and about 300 mg/mL.

### Embodiment 29

The dispersion of Embodiment 27, wherein said proline is present at a concentration between about 100 mg/mL and about 300 mg/mL.

### Embodiment 30

The dispersion of Embodiment 1 to 21, wherein said dispersion comprises two different viscosity lowering agents selected from proline, histidine, lysine, arginine, glutamic acid, betaine, glutamine, asparagine, and imidazole.

### Embodiment 31

The dispersion of Embodiment 30, wherein said two different viscosity lowering agents are arginine and glutamic acid.

### Embodiment 32

The dispersion of Embodiment 31, wherein said arginine is present at a concentration from about 20 mg/mL to about 150 mg/mL and glutamic acid is present at a concentration from about 20 mg/mL to about 150 mg/mL.

### Embodiment 33

The dispersion of Embodiment 31, wherein said arginine is present at a concentration from about 20 mg/mL to about 125 mg/mL and glutamic acid is present at a concentration from about 20 mg/mL to about 125 mg/mL.

### Embodiment 34

The dispersion of Embodiment 31, wherein said arginine is present at a concentration from about 20 mg/mL to about 100 mg/mL and glutamic acid is present at a concentration from about 20 mg/mL to about 100 mg/mL.

### Embodiment 35

The dispersion of Embodiment claim 31, wherein said arginine is present at a concentration from about 50 mg/mL to about 150 mg/mL and glutamic acid is present at a concentration from about 50 mg/mL to about 150 mg/mL.

### Embodiment 36

The dispersion of Embodiment 31, wherein said arginine is present at a concentration from about 50 mg/mL to about 125 mg/mL and glutamic acid is present at a concentration from about 50 mg/mL to about 125 mg/mL.

### Embodiment 37

The dispersion of Embodiment 31, wherein said arginine is present at a concentration from about 75 mg/mL to about 125 mg/mL and glutamic acid is present at a concentration from about 75 mg/mL to about 125 mg/mL.

### Embodiment 38

The dispersion of Embodiment 31 to 37, wherein said arginine is present at a concentration from about 20 mg/mL to about 150 mg/mL and glutamic acid is present at a concentration from about 20 mg/mL to about 150 mg/mL; and wherein said dispersion further comprises a saccharide at a concentration from about 20 mg/mL to about 75 mg/mL.

### Embodiment 39

The dispersion of Embodiment 31 to 37, wherein said arginine is present at a concentration from about 20 mg/mL to about 150 mg/mL and glutamic acid is present at a concentration from about 20 mg/mL to about 150 mg/mL; and wherein said dispersion further comprises trehalose at a concentration from about 20 mg/mL to about 75 mg/mL.

### Embodiment 40

A low viscosity dispersion comprising a protein and a viscosity lowering agent selected from the group consisting of proline, histidine, lysine, arginine, glutamic acid, betaine, glutamine, asparagine, imidazole, and a combination of two or more thereof; wherein the viscosity lowering agent is present at a concentration from about 10 mg/mL to about 400 mg/mL; the protein is present at a concentration greater than 200 mg/mL and within a plurality of protein nanoclusters; and the viscosity is about 1 cP to about 125 cP.

### Embodiment 41

The low viscosity dispersion of Embodiment 40 comprising a protein and proline as a viscosity lowering agent.

### Embodiment 42

The low viscosity dispersion of Embodiment 40 comprising a protein and histidine as a viscosity lowering agent.

### Embodiment 43

The low viscosity dispersion of Embodiment 40 comprising a protein and lysine as a viscosity lowering agent.

### Embodiment 44

The low viscosity dispersion of Embodiment 40 comprising a protein and arginine as a viscosity lowering agent.

### Embodiment 45

The low viscosity dispersion of Embodiment 40 comprising a protein and glutamic acid as a viscosity lowering agent.

### Embodiment 46

The low viscosity dispersion of Embodiment 40 comprising a protein and betaine as a viscosity lowering agent.

### Embodiment 47

The low viscosity dispersion of Embodiment 40 comprising a protein and glutamine as a viscosity lowering agent.

### Embodiment 48

The low viscosity dispersion of Embodiment 40 comprising a protein and asparagine as a viscosity lowering agent.

### Embodiment 49

The low viscosity dispersion of Embodiment 40 comprising a protein and imidazole as a viscosity lowering agent.

### Embodiment 50

The low viscosity dispersion of Embodiments 40 to 49 that only comprises one amino acid.

### Embodiment 51

Methods for preparing the low viscosity dispersions described herein by tangential flow filtration (TFF) comprises the steps of (a) regulating the feed flow rate and retentate back-pressure to maintain a transmembrane pressure (TMP) below 0.8 bar; and (b) implementing the TFF process in a hollow fiber filter module to minimize the axial pressure drop to 0.4 bar at 150 mg/mL protein or higher, and to generate more uniform TMP and wall shear stress profiles across the filter module.

### Embodiment 52

Methods to minimize membrane fouling and maintain high permeation rates during tangential flow filtration (TFF) to produce the low viscosity dispersions described herein by: (a) selecting from the claimed viscosity lowering agents to maintain a low dispersion viscosity above 200 mg/mL and minimize concentration polarization; (b) maintaining a low TMP below 0.8 bar to minimize formation of protein deposits or gel layer on the filter membrane; and (c) implementing the TFF process in a hollow fiber module to minimize the wall shear stress to below 50 Pa, to reduce shear-induced protein aggregation and gelation.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A low viscosity dispersion comprising a protein and a viscosity lowering agent selected from the group consisting of proline, histidine, lysine, arginine, glutamic acid, betaine, glutamine, asparagine, imidazole, and a combination of two or more thereof; wherein
the viscosity lowering agent is present at a concentration from about 10 mg/mL to about 400 mg/mL;
the protein is present at a concentration greater than 200 mg/mL and within a plurality of protein nanoclusters; and
the viscosity is about 1 cP to about 125 cP.

2. The dispersion of claim 1, wherein the protein is present at a concentration greater than 200 mg/mL to about 500 mg/mL; or from about 225 mg/mL to about 300 mg/mL.

3. The dispersion of claim 1, wherein the protein is an antibody.

4. The dispersion of claim 1, wherein the viscosity is about 1 cP to about 100 cP; or from about 1 cP to about 50 cP; or from about 5 cP to about 25 cP.

5. The dispersion of claim 1, wherein the viscosity is with a shear rate of about 5 sec⁻¹ to about 200,000 sec⁻¹; or a shear rate of about 5 sec⁻¹ to about 150,000 sec⁻¹; or a shear rate of about 100 sec⁻¹ to about 10,000 sec⁻¹; or a shear rate of about 1,000 sec⁻¹ to about 5,000 sec⁻¹; or a shear rate of about 2,000 sec⁻¹ to about 4,000 sec⁻¹.

6. The dispersion of claim 1 having a pH from about 4.0 to about 10.0; or from about 5 to about 9; or from about 5 to about 7; or from about 5 to about 6; or from about 5.5 to about 6.5; or about 5.5.

7. The dispersion of claim 1 further comprising a saccharide.

8. The dispersion of claim 7, wherein the saccharide is trehalose, sucrose, or a combination thereof.

9. The dispersion of claim 7, wherein the saccharide is present at a concentration from about 10 mg/mL to about 100 mg/mL.

10. The dispersion of claim 1, wherein the viscosity lowering agent is histidine, proline, or arginine.

11. The dispersion of claim 1, wherein the viscosity lowering agent is a combination of arginine and glutamic acid.

12. The dispersion of claim 1, wherein the viscosity lowering agent contains two different compounds selected from the group consisting of proline, histidine, lysine, arginine, glutamic acid, betaine, glutamine, asparagine, imidazole; and the two different compounds are present in a ratio of 2:1 to 1:2; or a ratio of 1.5:1 to 1:1.5, or a ratio of about 1:1.

13. The dispersion of claim 1, wherein the hydrodynamic diameter of the protein nanoclusters is from about 1 nm to about 1,000 nm; or from about 10 nm to about 100 nm; or from about 20 nm to about 60 nm.

14. The dispersion of claim 1, wherein the viscosity lowering agent is present at a concentration from about 10 mg/mL to about 300 mg/mL; or from about 100 mg/mL to about 300 mg/mL.

15. The dispersion of claim 1, wherein the viscosity lowering agent is present in an amount from about 10 mg/mL to about 150 mg/mL or from about 75 mg/mL to about 150 mg/mL.

16. The dispersion of claim 11, wherein the arginine is present at a concentration from about 50 mg/mL to about 150 mg/mL and the glutamic acid is present at a concentration from about 50 mg/mL to about 150 mg/mL.

17. A pharmaceutical composition comprising the dispersion of claim 1 and a pharmaceutically acceptable excipient.

18. A pharmaceutical composition comprising a monoclonal antibody and a viscosity lowering agent selected from the group consisting of proline, histidine, lysine, arginine, glutamic acid, betaine, glutamine, asparagine, imidazole, and a combination of two thereof; wherein
the viscosity lowering agent is present at a concentration from 50 mg/mL to 300 mg/mL;
the monoclonal antibody is present at a concentration from 200 mg/mL to 300 mg/mL and within a plurality of monoclonal antibody nanoclusters having a hydrodynamic diameter of 20 nm to 60 nm;
the viscosity is 1 cP to 25 cP; and
the pH is 5 to 9.

19. A method for preparing the dispersion of claim 1 by tangential flow filtration (TFF) comprising the steps of:
(a) regulating the feed flow rate and retentate back-pressure to maintain a transmembrane pressure (TMP) below 0.8 bar; and
(b) implementing the TFF process in a hollow fiber filter module to minimize the axial pressure drop to 0.4 bar at 150 mg/mL protein or higher, and to generate more uniform TMP and wall shear stress profiles across the filter module.

20. A method to minimize membrane fouling and maintain high permeation rates during tangential flow filtration (TFF) to produce the dispersion of claim 1 by:
(a) selecting from the claimed viscosity lowering agents to maintain a low dispersion viscosity above 200 mg/mL and minimize concentration polarization;
(b) maintaining a low TMP below 0.8 bar to minimize formation of protein deposits or gel layer on the filter membrane; and
(c) implementing the TFF process in a hollow fiber module to minimize the wall shear stress to below 50 Pa, to reduce shear-induced protein aggregation and gelation.
